(19) United States
(12) Patent Application Publication
(10) Pub. No.: US 2003/0138771 A1
(43) Pub. Date: Jul. 24, 2003

(54) DNA sequences from S. pneumoniae bacteriophage DP1 that encode anti-microbal polypeptides

(76) Inventors: Jerry Pelletier, Baie-D'Urfe (CA); Philippe Gros, St. Lambert (CA); Michael DuBow, Montreal (CA)

Correspondence Address: Wesley B. Ames, FOLEY & LARDNER, P.O. Box 80278, San Diego, CA, 92138-0278, US

(21) Appl. No.: 10/097,111

(22) Filed: Jul. 17, 2002

Related U.S. Application Data
(63) Continuation-in-part of application No. 09/676,412, filed on Sep. 29, 2000.
(60) Provisional application No. 60/157,218, filed on Sep. 30, 1999.

(51) Int. Cl. C12Q 1/70; C07H 21/04; C12P 21/06; C12P 19/34

(52) U.S. Cl. 435/5; 435/91.1; 435/91.2; 435/69.1; 536/23.72

(57) ABSTRACT

The disclosure concerns particular bacteriophage open reading frames, and portions and products of those open reading frames which have antimicrobial activity. Methods of using such products are also described.

### CROSS-REFERENCE TO RELATED PATENT APPLICATIONS

This application is a continuation-in-part of U.S. application Ser. No. 09/676,412, filed Sep. 29, 2000, which claims the benefit of U.S. Provisional application No. 60/157,218, filed Sep. 30, 1999, all of which are hereby incorporated by reference in its entireties, including drawings.

### BACKGROUND OF THE INVENTION

The present invention relates to the development of antimicrobials based on Streptococcus pneumoniae (S. pneumoniae) bacteriophages. In addition, the present invention relates to DNA sequences from S. pneumoniae bacteriophage that encode antimicrobial polypeptides or act as antimicrobial per se. More specifically, the present invention is concerned with the identification of several antimicrobial agents and of targets of such agents, and in particular to the isolation of bacteriophage DNA sequences, and their translated protein products, showing antimicrobial activity. The DNA sequences can be expressed in expression vectors. These expression constructs and the proteins produced therefrom can be used for a variety of purposes including therapeutic methods and identification of microbial targets.

The following description is provided to assist the understanding of the reader. None of the information provided or references cited is admitted to be prior art to the present invention.

The frequency and spectrum of antibiotic-resistant infections have, in recent years, increased in both the hospital and community. Certain infections have become essentially untreatable and are growing to epidemic proportions in the developing world as well as in institutional settings in the developed world. The staggering spread of antibiotic resistance in pathogenic bacteria has been attributed to microbial genetic characteristics, widespread use of antibiotic drugs and changes in society that enhance the transmission of drug-resistant organisms (for a review, see Cohen, M. L. (1992). Science 257: 1050-1055). This spread of drug resistant microbes is leading to ever-increasing morbidity, mortality and health-care costs.

There are over 160 antibiotics currently available for treatment of microbial infections, all based on a few basic chemical structures and targeting a small number of metabolic pathways: bacterial cell wall synthesis, protein synthesis, and DNA replication. Despite all these antibiotics, a person could succumb to an infection as a result of a resistant bacterial infection. Resistance now reaches all classes of antibiotics currently in use, including: β-lactams, fluoroquinolones, aminoglycosides, macrolide peptides, chloramphenicol, tetracyclines, rifampicin, folate inhibitors, glycopeptides, and mupirocin. There is thus a need for new antibiotics, and this need will not subside given the ability bacteria have to overcome each new agent synthesized. It is also likely that targeting new pathways will play an important role in discovery of these new antibiotics. In fact, a number of crucial cellular pathways, such as secretion, cell division, and many metabolic functions, remain untargeted to date.

Most major pharmaceutical companies have on-going drug discovery programs for novel antimicrobials. These are based on screens for small molecule inhibitors (e.g., natural products, bacterial culture media, libraries of small molecules, combinatorial chemistry) of crucial metabolic pathways of the micro-organism of interest. The screening process is largely for cytotoxic compounds and in most cases is not based on a known mechanism of action of the compounds. Classical drug screening programs are being exhausted and many of these pharmaceutical companies are looking towards rational drug design programs. Several small to mid-size biotechnology companies, as well as large pharmaceutical companies, have developed systematic high-throughput sequencing programs to decipher the genetic code of specific micro-organisms of interest. The goal is to identify, through sequencing, unique biochemical pathways or intermediates that are unique to the microorganism. Knowledge of the function of these bacterial genes may form the rationale for a drug discovery program based on the mechanism of action of the identified enzymes/proteins. However, one of the most important steps in this approach is the ascertainment that the identified proteins and biochemical pathways are 1) non-redundant and essential for bacterial survival, and 2) constitute suitable and accessible targets for drug discovery. These two issues are not easily addressed since to date, 41 prokaryotic genomes have been sequenced. For a majority of the sequenced genomes, less than 50% of the open reading frames (ORFs) have been linked to a known function. Even with the genome of Escherichia coli (E. coli), the most extensively studied bacterium, less than two-thirds of the annotated protein coding genes showed significant similarity to genes with ascribed functions (Rusterholtz, K., and Pohlschroder, M. (1999). Cell 96, 469-470). Thus considerable work must be undertaken to identify appropriate bacterial targets for drug screening.

There thus remains a need to the identification of antimicrobial agents and of microbial targets of such agents.

The present description refers to a number of documents, the content of which is herein incorporated by reference in their entireties, including any drawings and tables.

### SUMMARY OF THE INVENTION

The present invention is based on the identification of specific DNA sequences of a bacteriophage that kill or inhibit growth of the host bacterium when introduced into a host cell. Thus, these DNA sequences are anti-microbial agents. Information based on these DNA sequences can be utilized to develop peptide mimetics that can also function as anti-microbials. The identification of the host bacterial proteins targeted by the anti-microbial bacteriophage DNA sequences also provides targets for drug design and compound screening for the development of antibacterial agents.

As used herein, the terms “bacteriophage” and “phage” are used interchangeably to refer to a virus which can infect a bacterial strain or a number of different bacterial strains.

In this regard, the terns “inhibit”, “inhibition”, “inhibitory”, and “inhibitor” all refer to a function of reducing a biological activity or function. Such reduction in activity or function can, for example, be in connection with a cellular component (e.g., an enzyme), or in connection with a cellular process (e.g., synthesis of a particular protein), or in connection with an overall process of a cell (e.g., cell growth). In reference to cell growth, the inhibitory effects may be bactericidal (killing of bacterial cells) or bacteriostatic (i.e., stopping or at least slowing bacterial cell growth). The latter term refers to slowing or preventing cell growth such that fewer cells of the strain are produced relative to uninhibited cells over a given time period. From a molecular standpoint, such inhibition may equate with a reduction in the level of, or elimination of, the transcription and/or translation of a specific bacterial target(s), or reduction or elimination of activity of a particular target biomolecule.

In a first aspect, the invention provides methods for identifying a target for antibacterial agents by identifying the bacterial target(s) of at least one inhibitory gene product, e.g., polypeptide having the sequence of dp1ORF17 or dp1ORF88 product, or a homologous product. Such identification allows the development of antibacterial agents active on such targets. Preferred embodiments for identifying such targets involve the identification and/or assessment of the binding between a target and a phage ORF product. The target molecule may be a bacterial protein or other bacterial biomolecule, e.g., a nucleoprotein, a nucleic acid, a lipid or lipid-containing molecule, a nucleoside or nucleoside derivative, a polysaccharide or polysaccharide-containing molecule, or a peptidoglycan. The phage ORF products may be subportions of a larger ORF product that also bind the host target, e.g., fragments of a bacteriophage-encoded polypeptide. Exemplary approaches are described below in the Description of Preferred Embodiment.

Additionally, the invention provides methods for identifying targets for antibacterial agents by identifying homologs of a S. pneumoniae target of a bacteriophage ORF product. Non-limiting examples of such bacteriophage ORF products include dp1ORF17 and dp1ORF88 products. Such homologs may be utilized in the various aspects and embodiments described herein.

The term “fragment” refers to a portion of a larger molecule or assembly. For proteins, the term “fragment” refers to a molecule which includes at least 5 contiguous amino acids from the reference polypeptide or protein, preferably at least 6, 8, 10, 12, 15, 20, 30, 50 or more contiguous amino acids. In connection with oligo- or polynucleotides, the term “fragment” refers to a molecule which includes at least 15 contiguous nucleotides from a reference polynucleotide, preferably at least 18, 21, 24, 30, 36, 45, 60, 90, 150, or more contiguous nucleotides. Also in preferred embodiments, the fragment has a length in a range with the minimum as described above and a maximum which is no more than 90% of the length (or contains that percent of the contiguous amino acids or nucleotides) of the larger molecule (e.g., of the specified ORF), in other embodiments, the upper limit is no more than 60, 70, or 80% of the length of the larger molecule.

Stating that an agent or compound is “active on” a particular cellular target, such as the product of a particular gene, means that the target is an important part of a cellular pathway which includes that target and that the agent interacts on that pathway. Such interactions can be, for example, protein:protein interactions wherein the agent or compound down regulates the activity of the cellular target where the cellular target is vital for cell survival or growth, or nucleic acid:protein interactions wherein the agent or compound interacts as a protein with nucleic acid sequences causing a down regulation of the nucleic acid sequence encoded product, or a product downstream of the nucleic acid sequence. Furthermore, interactions between an agent or compound and a particular cellular target may be indirect, as the agent or compound may interact with a cellular target which in turn is responsible for initiating other physiological changes within the cell which ultimately result in cell inhibition. Thus, in some cases the agent may act on a component upstream or downstream of the stated target, including a regulator of that pathway or a component of that pathway. In general, an antibacterial agent is active on an essential cellular function, often on a product of an essential gene.

By “essential”, in connection with a gene or gene product, is meant that the host is significantly growth compromised in the absence or depletion of functional product, and preferably cannot survive without the functional product. An “essential gene” is thus one that encodes a product that is highly beneficial, or preferably necessary, for cellular growth in vitro in a medium appropriate for growth of an isogeneic strain having a wild-type allele corresponding to the particular gene in question. Therefore, if an essential gene is inactivated or inhibited, that cell will grow significantly more slowly or even not at all. Preferably growth of a strain in which such a gene has been inactivated will be less than 20%, more preferably less than 10%, most preferably less than 5% of the growth rate of the wild-type, or not at all, in the growth medium. Preferably, in the absence of activity provided by a product of the gene, the cell will not grow at all or will be non-viable, at least under culture conditions similar to normal in vivo growth conditions. For example, absence of the biological activity of certain enzymes involved in bacterial cell wall synthesis can result in the lysis of cells under normal osmotic conditions, even though protoplasts can be maintained under controlled osmotic conditions. Preferably, but not necessarily, if such a gene is inhibited, e.g., with an antibacterial agent or a phage product, the growth rate of the inhibited bacteria will be less than 50%, more preferably less than 30%, still more preferably less than 20%, and most preferably less than 10% of the growth rate of the uninhibited bacteria. As recognized by those skilled in the art, the degree of growth inhibition will generally depend on the concentration of the inhibitory agent. In the context of the invention, essential genes are generally the preferred targets of antimicrobial agents. Essential genes can encode target molecules directly or can encode a product involved in the production, modification, or maintenance of a target molecule. A “strictly essential” gene is one that is necessary for cellular growth in vitro under growth conditions in a medium appropriate for growth of an isogeneic strain having a wild-type allele corresponding to the particular gene in question.

A “target” refers to a biomolecule that can be acted on by an exogenous agent, thereby modulating, preferably inhibiting, growth or viability of a cell. In most cases such a target will be a nucleic acid sequence or molecule, or a polypeptide or protein. However, other types of biomolecules can also be targets, such as for example, membrane lipids and cell wall structural components. One of skill in the art would recognize that determining the amino acid sequence of a particular polypeptide target also provides information regarding the nucleic acid sequence which encodes the target polypeptide. The determination of the nucleic acid sequence from a given amino acid sequence, or determining the amino acid sequence from a given nucleic acid sequence requires routine skill to those in the art.

The term “bacterium” refers to a single bacterial strain, and includes a single cell, and a plurality or population of cells of that strain unless clearly indicated to the contrary.

In reference to bacteria or bacteriophage, the term “strain” refers to bacteria or phage having a particular genetic content. The genetic content includes genomic content as well as recombinant vectors. Thus, for example, two otherwise identical bacterial cells would represent different strains if each contained a vector, e.g., a plasmid, with different phage ORF inserts.

In the context of the phage nucleic acid sequences, e.g., gene or coding sequences, of this invention, the terms “homolog” and “homologous” denote nucleotide sequences from different bacteria or phage strains or species or from other types of organisms that have significantly related nucleotide sequences, and consequently significantly related encoded gene products, preferably having related function. Homologous gene sequences or coding sequences have at least 70% sequence identity (as defined by the maximal base match in a computer-generated alignment of two or more nucleic acid sequences) over at least one sequence window of 48 nucleotides (or at least 99, 150, 200, or even the entire ORF or other sequence of interest), more preferably at least 80% or 85%, still more preferably at least 90%, and most preferably at least 95%. The polypeptide products of homologous genes have at least 35% amino acid sequence identity over at least one sequence window of 18 amino acid residues (or 24, 30, 33, 50, 100, or an entire polypeptide), more preferably at least 40%, still more preferably at least 50% or 60%, and most preferably at least 70%, 80%, or 90%. Alternatively, for polypeptides, a homolog has at least 50% similarity, more preferably at least 60, 70, 80, 90, or 95%. Preferably, the homologous gene product is also a functional homolog, meaning that the homolog will functionally complement one or more biological activities of the product being compared.

For nucleotide or amino acid sequence comparisons where a homology is defined by a % sequence identity (or percent similarity), the percentage may be determined using BLAST programs with default parameters (Altschul et al., 1997, “Gapped BLAST and PSI-BLAST: a new generation of protein database search programs, Nucleic Acid Res. 25:3389-3402)). Any of a variety of algorithms known in the art which provide comparable results can also be used with parameters set to provide equivalent results. Performance characteristics for three different algorithms in homology searching is described in Salamov et al., 1999, “Combining sensitive database searches with multiple intermediates to detect distant homologues.” Protein Eng. 12:95-100. Another exemplary program package is the GCG™ package from the University of Wisconsin.

In reference to amino acids and the homology amino acid sequences, the term “similarity” or the like is used herein to refer, as well-known to a person skilled in the art, to a measure of homology which includes identical amino acids and conservatively changed amino acids as matches in sequence comparisons. As known, the term “similar” refers in that context to a protein sequence, in which the substituting amino acid has chemico-physical properties which are similar to that of the substituted amino acid. The similar chemico-physical properties include similarities in charge, bulkiness, hydrophobicity, hydrophilicity and the like. The terms “identity” or “identical” refer to identical nucleic acid or amino acid residues between two compound sequences.

Homologs may also, or in addition, be characterized by the ability of two complementary nucleic acid strands to hybridize to each other under appropriately stringent conditions that allow hybridization at the levels of identity as stated above. Hybridizations are typically and preferably conducted with probe-length nucleic acid molecules, preferably 20-100 nucleotides in length. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook et al. (1989) Molecular Cloning: A Laboratory Manual, Cold Spring Harbor University Press, Cold Spring, N.Y.; Ausubel, F. M. et al. (1994) Current Protocols in Molecular Biology. John Wiley & Sons, Secaucus, N.J. Homologs and homologous gene sequences may thus be identified using any nucleic acid sequence of interest, including the phage ORFs and bacterial target genes of the present invention.

A typical hybridization, for example, utilizes, besides the labeled probe of interest, a salt solution such as 6× SSC (NaCl and Sodium Citrate base) to stabilize nucleic acid strand interaction, a mild detergent such as 0.5% SDS, together with other typical additives such as Denhardt's solution and salmon sperm DNA. The solution is added to the immobilized sequence to be probed and incubated at suitable temperatures to preferably permit specific binding while minimizing nonspecific binding. The temperature of the incubations and ensuing washes is critical to the success and clarity of the hybridization. Stringent conditions employ relatively higher temperatures, lower salt concentrations, and/or more detergent than do non-stringent conditions. Hybridization temperatures also depend on the length, complementarity level, and nature (i.e., “GC content”) of the sequences to be tested. Typical stringent hybridizations and washes are conducted at temperatures of at least 40° C., while lower stringency hybridizations and washes are typically conducted at 37° C. down to room temperature (˜25° C.). One of ordinary skill in the art is aware that these conditions may vary according to the parameters indicated above, and that certain additives such as formamide and dextran sulphate may also be added to affect the conditions.

By “stringent hybridization conditions” is meant hybridization conditions at least as stringent as the following: hybridization in 50% formamide, 5× SSC, 50 mM NaH₂PO₄, pH 6.8, 0.5% SDS, 0.1 mg/mL sonicated salmon sperm DNA, and 5× Denhart's solution at 42° C. overnight; washing with 2× SSC, 0.1% SDS at 45° C.; and washing with 0.2× SSC, 0.1% SDS at 45° C. In another example, stringent hybridization conditions should not allow for hybridization of two nucleic acids which differ over a stretch of 20 contiguous nucleotides by more than two bases.

Homologous nucleotide sequences will distinguishably hybridize with a reference sequence with up to three mismatches in ten (i.e., at least 70% base match in two sequences of equal length). Preferably, the allowable mismatch level is up to two mismatches in 10, or up to one mismatch in ten, more preferably up to one mismatch in twenty. (Those ratios can, of course, be applied to longer sequences.)

Preferred embodiments involve identification of binding between ORF product and bacterial cellular component that include methods for distinguishing bound molecules, for example, affinity chromatography, immunoprecipitation, crosslinking, and/or genetic screen methods that permit protein:protein interactions to be monitored. One of skill in the art is familiar with these techniques and common materials utilized (see, e.g., Coligan, J. et al. (eds.) (1995) Current Protocols in Protein Science, John Wiley & Sons, Secaucus, N.J. and; Golemis, E. (2002) A molecular approach: Protein-protein interactions, Cold Spring Harbor Laboratory Press, Cold Spring Harbor, N.Y.).

Other embodiments involve the identification and/or utilization of a target which is mutated at the site of phage protein interaction but still functional in the cell, by virtue of their host's relatively unresponsive nature in the presence of expression of ORFs previously identified as inhibitory to the non-mutant or wild-type strain. Such mutants have the effect of protecting the host from an inhibition that would otherwise occur by, for example, competing for binding with the phage ORF product and indirectly allow identification of the precise responsible target. The identified target can then be used for, for example, follow-up studies and anti-microbial development. In certain embodiments, rescue and/or protection from inhibition occurs under conditions in which a bacterial target or mutant target is highly expressed. This is performed, for example, through coupling of the sequence with regulatory element promoters, as known in the art, which regulate expression at levels higher than wild-type at, for example, a level sufficiently higher than the inhibitor can be competitively bound to the highly expressed target such that the bacterium is detectably less inhibited.

Identification of the bacterial target can involve identification of a phage ORF-specific site of action. This can involve a newly identified target, or a target where the phage site of action differs from the site of action of a previously known antibacterial agent or inhibitor. For example, phage T7 genes 0.7 and 2.0 target the host RNA polymerase, which is also the cellular target for the antibacterial agent, rifampin. To the extent that a phage product is found to act at a different site than previously described inhibitors, aspects of the present invention can utilize those new phage-specific sites for identification and use of new antibacterial agents. The site of action can be identified by techniques known to those skilled in the art, for example, by mutational analysis, binding competition analysis, and/or other appropriate techniques.

Once a bacterial host target or mutant target sequence has been identified, it too can be conveniently sequenced, sequence analyzed (e.g., by computer), and the underlying gene(s) and corresponding translated product(s) further characterized. Preferred embodiments include such analysis and identification. Preferably, such a target has not previously been identified as an appropriate target for antibacterial action.

Also in preferred embodiments in which the bacterial target is a polypeptide or nucleic acid molecule, the identification of a bacterial target of a phage ORF product or fragment includes identification of a cellular and/or biochemical function of the bacterial target. As understood by those skilled in the art, this can, for example, include identification of function by identification of homologous polypeptides or nucleic acid molecules having known function, or identification of the presence of known motifs or sequences corresponding to known function. Such identifications can be readily performed using sequence comparison computer software, such as the BLAST programs and similar other programs and sequence and motif databases. Those skilled in the art are familiar with determining function, with the particular methods selected as appropriate for the type of molecule of interest.

Other embodiments involve expression of a phage ORF in a bacterial strain, in preferred embodiments the expression thereof is inducible. By “inducible” is meant that expression is absent or occurs at a low level until the occurrence of an appropriate environmental stimulus provides otherwise. For the present invention such induction is preferably controlled by an artificial environmental change, such as by contacting a bacterial strain population with an inducing compound (i.e., an inducer). However, induction could also occur, for example, in response to build-up of a compound produced by the bacteria in the bacterial culture, e.g., in the medium. As uncontrolled or constitutive expression of inhibitory ORFs can severely compromise bacteria to the point of eradication, such expression is therefore undesirable in many cases because it would prevent effective evaluation of the strain and inhibitor being studied. For example, such uncontrolled expression could prevent any growth of the strain following insertion of a recombinant ORF, thus preventing a determination of transfection or transformation. A controlled or inducible expression is therefore advantageous and is generally provided through the provision of suitable regulatory elements, e.g., promoter/operator sequences that can be conveniently transcriptionally linked to a coding sequence to be evaluated. In most cases, the vector will also contain sequences suitable for efficient replication of the vector in the same or different host cells and/or sequences allowing selection of cells containing the vector, i.e., “selectable markers.” Further, preferred vectors include convenient primer sequences flanking the cloning region from which PCR and/or sequencing may be performed. In preferred embodiments where the purification of phage product is desired, preferably the bacterium or other cell type does not produce a target for the inhibitory product, or is otherwise resistant to the inhibitory product.

In preferred embodiments, the target of the phage ORF product or fragment is identified from a bacterial animal pathogen, preferably a mammalian pathogen, more preferably a human pathogen, and is preferably a gene or gene product of such a pathogen. Also in preferred embodiments, the target is a gene or gene product, where the sequence of the target is homologous to a gene or gene product from such a pathogen as identified above.

Other aspects of the invention provide isolated, purified, or enriched specific phage nucleic acid and amino acid sequences, subsequences, and homologs thereof from or corresponding to bacteriophage Dp1ORF17 and dp1ORF88. Such nucleotide sequences are at least 15 nucleotides in length,, preferably at least 18, 21, 24, or 27 nucleotides in length, more preferably at least 30, 50, or 90 nucleotides in length. In certain embodiments, longer nucleic acids are preferred, for example those of at least 120, 150, 200, 300, 600, 800 or more nucleotides. Such sequences can, for example, be amplification oligonucleotides (e.g., PCR primers), oligonucleotide probes, sequences encoding a portion or all of a phage-encoded protein, or a fragment or all of a phage-encoded protein. In preferred embodiments, the nucleic acid sequence or amino acid sequence contains a sequence which has a lower length as specified above, and an upper-length limit which is no more than 50, 60, 70, 80, or 90% of the length of the full-length ORF or ORF product. The upper-length limit can also be expressed in terms of the number of base pairs of the ORF (coding region).

As it is recognized that alternate codons will encode the same amino acid for most amino acids due to the degeneracy of the genetic code, the sequences of the present invention include nucleic acid sequences utilizing such alternate codon usage for one or more codons of a coding sequence. For example, all four nucleic acid sequences GCT, GCC, GCA, and GCG encode the amino acid, alanine. Therefore, if for an amino acid there exists an average of three codons, a polypeptide of 100 amino acids in length will, on average, be encoded by 3¹⁰⁰, or 5×10⁴⁷, nucleic acid sequences. Thus, a first nucleic acid sequence can be modified (e.g., a nucleic acid sequence from a phage as specified above) to create a second nucleic acid sequence encoding the same polypeptide as encoded by the first nucleic acid sequence using routine procedures and without undue experimentation. Consequently, the present invention also relates to all possible nucleic acid sequences encoding the bacteriophage dp1ORF17 or dp1ORF88 as if all were written out in full. Thus, these nucleotide sequences should not be limited SEQ ID NOs:1 and 2, to take into account the codon usage. Preferred sequences are those encoding codons which are preferred in the host bacterium.

The alternate codon descriptions are available in common textbooks, for example, Stryer, BIOCHEMISTRY 3^{rd} ed., and Lehninger, BIOCHEMISTRY 3^{rd} ed. Codon preference tables for various types of organisms are available in the literature. Because of the number of sequence variations involving alternate codon usage, for the sake of brevity, individual sequences are not separately listed herein. Instead the alternate sequences are described by reference to the natural sequence with replacement of one or more (up to all) of the degenerate codons with alternate codons from the alternate codon table (Table 1), preferably with selection according to preferred codon usage for the normal host organism or a host organism in which a sequence is intended to be expressed. Those skilled in the art also understand how to alter the alternate codons to be used for expression in organisms where certain codons code differently than shown in the “universal” codon table.

For amino acid sequences, sequences contain at least 5 peptide-linked amino acid residues, and preferably at least 6, 7, 10, 15, 20, 30, or 40, amino acids having identical amino acid sequence as the same number of contiguous amino acid residues in a bacteriophage dp1ORF17 or dp1ORF88. In some cases longer sequences maybe preferred, for example, those of at least 50, 70, 100, 200 or 270 amino acids in length. In preferred embodiments, the sequence has bacteria-inhibiting function when expressed or otherwise present in a bacterial cell which is a host for the bacteriophage from which the sequence was derived.

In particular embodiments, the isolated, purified or enriched polypeptide of the present invention comprises or consists of an amino acid sequence having at least 40%, at least 50%, at least 60%, more preferably at least 80%, and more preferably at least 90% or at least 99% similarity to an amino acid sequence encoded by dp1ORF17 or dp1ORF88.

By “isolated” in reference to a nucleic acid is meant that a naturally occurring sequence has been removed from its normal cellular (e.g., chromosomal) environment or is synthesized in a non-natural environment (e.g., artificially synthesized). Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

The term “enriched” means that the specific DNA or RNA sequence constitutes a significantly higher fraction (2-5 fold) of the total DNA or RNA present in the cells or solution of interest than in normal or diseased cells or in cells from which the sequence was originally taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased.

The term “significant” is used to indicate that the level of increase is useful to the person making such an increase and an increase relative to other nucleic acids of about at least 2-fold, more preferably at least 5- to 10-fold or even more. The term also does not imply that there is no DNA or RNA from other sources. The other source of DNA may, for example, comprise DNA from a yeast or bacterial genome, or a cloning vector such as pUC19. This term distinguishes from naturally occurring events, such as viral infection, or tumor type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does not require absolute purity (such as a homogeneous preparation). Instead, it represents an indication that the sequence is relatively more pure than in the natural environment (compared to the natural level, this level should be at least 2-5 fold greater, e.g., in terms of mg/mL). Individual clones isolated from a genomic or cDNA library may be purified to electrophoretic homogeneity. The claimed DNA molecules obtained from these clones could be obtained directly from total DNA or from total RNA. cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. The process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated. A genomic library can be used in the same way and yields the same approximate levels of purification.

The terms “isolated”, “enriched”, and “purified” with respect to the nucleic acids, above, may similarly be used to denote the relative purity and abundance of polypeptides (multimers of amino acids joined one to another by α-carboxyl:α-amino group (peptide) bonds). These, too, may be stored in, grown in, screened in, and selected from libraries using biochemical techniques familiar in the art. Such polypeptides may be natural, synthetic or chimeric and may be extracted using any of a variety of methods, such as antibody immunoprecipitation, other “tagging” techniques, conventional chromatography and/or electrophoretic methods. Some of the above utilize the corresponding nucleic acid sequence.

As indicated above, aspects and embodiments of the invention are not limited to entire genes and proteins. The invention also provides and utilizes fragments and portions thereof, preferably those which are “active” in the inhibitory sense described above. Such peptides or oligopeptides and oligo or polynucleotides have preferred lengths as specified above for nucleic acid and amino acid sequences from phage; corresponding recombinant constructs can thus be designed to express such fragments and portions and preferably such active fragments and portions. Also included are homologous sequences and fragments thereof.

Thus, in another aspect of the present invention, there is provided an isolated, purified or enriched nucleic acid sequence, selected from the group consisting of: a) a nucleotide sequence encoding dp1ORF17 or dp1ORF88 product; b) a sequence at least 70% identical to a); c) a complement of a) or b); and d) a sequence which hybridizes to a), b) or c) under high stringency conditions.

In another aspect, the present invention provides an isolated, purified or enriched polypeptide comprising a sequence selected from the group consisting of: a) an amino acid sequence encoded by dp1ORF17 or dp1ORF88; b) an amino acid sequence having at least 40% identity to the sequence of a); and c) an active fragment of a) or b), wherein the active fragment retains its bacterial inhibitory function.

In accordance with yet another aspect, there is provided a method for identifying a target for antibacterial agents, involving determining the bacterial target of a product of a bacteriophage dp1ORF17 or dp1ORF88 and functional fragments thereof.

Additionally, in another aspect, the present invention provides a method for identifying a compound active on a bacterial target protein of a bacteriophage dp1ORF17 or dp1ORF88 product or a fragment thereof which retains its activity on the bacterial target protein, by: a) contacting the bacterial target protein with a test compound; and b) determining whether the compound binds to or reduces the level of activity of the target protein, where binding of the compound with the target protein or a reduction of the level of activity of the protein is indicative that the compound is active on the target.

Also, another aspect provides a method for inhibiting a bacterium as part of a therapy or as a prophylaxy. The method involves contacting the bacterium with a compound active on a target of a bacteriophage dp1ORF17 or dp1ORF88 product or an active fragment thereof, wherein the target or the target site is preferably uncharacterized.

The nucleotide and amino acid sequences identified herein are believed to be correct, however, certain sequences may contain a small percentage of errors, e.g., 1-5%. In the event that any of the sequences have errors, the corrected sequences can be readily provided by one skilled in the art using routine methods. For example, the nucleotide sequences can be confirmed or corrected by obtaining and culturing the relevant phage, and purifying phage genomic nucleic acids. A region or regions of interest can be amplified, e.g., by PCR from the appropriate genomic template, using primers based on the described sequence. The amplified regions can then be sequenced using any of the available methods (e.g., a dideoxy termination method, for example, using commercially available products). This can be done redundantly to provide the corrected sequence or to confirm that the described sequence is correct. Alternatively, a particular sequence or sequences can be identified and isolated as an insert or inserts in a phage genomic library and isolated, amplified, and sequenced by standard methods. Confirmation or correction of a nucleotide sequence for a phage gene provides an amino acid sequence of the encoded product by merely reading off the amino acid sequence according to the normal codon relationships and/or expressed in a standard expression system and the polypeptide product sequenced by standard techniques. The sequences described herein thus provide unique identification of the corresponding genes and other sequences, allowing those sequences to be used in the various aspects of the present invention. Confirmation of a phage ORF encoded amino acid sequence can also be done by constructing a recombinant vector from which the ORF can be expressed in an appropriate host (e.g., E. coli), purified, and sequenced by conventional protein sequencing methods.

In other aspects the invention provides recombinant vectors and cells harboring bacteriophage ORF encoding dp1ORF17 or dp1ORF88 or portions thereof, or bacterial target sequences described herein. As understood by those skilled in the art, vectors may assume different forms, including, for example, plasmids, cosmids, and virus-based vectors. See, e.g., Sambrook et al. (1989) Molecular Cloning: A Laboratory Manual, Cold Spring Harbor University Press, Cold Spring, N.Y.; See also, Ausubel, F. M. et al. (eds.) (1994) Current Protocols in Molecular Biology. John Wiley & Sons, Secaucus, N.J.

In preferred embodiments, the vectors will be expression vectors, preferably shuttle vectors (which enable replication and/or expression in more than one type of host [e.g. prokaryotic and/or eucaryotic]) that permit cloning, replication, and expression within bacteria. An “expression vector” is one having regulatory nucleotide sequences containing transcriptional and translational regulatory information that controls expression of the nucleotide sequence in a host cell. Preferably, the vector is constructed to allow amplification from vector sequences flanking an insert locus. In certain embodiments, the expression vectors may additionally or alternatively support expression, and/or replication in animal, plant and/or yeast cells due to the presence of suitable regulatory sequences, e.g., promoters, enhancers, 3′ stabilizing sequences, primer sequences, etc. In preferred embodiments, the promoters are inducible and specific for the system in which expression is desired, e.g., bacteria, animal, plant, or yeast. The vectors may optionally encode a “tag” sequence or sequences to facilitate protein purification or protein detection. Convenient restriction enzyme cloning sites and suitable selective marker(s) are also optionally included. Such selective markers can be, for example, antibiotic resistance markers or markers which supply an essential nutritive growth factor to an otherwise deficient mutant host, e.g., tryptophan, histidine, or leucine in the Yeast Two-Hybrid systems described below.

The term “recombinant sequence” refers to a DNA sequence that has been transferred to a non-natural genetic environment or location by intervention by humans using molecular biological methods. The term does not include results of natural recombination and the like.

The term “recombinant vector” refers to a single- or double-stranded circular nucleic acid molecule that contains at least one recombinant DNA sequence that can be transfected into cells and replicated within or independently of a cell genome. A circular double-stranded nucleic acid molecule can be cut and thereby linearized upon treatment with appropriate restriction enzymes. An assortment of nucleic acid vectors, restriction enzymes, and the knowledge of the nucleotide sequences cut by restriction enzymes are readily available to those skilled in the art. A nucleic acid molecule encoding a desired product can be inserted into a vector by cutting the vector with restriction enzymes and ligating the two pieces together. Preferably the vector is an expression vector, e.g., a shuttle expression vector as described above.

By “recombinant cell” is meant a cell containing a recombinant nucleic acid sequence according to the present invention. The sequence may be in the form of or part of a vector or may be integrated into the host cell genome. Preferably the cell is a bacterial cell.

In preferred embodiments, the inserted nucleic acid sequence, encoding at least a portion of a bacteriophage dp1ORF17 or dp1ORF88, has a length as specified for the isolated purified or enriched nucleic acid sequences described above.

In another aspect, the invention also provides methods for identifying and/or screening compounds “active on” at least one bacterial target of a bacteriophage inhibitor protein or RNA. Preferred embodiments involve contacting bacterial target proteins with a test compound, and determining whether the compound binds to or reduces the level of activity of the bacterial target, e.g., a bacterial biomolecule, preferably a bacterial protein. Preferably this is done in vivo under approximately physiological conditions. The compounds that can be used may be large or small, synthetic or natural, organic or inorganic, proteinaceous or non-proteinaceous. In preferred embodiments, the compound is a peptidomimetic, as described herein, a bacteriophage inhibitor protein or fragment or derivative thereof, and preferably an “active portion”, or a small molecule. In particular embodiments, the methods include the identification of bacterial targets as described above or otherwise described herein. Preferably, the fragment of a bacteriophage inhibitor protein includes less than 80% of an intact bacteriophage inhibitor protein. Preferably, the at least one target includes a plurality of different targets of bacteriophage inhibitor proteins, preferably a plurality of different targets. The plurality of targets can be in or from a plurality of different bacteria, but preferably is from a single bacterial species.

In embodiments involving binding assays, binding is preferably to a fragment or portion of a bacterial target protein, where the fragment includes less than 90%, 80%, 70%, 60%, 50%, 40%, or 30% of an intact bacterial target protein. Preferably, the at least one bacterial target includes a plurality of different targets of bacteriophage inhibitor proteins, preferably a plurality of different targets. The plurality of targets can be in or from a plurality of different bacteria, but preferably is from a single bacterial species. The plurality of targets can correspond to a plurality of different portions or binding sites of a bacterial target protein.

As used herein, the term “binding” in the context of the interaction of two polypeptides means that the two polypeptides physically interact via discrete regions or domains on the polypeptides, wherein the interaction is dependent upon the amino acid sequences of the interacting domains. Generally, the equilibrium binding concentration of a polypeptide that specifically binds another is in the range of about 1 uM or lower, preferably 100 nM or lower, 10 nM or lower, 1 nM or lower, 100 pM or lower, and even 10 pM or lower.

A “method of screening” refers to a method for evaluating a relevant activity or property of a large plurality of compounds, rather than just one or a few compounds. For example, a method of screening can be used to conveniently test at least 100, more preferably at least 1000, still more preferably at least 10,000, and most preferably at least 100,000 different compounds, or even more. In a particular embodiment, the method is amenable to automated, cost-effective high throughput screening on libraries of compounds for lead development.

In the context of this invention, the term “small molecule” refers to compounds having molecular mass of less than 3000 Daltons, preferably less than 2000 or 1500, still more preferably less than 1000, and most preferably less than 600 Daltons, or even less than 500, 400, or even 350 Daltons. Preferably but not necessarily, a small molecule is not an oligopeptide.

As used herein, the term “simultaneously” when used in connection with the assays of the present invention, refers to the fact that the specified components or actions at least overlap in time, and is thus not restricted to the fact that the initiation and termination points are identical. For certainty, a simultaneous contact of a bacterial target polypeptide with a candidate compound and a bacteriophage polypeptide, for example, is an overlap in contact periods, which can, but does not necessarily reflect the fact that the latter two are introduced into an assay mixture at the exact same time.

The term “compounds” includes, but is not limited to, small organic molecules, peptides, polypeptides and antibodies that bind to a polynucleotide and/or polypeptide of the invention, such as for example inhibitory ORF gene product or target thereof, and thereby inhibit, extinguish or enhance its activity or expression. Potential compounds may be small organic molecules, a peptide, a polypeptide such as a closely related protein or antibody that binds the same site(s) on a binding molecule, such as a bacteriophage gene product, thereby preventing bacteriophage gene product from binding to bacterial target polypeptides.

The term “compounds” is also meant to include small molecules that bind to and occupy the binding site of a polypeptide, thereby preventing binding to cellular binding molecules, such that normal biological activity is prevented. Examples of small molecules include but are not limited to small organic molecules, peptides or peptide-like molecules. Preferred potential compounds include compounds related to and variants of inhibitory ORF encoded by a bacteriophage and of bacterial target of inhibitory ORF and any homologues and/or peptido-mimetics and/or fragments thereof. Other examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypeptide of the present invention but do not elicit a response, so that the activity of the polypeptide is prevented. Other potential compounds include antisense molecules (see Okano, 1991 J. Neurochem. 56, 560; see also “Oligodeoxynucleotides as Antisense Inhibitors of Gene Expression”, CRC Press, Boca Raton, Fla. (1988), for a description of these molecules).

As used herein, the term “library” refers to a collection of 100 compounds, preferably of 1000, still more preferably 5000, still more preferably 10,000 or more, and most preferably of 50,000 or more compounds.

As used herein, the term “physical association” refers to an interaction between two moieties involving contact between the two moieties.

As used herein, the term “fusion protein(s)” refers to a protein encoded by a gene comprising amino acid coding sequences from two or more separate proteins fused in frame such that the protein comprises fused amino acid sequences from the separate proteins.

As used herein, the term “artificially synthesized” when used in reference to a peptide, polypeptide or polynucleotide means that the amino acid or nucleotide subunits were chemically joined in vitro without the use of cells or polymerizing enzymes. The chemistry of polynucleotide and peptide synthesis is well known in the art.

As used herein, the term “decrease in the binding” refers to a drop in the signal that is generated by the physical association between two polypeptides under one set of conditions relative to the signal under another set of reference conditions. The signal is decreased if it is at least 10% lower than the level under reference conditions, and preferably 20%, 40%, 50%, 75%, 90%, 95% or even as much as 100% lower (i.e., no detectable interaction).

In a related aspect or in preferred embodiments, the invention provides a method of screening for potential antibacterial agents by determining whether any of a plurality of compounds, preferably a plurality of small molecules, is active on at least one target of a bacteriophage inhibitor protein or RNA. Preferred embodiments include those described for the above aspect, including embodiments which involve determining whether one or more test compounds bind to or reduce the level of activity of a bacterial target, and embodiments which utilize a plurality of different targets as described above.

The identification of bacteria-inhibiting phage ORFs and their encoded products also provides a method for identifying an active portion of such an encoded product. This also provides a method for identifying a potential antibacterial agent by identifying such an active portion of a phage ORF or ORF product. In preferred embodiments, the identification of an active portion involves one or more of mutational analysis, deletion analysis, or analysis of fragments of such products or the like, as well-known in the art. The method can also include determination of a 3-dimensional structure of an active portion, such as by analysis of crystal diffraction patterns. In further embodiments, the method involves constructing or synthesizing a peptidomimetic compound, where the structure of the peptidomimetic compound corresponds preferably to the structure of the active portion.

In this context, “corresponds” means that the peptidomimetic compound structure has sufficient similarities to the structure of the active portion that the peptidomimetic will interact with the same molecule as the phage protein and preferably will elicit at least one cellular response in common which relates to the inhibition of the cell by the phage protein.

The methods for identifying or screening for compounds or agents active on a bacterial target of a phage-encoded inhibitor can also involve identification of a phage-specific site of action on the target.

An “active portion” as used herein denotes an epitope, a catalytic or regulatory domain, or a fragment of a bacteriophage inhibitor protein that is responsible for, or a significant factor in, bacterial target inhibition. The active portion preferably may be removed from its contiguous sequences and, in isolation, still effect inhibition.

By “mimetic” is meant a compound structurally and functionally related to a reference compound that can be natural, synthetic, or chimeric. In terms of the present invention, a “peptidomimetic,” for example, is a compound that mimics the activity-related aspects of the 3-dimensional structure of a peptide or polypeptide in a non-peptide compound, for example one that mimics the structure of a peptide or active portion of a phage- or bacterial ORF-encoded polypeptide.

The present invention also provides a method for inhibiting a bacterial cell by contacting the bacterial cell with a compound active on a bacterial target of dp1ORF17 or dp1ORF88, or portion thereof. Such a method can be used in cases where the target is characterized or uncharacterized. In preferred embodiments, the compound is selected from the group consisting of a protein, or a fragment or derivative thereof; a structural mimetic, e.g., a peptidomimetic, of such a protein or fragment; a small molecule. The contacting can be performed in vitro, or in vivo in an infected or at risk organism, e.g., an animal such as a mammal or bird, for example, a human, or other mammal described herein, or in a plant.

In the context of this invention, the term “bacteriophage inhibitor protein” refers to a protein encoded by a bacteriophage nucleic acid sequence which inhibits bacterial function in a host bacterium. It should be understood that the present invention also relates to “bacteriophage inhibitor sequences” which refer to bacteriophage nucleic acid sequences which inhibit bacterial function in a host bacterium. Thus, these terms refer to bacteria-inhibiting phage products.

In the context of this invention, the phrase “contacting the bacterial cell with a compound active on a bacterial target of a bacteriophage inhibitor protein” or equivalent phrases refer to contacting with an isolated, purified, or enriched compound or a composition including such a compound, but specifically does not rely on contacting the bacterial cell with an intact naturally occurring phage which encodes the compound. Preferably no intact phage are involved in the contacting.

Related aspects provide methods for prophylactic or therapeutic treatment of a bacterial infection by administering to an infected, challenged, or at risk organism a therapeutically or prophylactically effective amount of a compound active on a target of bacteriophage dp1ORF17 or dp1ORF88, e.g., as described for the previous aspect. Preferably the bacterium involved in the infection or risk of infection produces the identified target of the bacteriophage inhibitor protein or alternatively produces a homologous target compound. In preferred embodiments, the host organism is a plant or animal, preferably a mammal or bird, and more preferably, a human or other mammal described herein. Preferred embodiments include, without limitation, those as described for the preceding aspect.

Compounds useful for the methods of inhibiting, methods of treating, and pharmaceutical compositions can include novel compounds, but can also include compounds which had previously been identified for a purpose other than inhibition of bacteria or for the purpose of inhibiting new families, genus, species, or strains of bacteria. Such compounds can be utilized as described and can be included in pharmaceutical compositions.

By “treatment” or “treating” is meant administering a compound or pharmaceutical composition for prophylactic and/or therapeutic purposes. The term “prophylactic treatment” refers to treating a patient or animal that is not yet infected but is susceptible to or otherwise at risk of a bacterial infection. The term “therapeutic treatment” refers to administering treatment to a patient already suffering from infection.

The term “bacterial infection” refers to the invasion of the host organism, animal or plant, by pathogenic bacteria. This includes the excessive growth of bacteria which are normally present in or on the body of the organism, but more generally, a bacterial infection can be any situation in which the presence of a bacterial population(s) is damaging to a host organism. Thus, for example, an organism suffers from a bacterial infection when excessive numbers of a bacterial population are present in or on the organism's body, or when the effects of the presence of a bacterial population(s) is damaging to the cells, tissue, or organs of the organism.

The terms “administer”, “administering”, and “administration” refer to a method of giving a dosage of a compound or composition, e.g., an antibacterial pharmaceutical composition, to an organism. Where the organism is a mammal, the method is, e.g., topical, oral, intravenous, transdermal, intraperitoneal, intramuscular, or intrathecal. The preferred method of administration can vary depending on various factors, e.g., the components of the pharmaceutical composition, the site of the potential or actual bacterial infection, the bacterium involved, and the infection severity.

The term “mammal” has its usual biological meaning, referring to any organism of the Class Mammalia of higher vertebrates that nourish their young with milk secreted by mammary glands, e.g., mouse, rat, and, in particular, human, bovine, sheep, swine, dog, and cat.

In the context of treating a bacterial infection a “therapeutically effective amount” or “pharmaceutically effective amount” indicates an amount of an antibacterial agent, e.g., as disclosed for this invention, which has a therapeutic effect. This generally refers to the inhibition, to some extent, of the normal cellular functioning of bacterial cells that renders or contributes to bacterial infection.

The dose of antibacterial agent that is useful as a treatment is a “therapeutically effective amount.” Thus, as used herein, a therapeutically effective amount means an amount of an antibacterial agent that produces the desired therapeutic effect as judged by clinical trial results and/or animal models. This amount can be routinely determined by one skilled in the art and will vary depending on several factors, such as the particular bacterial strain involved and the particular antibacterial agent used.

As used in the context of treating a bacterial infection, contacting or administering the antimicrobial agent “in combination with existing antimicrobial agents” refers to a concurrent contacting or administration of the active compound with antibiotics to provide a bactericidal or growth inhibitory effect beyond the individual bactericidal or growth inhibitory effects of the active compound or the antibiotic. Existing antibiotic refers to the group consisting of penicillins, cephalosporins, imipenem, monobactams, aminoglycosides, tetracyclines, sulfonamides, trimethoprim/sulfonamide, fluoroquinolones, macrolides, vancomycin, polymyxins, chloramphenicol and lincosamides.

In connection with claims to methods of inhibiting bacteria and therapeutic or prophylactic treatments, “a compound active on a target of a bacteriophage inhibitor protein” or terms of equivalent meaning differ from administration of or contact with an intact phage naturally encoding the full-length inhibitor compound. While an intact phage may conceivably be incorporated in the present methods, the method of the present invention at least includes the use of an active compound as specified herein but different from a full length inhibitor protein naturally encoded by a bacteriophage and/or a delivery or contacting method different from administration of or contact with an intact phage naturally encoding the full-length protein. Similarly, pharmaceutical compositions described herein at least include an active compound or composition different from a phage naturally coding the full-length inhibitor protein, or such a full-length protein is provided in the composition in a form different from being encoded by an intact phage. Preferably the methods and compositions do not include an intact phage.

In accordance with the above aspects, the invention also provides antibacterial agents and compounds active on a bacterial target of bacteriophage dp1ORF17 or dp1ORF88, where the target was preferably uncharacterized as indicated above. As previously indicated, such active compounds include both novel compounds and known compounds, preferably such known compounds were not known previously to find utility in which had previously been identified for a purpose other than inhibition of bacteria. Such previously identified biologically active compounds can be used in embodiments of the above methods of inhibiting and treating. In preferred embodiments, the targets, bacteriophages, and active compounds are as described herein for methods of inhibiting and methods of treating. Preferably the agent or compound is formulated in a pharmaceutical composition which includes a pharmaceutically acceptable carrier, excipient, or diluent. In addition, the invention provides agents, compounds, and pharmaceutical compositions wherein an active compound is active on an uncharacterized phage-specific site on the target.

In preferred embodiments of this aspect, the bacterial target is as described for embodiments of aspects above.

Likewise, the invention provides a method of making an antibacterial agent. The method involves identifying a target of bacteriophage dp1ORF17 or dp1ORF88, screening a plurality of compounds to identify a compound active on the target, and synthesizing the compound in an amount sufficient to provide a therapeutic effect when administered to an organism infected by a bacterium naturally producing the target, or at risk of being infected therewith.

In preferred embodiments, the identification of the target and identification of active compounds include steps or methods and/or components as described above (or otherwise herein) for such identification. Likewise, the active compound can be as described above, including fragments and derivatives of phage inhibitor proteins, peptidomimetics, and small molecules. As recognized by those skilled in the art, peptides can be synthesized by expression systems and purified, or can be synthesized artificially by methods well known in the art.

In the context of nucleic acid or amino acid sequences of this invention, the term “corresponding” and “correspond” indicates that the sequence is at least 95% identical, preferably at least 97% identical, and more preferably at least 99% identical to a sequence from the specified phage genome or bacterial genome, a ribonucleotide equivalent, a degenerate equivalent (utilizing one or more degenerate codons), or a homologous sequence, where the homolog provides functionally equivalent biological function.

In preferred embodiments the bacterial target of a bacteriophage inhibitor ORF product, e.g., an inhibitory protein or polypeptide, is preferably encoded by a nucleic acid coding sequence from such a bacterial host enabling infection by bacteriophage dp1, namely S. pneumoniae. In embodiments where the bacteriophage ORF product inhibits the growth of bacteria other than the host bacterium for dp1, the target could also be encoded by a bacterial nucleic acid sequence from bacteria other than the bacterial host. Target sequences are described herein by reference to sequence source sites and scientific publications. Non-limiting examples thereof include (1) S. pneumoniae (GenBank gi: 15902044 and 15899949; Tettelin H. et al. 2001, Science, 293: 498-506) sequences deposited in GenBank and (2) S. pneumoniae sequences available from TIGR at the World Wide Web site having the remaining address tigr.org/tdb/mdb/mdb.html.

The amino acid sequence of a polypeptide target is readily provided by translating the corresponding coding region. For the sake of brevity, the sequences are not reproduced herein. Again, for the sake of brevity, the sequences are described in GenBank. In cases where an entry for a coding region is not complete, the complete sequence can be readily obtained by routine methods, such as by isolating a clone in a phage dp1 host genomic library and sequencing the clone insert to provide the relevant coding region. The boundaries of the coding region can be identified by conventional sequence analysis and/or by expression in a bacterium in which the endogenous copy of the coding region has been inactivated and using subcloning to identify the functional start and stop codons for the coding region.

In an additional aspect, the present invention provides a nucleic acid segment which encodes a protein and corresponds to a segment of the nucleic acid sequence of an ORF (open reading frame) from S. pneumoniae bacteriophage dp1. Preferably, the protein is a functional protein. One of ordinary skill in the art would recognize that bacteriophage possess genes which encode proteins which may be beneficial, detrimental or neutral to a bacterial cell. Such proteins act to replicate DNA, translate RNA, manipulate DNA or RNA, and enable the phage to integrate into the bacterial genome. Proteins from bacteriophage can function as, for example, a polymerase, kinase, phosphatase, helicase, nuclease, topoisomerase, endonuclease, reverse transcriptase, endoribonuclease, dehydrogenase, gyrase, integrase, carboxypeptidase, proteinase, amidase, transcriptional regulators and the like, and/or the protein may be a functional protein such as a chaperone, capsid protein, head and tail proteins, a DNA or RNA binding protein, or a membrane protein, all of which are provided as non-limiting examples. Proteins with functions such as these are useful as tools for the scientific community.

Thus, the present invention provides a group of novel proteins from bacteriophages which can be used as tools for biotechnical applications such as, for example, DNA and/or RNA sequencing, polymerase chain reaction and/or reverse transcriptase PCR, cloning experiments, cleavage of DNA and/or RNA, reporter assays and the like. Preferably, the protein is encoded by an open reading frame in the nucleic acid sequences of bacteriophages dp1. Within the scope of the present invention are fragments of proteins and/or truncated portions of proteins which have been either engineered through automated protein synthesis, or prepared from nucleic acid segments which correspond to segments of the nucleic acid sequences of bacteriophages dp1, and which are then inserted into cells via vectors (e.g. plasmid) which can be induced to express the protein. It is understood by one of skill in the art that mutational analysis of proteins has been known to help provide proteins which are more stable and which have higher and/or more specific activities. Such mutations are also within the scope of the present invention, hence, the present invention provides a mutated protein and/or the mutated nucleic acid segment from bacteriophages dp1 which encodes the protein.

In another aspect, the invention provides antibodies which bind proteins encoded by a nucleic acid segment which corresponds to the nucleic acid sequence of an ORF (open reading frame) from bacteriophage dp1.

Bacteriophages are bacterial viruses which contain nucleic acid sequences which encode proteins that can correspond to proteins of other bacteriophages and other viruses. Antibodies targeted to proteins encoded by nucleic acid segments of phages dp1 can serve to bind proteins encoded by nucleic acid segments from other viruses which correspond to SEQ ID NO: 1 or 2. Furthermore, antibodies to proteins encoded by nucleic acid segments of phage dp1 can also bind to proteins from other viruses that share similar functions but may not share corresponding sequences. It is understood in the art that proteins with similar activities/functions from a variety of sources generally share conserved motifs, regions, domains or structures. Thus, antibodies to motifs, regions, domains or structures of functional proteins from phage dp1 should be useful in detecting corresponding proteins in other bacteriophages and viruses. Such antibodies can also be used to detect the presence of a virus sharing a similar protein. Preferably the virus to be detected is pathogenic to a mammal, such as a dog, cat, bovine, sheep, swine, or a human.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Additional features and embodiments of the present invention will be apparent from the following Description of Preferred Embodiment and from the claims, all within the scope of the present invention.

Additional aspects and embodiments will be apparent from the following Detailed Description and from the claims.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the characteristics of the S. pneumoniae pZ vector harboring a nisin-inducible promoter (P_{nisA}) and a multicloning site;

FIG. 2 shows a schematic representation of the functional assays used to characterize the bactericidal and bacteriostatic potential of predicted ORFs (>33 amino acids) encoded by bacteriophage dp1. a) Functional assay on semi-solid support media. b) Functional assay in liquid culture;

FIG. 3 corresponds to the graphs of colony forming units (CFU) over time showing the results of functional assay in liquid media to assess bacteriostatic or bactericidal activity of bacteriophage dp1ORF17 or 88. Growth inhibition assays were performed as detailed in the Description of Preferred Embodiment. The number of CFU was determined from cultures of S. pneumoniae transformants harboring a given bacteriophage inhibitory ORF, in the absence or presence of the inducer (nisin). The colony plating was done in the presence (panel A) and in the absence (panel B) of the antibiotics necessary to maintain the selective pressure for the plasmid encoding the ORFs (chloramphenicol and erythromicin). The identity of the subcloned ORF harbored by the S. pneumoniae is given at the top of the each graph. The number of CFU was also determined from non-induced and induced control cultures of S. pneumoniae transformants harboring a non-inhibitory phage ORF cloned into the same vector. Each graph represents the average obtained from three S. pneumoniae transformants;

FIG. 4 shows the pattern of protein expression of the inhibitory ORF in S. pneumoniae in the presence or in the absence of inducer. HA epitope tag was added to individual inhibitory ORF subcloned into the pZ vector. In the final construction, the HA tag is directly set inframe at the carboxy terminus of each ORF. An anti-HA tag antibody was used for the detection of the ORF expression. The identity of the subcloned ORF harbored by the S. pneumoniae transformants is given at the top of the panel. T1 and T2 represent protein expression at 1.5 and 3 hrs following induction; and

Other objects, advantages and features of the present invention will become more apparent upon reading of the following non-restrictive description of preferred embodiments with reference to the accompanying drawing which is exemplary and should not be interpreted as limiting the scope of the present invention.

### DETAILED DESCRIPTION OF THE PREFERRED EMBODIMENTS

Preliminarily the tables will be briefly described.

Table 1 is a table from Alberts et al., MOLECULAR BIOLOGY OF THE CELL 3^{rd} ed., showing the redundancy of the “universal” genetic code.

Table 2 shows the nucleotide (SEQ ID NO: 1 and 2) and amino acid (SEQ ID NO: 3 and 4) sequences of indicated inhibitory ORFs derived from S. pneumoniae phage dp1.

Table 3 shows the sequence similarity analyses that have been performed with bacteriophage dp1ORF17 and 88. These results indicate that dp1ORF17 and 88 have no significant homology to any genes in the NCBI non-redundant nucleotide database.

Table 4 shows the genomic sequence of bacteriophage Dp-1 (SEQ ID NO. 10).

Table 5 shows the nucleotide and amino acid sequences for all ORFs identified in bacteriophage Dp-1.

The present invention is based on the identification of naturally-occurring DNA sequence elements encoding RNA or proteins with anti-microbial activity. Bacteriophages or phages, are viruses that infect and kill bacteria. They are natural enemies of bacteria and, over the course of evolution have perfected enzymes and proteins (products of DNA sequences) which enable them to infect a host bacteria, replicate their genetic material, usurp host metabolism, and ultimately kill their host. The scientific literature documents well the fact that many known bacteria can be hosts for a large number of such bacteriophages that can infect and kill them (for example, see the ATCC bacteriophage collection at the Web site having the remaining address atcc.org) (Ackermann, H.-W. and DuBow, M. S. (1987). Viruses of Prokaryotes. CRC Press. Volumes 1 and 2). Although we know that many bacteriophages encode proteins which can significantly alter their host's metabolism, determination of the killing potential of a given bacteriophage gene product can be reliably assessed by expressing the gene product in the target bacterial strain.

As indicated above in one embodiment, the present invention is concerned with the use of bacteriophage dp1 coding sequences and the encoded polypeptides or RNA transcripts, to identify bacterial targets for potential new antibacterial agents. Thus, the invention concerns the selection of relevant bacteria. Particularly relevant bacteria are those which are pathogens of a complex organism such as an animal (e.g., mammals, reptiles, and birds) and plants. However, the invention can be applied to any bacterium (whether pathogenic or not) for which bacteriophage are available or which are found to have cellular components closely homologous to components targeted by bacteriophage dp1ORF17 or dp1ORF88.

Identification of bacteriophage dp1ORF17 or dp1ORF88 which inhibit the host bacterium provides (1) an inhibitor compound and (2) allows identification of the bacterial target affected by the phage-encoded inhibitor. Such a target is thus identified as a potential target for development of other antibacterial agents or inhibitors and the use of those targets to inhibit those bacteria. As indicated above, even if such a target is not initially identified in a particular bacterium, such a target can still be identified if a homologous target is identified in another bacterium. Usually, but not necessarily, such another bacterium would be a genetically closely related bacterium. Indeed, in some cases, an inhibitor encoded by bacteriophage dp1ORF17 or dp1ORF88 can also inhibit a homologous bacterial cellular component.

The demonstration that bacteriophages have adapted to inhibiting a host bacterium by acting on a particular cellular component or target provides a strong indication that that component is an appropriate target for developing and using antibacterial agents, e.g., in therapeutic treatments. Thus, the present invention also provides additional guidance over mere identification of bacterial essential genes, as the present invention also provides an indication of accessibility of the target to an inhibitor, and an indication that the target is sufficiently stable over time (e.g., not subject to high rates of mutation) as phage acting on that target were able to develop and persist. The present invention therefore identifies a particular subset of essential cellular components which are particularly likely to be appropriate targets for development of antibacterial agents.

The invention also, therefore, concerns the development or identification of inhibitors of bacteria, in addition to the phage-encoded inhibitory proteins (or RNA transcripts), which are active on the targets of bacteriophage-encoded inhibitors. As described herein, such inhibitors can be of a variety of different types, but are preferably small molecules.

In addition to the inhibitory ORFs from the bacteriophage, the entire genome of S. pneumoniae phage dp1 was determined, and the other ORFs identified. The full genomic sequence is provided in Table 4, and the ORFs and encoded polypeptides are provided in Table 5. Those other ORFs encode additional useful gene products, including structural components and a number of different enzymes. Examples of such enzymes include restriction endonucleases and DNA polymerases. Such phage-derived enzymes provide reagents useful in a variety of different molecular biology techniques. Thus, the invention also includes isolated, enriched, or purified nucleic acid and/or polypeptides or active portions thereof corresponding to a gene (or ORF) from S. pneumoniae phage dp1; the expression of such products from recombinant coding sequences; and the use of such products, e.g., enzymes, in molecular biology techniques (for example, creation of restriction digests, cloning, and other techniques). The ORF sequences can be isolated directly from the phage, or can be synthesized by conventional methods.

The following description provides preferred methods for implementing the various aspects of the invention. However, as those skilled in the art will readily recognize, other approaches can be used to obtain and process relevant information. Thus, the invention is not limited to the specifically described methods. In addition, the following description provides a set of steps in a particular order. That series of steps describes the overall development involved in the present invention. However, it is clear that individual steps or portions of steps may be usefully practiced separately, and, further, that certain steps may be performed in a different order or even bypassed if appropriate information is already available or is provided by other sources or methods.

Identification of Inhibitory ORF

The methodology previously described in PCT Application No. PCT/IB99/02040 filed Dec. 3, 1999, international publication WO032825, was used to identify and characterize DNA sequences from S. pneumoniae bacteriophage dp1 that can act as anti-microbials.

Briefly, the S. pneumoniae propagating strain was used as a host to propagate its phage. Individual ORFs were resynthesized from the phage genomic DNA by the polymerase chain reaction (PCR) using oligonucleotide primers flanking the ORF and subcloned into a shuttle vector containing regulatory sequences that allow inducible expression of the introduced ORF. Individual phage ORFs were then expressed in S. pneumoniae in an inducible fashion by adding to the culture medium non-toxic concentrations of inducer during the growth of individual bacterial clones expressing such individual phage ORFs. Toxicity of the phage inhibitory ORF towards the host was monitored by reduction or arrest of growth under induction conditions, as measured by optical density in liquid culture or after plating the induced cultures on solid medium.

The present invention provides nucleic acid segments isolated from S. pneumoniae bacteriophage dp1 encode proteins, whose genes are referred to respectively as ORF (open reading frame) 17 or 88 from phage dp1. Thus, the present invention provides a nucleic acid sequence isolated from S. pneumoniae (S. pneumoniae) bacteriophages dp1 comprising at least a portion of a gene encoding dp1ORF 17 or dp1ORF88 with anti-microbial activity. The nucleic acid sequence can be isolated using a method similar to those described herein, or using another method. In addition, such a nucleic acid sequence can be chemically synthesized. Having the anti-microbial nucleic acid sequence of the present invention, parts thereof or oligonucleotides derived therefrom, other anti-microbial sequences from other bacteriophage sources using methods described herein or other methods can be isolated, including screening methods based on nucleic acid sequence hybridization.

The present invention provides the use of bacteriophages dp1 anti-microbial DNA segments encoding dp1ORF17 or dp1ORF88, as a pharmacological agent, either wholly or in part, as well as the use of peptidomimetics, developed from amino acid or nucleotide sequence knowledge of such bacteriophage ORF products. This can be achieved where the structure of the peptidomimetic compound corresponds to the structure of the active portion of a bacteriophage ORF product of the present invention. In this analysis, the peptide backbone is transformed into a carbon-based structure that can retain cytostatic or cytocidal activity for the bacterium. This is done by standard medicinal chemistry methods, measuring growth inhibition of the various molecules in liquid cultures or on solid medium. These mimetics also represent lead compounds for the development of novel antibiotics.

In this context, “corresponds” means that the peptidomimetic compound structure has sufficient similarities to the structure of the active portion of bacteriophage dp1ORF17 or dp1ORF88 that the peptidomimetic will interact with the same molecule as the bacteriophage ORF product and preferably will elicit at least one cellular response in common with that triggered by the phage protein.

The invention also provides bacteriophage anti-microbial DNA segments from other phages based on nucleic acids and sequences hybridizing to the presently identified inhibitory ORF or a sequence perfectly complementary theretof under high stringency conditions or sequences which are homologous as described above. The bacteriophage anti-microbial DNA segment from bacteriophage ORF having SEQ ID NO: 1 or 2, or fragments or derivatives thereof can be used to identify a related segment from a related or unrelated phage based on conditions of hybridization or sequence comparison.

Identification of Bacterial Targets

The present invention provides the use of bacteriophage dp1ORF17 or dp1ORF88 with anti-microbial activity to identify essential host bacterium interacting proteins or other targets that could, in turn, be used for drug design and/or screening of test compounds. Thus, the invention provides a method of screening for antibacterial agents by determining whether test compounds interact with (e.g., bind to) the bacterial target. The invention also provides a method of making an antibacterial agent based on production and purification of the protein or RNA product of a bacteriophage ORF of the present invention and more particularly of dp1ORF17 or dp1ORF88. The method involves identifying a bacterial target of the bacteriophage dp1ORF17 or dp1ORF88 (or part or fragment thereof), screening a plurality of compounds to identify one which is active on the target, and synthesizing the compound in an amount sufficient to provide a therapeutic effect when administered to an organism infected by a bacterium naturally producing the target. The rationale is that the bacteriophage dp1ORF17 or dp1ORF88, or part thereof can physically interact and/or modify certain microbial host components to block their function.

A variety of methods are known to those skilled in the art for identifying interacting molecules and for identifying target cellular components (Review in: Golemis, E. (2002) Protein-protein interaction: A molecular approach, Cold spring Harbor Laboratory Press, Cold Spring Harbor, N.Y.). Several non-limiting approaches and techniques are described below and can be used to identify the host bacterial pathway and protein that interact or are inhibited by bacteriophage ORF products of the present invention.

The first approach is based on identifying protein:protein interactions between the bacteriophage dp1ORF17 or dp1ORF88 and S. pneumoniae host proteins, using a biochemical approach based on affinity chromatography. This approach has been used to identify interactions between lambda phage proteins and proteins from their E. coli host (Sopta, M., Carthew, R. W., and Greenblatt, J. (1995) J. Biol. Chem. 260: 10353-10369). The product of such bacteriophage ORF products is fused to a tag (e.g. -glutathione-S-transferase) following insertion in a commercially available plasmid vector which directs high-level expression thereof after induction of the responsive promoter to which the bacteriophage ORF is operably linked, thereby driving the expression of the fusion protein. The fusion protein is expressed in E. coli, purified, and immobilized on a solid phase matrix. Total cell extracts from S. pneumoniae, or other bacteria susceptible to inhibition by the ORF are then passed through the affinity matrix containing the immobilized phage ORF fusion protein; proteins retained on the column are then eluted under different conditions of ionic strength, pH, and detergents and separated by gel electrophoresis. They are recovered from the gel and the proteins are individually digested to completion with a protease (e.g.-trypsin) and either molecular mass or the amino acid sequence of the tryptic fragments can be determined by mass spectrometry using, for example, MALDI-TOF technology (Qin et al. (1997). Anal. Chem. 69: 3995-4001). The sequence of the individual peptides from a single protein is then analyzed by a bioinformatics approach to identify the S. pneumoniae protein interacting with the phage ORF. This is performed by a computer search of the S. pneumoniae genomes for the identified sequence.

Alternatively, tryptic peptide fragments of the bacterial genome can be predicted by computer software based on the nucleotide sequence of the genome, and the predicted molecular mass of peptide fragments generated in silico compared to the molecular mass of the peptides obtained from each interacting protein eluted from the affinity matrix.

Another approach is a genetic screen for protein:protein interaction, (e.g., some form of two hybrid screen or some form of suppressor screen). In one form of the two hybrid screen involving the yeast two hybrid system, the nucleic acid segment encoding a bacteriophage dp1ORF17 or dp1ORF88, or a portion thereof, is fused to the carboxyl terminus of the yeast Gal4 DNA binding domain to create a bait vector. A genomic DNA library of cloned S. pneumoniae sequences which have been engineered into a plasmid where the bacterial sequences are fused to the carboxyl terminus of the yeast of Gal4 activation domain II (amino acids 768-881), is also generated to create a prey vector. The two plasmids bearing such constructs are introduced sequentially, or in combination, into a yeast cell line, for example AH109 (Clontech Laboratories), previously engineered to contain chromosomally-integrated copies of E. coli lacZ and the selectable HIS3 and ADE2 genes (Durfee et al. (1993). Genes & Dev. 7: 555-569). The lacZ, HIS, and ADE2 reporter genes, each driven by a promoter containing Gal4 binding sites, are used for measuring protein-protein interactions. If the two expressed proteins interact within the yeast cell, the resulting protein:protein complex (prey and bait) will activate transcription from promoters containing Gal4 binding sites. Expression of HIS3, and ADE2 genes is manifested by relief of histidine and adenine auxotrophy. Such a system provides a physiological environment in which to detect potential protein interactions.

This system has been extensively used to identify novel protein-protein interaction partners and to map the sites required for interaction [for example, to identify interacting partners of translation factors (Qiu et al., 1998, Mol Cell Biol. 18:2697-2711), transcription factors (Katagiri et al., 1998, Genes, Chromosomes & Cancer 21:217-222) and proteins involved in signal transduction (Endo et al., 1997, Nature 387:921-924)]. Alternatively, a bacterial two-hybrid screen can be utilized to circumvent the need for the interacting proteins to be targeted to the nucleus, as is the case in the yeast system (Karimova et al., 1998, Proc. Natl. Acad. Sci. 95:5752-5756).

The protein targets of bacteriophage ORF products of the present invention can also be identified using bacterial genetic screens. One approach involves the overexpression of bacteriophage dp1ORF 17 or dp1ORF88 or a part thereof, in mutagenized S. pneumoniae followed by plating the cells and searching for colonies that can survive the anti-microbial activity of the bacteriophage ORF products. These colonies are then grown, their DNA extracted, and cloned into an expression vector that contains a replicon of a different incompatibility group from the plasmid expressing the bacteriophage ORF products. This library is then introduced into a wild-type bacterium in conjunction with an expression vector driving synthesis of the bacteriophage ORF products, followed by selection for surviving bacteria. Thus, bacterial DNA fragments from the survivors presumably contain a DNA fragment from the original mutagenized bacterial genome that can protect the cell from the antimicrobial activity bacteriophage dp1ORF17 or dp1ORF88 or part thereof. This fragment can be sequenced and compared with that of the bacterial host to determine in which gene the mutation lies. This approach enables one to determine the targets and pathways that are affected by the killing function of the bacteriophage ORF product.

Alternatively, the bacterial targets can be determined in the absence of selecting for mutations using the approach known as “multicopy suppression”. In this approach, the DNA from the wild type bacterial host is cloned into an expression vector that can coexist with the one containing the bacteriophage ORF product having the killing or inhibitory effect on the bacterial strain. Those plasmids that contain host DNA fragments and genes which protect the host from the anti microbial activity of the bacteriophage ORF products can then be isolated and sequenced to identify putative targets and pathways in the host bacteria.

In addition, an oligonucleotide cocktail can be synthesized based on the primary amino acid sequence determined for an interacting S. aureus or S. pneumoniae protein fragment. This oligonucleotide cocktail would comprise a mixture of oligonucleotides based on the nucleotide sequences of the primary amino acid of the predicted peptide, but in which all possible codons for a particular amino acid sequence are present in a subset of the oligonucleotide pool. This cocktail can then be used as a degenerate probe set to screen, by hybridization to genomic or cDNA libraries, to isolate the corresponding gene.

Alternatively, antibodies raised to peptides which correspond to an interacting S. pneumoniae protein fragment can be used to screen expression libraries (genomic or cDNA) to identify the gene encoding the interacting protein.

Screening Assays According to the Invention

It is desirable to devise screening methods to identify compounds which stimulate or which inhibit the function of the a bacterial target of a bacteriophage dp1ORF17 or 88 polypeptide or polynucleotide of the invention. Accordingly, the present invention provides for a method of screening compounds to identify those that modulate the function of a bacterial target of a bacteriophage dp1ORF17 or 88.

The invention is based in part on the discovery of the bacterial target of a bacteriophage dp1ORF17 or 88 inhibitory factors. Applicants have recognized the utility of the interaction in the development of antibacterial agents. Specifically, the inventors have recognized that 1) dp1 ORF 17 or 88 or derivatives or functional mimetics thereof are useful for inhibiting bacterial growth; 2) therefore, a bacterial target of a bacteriophage dp1ORF17 or 88 is a critical target for bacterial inhibition; and 3) the interaction between a S. pneumoniae bacterial target or fragment thereof and dp1ORF17 or 88 may be used as a basis for the screening and rational design of drugs or antibacterial agents. In addition to methods of directly inhibiting a bacterial target of a bacteriophage dp1ORF17 or 88 activity, methods of inhibiting a bacterial target expression are also attractive for antibacterial activity.

In preferred embodiments, the method involves the interaction of an inhibitory ORF product or fragment thereof with the corresponding bacterial target or fragment thereof that maintains the interaction with the ORF product or fragment. Interference with the interaction between the components can be monitored, and such interference is indicative of compounds that may inhibit, activate, or enhance the activity of the target molecule.

In more than one embodiment of the binding assay methods of the present invention, it may be desirable to immobilize either bacterial target of a bacteriophage dp1ORF17 or 88 or the corresponding inhibitory dp1 ORF to facilitate separation of complexed from uncomplexed forms of one or both of the proteins or polypeptides, as well as to accommodate automation of the assay. Binding of a test compound to a bacterial target (or fragment, or variant thereof) or interaction of a bacterial target to inhibitory dp1 ORF in the presence and absence of a candidate compound, can be accomplished in any vessel suitable for containing the reactants. Examples of such vessels include microtitre plates, test tubes and micro-centrifuge tubes.

In one embodiment a fusion protein can be provided which adds a domain that allows one or both of the proteins to be bound to a matrix. For example, glutathione-S-transferase (GST)/bacterial target fusion proteins or GST/ORF fusion proteins (e.g. GST/dp1 ORF 17 or 88) can be adsorbed onto glutathione sepharose beads (Sigma Chemical, St. Louis, Mo.) or glutathione derivatized microtitre plates, which are then combined with the test compound or the test compound and either the non-adsorbed bacterial target of a bacteriophage dp1ORF17 or 88 protein, and the mixture incubated under conditions conducive to complex formation (e.g. at physiological conditions for salt and pH). Following incubation the beads or microtitre plate wells are washed to remove any unbound components, the matrix immobilized in the case of beads, and complex determined either directly or indirectly. Alternatively, the complexes can be dissociated from the matrix, and the level of binding or activity of bacterial target of a bacteriophage dp1ORF17 or 88 determined using standard techniques.

Binding Assays

There are a number of methods of examining binding of a candidate compound to a protein target. Screening methods that measure the binding of a candidate compound to a bacterial target polypeptide or polynucleotide, or to cells or supports bearing the polypeptide or a fusion protein comprising the polypeptide, by means of a label directly or indirectly associated with the candidate compound, are useful in the invention.

The screening method may involve competition for binding of a labeled competitor such as dp1 ORF 17 or 88 or a fragment that is competent to bind a bacterial target or fragment thereof.

Non-limiting examples of screening assays in accordance with the present invention include the following [Also reviewed in Sittampalam et al. 1997 Curr. Opin. Chem. Biol. 3:384-91]:

i.) Time-Resolved Fluorescence Resonance Energy Transfer (TR-FRET)

One method of measuring inhibition of binding of two proteins is fluorescence resonance energy transfer [FRET; de Angelis, 1999, Physiological Genomics]. FRET is a quantum mechanical phenomenon that occurs between a fluorescence donor (D) and a fluorescence acceptor (A) in close proximity (usually <100 A of separation) if the emission spectrum of D overlaps with the excitation spectrum of A. Variants of the green fluorescent protein (GFP) from the jellyfish Aequorea Victoria are fused to a polypeptide or protein and serve as D-A pairs in a FRET scheme to measure protein-protein interaction. Cyan (CFP: D) and yellow (YFP: A) fluorescence proteins are linked with a bacterial target polypeptide, or a fragment thereof, and a dp1 ORF 17 or 88 polypeptide respectively. Under optimal proximity, interaction between the bacterial target polypeptide and the dp1 ORF polypeptide causes a decrease in intensity of CFP fluorescence concomitant with an increase in YFP fluorescence.

The addition of a candidate modulator to the mixture of appropriately labeled bacterial target and dp1 inhibitory ORF polypeptide, will result in an inhibition of energy transfer evidenced, for example, by a decease in YFP fluorescence at a given concentration of dp1 inhibitory ORF polypeptide relative to a sample without the candidate inhibitor.

ii.) Fluorescence Polarization

Fluorescence polarization measurement is another useful method to quantitate molecular interaction, including protein-protein binding. The fluorescence polarization value for a fluorescently-tagged molecule depends on the rotational correlation time or tumbling rate. Protein complexes, such as those formed by a S. pneumoniae target of a bacteriophage dp1 inhibitory ORF, or a fragment thereof, associating with a fluorescently labeled polypeptide (e.g., dp1 ORF 17 or 88 or a binding fragment thereof), have higher polarization values than does the fluorescently labeled polypeptide. Inclusion of a candidate inhibitor of the bacterial target-dp1 ORF interaction results in a decrease in fluorescence polarization relative to a mixture without the candidate inhibitor if the candidate inhibitor disrupts or inhibits the interaction of bacterial target with its polypeptide binding partner. It is preferred that this method be used to characterize small molecules that disrupt the formation of polypeptide or protein complexes.

iii.) Surface Plasmon Resonance

Another powerful assay to screen for inhibitors of a protein: protein interaction is surface plasmon resonance. Surface plasmon resonance is a quantitative method that measures binding between two (or more) molecules by the change in mass near a sensor surface caused by the binding of one protein or other biomolecule from the aqueous phase (analyte) to a second protein or biomolecule immobilized on the sensor (ligand). This change in mass is measured as resonance units versus time after injection or removal of the second protein or biomolecule (analyte) and is measured using a Biacore Biosensor (Biacore AB) or similar device. A bacterial target of bacteriophage dp1 inhibitory ORF, or a polypeptide comprising a fragment of it, could be immobilized as a ligand on a sensor chip (for example, research grade CM5 chip; Biacore AB) using a covalent linkage method (e.g. amine coupling in 10 mM sodium acetate [pH 4.5]). A blank surface is prepared by activating and inactivating a sensor chip without protein immobilization. Alternatively, a ligand surface can be prepared by noncovalent capture of ligand on the surface of the sensor chip by means of a peptide affinity tag, an antibody, or biotinylation. The binding of dp1 ORF 17 or 88 to bacterial target, or a fragment thereof, is measured by injecting purified dp1 ORF 17 or 88 over the ligand chip surface. Measurements are performed at any desired temperature between 4° C. and 37° C. Preincubation of the sensor chip with candidate inhibitors will predictably decrease the interaction between dp1 ORF 17 or 88 and its bacterial target. A decrease in dp1 ORF 17 or 88 binding, detected as a reduced response on sensorgrams and measured in resonance units, is indicative of competitive binding by the candidate compound.

v.) Bio Sensor Assay

ICS biosensors have been described by AMBRI (Australian Membrane Biotechnology Research Institute; http//www.ambri.com.au/). In this technology, the self-association of macromolecules such as a bacterial target, or fragment thereof, and bacteriophage dp1 ORF 17 or 88 or fragment thereof, is coupled to the closing of gramacidin-facilitated ion channels in suspended membrane bilayers and hence to a measurable change in the admittance (similar to impedence) of the biosensor. This approach is linear over six order of magnitude of admittance change and is ideally suited for large scale, high through-put screening of small molecule combinatorial libraries.

vi.) Phage Display

Phage display is a powerful assay to measure protein:protein interaction. In this scheme, proteins or peptides are expressed as fusions with coat proteins or tail proteins of filamentous bacteriophage. A comprehensive monograph on this subject is Phage Display of Peptides and Proteins. A Laboratory Manual edited by Kay et al. (1996) Academic Press. For phages in the Ff family that include M13 and fd, gene III protein and gene VIII protein are the most commonly-used partners for fusion with foreign protein or peptides. Phagemids are vectors containing origins of replication both for plasmids and for bacteriophage. Phagemids encoding fusions to the gene III or gene VIII can be rescued from their bacterial hosts with helper phage, resulting in the display of the foreign sequences on the coat or at the tip of the recombinant phage.

In one example of a simple assay, purified recombinant bacterial target protein, or fragment thereof, could be immobilized in the wells of a microtitre plate and incubated with phages displaying a dp1 ORF 17 or 88 sequence in fusion with the gene III protein. Washing steps are performed to remove unbound phages and bound phages are detected with monoclonal antibodies directed against phage coat protein (gene VIII protein). An enzyme-linked secondary antibody allows quantitative detection of bound fusion protein by fluorescence, chemiluminescence, or colourimetric conversion. Screening for inhibitors is performed by the incubation of the compound with the immobilized target before the addition of phages. The presence of an inhibitor will specifically reduce the signal in a dose-dependent manner relative to controls without inhibitor.

It is important to note that in assays of protein-protein interaction, it is possible that a modulator of the interaction need not necessarily interact directly with the domain(s) of the proteins that physically interact. It is also possible that a modulator will interact at a location removed from the site of protein-protein interaction and cause, for example, a conformational change in the bacterial target polypeptide. Modulators (inhibitors or agonists) that act in this manner can be termed allosteric effectors and are of interest since the change they induce may modify the activity of the bacterial target polypeptide.

Testing for inhibitors is performed by the incubation of the compound with the reaction mixtures. The presence of an inhibitor will specifically reduce the signal in a dose-dependent manner relative to controls without inhibitor. Compounds selected for their ability to inhibit interactions between bacterial target-dp1 ORF 17 or 88 are further tested in secondary screening assays.

In another aspect, the present invention relates to a screening kit for identifying agonists, antagonists, ligands, receptors, substrates, enzymes, etc. for a polypeptide and/or polynucleotide of the present invention; or compounds which decrease or enhance the production of such polypeptides and/or polynucleotides, which comprises: (a) a polypeptide and/or a polynucleotide of the present invention; (b) a recombinant cell expressing a polypeptide and/or polynucleotide of the present invention; (c) a cell membrane associated with a polypeptide and/or polynucleotide of the present invention; or (d) an antibody to a polypeptide and/or polynucleotide of the present invention.

It will be appreciated that in any such kit, (a), (b), (c) or (d) may comprise a substantial component.

It will be readily appreciated by the skilled artisan that a polypeptide and/or polynucleotide of the present invention may also be used in a method for the structure-based design of an agonist, antagonist or inhibitor of the polypeptide and/or polynucleotide, by: (a) determining in the first instance the three-dimensional structure of the polypeptide and/or polynucleotide, or complexes thereof; (b) deducing the three-dimensional structure for the likely reactive site(s), binding site(s) or motif(s) of an agonist, antagonist or inhibitor; (c) synthesizing candidate compounds that are predicted to bind to or react with the deduced binding site(s), reactive site(s), and/or motif(s); and (d) testing whether the candidate compounds are indeed agonists, antagonists or inhibitors. It will be further appreciated that this will normally be an iterative process, and this iterative process may be performed using automated and computer-controlled steps.

Each of the polynucleotide sequences provided herein may be used in the discovery and development of antibacterial compounds. The encoded protein, upon expression, can be used as a target for the screening of antibacterial drugs. Additionally, the polynucleotide sequences encoding the amino terminal regions of the encoded protein or Shine-Dalgarno or other sequence that facilitate translation of the respective mRNA can be used to construct antisense sequences to control the expression of the coding sequence of interest.

Vectors

The invention also provides vectors, preferably expression vectors, harboring the anti-microbial DNA nucleic acid segment of the invention in an expressible form, and cells transformed with the same. Such cells can serve a variety of purposes, such as in vitro models for the function of the anti-microbial nucleic acid segment and screening for downstream targets of the anti-microbial nucleic acid segment, as well as expression to provide relatively large quantities of the inhibitory product.

Thus, an expression vector harboring the anti-microbial nucleic acid segment or parts thereof (e.g. SEQ ID NO: 1 or 2) can also be used to obtain substantially pure protein. Well-known vectors, such as the pGEX series (available from Pharmacia), can be used to obtain large amounts of the protein which can then be purified by standard biochemical methods based on charge, molecular mass, solubility, or affinity selection of the protein by using gene fusion techniques (such as GST fusion, which permits the purification of the protein of interest on a glutathione column). Other types of purification methods or fusion proteins could also be used as recognized by those skilled in the art.

Likewise, vectors containing a sequence encoding a bacteriophage dp1ORF17 or dp1ORF88, or part thereof can be used in methods for identifying targets of the encoded antibacterial ORF product, e.g., as described above, and/or for testing inhibition of homologous bacterial targets or other potential targets in bacterial species other than S. pneumoniae.

Antibodies

Antibodies, both polyclonal and monoclonal, can be prepared against the protein encoded by a bacteriophage anti-microbial DNA segment of the invention (e.g bacteriophage dp1ORF17 or dp1ORF88) by methods well known in the art. Protein for preparation of such antibodies can be prepared by purification, usually from a recombinant cell expressing the specified ORF or fragment thereof. Those skilled in the art are familiar with methods for preparing polyclonal or monoclonal antibodies (See, e.g., Antibodies: A Laboratory Manual, Harlow and Lane, Cold Spring Harbor Laboratory, CSHL Press, N.Y., 1988).

Such antibodies can be used for a variety of purposes including affinity purification of the protein encoded by the bacteriophage anti-microbial DNA segment, tethering of the protein encoded by the bacteriophage anti-microbial DNA segment to a solid matrix for purposes of identifying interacting host bacterium proteins, and for monitoring of expression of the protein encoded by the bacteriophage anti-microbial DNA segment.

Recombinant Cells

Bacterial cells containing an inducible vector regulating expression of the bacteriophage anti-microbial DNA segment can be used to generate an animal model system for the study of infection by the host bacterium. The functional activity of the proteins encoded by the bacteriophage anti-microbial DNA segments, whether native or mutated, can be tested in animal in vitro or in vivo models.

While such cells containing inducible expression vectors is preferred, other recombinant cells containing a recombinant bacteriophage dp1ORF17 or dp1ORF88 or portion thereof are also provided by the present invention.

Also, a recombinant cell may contain a recombinant sequence encoding at least a portion of a protein which is a target of a phage inhibitory dp1ORF17 or dp1ORF88 or a portion thereof.

In the context of this invention, in connection with nucleic acid sequences, the term “recombinant” refers to nucleic acid sequences which have been placed in a genetic location by intervention using molecular biology techniques, and does not include the relocation of phage sequences during or as a result of phage infection of a bacterium or normal genetic exchange processes such as bacterial conjugation.

Derivatization of Identified Anti-Microbials

In cases where the identified anti-microbials above are peptidic compounds, the in vivo effectiveness of such compounds may be advantageously enhanced by chemical modification using the natural polypeptide as a starting point and incorporating changes that provide advantages for use, for example, increased stability to proteolytic degradation, reduced antigenicity, improved tissue penetration, and/or improved delivery characteristics.

In addition to active modifications and derivative creations, it can also be useful to provide inactive modifications or derivatives for use as negative controls or introduction of immunologic tolerance. For example, a biologically inactive derivative which has essentially the same epitopes as the corresponding natural antimicrobial can be used to induce immunological tolerance in a patient being treated. The induction of tolerance can then allow uninterrupted treatment with the active anti-microbial to continue for a significantly longer period of time.

Modified anti-microbial polypeptides and derivatives can be produced using a number of different types of modifications to the amino acid chain. Many such methods are known to those skilled in the art. The changes can include, for example, reduction of the size of the molecule, and/or the modification of the amino acid sequence of the molecule. In addition, a variety of different chemical modifications of the naturally occurring polypeptide can be used, either with or without modifications to the amino acid sequence or size of the molecule. Such chemical modifications can, for example, include the incorporation of modified or non-natural amino acids or non-amino acid moieties during synthesis of the peptide chain, or the post-synthesis modification of incorporated chain moieties.

The oligopeptides of this invention can be synthesized chemically or through an appropriate gene expression system. Synthetic peptides can include both naturally occurring amino acids and laboratory synthesized, modified amino acids.

Also provided herein are functional derivatives of anti-microbial proteins or polypeptides. By “functional derivative” is meant a “chemical derivative,” “fragment,” “variant,” “chimera,” or “hybrid” of the polypeptide or protein, which terms are defined below. A functional derivative retains at least a portion of the function of the protein, for example, reactivity with a specific antibody, enzymatic activity or binding activity.

A “chemical derivative” of the complex contains additional chemical moieties not normally a part of the protein or peptide. Such moieties may improve the molecule's solubility, absorption, biological half-life, and the like. The moieties may alternatively decrease the toxicity of the molecule, eliminate or attenuate any undesirable side effect of the molecule, and the like. Moieties capable of mediating such effects are disclosed in Genaro, 1995, Remington's Pharmaceutical Science. Procedures for coupling such moieties to a molecule are well known in the art. Covalent modifications of the protein or peptides are included within the scope of this invention. Such modifications may be introduced into the molecule by reacting targeted amino acid residues of the peptide with an organic derivatizing agent that is capable of reacting with selected side chains or terminal residues, as described below.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, chloroacetyl phosphate, N-alkylmaleimides, 3-nitro-2-pyridyl disulfide, methyl 2-pyridyl disulfide, p-chloro-mercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing primary amine-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine alpha-amino group.

Tyrosyl residues are well-known targets of modification for introduction of spectral labels by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction carbodiimide (R′—N—C—N—R′) such as 1-cyclohexyl-3-(2-morpholinyl(4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Derivatization with bifunctional agents is useful, for example, for cross-linking component peptides to each other or the complex to a water-insoluble support matrix or to other macromolecular carriers. Commonly used cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl) dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the alpha-amino groups of lysine, arginine, and histidine side chains (Creighton, T. E., Proteins: Structure and Molecular Properties, W. H. Freeman & Co., San Francisco, pp. 79-86 (1983)), acetylation of the N-terminal amine, and, in some instances, amidation of the C-terminal carboxyl groups.

Such derivatized moieties may improve the stability, solubility, absorption, biological half-life, and the like. The moieties may alternatively eliminate or attenuate any undesirable side effect of the protein complex. Moieties capable of mediating such effects are disclosed, for example, in Genaro, 1995, Remington's Pharmaceutical Science.

The term “fragment” is used to indicate a polypeptide derived from the amino acid sequence of the protein or polypeptide having a length less than the full-length polypeptide from which it has been derived. Such a fragment may, for example, be produced by proteolytic cleavage of the full-length protein. Preferably, the fragment is obtained recombinantly by appropriately modifying the DNA sequence encoding the proteins to delete one or more amino acids at one or more sites of the C-terminus, N-terminus, and/or within the native sequence.

Another functional derivative intended to be within the scope of the present invention is a “variant” polypeptide which either lacks one or more amino acids or contains additional or substituted amino acids relative to the native polypeptide. The variant may be derived from a naturally occurring polypeptide by appropriately modifying the protein DNA coding sequence to add, remove, and/or to modify codons for one or more amino acids at one or more sites of the C-terminus, N-terminus, and/or within the native sequence.

A functional derivative of a protein or polypeptide with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et al., 1983, DNA 2:183; Sambrook, J., Fritsch, E. F. and Maniatis, T (1989). Molecular cloning: A laboratory Manual. Cold Spring Harbor Laboratory, New York. Cold Spring Harbor Laboratory Presswherein nucleotides in the DNA coding sequence are modified such that a modified coding sequence is produced, and thereafter expressing this recombinant DNA in a prokaryotic or eukaryotic host cell, using techniques such as those described above. Alternatively, components of functional derivatives of complexes with amino acid deletions, insertions and/or substitutions may be conveniently prepared by direct chemical synthesis, using methods well-known in the art.

Of course, a person skilled in the art will understand how to adapt the terms “fragment” or “variant” similarly when referring to a nucleic acid sequence.

Insofar as other anti-microbial inhibitor compounds identified by the invention described herein may not be peptidal in nature, other chemical techniques exist to allow their suitable modification, as well, and according the desirable principles discussed above.

Administration and Pharmaceutical Compositions

For the therapeutic and prophylactic treatment of infection, the preferred method of preparation or administration of anti-microbial compounds will generally vary depending on the precise identity and nature of the anti-microbial being delivered. Thus, those skilled in the art will understand that administration methods known in the art will also be appropriate for the compounds of this invention. Pharmaceutical compositions are prepared, as understood by those skilled in the art, to be appropriate for therapeutic use. Thus, generally the components and composition are prepared to be sterile and free of components or contaminants which would pose an unacceptable risk to a patient. For compositions to be administered internally, it is generally important that the composition be pyrogen free, for example.

The particularly desired anti-microbial can be administered to a patient either by itself, or in pharmaceutical compositions where it is mixed with suitable carriers or excipient(s). In treating an infection, a therapeutically effective amount of an agent or agents is administered. A therapeutically effective dose refers to that amount of the compound that results in amelioration of one or more symptoms of bacterial infection and/or a prolongation of patient survival or patient comfort.

Toxicity, therapeutic and prophylactic efficacy of anti-microbials can be determined by standard pharmaceutical procedures in cell cultures and/or experimental organisms such as animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any compound identified and used in the method of the invention, the therapeutically effective dose can be estimated initially from cell culture assays. Such information can be used to more accurately determine useful doses in organisms such as plants and animals, preferably mammals, and most preferably humans. Levels in plasma may be measured, for example, by HPLC or other means appropriate for detection of the particular compound.

The exact formulation, route of administration and dosage can be chosen by the individual physician in view of the patient's condition (see e.g. Fingl et. al., in The Pharmacological Basis of Therapeutics, 1975, Ch. 1 p.1).

It should be noted that the attending physician would know how and when to terminate, interrupt, or adjust administration due to toxicity, organ dysfunction, or other systemic malady. Conversely, the attending physician would also know to adjust treatment to higher levels if the clinical response were not adequate (precluding toxicity). The magnitude of an administered dose in the management of the disorder of interest will vary with the severity of the condition to be treated and the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary or phyto medicine.

Depending on the specific infection target being treated and the method selected, such agents may be formulated and administered systemically or locally, i.e., topically. Techniques for formulation and administration may be found in Genaro, 1995, Remington's Pharmaceutical Science. Suitable routes may include, for example, oral, rectal, transdermal, vaginal, transmucosal, intestinal, parenteral, intramuscular, subcutaneous, or intramedullary injections, as well as intrathecal, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate identified anti-microbials of the present invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active anti-microbial compounds in water-soluble form. Alternatively, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The above methodologies may be employed either actively or prophylactically against an infection of interest.

To identify DNA segments of bacteriophage dp1 capable of acting as anti-microbial agents, a strategy described briefly above and in International Application No. PCT/IB99/02040, international publication WO032825, was employed. In essence, the procedure involved sequence characterization of the bacteriophage, identification of protein coding regions (open reading frames or ORFs), subcloning of all ORFs into an appropriate inducible expression vector, transfer of the ORF subclones into S. aureus, followed by induction of ORF expression and assessment of effect on bacterial growth. The following exemplary discovery steps were employed.

The present invention is illustrated in further detail by the following non-limiting examples.

### EXAMPLE 1

### Growth of Streptococcus pneumoniae Bacteriophage dp1

The S. pneumoniae propagating strain R6, obtained from Dr. Pedro Garcia, (Madrid, Spain), was used as a host to propagate phage dp1. Phage dp1was also obtained from Dr. Pedro Garcia.

The stock and 10-fold dilutions of the first plaque purification were titrated against exponentially growing R6 on K-CAT agar plates using the sandwich procedure described above. After two plaque purifications, the phage was amplified by infecting 1.5 ml of exponentially growing R6st with 200 ul of the second plaque-purified eluate. The mixture was incubated at 37° C. for 15 minutes and 7.5 ml of K-CAT soft agar was added. The entire mixture was overlaid on a 150 mm petri dish containing K-CAT agar. The soft agar was allowed to harden for 20 minutes and the plate was incubated at 37° C. overnight. The next morning, the phage lysate was eluted with 8 ml of K-CAT medium at room temperature for 3-4 hours on a rotary shaker. The eluate was collected and flitered through a 0.45 uM filter. The filtrate was stored at 4° C. as a homestock.

A dilution of dp1 phage homestock was used to infect exponentially growing S. pneumoniae propagating strain (R6) to give about 90% lysis on 150 mm K-CAT plates. Twenty (20) such plates were obtained and each plate was eluted with 8 ml of K-CAT medium at room tempeature for 3-4 hours on a rotary shaker (60 rpm, Roto Mix™, Thermolyne). The phage suspension was collected and centrifuged at 10,000 rpm (JA-20 rotor, Beckman) for 15 minutes at 4° C. to pellet bacteria.

The phage suspension was further purified by centrifugation on a preformed cesium chloride step gradient as described in Sambrook, J., Fritsch, E. F. and Maniatis, T (1989). Molecular cloning: A laboratory Manual. Cold Spring Harbor Laboratory, New York. Cold Spring Harbor Laboratory Press, using a TLS 55 rotor (Beckman) for 2 hrs at 28,000 rpm at 4° C. Banded phage was collected and ultracentrifuged again on an isopycnic cesium chloride gradient (1.5 g/ml) at 42,000 rpm for 24 hrs at 4° C. using a TLS 55 rotor (Beckman). The phage was harvested and dialyzed overnight at 4° C. against 2 L of dialysis buffer consisting of 10 mM NaCl, 50 mM Tris-HCl [pH 8.0] and 10 mM MgCl₂. Phage DNA was prepared from the phage suspension by adding 20 mM EDTA, 50 μg/ml Proteinase K and 0.5% SDS and incubating for 1 hr at 55° C., followed by successive extractions with 1 volume of phenol, 1 volume of phenol-chloroform and 1 volume of chloroform. The DNA was then dialyzed overnight at 4° C. against 4 L of TE (10 mM Tris-HCl [pH 8.0], 1 mM EDTA).

### EXAMPLE 2

### DNA Sequencing of the Bacteriophage Genomes

Twenty μg of phage DNA were diluted in 200 μl of TE [pH 8.0] in a 1.5 ml eppendorf tube and sonication was performed (550 Sonic Dismembrator, Fisher Scientific). Samples were sonicated under an amplitude of 3 μm with bursts of 10 s spaced by 15 s cooling in ice/water for 2 to 3 cycles and size fractionated on 0.7% agarose gels in TAE buffer (1× TAE is: 40 mM Tris-acetate, 1 mM EDTA [pH 8.0]). The sonicated DNA was then size fractionated by agarose gel electrophoresis. Fractions ranging from 1 to 2 kbp were excised from the agarose gel and purified using a commercial DNA extraction system according to the instructions of the manufacturer (Qiagen) and eluted in 110 μl of 1 mMTris-HCl [pH 8.5].

The ends of the sonicated DNA fragments were repaired with a combination of T4 DNA polymerase and the Klenow fragment of E. coli DNA polymerase 1 as follows: reactions were performed in a final volume of 200 μl containing sonicated phage DNA, 10 mM Tris-HCl [pH 8.0], 50 mM NaCl, 10 mM MgCl₂, 1 mM DTT, 50 μg/ml BSA, 100 μM of each dNTP and 30 units of T4 DNA polymerase (New England Biolabs) for 20 min at 12° C. followed by addition of 25 units of Klenow fragment (New England Biolabs) for 15 min at room temperature. The reaction was stopped and purified by Quiagen PCR purification column.

The cloning of the sonicated phage DNA into pKSII vector and transformation were done as follows: blunt-ended DNA fragments were cloned by ligation directly into the HincII site of the pKSII vector (Stratagene) dephosphorylated with calf intestinal alkaline phosphatase (New England Biolabs). A typical reaction contained 100 ng of vector, 300 ng of repaired sonicated phage DNA in a final volume of 20 μl containing 800 units of T4 DNA ligase (New England Biolabs) and incubated overnight at 16° C. Transformation and selection of positive clones was performed in the host strain DH10 β of E. coli using ampicillin as a selective antibiotic as described in Sambrook, J., Fritsch, E. F. and Maniatis, T (1989). Molecular cloning: A laboratory Manual. Cold Spring Harbor Laboratory, New York. Cold Spring Harbor Laboratory Press.

Recombinant clones were picked from agar plates into 96-well plates containing 180 μl LB and 100 μg/ml ampicillin and incubated at 37° C. The presence of phage DNA insert was confirmed by PCR amplification using T3 and T7 primers flanking the HincII cloning site of the pKSII vector. PCR amplification of the potential foreign inserts was performed in a 15 μl reaction volume containing 20 mM Tris-HCl [pH 8.4], 50 mM KCl, 1.5 mM MgCl₂, 0.02% gelatin, 1 μM primer, 187.5 μM each dNTP, and 0.75 units Taq polymerase (BRL). The thermocycling parameters were as follows: 2 min initial denaturation at 94° C., followed by 20 cycles of 30 sec denaturation at 94° C., 30 sec annealing at 58° C., and 2 min extension at 72° C., followed by a single extension step at 72° C. for 10 min. Clones with insert sizes of 1 to 2 kbp were selected and plasmid DNA was prepared from the selected clones using the QIAprep™ spin miniprep kit (Qiagen).

The nucleotide sequence of the extremities of each recombinant clone was determined using an ABI 377-36 automated sequencer with ABI prism BigDye™ terminator cycle sequencing ready reaction kit (Applied Biosystems; #4303152). To ensure co-linearity of the sequence data and the genome, all regions of the phage genome were sequenced at least once from both directions on two separate clones. In areas that this criterion was not initially met, a sequencing primer was selected and phage DNA was used directly as sequencing template employing ABI prism BigDye™ terminator cycle sequencing ready reaction kit.

### EXAMPLE 3

### Bioinformatic Management of Primary Nucleotide Sequence

Sequence contigs were assembled using Sequencher™ 3.1 software (GeneCodes). To close contig gaps, sequencing primers were selected near the edge of the contigs. Phage DNA was used directly as sequencing template employing ABI prism BigDye™ terminator cycle sequencing ready reaction kit (Applied Biosystems; #4303152).

A software program was used on the assembled sequence of bacteriophages to identify all putative ORFs larger than 33 codons. The software scans the primary nucleotide sequence starting at nucleotide #1 for an appropriate start codon. Three possible selections can be made for defining the nature of the start codon; I) selection of ATG; II) selection of ATG or GTG; and III) selection of either ATG, GTG, TTG, CTG, ATT, ATC, and ATA. This latter initiation codon set corresponds to the one reported by the NCBI (at the Web site with the remaining address being ncbi.nlm.nih.gov/htbin-post/Taxonomy/wprintgc?mode=c) for the bacterial genetic code. When an appropriate start codon is encountered, a counting mechanism is employed to count the number of codons (groups of three nucleotides) between this start codon and the next stop codon downstream of it. If a threshold value of 33 is reached, or exceeded, then the sequence encompassed by these two codons is defined as an ORF. This procedure is repeated, each time starting at the next nucleotide following the previous stop codon found, in order to identify all the other putative ORFs. The scan is performed on all three reading frames of both DNA strands of the phage sequence.

Sequence homology searches for each ORF were carried out using an implementation of blast programs. Downloaded public databases used for sequence analysis include:

i) non-redundant GenBank (nr) (Web site with remaining address as: ncbi.nlm.nih.gov)

ii) pdbaa database (Web site with remaining address as: ncbi.nlm.nih.gov)

iii) PRODOM (http site with address as:protein.toulouse.inra.fr/protein.html)

iv) Swissprot and TREMBL (Web site with remaining address as: expasy.ch)

v) Block plus and Block prints (http site with address as: blocks.fhcrc.org)

vi) Pfam (http site with address as: wustl.edu)

vii) Prosite (Web site with remaining address as: expasy.ch)

viii) Bacterial genomes (Web site with remaining address as: tigr.org).

### EXAMPLE 4

### Inducible Expression Vector

In an example presented below, regulatory sequences from the Lactococcus lactis nisin gene cluster are used to direct individual ORF expression in S. pneumoniae. The nisin operon of L. lactis encodes a series of proteins which normally mediate the autoregulated production of nisin, an antimicrobial peptide (Kuipers et al., 1995, J. Biol. Chem. 270:27299-27304). The operon encoding this regulated biosynthetic capacity is normally silent and only induced when nisin is present. By exchanging the structural gene for nisin (nisA) with a gene of interest (geneX), high level production of protein X can be achieved upon induction with nisin. In the lactococcal system, the nisA and nisF genes are induced by nisin via a two-component signal transduction pathway consisting of a histidine protein kinase, NisK, and a response regulator, NisR. Nisin acts as an inducer on the outside of the cell and is sensed by NisK which in turn activates NisR to stimulate transcription from the nisA promoter. Expression of both nisR and nisK is driven from the constitutive nisR promoter. Recently, it has been reported that a two-plasmid system, in which the nisA promoter drives the inducible expression of genes of interest and the regulatory genes nisR and nisK are expressed constitutively, allows efficient control of gene expression by nisin in a variety of lactic acid bacteria including S. pneumoniae and other Gram-positive bacteria including Enterococcus faecalis and Bacillus subtilis (Eichenbaum et al., 1998, Applied Env. Microb. 64:2763-2769). The dual plasmid system permits nisin-inducible expression in a variety of bacteria by supplying the two-component regulators NisRK in trans since these proteins are present only in the natural host L. lactis. Following induction of ORF expression by the addition of nisin at non-toxic concentrations, toxicity of the phage ORF of interest in the host is monitored by reduction or arrest of bacterial growth under induction conditions, as measured by optical density in liquid culture or after plating the induced cultures on solid medium.

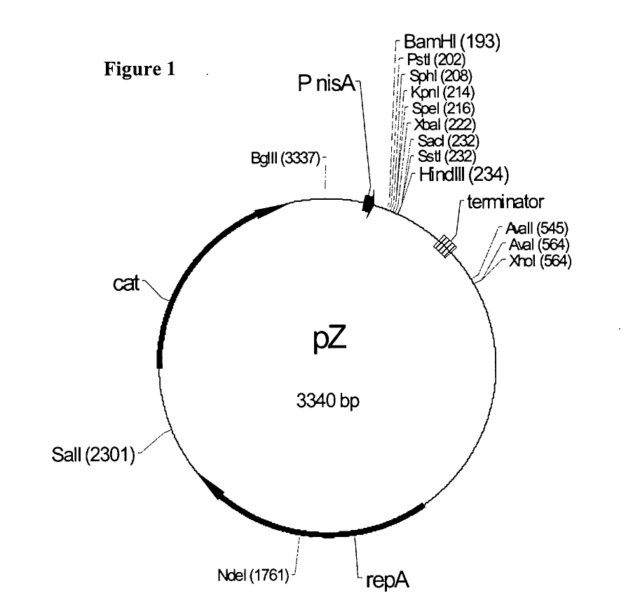

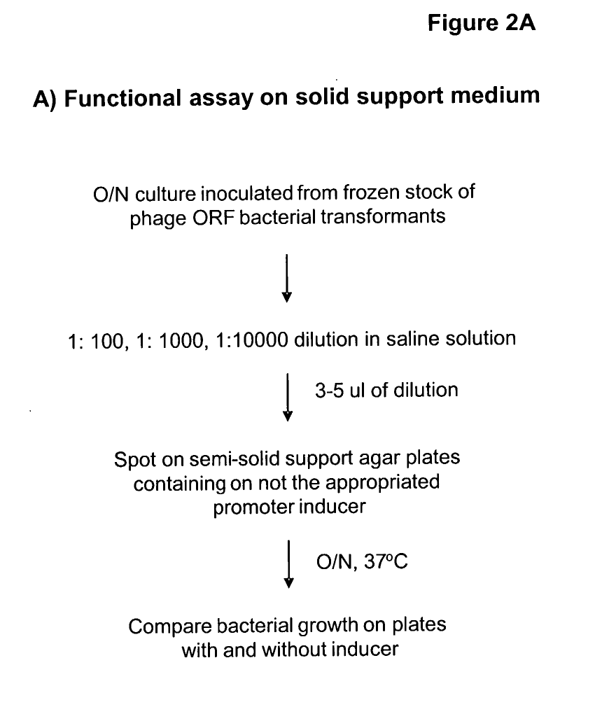

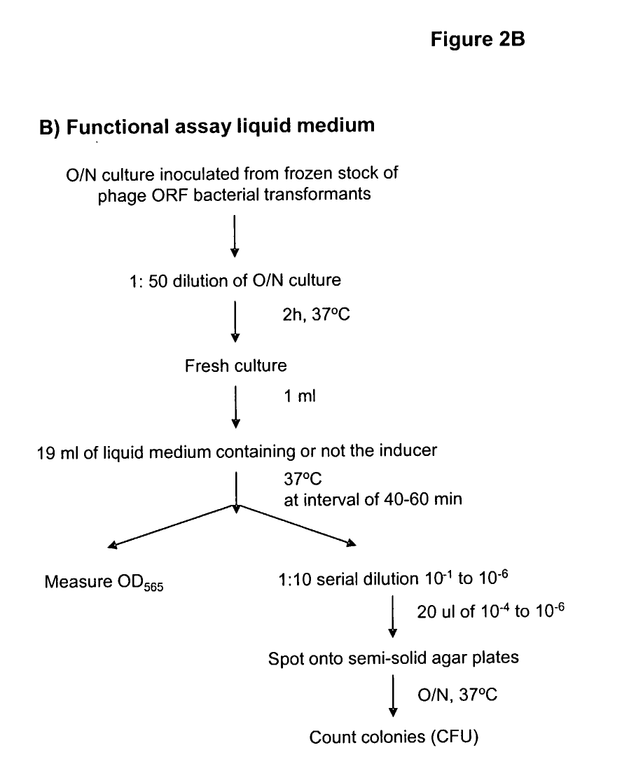

The plasmid pNZ8048 replicates in S. pneumoniae, in E. coli, and in L. lactis and was obtained from NIZO, Ede, The Netherlands. By the following strategy, the NcoI site at nucleotide 198 of pNZ8048 (3349 bp) was replaced with a BamHI site to enable BamHI/HindIII cloning of phage ORFs downstream of the nisin-regulated nisA promoter. The pNZ8048 vector was digested with BstBI and PstI and the resulting 3298 bp vector fragment was purified from the 51 bp BstBI-RBS-NcoI-PstI fragment by gel purification using a QIAquick gel extraction kit (Qiagen). The purified vector fragment was ligated to an annealed synthetic replacement oligonucleotide consisting of the following two single-stranded sequences: 5′-cgaaggaactacaaaataaattataaggaggcggatcctgca-3′ (SEQ ID NO: 5), with BstI- and PstI-compatible ends underlined and the nisA ribosome binding sequence (RBS) in bold; 3′-ttccttgatgttttatttaatattcctccgcctagg-5′ (SEQ ID NO: 6), with the newly-introduced BamHI site in italics. The candidate plasmid pZ (3340 bp) was sequenced using primer 8048F (5′-attgtcgataacgcgagc-3′ (SEQ ID NO: 7)) and was verified to have incorporated faithfully the replacement oligonucleotide. As shown in FIG. 1, the final vector, pZ, allows the cloning of ORF downstream of the nisin-inducible promotor in a multi cloning site.

### EXAMPLE 5

### Cloning of ORF Associated with a Shine-Dalgarno Sequence

ORFs with a Shine-Dalgarno sequence were selected for functional analysis of bacterial growth inhibition. Each ORF, from initiation codon to termination codon, was amplified by PCR from phage genomic DNA and cloned in pZ. Recombinant clones were then picked and the sequence fidelity of cloned ORFs was verified by DNA sequencing. In cases where verification of ORFs could not be achieved by one path, by sequencing using primers flanking the cloning sites, internal primers were selected and used for sequencing. Recombinant plasmids were introduced into a S. pneumoniae R6 strain containing pNZ9530 for constitutive expression of NisRK (R6RK strain), as described previously (Diaz et al., 1990, Gene 90:163-167).

### EXAMPLE 6

### Screening for Phage-Derived Inhibitory ORFs

Nisin (1 ug/mL) available from Sigma (Sigma-Aldrich Canada LTD, Oakville) was used to induce bacteriophage ORFs expression from the nisin-inducible promotor in functional assays. The anti-microbial activity of individual ORF from phage dp1 was monitored in S. pneumoniae R6RK by two growth inhibitory assays, one on solid agar medium, the other in liquid medium broth.

i) Dot Screening on Agar Plates

The functional identification of inhibitory ORFs was performed by dotting 5 μl aliquots of dilutions of S. pneumoniae R6RK transformant cells harboring phage ORFs onto Todd-Hewitt medium containing nisin (1 μg/mL) and supplemented with catalase (260 U/mL) as well as the appropriate antibiotics for maintenance of pNZ9530 (0.5 μg/mL erythromycin) and recombinant pZ (2 μg/mL chloramphenicol). Aliquots of the culture (same dilutions) were also plated on control plates of the same composition but without nisin. The plates were incubated overnight at 37° C.; any inhibition of growth of the ORF transformants on plates that contain nisin was discerned by comparison of growth of the same transformants on plates without nisin. Two ORFs derived from dp1 phage (SEQ ID NO: 1 and 2) were demonstrated to inhibit the S. pneumoniae bacterial growth (results not shown).

ii) Quantification of Growth Inhibition of Phage ORFs in Liquid Medium

S. pneumoniae R6RK cells containing ORFs corresponding to SEQ ID NO: 1 and 2 were grown overnight at 37° C. in Todd-Hewitt medium supplemented with catalase (260 U/mL) and the appropriate antibiotics for maintenance of pNZ9530 (0.5 μg/mL erythromycin) and recombinant pZ (2 μg/mL chloramphenicol). Cells were diluted with fresh selective medium and growth was allowed to proceed into mid log phase (OD₆₀₀=0.2). Dilutions of each culture (three independent transformants harbouring the ORF under study; negative control; positive control) were made in duplicate into tubes containing fresh Todd-Hewitt catalase medium with selective antibiotics and with or without inducer (nisin 1 μg/mL). Dilutions were chosen to normalize the initial optical densities of all cultures. At time zero and at each 1 hour interval for four hours, the number of colony forming units (CFU) present in each culture was assessed by diluting an aliquot of cells and dotting the dilutions on agar plates with or without selective antibiotics. After 48 h growth at 37° C., the colonies were counted and the number of CFU present in each culture at each timepoint was plotted.

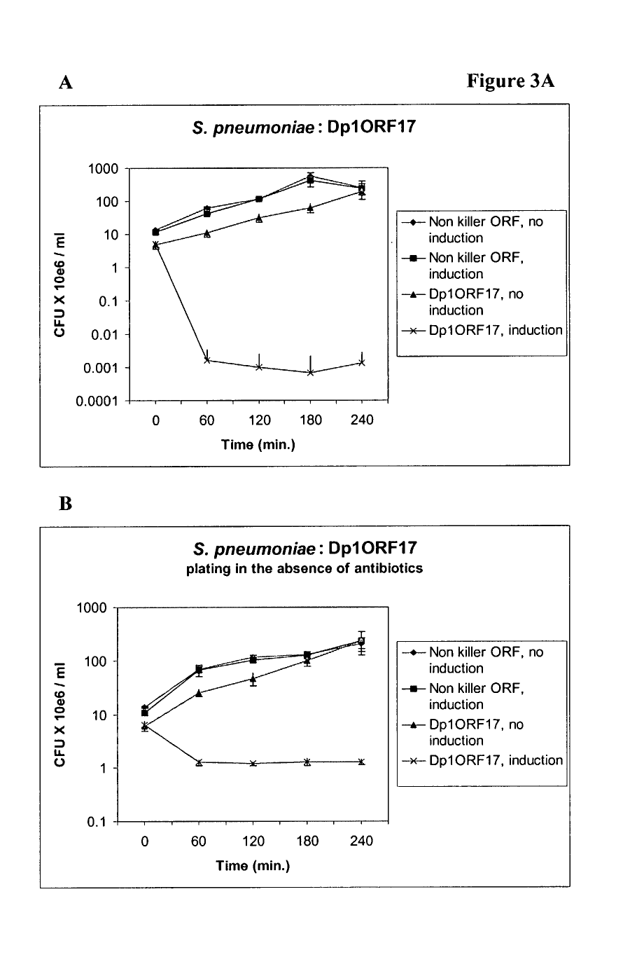

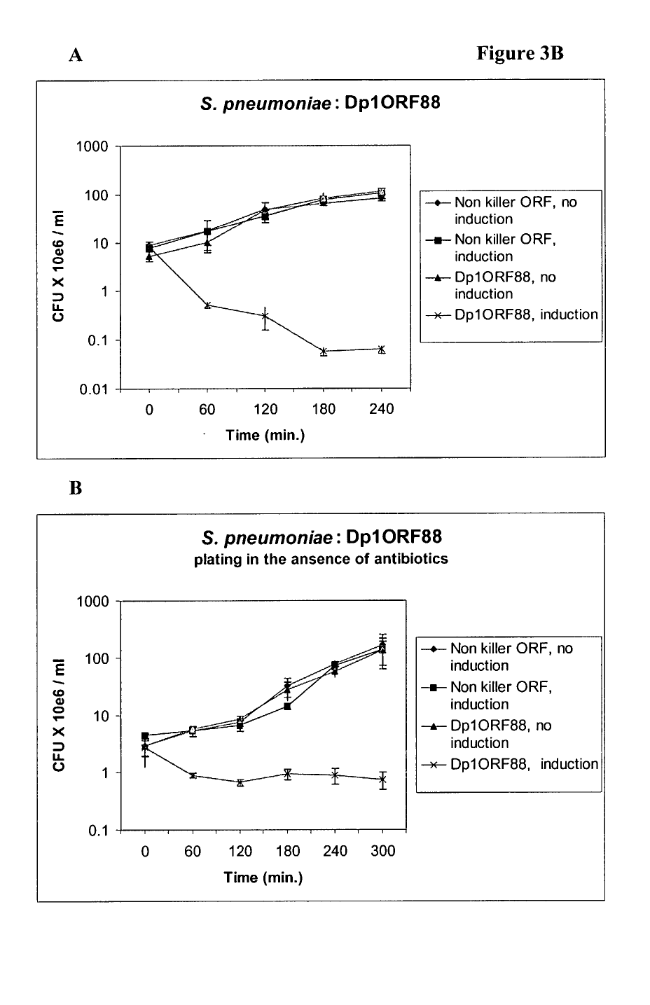

As presented in FIG. 3 and as evaluated at 4 h following ORF expression, dp1ORF17 and dp1ORF88 exhibit a bacteriocidal activity as they induce a 4 log and 2.5 log reduction, respectively, on the CFU number compared to CFU initially present in the same culture. In parallel cultures, the number of CFU increased over time under non-induced conditions with the same logarithmic expansion as observed in both uninduced and induced control cultures. When colony plating was done in the absence of the antibiotics necessary to maintain the selective pressure for the plasmids (chloramphenicol 2 μg/ml, erythromycin 0.5 μg/ml), the extent of growth inhibition was slighty reduced compared to plating in the presence of antibiotics (Graphs indicated ‘plating in the absence of antibiotics’ in FIG. 3).

### EXAMPLE 7

### Measurement of ORF Expression in S. pneumoniae

For the analysis of the inhibitory ORFs expression in S. pneumoniae, the HA tag was fused to the N-terminal end of the ORF. Two oligonucleotides corresponding to a short antigenic peptide derived from the heamaglutinin protein of influenza virus (HA epitope tag) were synthesized (Field et al., 1988). The sense strand HA tag sequence (with BamHI, SalI and HindIII cloning sites) is: 5′-GATCATGTACCCATACGACGTCCCAGACTACGCCAGCGGATCCCGTGCTACGA AGCTTCG-3′ (SEQ ID NO: 8); the antisense strand HA tag sequence (with a HindIII cloning site) is: 5′-TCGAGTCGACACGAAGCTTCGTAGCACGGGATCCGCTGGCGTAGTCTGGGACG TCGTATG-3′ (SEQ ID NO: 9) (where upper case letters denote the sequence of the HA tag). The two HA tag oligonucleotides were annealed and ligated to pZ to generate pZHN. dp1ORF17 and dp1ORF88 were cloned into cloned in pZHN.

S. pneumoniae R6RK cells containing individual fusion proteins were grown overnight at 37° C. in Todd-Hewitt medium supplemented with catalase (26 U/mL) and the appropriate antibiotics for maintenance of pNZ9530 (0.5 μg/mL erythromycin) and recombinant pZHN (2 μg/mL chloramphenicol). The overnight cultures were diluted 50-fold into fresh medium containing erythromycin and chloramphenicol and their growth continued for 2 h at 37° C. At the end of this time period, cells were diluted with fresh medium with or without the nisin and incubated at 37° C. for an additional 3 h. Bacterial pellets were lysed in a solution of 50 mM Tris-HCl [pH 7.6] 1 mM EDTA, 3 mM gluthathione, 10 mM sodium fluoride, 50 mM sodium chloride and 0.1% sodium deoxycholate at 30° C. for 10 minutes.

The level of expression of the inhibitory ORF was measured by performing Western blot analyses. Cell lysates were boiled for 10 min, centrifuged for 10 min at 13,000 g and 10-15 μl of the lysates loaded onto a 15-18% SDS-PAGE gel using Tris-glycine-SDS as a running buffer (3.03 g of Tris HCl, 14.4 g of glycine and 0.1% SDS per liter). After migration, proteins were transferred onto a PVDF membrane (immobilon-P; Millipore) using Tris-glycine-methanol as a transfer buffer (3.03 g Tris, 14.4 glycine and 200 ml methanol per liter) for 2 hrs at 4° C. at 100 V.

After the transfer, the membranes were blocked in 20 ml of TBS containing 0.05% Tween-20 (TBST), 5% skim milk and 0.5% gelatin for 1 hr at room temperature and then, a pre-blocking antibody (ChromPureRabbit IgG, Jackson immunoResearch lab. #011-000-003) was added at a dilution of 1/750 and incubated for 1 hr at room temperature or O/N at 4° C. The membrane was washed six times for 5 min each in TBST at room temperature. The primary antibody (murine monoclonal-HA anti-antibody, Babco #MMS-101 P) directed against the HA epitope tag and diluted 1/1000 was then added and incubated for 3 hrs at room temperature in the presence of 5% skim milk and 0.5% gelatin. The membrane was washed six times for 5 min each in TBST at room temperature. A secondary antibody (anti-mouse IgG, peroxidase-linked species-specific whole antibody, Amersham #NA 931) diluted 1/1500 (7.5 μl in 10 ml) was then added and incubated for 1 hr at room temperature. After six washes in TBST, the membrane was briefly dried and then, the substrate (Chemiluminescence reagent plus, Mandel #NEL104) was added to the membrane and incubated for 1 min at room temperature. The membrane was blotted to remove excess substrate and exposed to x-ray film (Kodak, Biomax MS/MR) for different periods of time (30 s to 10 min).

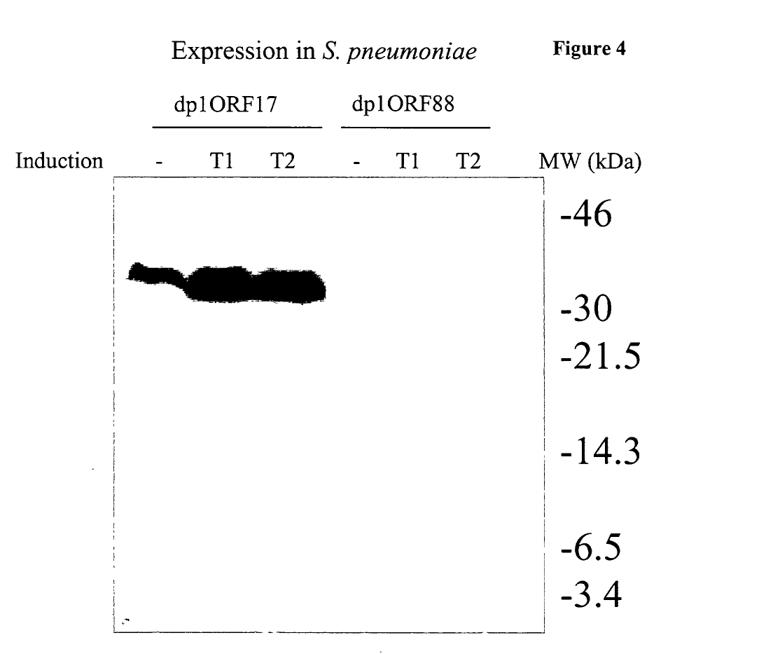

As shows in FIG. 4, the presence of the inducer in the cultures results in the expression of dp1ORF17 and dp1ORF88.

### EXAMPLE 8

### Identification of a S. pneumoniae Protein Targeted by dp1 ORF 17 or 88

To identify the S. pneumoniae protein(s) that interacts with inhibitory ORF 17 or 88 of S. pneumoniae bacteriophage dp1, tag-fusion dp1 ORF 17 or 88 are generated. Bacteriophage ORF is sub-cloned into pGEX 4T-1 (Pharmacia), an expression vector for in-frame translational fusions with GST and which contains regulatory sequences that allow inducible expression of the fusion GST/ORF protein. Recombinant expression vectors are identified by restriction enzyme analysis of plasmid minipreps. Large-scale DNA preparations are performed with Qiagen columns, and the resulting plasmid is sequenced. Test expressions in E. coli cells containing the expression plasmids are performed to identify optimal protein expression conditions. E. coli DH5 cells containing the expression constructs are grown at 37° C. in 2 L Luria-Bertani broth to an OD₆₀₀ of 0.4 to 0.6 (1 cm pathlength) and induced with 1 mM IPTG for the optimized time and temperature.

Cells containing GST/ORF fusion protein are suspended in 10 ml GST lysis buffer/liter of cell culture (GST lysis buffer: 20 mM Hepes pH 7.2, 500 mM NaCl, 10% glycerol, 1 mM DTT, 1 mM EDTA, 1 mM benzamidine, and 1 PMSF) and lysed by French Pressure cell followed by three bursts of twenty seconds with an ultra-sonicator at 4° C. The lysate is centrifuged at 4° C. for 30 minutes at 10 000 rpm in a Sorval SS34 rotor. The supernatant is applied to a 4 ml glutathione sepharose column pre-equilibrated with lysis buffer and allowed to flow by gravity. The column is washed with 10 column volumes of lysis buffer and eluted in 4 ml fractions with GST elution buffer (20 mM Hepes pH 8.0, 500 mM NaCl, 10% glycerol, 1 mM DTT, 0.1 mM EDTA, and 25 mM reduced glutathione). The fractions are analyzed by 15% SDS-PAGE (Laemmli) and visualized by staining with Coomassie Brilliant Blue R250 stain to assess the amount of eluted GST/ORF protein.

A S. pneumoniae extract is prepared by incubating the cell pellets in a solution of 50 mM Tris-HCl [pH 7.6] 1 mM EDTA, 3 mM gluthathione, 10 mM sodium fluoride, 50 mM sodium chloride and 0.1% sodium deoxycholate at 30° C. for 10 minutes. The lysate is centrifuged at 20 000 rpm for 1 hr in a Ti70 fixed angle Beckman rotor. The supernatant is removed and dialyzed overnight in a 10 000 Mᵣ dialysis membrane against Affinity Chromatography Buffer (ACB; 20 mM Hepes pH 7.5, 10% glycerol, 1 mM DTT, and 1 mM EDTA) containing 100 mM NaCl, 1 mM benzamidine, and 1 mM PMSF. The dialyzed protein extract is removed from the dialysis tubing and frozen in one ml aliquots at −70° C.

Control GST and GST/ORF proteins are dialyzed overnight against ACB buffer containing 1 M NaCl. Protein concentrations are determined by Bio-Rad Protein Assay and proteins are crosslinked to Affigel 10 resin (Bio-Rad) at protein/resin concentrations of 0, 0.1, 0.5, 1.0, and 2.0 mg/ml. The crosslinked resin is sequentially incubated in the presence of ethanolamine and bovine serum albumin (BSA) prior to column packing and equilibration with ACB containing 100 mM NaCl. S. pneumoniae extracts are centrifuged at 4° C. in a micro-centrifuge for 15 minutes and diluted to 5 mg/ml with ACB containing 100 mM NaCl. Aliquots of 400 μl of extract are applied to 40 μl columns containing 0, 0.1, 0.5, 1.0, and 2.0 mg/ml ligand and ACB containing 100 mM NaCl (400 μl) is applied to an additional column containing 2.0 mg/ml ligand. The columns are washed with ACB containing 100 mM NaCl (400 μl) and sequentially eluted with ACB containing 0.1% Triton X-100 and 100 mM NaCl (100 μul), ACB containing 1 M NaCl (160 μl), and 1% SDS (160 μl). For further analysis, 80 μl of each eluate is resolved by 16 cm 14% SDS-PAGE (Laemmli, U. K. (1970) Nature 227: 680-685) and the protein is visualized by silver stain.

The selected S. pneumoniae interacting polypeptides are excised from the SDS-PAGE gels and prepared for tryptic peptide mass determination by mass spectrometry using, for example, MALDI-ToF technology (Qin, J., et al. (1997) Anal. Chem. 69:3995-4001). Computational analysis of the mass spectrum obtained identifies the corresponding ORF in the S. pneumoniae nucleotide sequence.

Sequence homology (BLAST) and Hidden Markov Model (HMM) searches are then carried out with the identified bacterial sequences using an implementation of both programs. Downloaded public databases used for sequence analysis include those listed in Example 3.

The interaction between the bacterial target and the dp1 ORF is further characterized by using yeast two-hybrid assay. The polynucleotide sequence of the bacterial target is obtained from S. pneumoniae genomic DNA by PCR utilizing oligonucleotide primers that targeted the predicted translation initiation and termination codons of the gene. The PCR product is purified using the Qiagen PCR purification kit and cloned in fusion with the Gal4 activating domain into the pGADT7 vector (Clontech Laboratories). A similar strategy is used for the cloning of dp1 inhibitory ORF to the carboxyl terminus of the yeast Gal4 DNA binding domain (encoded by the pGBKT7 vector) or to the yeast Gal4 activation domain (encoded by pGADT7).

The pGAD and pGBK plasmids bearing different combinations of constructs are introduced into a yeast strain (AH109, Clontech Laboratories), previously engineered to contain chromosomally-integrated copies of E. coli lacZ and the selectable HIS3 and ADE2 genes. Co-transformants are plated in parallel on yeast synthetic medium (SD) supplemented with amino acid drop-out lacking tryptophan and leucine (TL minus) and on SD supplemented with amino acid drop-out lacking tryptophan, histidine, adenine and leucine (THAL minus). An interaction between bacterial target and dp1 inhibitory ORF results in induction of the reporter HIS3 and ADE2 genes and growth of yeast on THAL medium.

### CONCLUSION

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The specific methods and compositions described herein as presently representative of preferred embodiments are exemplary and are not intended as limitations on the scope of the invention. One of ordinary skill in the art would recognize that, bacteriophages dp1 ORFs described herein are provided and discussed by way of example are within the scope of the present invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention. For example, those skilled in the art will recognize that the invention may suitably be practiced using a variety of different expression vectors and sequencing methods within the general descriptions provided.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is not intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group. For example, if there are alternatives A, B, and C, all of the following possibilities are included: A separately, B separately, C separately, A and B, A and C, B and C, and A and B and C.

Thus, additional embodiments are within the scope of the invention and within the following claims.

Although the present invention has been described hereinabove by way of preferred embodiments thereof, it can be modified without departing from the spirit and nature of the subject invention as defined in the appended claims.

TABLE 1

| | | |
|---|---|---|
| 1st | | 3rd |
| position | 2nd position | position |

| (5′ end) | U | C | A | G | (3′ end) |
|---|---|---|---|---|---|
| U | Phe | Ser | Tyr | Cys | U |
| | Phe | Ser | Tyr | Cys | C |
| | Leu | Ser | Stop | Stop | A |
| | Leu | Ser | Stop | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

TABLE 2

List of nucleotide and amino acid sequences of
inhibitory ORFs from phage dpi.

| | |
|---|---|
| dp1ORF17 nucleotide sequence: | SEQ ID NO: 1 |

ATGATTGGACAGGGACTTGTTAAATCTACCATTTCGAAATGGAAACAACT
TCCAAAATATATAATCGTCGAAGGTGAAGTAGGTTCAGGACGGAAGACCT
TAATCCGTTATATTGCTTCGAAATTTGACGCTGATTCTATTGTAGTAGGA
ACGAGTGTAGATGACATTCGAAACATCATTCAGGATGCACAGACTATTTT
CAAGGCGAGAATCTACGTGATAGACGGAAATAGCCTGTCAATGTCAGCTC
TTAACTCGCTTTTGAAGATAGCGGAAGAGCCACCTTTAAACTGTCATATA
GCCATGACTGTTGATAGCATCAATAATGCTTTACCTACGCTTGCAAGTAG
AGCAAAAGTTCTAACCATGCTACCTTATACTAATGAAGAGAAAATGCAGT
TTGTCAAGTCCTACAAGAAGGTAGATACTTCAGGAATTGACGACCGAGCG
ATTGTAGACTATTGCAATCTTGCCAGCAATCTTCAAATGCTTGAAGACAT
ATTAGAATATGGCGCAGAAGAGCTATTTGAAAAGGTTACAACATTTTATG
ACTTAATATGGGAGGCAAGTGCTAGCAATTCGCTAAAGGTTACTAATTGG
CTCAAATTTAAGGAAACTGATGAAGGAAAAATTGAGCCTAAACTTTTCCT
CAACTG3TCTTTTAAATTGGTCGACAGTTGTCATCAGGAAGCACTATGTA
GAAATGTCTTTCGAAGAACTTGAGGCCCATGACCTTTTAGTGAGGGAAGC
ATCTAGGTGTTTGCGAAAGGTATCTAAAAAGGGCTCAAATGCGCGTGTCT
GCGTGAACGAATTTATCAGGAGGGTCAAACAAGTTGAGTGA

| | |
|---|---|
| dp1ORF88 nucleotide sequence: | SEQ ID NO: 2 |

ATGAAAAAAGTTCAAACTTATCAAGAATATCTAAAACTAGTTGAGTTCAA
ACGTCAACTTTCTTTAAATCTTCGAGAAGGAAAAATAGGAGTCGATGAAG
CGGTTATTCAATTATTCACCTTCTATAGTTTCAACAATATCGAGGAACCT
CCTTTCATTGTACTCAAAATGCAAGAGGCTGCCGTGAACGGGACTTATGA
AGCAAAACTCAATATGCTTAAAAGATTTAAAATTATTTAG

| | |
|---|---|
| dp1ORFl7 amino acid sequence: | SEQ ID NO: 3 |

MIGQGLVKSTISKWKQLPKYIIVEGEVGSGRKTLIRYIASKFDADSIVVG
TSVDDIRNIIQDAQTIFKARIYVIDGNSLSMSALNSLLKIAEEPPLNCHI
AMTVDSINNALPTLASRAKVLTMLPYTNEEKMQFVKSYKKVDTSGIDDRA
IVDYCNLASNLQMLEDILEYGAEELFEKVTTFYDLIWEASASNSLKVTNW
LKFKETDEGKIEPKLFLNCLLNWSTVVIRKHYVEMSFEELEAHDLLVREA
SRCLRKVSKKGSNARVCVNEFIRRVKQVE

| | |
|---|---|
| dp1ORF88 amino acid sequence: | SEQ ID NO: 4 |

MKKVQTYQEYLKLVEFKRQLSLNLREGKIGVDEAVIQLFTFYSFNNIEEP
PFIVLKMQEAAVNGTYEAKLNMLKRFKII

TABLE 3

Blast Analysis

| | |
|---|---|
| Database: | nr (AA) from GenBank |
| | 884,779 sequences; |
| | 277,083,049 total letters |

1. SEQ ID NO: 3 dp1ORF017
Query: SEQ ID NO: 3

| Sequences producing | Score | E |
|---|---|---|
| significant alignments: | (bits) | Value |
| >gi\|9632638 DNA polymerase | 42 | 0.012 |
| accessory... | | |
| >gi\|3913513 DNA POLYMERASE | 40 | 0.034 |
| ACCESSORY PROTEIN... | | |
| >gi\|17554064 NADH dehydrogenase | 39 | 0.099 |
| [Cae... | | |
| >gi\|16801912 highly similar to | 39 | 0.099 |
| DNA p... | | |
| >gi\|16804741 highly similar to | 39 | 0.099 |
| DNA p... | | |

2. SEQ ID NO: 4 dp1ORF088
Query: SEQ ID NO: 4

| Sequences producing | Score | E |
|---|---|---|
| significant alignments: | (bits) | Value |
| >gi\|13186336 transaldolase | 32 | 1.0 |
| [Candidatus... | | |
| >gi\|13186344 transaldolase | 32 | 1.7 |
| [Candidatus... | | |
| >gi\|13186340 transaldolase | 30 | 3.8 |
| [Candidatus... | | |
| >gi\|15965530 PUTATIVE | 30 | 5.0 |
| TRANSCRIPTION... | | |
| >gi\|2625021 DNA helicase II | 30 | 5.0 |
| [Serratia m... | | |

TABLE 4

Phage Dp1 complete genome sequence. 56506 nucleotides

(SEQ ID NO. 10)

| | |
|---|---|
| 1 | ataataaaaa tatgaagcag atattgggtt aattattgct taacaaaatg caccgaattt gtgtataata |
| 71 | taagtgaagc agttttgtaa acctgacatc ctgctaaata aaaataaagg aggctcgaac atgagtcaaa |
| 141 | acactacacg cactgacgct gaattgacag gcgttactct tttaggaaac caagacacca aatacgatta |
| 211 | tgactataat ccagacgtcc ttgaaacttt ccctaacaaa catcctgaaa ataattacct agtaacattt |
| 281 | gacggatatg aattcacttc cctttgccct aaaacaggac agcctgactt cgcgaatgtt ttcattagtt |
| 351 | acattccaaa cgaaaagatg gttgaatcta aatcattgaa attgtactta ttcagtttcc gtaaccacgg |
| 421 | tgacttccac gaagattgca tgaacattat tttgaatgac ttgtatgaat tgatggaacc taagtacatt |
| 491 | gaagtcatgg gcctattcac tcctcgtggt ggaatttcaa tttacccatt cgtcaacaaa gtgaatcctc |
| 561 | aatttgcaac tcctgaactt gaacagcttc aacttcaacg caaattgaac ttccttggaa atgttcaagg |
| 631 | tcttggacga gctattcgat aggaggctgg aatgaaatca gtagttttat tatccggcgg agtcgactca |
| 701 | gccacttgtt tagcaattga agttgacaag tggggttcta aaaatgttca tgctatagca ttcaattacg |
| 771 | gacaaaagca tgaagcagaa cttgaaaatg ctgctaatgt tgcaatgttc tacggagtca agttcaccat |
| 841 | tcttgaaatt gactcgaaaa tctactcaag ctctagctct tccttattac aaggaaaagg cgaaatttca |
| 911 | catggaaaat cttacgctga aatcctagca gagaaggaag tagttgacac ctatgttcca tttagaaatg |
| 981 | gactaatgct ttcacaggct gcggcttatg cttattcggt tggagcttct tacgtcgtat atggtgctca |
| 1051 | cgcagacgat gcggctggag gtgcttaccc tgattgcact cctgagttct ataattcaat gtcaaatgca |
| 1121 | atggaatatg gaactggagg caaggtaacc cttgtcgctc ctctacttac tctaaccaag gcgcaagtcg |
| 1191 | ttaaatgggg aattgattta gatgttcctt atttcttgac tcgttcatgt tatgaaagtg acgctgaaag |
| 1261 | ttgtggaact tgcgcaactt gtatcgaccg caaaaaggca ttcgaagaaa atggaatgac tgaccctatt |
| 1331 | cattataagg agaattgata tgagagtttc taaaacctta acattcgacg cagctcatca actagttgga |
| 1401 | cattttggaa aatgcgcaaa tttgcacggg catacttaca aagtcgaaat ttcattagca ggcggaactt |
| 1471 | atgaccacgg ttcgagtcaa gggatggttg ttgactttta tcacgtcaag aaaatcgcag gtacattcat |
| 1541 | tgacagactt gaccacgctg ttcttcttca agggaatgaa ccaatcgctt tagcaaatgc agttgacacc |
| 1611 | aagcgagttc tatttggatt tagaactacg gctgagaata tgtcaagatt ccttacctgg actctcacgg |
| 1681 | agcttatgtg gaagcatgct cgtatcgact ctatcaaact atgggaaact cctacaggtt gcgcagaatg |
| 1751 | tacttactac gagattttca cagaagacga gattgaaatg ttcaagaacg taacctttat cgacaaagac |
| 1821 | gaaaagatta ctgtccgcga aattttagag caggagcagg ataatggtta atcaatacaa tcagcctgaa |
| 1891 | agaggcaaga ttcgaatcaa tgttcgcgac cctgagaaaa tgcctatcat ggaaattttc ggtcctacaa |
| 1961 | ttcaaggtga aggaatggtt ataggtcaaa agactatttt cattcgaact ggtggatgcg actatcattg |
| 2031 | caactggtgt gactcagcct ttacctggaa cggtactact gagccggaat atatcacagg caaagaagct |
| 2101 | gctagtcgaa tcttgaaact agctttcaat gataaaggtg aacagatttg taaccacgtg acattgactg |
| 2171 | gaggaaatcc tgccttaatc aacgagccta tggctaagat gatttcgatt ctaaaagaac atggattcaa |
| 2241 | gtttggtctc gaaactcaag gaactcgatt ccaagaatgg ttcaaagaag taagcgatat cactattagt |
| 2311 | cctaaaccgc cttcaagtgg aatgagaact aatatgaaaa ttcttgaagc tattgtagat agaatgaatg |
| 2381 | atgaaaacct tgactggtca tttaaaatcg ttatctttga cgaaaatgac ctagcttatg cgcgtgatat |
| 2451 | gtttaaaact ttcgaaggca agttacgtcc agtgaactac ctttcagttg ggaatgcaaa cgcatacgaa |
| 2521 | gaaggaaaaa tcagtgatag gcttcttgaa aagttgggat ggctttggga taaagtgtat gaagacccag |
| 2591 | ctttcaacaa tgttcgacct ttaccgcaac ttcatacact tgtttatgat aataaaagag gagtataaaa |
| 2661 | tgaaaattga gcatctagat aaaatcggta acgtattagg gagagagaac ggatgggctt cccttaagcc |
| 2731 | ggatgaaatt gtaaccttgg acaatactga ggcagccgtt caaagacttt ttggtctatt aggcgaggac |
| 2801 | gcagaacgtg acgggttgca agatactcca ttccgttttg ttaaagcact cgctgaacat accgtagggt |
| 2871 | atcgagaaga ccctaaactt catctcgaaa aaacattcga cgtcgaccat gaagaccttg ttcttgtgaa |
| 2941 | agacattcca ttcaattctt tatgtgagca tcatttagct ccgttcgtag ggaaggtgca tattgcatac |
| 3011 | attcctaagg ataagattac aggtctttca aaattcggtc gagtggttga aggatacgct aaacgacttc |
| 3081 | aagtacaaga gcgcttgact caacaaatcg ctgacgctat tcaggaagtt ctaaatcctc aagcagttgc |
| 3151 | ggtcatcgta gaggctgagc atacttgcat gagcggacgc ggtattaaga agcacggggc aacgacagtg |
| 3221 | acttcaacta tgcgaggtct tttccaagat gacgcatctg ctcgagcaga attgcttcag ttgattaaaa |
| 3291 | agtaggaggc ggaaaatgaa taaaagtgca accttttggc ttgttcgaac agctcttatt gcggctctat |
| 3361 | atgtgacatt gaccgttgca ttttctgcta ttagttatgg acctattcaa tttagagtca gtgaagcctt |
| 3431 | gattcttcta cctttatgga accatagatg gactccgggg attgtattag gaacaattat tgcaaacttc |
| 3501 | ttttcacctc ttggactgat tgacgtttta ttcggttcac ttgctacctt ccttggagta gtggcaatgg |
| 3571 | tgaaagttgc taagatggca agtcctctat attcacttat ctgtccagtt cttgctaatg cttaccttat |
| 3641 | tgcgctggaa cttcgaatag tttactcttt acctttttgg gaatctgtca tctatgtagg aattagtgaa |
| 3711 | gcgattatcg ttttaatttc atacttcctt atttccacgc tggcgaagaa caatcatttt agaacactga |
| 3781 | taggagcgaa aaatgggatt taatctatac ttcgcaggag gtcacgctat tagcactgac gattatttga |
| 3851 | aggaaagagg agccaatcgc ctattcaatc aactgtacga aagaaacggg attggcaaaa ggtggattga |
| 3921 | gcataagaaa accaatccaa gcactacttc aaaactattc gtcgactcta gtgcatattc tgctcatacc |
| 3991 | aaaggggctg aagttgacat tgacgcctat atcgaatacg tgaatgataa cgtgggaatg tttgactgta |
| 4061 | tcgccgaact cgataaaatt cctggtgtat ttagacagcc taagacacgt gaacagcttt tggaagcacc |
| 4131 | acaaatttct tgggataatt atctatacat gcgcgagcga atggttgaga aagacaagct cttacctatt |
| 4201 | ttccatatgg gagaagactt taaatggctc aacttgatgc tcgaaactac attcgaaggc ggaaagcata |
| 4271 | ttccttacat tggaatttca ccagccaatg actcgactac gaagcataaa gacaagtgga tggaaagagt |
| 4341 | attcgaagtt attcgaaaca gttctaatcc agacgttaag actcacgcat ttgggatgac agttactagc |
| 4411 | caattagagc gtcacccatt ctatagcgcc gactctactt ctgtactgct cacaggagcg atgggaaaca |
| 4481 | ttatgacgtc aaaaggatta gttgacttgt cacagaagaa tggaggaatt gatgctgtcc gtaggctgcc |
| 4551 | aaaaccggtt caagttgaaa ttgaatccat tatcgaagaa actggagcgc attttagcct agagcaatta |
| 4621 | gttgaggact ataaacttcg agcattgttc aatgttcaat acatgctgaa ttgggcagag aactatgaat |
| 4691 | tcaagggaat taaaaatcgt caacgtcgac tattttagat aagagctttt cgctcttatt ttttttaaaa |
| 4761 | aaaaatgaac tttttataca aaaacgcttg actttattca ctcattatcg tataatcata atataaataa |
| 4831 | aacgaataag aggtaaataa aatgacagca gttcaacaag ttaagttcta cttagaagaa gccggcgctc |
| 4901 | actttctaaa agatgttgag tacagtgaca acttagagca agcaattatg aaagatattc ttaaatggaa |
| 4971 | tggcgctcat agagatgagc acgatatgaa aataacttca tacgaagtat tatagagagg ggtaaggcta |
| 5041 | tgaaaaaagt tcaaacttat caagaatatc taaaactagt tgagttcaaa cgtcaacttt ctttaaatct |
| 5111 | tcgagaagga aaaataggag tcgatgaagc ggttattcaa ttattcacct tctatagttt caacaatatc |
| 5181 | gaggaacctc ctttcattgt actcaaaatg caagaggctg ccgtgaacgg gacttatgaa gcaaaactca |
| 5251 | atatgcttaa aagatttaaa attatttaga aacggcttta caaactcgcg ataattcgtg tatattatat |
| 5321 | atatcaaaaa aaggaggctc atattatgag tattaagttc aaaaccgaag aactttcaaa aattgtttct |
| 5391 | cagctcaata agttgaagcc tagcaagttg ctagaaatca caaactattg gcatattttt ggtgacggcg |
| 5461 | aatgcgtcat gtttacagcg tatgatggct caaacttcct tcgatgcatt atcgacagcg atgttgaaat |
| 5531 | tgacgtgatt gtgaaagcag agcagtttgg aaaacttgta gaaaagacca cggccgcaac cgtcacatta |
| 5601 | gttcctgaag aatcttcgct aaaagttatt gggaatggtg agtacaatat tgatattgtt acagaagatg |
| 5671 | aagagtaccc tacattcgac cacttgctcg aagacgtgag tgaagaaaat gctctcactt tgaaaagctc |
| 5741 | gctgttctac ggaatcgcca atatcaacga ttctgcggta tctaaatcag gagcagatgg aatttatacc |
| 5811 | ggcttcctgt taaaaggcgg aaaagcaatt actacagaca tcattcgcgt atgtatcaac cctatcaagg |
| 5881 | aaaagggact agaaatgctc attccttaca acctaatgag tattttagca agtattcctg atgagaagat |
| 5951 | gtacttctgg caaattgacg atactactgt ctatatttca tcggcttcag tcgaaattta tggaaaattg |
| 6021 | atggaaggta tggaagatta tgaagacgtt tcacagcttg actcaattga gtttgaagat gatgcggcta |
| 6091 | tccctacagc agaaatcctg agcgtattag accgccttgt actattcact tcagcctttg acaaaggaac |
| 6161 | cgtcgaattc ttattcttga aagaccgact tcgaattaaa acttctacta gcagttatga agacatcatg |
| 6231 | tacgcatctg ctggcaagaa agtttcgaag aaagaattca cttgccacct taacagctta ctcttgaagg |
| 6301 | aaattgtatc aaccgtcacc gaagaaaact tcactgtctc ttatggaagc gaaaccgcaa ttaagatttc |
| 6371 | atcgaatggt gtcgtttact tcctagcact tcaagagccg gaagaataat ggccaagtcc aatttaacta |
| 6441 | gaattgcaaa gatggttaga gcaggaaaca gtgaaggtcc tgcttcatct tttgtcaatt cgctgacccg |
| 6511 | ggttattgaa cgaactcagc ctgaatataa tccttcgaca tattataagc ccagcggggt tggtggatgt |
| 6581 | attcgaaaaa tgtatttcga aagaatcggt gagtctatta tagataacgc agattctaac ctaattgcaa |
| 6651 | tgggcgaagc tggaacattt aggcacgaag ttctccaaga gtacatggtt aaaatggctg aaatcgatga |
| 6721 | ggactttgaa tggttgaatg tagcagagtt cttgaaagaa aatccagttg aaggaactat cgtcgacgag |
| 6791 | cgtttcaaga aaaacgatta tgaaacgaag tgtaagaacg aacttcttca actttcattc ttgtgtgacg |
| 6861 | gactagttcg atataaaggc aagctctaca ttttagagat taagactgaa accatgttca agttcactaa |
| 6931 | acatactgag ccctatgaag aacacaagat gcaagcaact tgctacggaa tgtgtctagg agtcgatgat |
| 7001 | gtcattttcc tttatgaaaa tcgagataac ttcgaaaaga aagcctacac gtttcacatc acagacgaga |
| 7071 | tgaaaaatca agtccttgga aaaattatga cctgcgaaga gtatgtagag aaaggcgaaa gtcctaaaat |
| 7141 | ctattgctct tcagcctatt gcccatattg tagaaaggaa ggtcgaaatc tgtgagctat actggaaaaa |
| 7211 | tgttcgagga agactttttc gaaggtgcaa aagactttga gaaagatgct ttcacggtcc gtctatatga |
| 7281 | taccactaat ggatttcgag gagttgcaaa tccctgcgat tatatagccg caactaactt tgggaccttg |
| 7351 | tttattgaac tgaaaactac taaagaagct tctttgagct ttaataacat cactgataat caatggttcc |
| 7421 | agctatcacg cgcagatgga tgcaaattta ttctcgccgg aattttagtg tatttccaaa agcatgaaaa |
| 7491 | gattatatgg tatccaattt caagccttga aaaaattaaa cggtctggag ttaaaagcgt caacccaaac |
| 7561 | ttcatcgatg cagggtatga agtttcttac aagaagcgtc gaactagatt gaccattcct ttccaaaatg |
| 7631 | ttctagatgc agttgagctt cattacaagg agaaaagcaa tggcaagacc taagttacct caaattgata |
| 7701 | ttcgagaaga agaaatacga gatgctcaag acgtagcaga ctcgtatggt gcgattatca ataaagtagt |
| 7771 | cgacgaaatt gttgaagcag cttgcggttc acttgaccag gcaatggaag aaattcaaat agttgtaagc |
| 7841 | caaaatcctg tcattatgga agaccttaac tactacattg gctatcttcc cactcttctt tatttcgccg |
| 7911 | cagatagggc ggaaatggtg ggaatacaaa tggattcaag ttctgctatc aggaaagaaa aatacgataa |
| 7981 | tctatacatt ttagccgccg ggaaaactat tcctgacaag caagcagaaa ctcgaaaact tgtcatgaat |
| 8051 | gaagaagtca tcgaaaatgc ttacaagcga gcctacaaga aagttcaatt aaagctagaa caggccgata |
| 8121 | aggtattagc atctttaaaa cgaattcaaa cctggcaact agcagagtta gaaactcagt caaataattc |
| 8191 | aaaaggagta ttattaaatg caaaaagacg tagacgtgaa aatgattgac cctaaacttg accgattaaa |
| 8261 | atacacaggt gattgggttg atgtacgaat tagttctatc actaaaattg acgccgacag cgccgatgtc |
| 8331 | tcaagatgtc gaaaagtgct tcaaaaggct caagtatatt cagtggcggc aggtgaatgc attaaaattg |
| 8401 | cacacggatt tgctcttgaa cttcctaagg gatatgaagc aatcttgcat cctcgttcca gtctttttaa |
| 8471 | gaaaactggt ctaatcttcg tttctagcgg agtgattgac gaaggttaca aaggtgacac tgatgaatgg |
| 8541 | ttctcagttt ggtatgctac tcgtgacgca gatatcttct acgaccaaag aattgcccaa tttagaattc |
| 8611 | aggaaaagca acctgctatc aagttcaatt tcgtagaatc tttaggaaat gcggctcgtg gaggccatgg |
| 8681 | aagtacaggt gatttctaat gaaattggaa cagttgatga aggactggaa taaggattcg aaagctcttg |
| 8751 | tagcagttca aggacttgaa cgtgaagcgc ttccaagaat ccctttttct gcgccttcta tgaattatca |
| 8821 | aacctacggc gggctccctc gaaaaagggt agttgaattc ttcggtcctg agtcaagtgg gaaaactact |
| 8891 | tcagctctcg acattgtcaa gaatgcgcaa atggtatttg agcaggaatg ggaacagaag actgaagaac |
| 8961 | tcaaggaaaa gctggaaaat gcgcgtgcat ccaaagctag caagactgct gtcaaggaac ttgaaatgca |
| 9031 | actcgatagt cttcaagagc ctcttaagat tgtatatctt gaccttgaga atacattaga cactgagtgg |
| 9101 | gctaaaaaga ttggagtcga tgttgacaat atttggatag ttcgccctga aatgaacagc gctgaagaaa |
| 9171 | tacttcaata tgttttagac attttcgaaa caggtgaagt tggcctagta gttctagatt ccttgcctta |
| 9241 | catggtcagt caaaacctta ttgatgaaga gttgactaaa aaggcctatg caggaatctc agcgcctttg |
| 9311 | actgaattta gtcgaaaggt tactcctctt cttactcgct acaatgcaat attcctaggc atcaatcaaa |
| 9381 | ttcgagaaga tatgaatagt cagtacaatg cctattcaac tccaggcgga aagatgtgga agcatgcttg |
| 9451 | tgcagttcga cttaaattta gaaaaggtga ctaccttgac gaaaacggtg catcattgac ccgtactgct |
| 9521 | cgaaaccctg cagggaatgt agtagagtca ttcgtcgaga agaccaaagc atttaagccg gacagaaaat |
| 9591 | tagtttccta tacgctttcc tatcatgatg gaattcaaat tgaaaatgac cttgtagatg tcgctgtcga |
| 9661 | atttggagtc attcaaaagg caggggcatg gttcagtatc gtcgaccttg aaactggaga aattatgaca |
| 9731 | gatgaagacg aagaaccatt gaagttccaa ggcaaggcaa atctagttcg acgcttcaag gaggatgact |
| 9801 | acttattcga catggtgatg actgcggttc acgaaattat cactcgagaa gaaggctaat gcaaaaatct |
| 9871 | ctatttggac ctaagctagt gcctgctagt tcaaggcgca agaaaagaac ggttccaaaa cctaaaccta |
| 9941 | aaatcgatga gcaagtggtt gagcttatga accgcagaga gcgtcaagtg cttgttcata gttgcatcta |
| 10011 | ttattatttt aatgactcaa ttatagcaga cgggcagtat gacaaatgga gccacgaact atattctctt |
| 10081 | atagtttcgc accctgatga gtttcgacag actgttctct ataacgagtt taaacagttt gacggaaata |
| 10151 | ctggaatggg tcttccatac gactgtcagt ttgctgtaag ggtcgcagaa aggcttttaa gaaaatgaat |
| 10221 | ttagcttcta aataccgtcc tcaaactttc gaggaagtgg tagctcaaga atatgtcaaa gaaattcttt |
| 10291 | tgaatcaatt acaaaatggc gctatcaaac acggctatct attctgtggt ggcgctggaa ctggtaaaac |
| 10361 | cactactgct cgaattttcg cgaaggatgt gaacaaagga cttggctctc ctattgaaat tgatgctgct |
| 10431 | tctaataatg gggtagaaaa tgttcgaaac attattgaag attctagata caagtctatg gacagcgagt |
| 10501 | tcaaagttta catcattgac gaggttcata tgctttcaac cggagcattt aatgcgctgt tgaaaacatt |
| 10571 | agaagagccc tcatcgggaa ccgtgttcat tctatgtact actgaccctc aaaagattcc tgacactatt |
| 10641 | ctcagtcgag ttcaacggtt tgactttact cgaattgata atgacgacat cgttaatcaa cttcaattta |
| 10711 | ttatcgaaag tgaaaatgaa gaaggagctg gttatagtta tgagcgtgac gccctttcgt ttattgggaa |
| 10781 | acttgcaaat ggaggaatgc gtgacagtat cacaaggctc gaaaaagtcc ttgattatag tcatcacgtt |
| 10851 | gacatggaag ccgtttctaa tgcactagga gttccggact acgaaacatt cgcttcactt gttgaagcta |
| 10921 | ttgccaacta tgacggctca aagtgtttag aaattgtaaa tgacttccac tactcaggaa aagacttgaa |
| 10991 | attagtgact cgaaacttta cagacttcct tttagaggtt tgtaagtatt ggctagttcg agatatttca |
| 11061 | atcactcaac ttcctgctca ttttgaaagt aagctagagc aattctgtga ggcttttcaa tatcctactc |
| 11131 | tattgtggat gctagaagaa atgaatgaac ttgctggagt tgttaaatgg gagcctaatg ctaaaccgat |
| 11201 | aattgaaacc aaacttcttt tgatgagcaa ggaggagtga catgattgga cagggacttg ttaaatctac |
| 11271 | catttcgaaa tggaaacaac ttccaaaata tataatcgtc gaaggtgaag taggttcagg acggaagacc |
| 11341 | ttaatccgtt atattgcttc gaaatttgac gctgattcta ttgtagtagg aacgagtgta gatgacattc |
| 11411 | gaaacatcat tcaggatgca cagactattt tcaaggcgag aatctacgtg atagacggaa atagcctgtc |
| 11481 | aatgtcagct cttaactcgc ttttgaagat agcggaagag ccacctttaa actgtcatat agccatgact |
| 11551 | gttgatagca tcaataatgc tttacctacg cttgcaagta gagcaaaagt tctaaccatg ctaccttata |
| 11621 | ctaatgaaga gaaaatgcag tttgtcaagt cctacaagaa ggtagatact tcaggaattg acgaccgagc |
| 11691 | gattgtagac tattgcaatc ttgccagcaa tcttcaaatg cttgaagaca tattagaata tggcgcagaa |
| 11761 | gagctatttg aaaaggttac aacattttat gacttaatat gggaggcaag tgctagcaat tcgctaaagg |
| 11831 | ttactaattg gctcaaattt aaggaaactg atgaaggaaa aattgagcct aaacttttcc tcaactgtct |
| 11901 | tttaaattgg tcgacagttg tcatcaggaa gcactatgta gaaatgtctt tcgaagaact tgaggcccat |
| 11971 | gaccttttag tgagggaagc atctaggtgt ttgcgaaagg tatctaaaaa gggctcaaat gcgcgtgtct |
| 12041 | gcgtgaacga atttatcagg agggtcaaac aagttgagtg atttagtatc atttcaaaaa gacattcgaa |
| 12111 | ccaataatct aaagccgttc tatatcttgt acggcgaaga aattggtctt atgaatgttt atctcaatca |
| 12181 | aatgggaaat gtagttcgag aaacttcggt ttcaacagtc tggaaaaccc tcactcaaaa agggctcgtt |
| 12251 | tctaatcatc gaatattcgc tgttcgagat gataaggagt ttctgtctaa tgagtcgagg tggaaaaggc |
| 12321 | ttccggatgt tagatatggg acacttgttt tgatggttac taaaattgac aagcgaagca agttgctaaa |
| 12391 | ggcctttcct gataattgtg ttgagtttga gaaaatgact gacgcgcagt tgaaaaggca ttttgtgtct |
| 12461 | aaatactcga ctattgatag cgacatgatt gacatggtta tccagttctg tctaaacgat tactctagaa |
| 12531 | ttgacaatga attggacaag ctgtcgcgat tgaaaaaggt tgacgcatca gtagttgaat ccattgtcaa |
| 12601 | gcacaagacc gaaattgaca ttttcagcct agttgatgat gtattggaat ataggccgga gcaggcaatt |
| 12671 | atgaaagtga ctgaactttt agccaaagga gaaagtccta ttggattgct taccttgctt tatcaaaatt |
| 12741 | ttaataacgc ttgtcttgtg ctaggagccg atgagcctaa agaagccaat ctaggcatta agcagttctt |
| 12811 | aatcaataag attgtctata actttcaata cgagctggac tcagcctttg aaggcatggc tattttaggt |
| 12881 | caagctatcg agggcataaa gaatggtcgc tatacagaaa gttcagtggt ctatatttct ttgtataaaa |
| 12951 | ttttttcact tacttaacaa ataagctgaa atctgtgtat attacagtat aagcaaagga ggacagccta |
| 13021 | tgacagaagt tgcggtaaat agcccgcaaa aggtgagagt agttatggtc gggaatattg aatttctcga |
| 13091 | atatttaaaa aggaagtacg gaacagaaac ttccatcagt tatattatag aaaatgaaag gggtctaata |
| 13161 | tgacagactt taaaaaacgc ttcaagaaag cagtaacaga aacaatcaat cgtgacggta tcgagaacct |
| 13231 | tatggattgg ctcgaaaatg ataccaattt cttctcaagt ccagcaagca ctcgatacca tggaagctat |
| 13301 | gaaggtggac ttgtcgagca ctcattaaac gtgttcaatc aactactttt cgaaatggat accatggtag |
| 13371 | gcaaaggctg ggaagacatt tacccaatgg aaacagttgc aatcgtagca ctatttcacg acctttgcaa |
| 13441 | agttggtcag tatcgtgaaa ctgaaaaatg gcgcaagaac agcgacggtg aatgggaaag ctatttagca |
| 13511 | tatgaatacg accctgagca acttacaatg ggacatggtg caaaatctaa tttccttctt caacgtttca |
| 13581 | ttcaactcac gccagttgaa gctcaagcaa ttttctggca tatgggagcc tatgatatta gtccttatgc |
| 13651 | aaatttgaat ggatgtggag cagccttcga aactaatcca cttgcattct taatccatcg cgcagatatg |
| 13721 | gccgcaactt atgtagtcga aaatgaaaac ttcgaatact ctcaaggtcc agttgaacaa gaggctgagg |
| 13791 | ttgaagaagt agttgaagaa aaacctaaga gttcaactcg taagaaacct gcgcctaagg aagaaaaagt |
| 13861 | tgaagaggct gaagaaaaac caaaagctgg aatcactcga cgtcgcaaac ctgcgccaaa agaggaagag |
| 13931 | gtagaagagc ctaaagaaga gcctaagaaa gcatcttcta aaattcgaat gcctaaaaag actgaaaagg |
| 14001 | tcgaagaggt agaaagcgca gacgagccga aagttgaaga agcagaggac gacaatgtgg tggtacctgc |
| 14071 | tggatatgtt cgagatgtct actacttcta cagtgaagtc gctgacgttt actacaagaa agatgtcgac |
| 14141 | gagcctgacg atgacagcga cattcttgta gacgaagaag agtacatgga cgcaatgtgt cctgtattag |
| 14211 | aagaagactt cttctacgaa cttgacggca aggttcacaa attagcaaaa ggtgaacgct tgccggaaga |
| 14281 | atacgacgaa gaaacttggg aacctatcac tgaagcagaa tacatcaagc gaacagaaaa acctaaagca |
| 14351 | gttgcaaaac ctactcgaaa aactccagcg ccttctcgtc gccctcgccc ttaaaagaaa ggttgaaata |
| 14421 | aaatgtgtga aaattgtcaa aacgaaacat tcaatactag aattttcaat gaagatgaaa gtggctatgt |
| 14491 | cgacgcctca ttcacttaca aggagattcg cgacaccgca gcagctatta gcaatcgagc ggtagaaaag |
| 14561 | aaagaccgtg acagcctttt agtcgctaca gttatggctc ttcccgtttc tcacgcagaa gatttaggca |
| 14631 | agagactttg tattgcaaat tctcgattgg aagcatttcg tgaagctgtt caagaggctc tcgagaatga |
| 14701 | aaaggctgaa gatttaaagg acgttatctt aggtcttatc gacgttgaca aaaaaattgg caaccttgca |
| 14771 | ttgcaattag ttgaatcagg agcattataa tggaacgaat aaagacgcta tttcacgtga tttatgctaa |
| 14841 | cggcactcat ttagaagtag cagctttgtt cgataccgtt gatgattatg atgacgttat agaggacatc |
| 14911 | caggggtata ttgatacccc tgacctttat aatcaaagga gcattagaat ggcgccttac aatcctgaca |
| 14981 | tcaatggtga cgctattgct actgacattt tactacgact agatgatatt atctacgtcg acgcaacttg |
| 15051 | tgaaactatt aaatacgagg agcctattgc atgaacaatc agcgaaagca aatgaacaaa cgaatcgtcg |
| 15121 | aacttcgcga agactatcaa cgtgcaagag gtcgaataaa cttccttctt gctgtaaagg accacggcga |
| 15191 | agaactcgaa aaccttgaag cctttgtggg atacattgac aatctagtcg aatgttttcc tgaaagccaa |
| 15261 | cgaaatgtct tgaggctatg tgtattagat gaccttccag tcactaatgc ggccgctgaa attggatacc |
| 15331 | actatacatg ggttcaccaa cttcgagaca aagcagttga aacacttgaa gaaattttag atggggataa |
| 15401 | cattattcgc tctaaacacg gaatcgaaat taaggagaaa cttgatgaat tatatggtaa aagtcattct |
| 15471 | agttagtgtc tttgtactgt cagccttttg catgacttgc tcaatggttt atttggttac aggtaagcaa |
| 15541 | gaggaccacc gtagtaccgt cgcccttgta tttggcgctc tcgtaagctc tgcggcgttc tattcgacac |
| 15611 | tctttatcct cgcctatctg ccatgacatc acgcgcatac aaaccaattc ccacgcgcag agctagtgct |
| 15681 | aaacaagaga aggcagttgc taagcagttg ggaggaaaag tacagcctaa ttcaggagcc actgactact |
| 15751 | acaaaggtga cgtcgtaaca gactcaatgc ttatagaatg caagacagtt atgaagccac aaagttcagt |
| 15821 | cagcttgaaa aaggaatggt tcctaaaaaa tgaacaggaa aggttcgctc aaaaactcga ctattctgct |
| 15891 | atcgctttcg actttggtga cggaggcgaa cagtatatag caatgtctat aagtcagttc aagcgaatat |
| 15961 | tagaggatag aaatgataac cttatttaaa ataaacagtg aaggaacagt tactccaatt aaagggtcag |
| 16031 | ccatgcaact gtacgcagac cttattccta tacaagagga cgatatacag ttcgttgata taactggact |
| 16101 | tgaccctatt gttcgagaaa acgtacttga gctcatttca cggagccgtg taggagtttc aaaatatggt |
| 16171 | acaaacctcg accagaatga tgtcgacgat ttcctacagc acgccaaaga agaagcgctc gactttgcta |
| 16241 | actacctaac caagctacaa agtcaacaaa agcaaaataa atagacctat ttctaggtct atttttatta |
| 16311 | ttgataaatt ccagcaattt gacgagcgca atcttctagc gcagatacta ggtggcggct ttcttgttta |
| 16381 | ccttgttcat ttcttgcttt aattctttcg ttaaggcgtt cgattcttgt agttaatttc ttgatgattt |
| 16451 | caattctagc atcaacttcc atgtcgcgag taagtgtgac tccagtttca gcgacaggac atgctttgaa |
| 16521 | tactgcaatg tcaagttcgc tctttctaat aactgagcct aggtctaagt acaagttagg attgattcca |
| 16591 | gtgaccttat attgtttctc agtttctttt acaggaatgc tttcatagtg gaaagtgtag ttcttgtgac |
| 16661 | cgtctttcca atctgctgta agataaccga aataaagtgt tgtttccata attgacctct ttctgcgtcc |
| 16731 | ttgacgcttg ttttatttat attatgatta tacgataata aaggaataaa gtcaagcact ttttacaaaa |
| 16801 | aagttgaact tttttaaata tttttttttg aaaataaaaa gccctaataa tagagctttt agtttagcag |
| 16871 | aaaattaagt tcatcttcat aagcaagaat ctgtccgtac tggtaagaaa tagctgattc aatatccggc |
| 16941 | atttcgtgga ctcctttttt aagttcgtcg atagtacagt tacaatgacc tattcttgac tgaagttcct |
| 17011 | caatcctttc gagtcgcttt tcattttgtg tatcaattgt tttcgagtct aggtgagtga aggaacttgc |
| 17081 | aatagtttga atggcttcaa aaaagtccgt tattgaaact cctttataag aaagctcatt ccgtgtatag |
| 17151 | caggaaagca aagcgttcca gctagtgatt tgaatttgag ggttaggaga gtttcgataa gctacaaaat |
| 17221 | ttagaatatc tttgtagtca atatcagctt cagtatgatt gttgataaat accttcattt tataaccctt |
| 17291 | ccaaatcttc gtcctcgtca tcgttttcat agcaggcgat aacttcaacc cactcgtcgt cctcaccttc |
| 17361 | gtttcgaact cgaatgctaa ggacttccat gtcctcaaca tcttcgaatc cttcattagg tgcatatcct |
| 17431 | tcccactcta aatcgtcgta gtcgaagata gttacaagac gtccgtcaaa ttttactgtt tcctttactg |
| 17501 | ttgccatttt agtttcctcc ttatgcgata tatagtttga taatttgaga ttcgatgtca ccatagttga |
| 17571 | tgaacttaac ttggtcgacc gtttcttcca tgtattcgcc catgtcttcg attcttccgt cttgaatcat |
| 17641 | ttggccgttt tcgttgataa tttcgtacca ccattcatca ccgaattgtt tgattgcttc tttaactgtt |
| 17711 | ttcattttac tacctccact ttttcgtcca ttagtgattc gttatcatag aaccgaatac gtccatcact |
| 17781 | aagacgttct aggcttaccc atttacgacc ttgacggtca gttactttaa attcagtacc ttttgcattt |
| 17851 | acaactttca ttcctacttg caaatcttta acttttacca ttttatatga ctcctttatt tgtttttctt |
| 17921 | tatagtatta ttatacgata atgagtgaat aaagtcaagt gtttttgtaa acttttttaa attttttaat |
| 17991 | tttttttttc aaaaaaataa cgagccgaag ctacgttatt tatttatctg ctcaagggct tgttgaattg |
| 18061 | cctcatagcc tttacgacgt gctacctttc cagctttaga gccgggtgaa aagtcccaaa cagtttcgtc |
| 18131 | tactttaaag tcatccgcct tggcatagtc gagcaggagc tggatagctt tttgccattt ccgccaattc |
| 18201 | ttggaaaact cacctatatt agcacaacgc aaaacaagtg ctctagtatg ctggctagac ataatgaact |
| 18271 | ctaaaaagtt gtccaaggtt ataggaaggt cctttggaaa ctcataaggc tctttgacat cgtatttgaa |
| 18341 | aaggctgaca atttcactgt ccttaaatag ttcaccgtct ttatacataa taccttgaac aatttcagta |
| 18411 | ggctctgctc cgctatctag tacatcgcca accgtgtgac aataggcttt aagaactgca aaaaaacctg |
| 18481 | gggcgtctgc acgcgcaacc tggagctcct taacagtcat ccaaggctga ggtttcttac aaacaatcct |
| 18551 | aattccttca aaatagctct tgtccgggtc aatagtgcct aacattgtca gcctgttttt atttatataa |
| 18621 | aggtcgaaat atacttgaat ttcatctgta ttaggcagcc acttaacagt gacttttcta taagcgattg |
| 18691 | cttttacatt tacttttttc gagagatttg tagggataag cattttcctt ttgacattta ctttttttcg |
| 18761 | ctttttgttc tttgccatgc tagtatctcc atttctgttg gtcttgcttt ttagctctgt tcagttcagc |
| 18831 | tgcttctcgc gatgcaatag tttcgagaat atgcctgttc ataggctcac aatattccgc caaagatttg |
| 18901 | ccagttatgg tggcgtcaat taagtaacca tctattgact ccttaccata aaatacaaaa tcgtcttggc |
| 18971 | atactagcct tttataatag ccatttcctg cgcgtgtttc aattttaact aagctcattt tcacccaaac |
| 19041 | ttgtagacga taaggagttc ctggaacttc gaacaggagc ctcctttttt catcgtctac ttgtttaata |
| 19111 | catgagtttt gaaaatggat aactttccat ttattttcca tagtttcacc ttattccatg tacccgtcaa |
| 19181 | caatccataa ttgaaaaggc ttatcttctc tataaggccg tgataatttt agtccagttc ccactacatt |
| 19251 | tgaaagcgcg attaggtcat ctaggctgtc tagctcgagt tcgattacaa ggttgccagt atcaatttca |
| 19321 | caaaagtaag cgacatttcc aactttctct agtgcttcac gatacctatc atatgtcgcc tcttcgtcaa |
| 19391 | atagtcgcgc agaataaact tcgaatttca ttttagttac cgccttccaa aatttcatcg ggcataatct |
| 19461 | ttgcattctc gccatgaaac cgcccttcaa tatacgcttc aagattgaag tcatgttgag gtctgtcaat |
| 19531 | tccttccttc tttaaatttc gaaatgtgtc ctgaagcgca ttttttgttt gctcgctagg taggaccata |
| 19601 | agtgaatatt cttccacctg ctttttaaat cgaatggcta aggctgacaa aaagcctttg aggtatgaat |
| 19671 | tcttgtagga aggttcgcga gtaggaagtc ggtcaatacg gtaacgaaga taaagcaaag cagcctcata |
| 19741 | tattttagac actaattcag cgtcttgttt ttcgccgaag aaaattattc gacttttatt caagcgcata |
| 19811 | tcacgctgat taatacaaaa gcacctaaaa ttagtcgcga gaatatgacc aagttcacgt tcccaccaaa |
| 19881 | atattcgacc tgcttctttc ccaacagctt gagaagtctc gaactgttta ggttcatcaa attgttcaac |
| 19951 | ttgagcaagt gcgatattat tctttagcat caacttttga gccataagaa gggcagtttg cccctcttcg |
| 20021 | tcactcgggt tgtcatttgc taattgaata agatttttaa ttttttcaat aattttttcg ttattcatat |
| 20091 | tagtcacttt ctatcatatt ttcgagcttt cgaaaagtca atgtcgtcta cttcaattgt cttgtcataa |
| 20161 | gtccaagcgc gacaagtgtc gaaatgaaat aggctacaaa acatcttttc attatggtcg aaactttcag |
| 20231 | tacatttttc aatatctact tcaagttcga gaacgacaat agtatcaaca tttcgaagcg ataaaaaggc |
| 20301 | tagagccttt tcataacttt ctgctaggta aataactcca gctgaaggct tcaatccttc agctagaatt |
| 20371 | ttaccaagat tatcaaaatc agtggcgtga taaagtttca ttagttactt ccttacatat ctagagtcac |
| 20441 | tacataaata gaagcagttt tatcttccaa gtcctactca atagcttcct cttcgctgag tttttcgagt |
| 20511 | tttaaaactg tcgcttcagc tacaacatta gcaaagttcg aaccgttgag aatgttttcg atatttcctg |
| 20581 | cgcctaagac ttcagcttgg tcattgttca ctaccattag gtattcatta gtaagtgctt tagcaaagtt |
| 20651 | tgaaaatttc attttatttt ccctttattt gtttttcttt atactattat tatacaataa tgattgaata |
| 20721 | aagtaaagca ttttttataa aaaagttgaa ctttttttac aattttttga actatttaaa aattataaaa |
| 20791 | tgggtggaaa atttaggcga caatttatac ccattttcaa cctcatttat aaacaatcta atatagaaaa |
| 20861 | ggacttaata agtaaataaa aaagcgccct gaaaatacct acaaatccca tagtccgtaa gtaaaaacaa |
| 20931 | aaattagggg cgacataaaa gtcgagcact atcttaatct attaccagtc tcatatacaa tcgacacaga |
| 21001 | tttagcaggc ttttagcaaa ctttcgaaca gcatgaaaaa gcatacaatt agaggaacag attatagaaa |
| 21071 | aagcacttcc acaaacaagt tctcaaaatg ctctcaaaaa ccgtaaaatt agtaagtttg aacttttcga |
| 21141 | acttctaaac ttttcgaata atcgagccta atttagaggt cgaaaaactc aatttctcga aaagtcgaac |
| 21211 | ctgctcgaaa acctcaaaac actcgaaaag tcgagcatag aaaggggtcg aaaagtcgag aatgctcgaa |
| 21281 | aaactcaacc ggttcgaaaa cctcaatcct tcgaaaagtc gaaccattcg aaaagttcaa aagttcgaaa |
| 21351 | aactcaacca ttcgagagta ggaattaagg acataccagt tcaacctttt tagcttcaaa atcactcttt |
| 21421 | ttctcattat aggactataa attcagtcaa ttgtaagtca cgcgcaaatt tgttacaatg taaacgataa |
| 21491 | aatataaagg agggtcaata aatggcgaaa gctactggac caaaagttcg aagaggaaaa actcctccac |
| 21561 | ggccaaaaga caaaaaagga atcaaagcaa atgcgcgtgt caataaagac cagttcgtag agtatgacta |
| 21631 | taaaggcatc aagatgacaa ttaaggaacg tgatgctaga atgaaattgg aatttattag aggcatgact |
| 21701 | attcaggaaa ttgcagcccg ctatggatta aatgaaaagc gtgttggcga aatacgggct cgcgataaat |
| 21771 | gggtgaaggc taagaaagag ttcgagaatg aaaaggctct tgttactaat gatacattga ctcaaatgta |
| 21841 | tgcagggttt aaagtctcag tcaatattaa atatcacgcc gcctgggaga aactaatgaa catcgtcgaa |
| 21911 | atgtgtttag ataatcctga cagatattta tttactaaag aaggaaatat tagatggggc gcattagatg |
| 21981 | tcctttcgaa ccttatagat agagctcaaa aaggacaaga aagagcgaat ggaatgcttc cggaagaggt |
| 22051 | tcgatataga ctacaaattg agcgcgagaa aattacattg ctccgggcca aaatgggcga ccaggaaatt |
| 22121 | gaaggcgagg ttaaagataa cttcgtagaa gcactagata aagcagctca agccgtttgg caagaattta |
| 22191 | gtgacgcaac aggttcctac attaaaggag tgactgataa tgacaataag cctgagaaat aaactaccta |
| 22261 | agttcaactt cgtccctttt agtaagaaac aactccagct cctaacatgg tggacaaagg gctcaccttt |
| 22331 | tcgaactttc gatatcgtca tagcagacgg ttccattcgt tcaggaaaaa cagtatcgat ggctctttca |
| 22401 | ttttcccttt gggccatgac ggaattcaac ggacaaaact ttgccatctg tggtaagaca attcactcag |
| 22471 | ctcgacgaaa tgttattcag cctctaaagc aaatgctcac aagtcgcggg tatgaaattc gagatgttcg |
| 22541 | aaatgaaaat ctacttatta ttagacactt tagaaatggc gaagaaattg tcaactactt ctatatattt |
| 22611 | ggaggaaaag atgagtcgag tcaagacctt atacaggggg taacattagc aggtatcttc tgtgatgagg |
| 22681 | tggcactgat gcctgaatcg tttgtcaacc aagcgacagg gcgctgttcc gtaacaggtt cgaaaatgtg |
| 22751 | gttctcttgt aacccggcca atcctaatca ctacttcaag aagaactgga ttgacaaaca ggtcgaaaag |
| 22821 | cgtatcttat atcttcactt tacaatggac gacaacccta gcttgacgga tagcattaaa aggcgctatg |
| 22891 | agaaaatgta tgctggagtc ttcaggaaaa gatttattct cggcctttgg gtaacagcag atggtctagt |
| 22961 | ttattcaatg ttcaatgaag agcagcatgt caaaaagctc aatatagaat tcgaccgttt attcgtagca |
| 23031 | ggcgactttg gtatctataa tgcaacaacc ttcggccttt atggattctc gaaacgtcat aagcgctacc |
| 23101 | atctaattga gtcatactac cactcagggc gcgaggcgga agagcaacta actgaggcgg atgttaattc |
| 23171 | gaatattcaa tttagttcag ttctacaaaa gactactaaa gagtacgcaa atgatttagt cgatatgata |
| 23241 | cgaggaaagc aaatcgaata tataattctc gacccgtctg cttctgctat gattgttgaa cttcaaaagc |
| 23311 | atccttatat agctagaaag aatatcccta tcattcctgc tcgaaatgac gtgacgcttg gcatttcatt |
| 23381 | tcacgctgaa ctcttggctg agaatagatt tacactcgac cctagcaaca cgcacgacat tgatgaatac |
| 23451 | tatgcttaca gctgggacag taaagcgagc caaacgggag aagatagagt cattaaagag catgaccact |
| 23521 | gcatggatag gaacagatat gcctgtctca ctgacgctct aatcaacgat gacttcggtt tcgaaataca |
| 23591 | aatattatcc ggaaaaggcg ctagaaacta actaaacact tttatagaaa ttagtgtata atataagtag |
| 23661 | gaggatttta aacatggcta aaaaatcaaa agctatctca cacacagacg aactgattag tcagtcgttt |
| 23731 | gacagcccct tggcaaagaa tcaaaagttc aagaaagagc ttcaggaagt tgaaaagtat tatcaatact |
| 23801 | tcgacggatt tgatgtcacg gacttgaata ctgactatgg gcaaacatgg aagattgacg aagactcagt |
| 23871 | cgactataaa cctactcgag aaattcgaaa ctatattcga caacttatca aaaagcaatc acgctttatg |
| 23941 | atgggtaaag agccagagct tatctttagt ccagttcaag acaatcaaga tgaacaggct gagaacaagc |
| 24011 | gtattctatt cgactctatt ttaaggaatt gtaaattctg gagcaaaagt acaaatgcat tagtcgacgc |
| 24081 | cacagtaggt aagcgggtat tgatgacagt agtagcaaat gccgctcaac aaattgacgt ccagttttat |
| 24151 | tcaatgcctc agttcaccta tacagttgac cctagaaacc cttccagctt gctttctgtt gacattgttt |
| 24221 | atcaggacga gcgtacaaaa ggaatgagca ctgaaaaaca actttggcat cattatagat atgaaatgaa |
| 24291 | agctggaaca agtcaatcag gaattgcaac agctttagaa gacattgaag aacaatgttg gctcacttat |
| 24361 | gccttaacgg atggagagtc gaaccaaatc tatatgacag aaagtggcca aactactatc aaggagacag |
| 24431 | aggctaaact tgtagaaatt gaagacaacc taggaaacaa gattgaagtt cctttaaaag ttcaagaatc |
| 24501 | cgccccaacc ggcttgaagc aaattccttg tcgagttatt cttaatgaac cattgactaa tgacatatac |
| 24571 | gggacaagcg atgtcaaaga ccttatcaca gtagcagata acttgaacaa aactattagt gacttacgag |
| 24641 | attcacttcg atttaaaatg ttcgagcagc ctgttatcat tgatggctct tctaagtcaa ttcaaggaat |
| 24711 | gaagattgcg ccaaacgctt tggtcgacct taagagtgac cctacttcct caatcggcgg tactggaggc |
| 24781 | aagcaagctc aagtcacttc catttcagga aacttcaact tccttccagc ggctgaatat tatttagagg |
| 24851 | gcgctaagaa agccatgtat gaactaatgg accagccaat gcctgaaaag gtacaggagg cgccatcagg |
| 24921 | aattgcaatg cagttcttat tctacgacct aatttctcga tgtgacggaa aatggattga gtgggatgat |
| 24991 | gctattcaat ggctcattca aatgctggaa gaaattttag caacagtgaa tgttgacttg ggaaatattc |
| 25061 | ctcaagatat tcaatcaagt tatcaaacac ttacgacaat gactatcgaa caccactatc caattcctag |
| 25131 | cgatgaactt tctgctaagc aacttgcgct cactgaagtt caaactaatg tacgcagcca ccaatcttac |
| 25201 | attgaagaat tcagtaagaa ggaaaaggcg gacaaggaat gggaacgcat tttggaagaa cttgctcagc |
| 25271 | ttgacgaaat ctcagctgga gcattgcctg tattagcaaa cgaattaaac gaacaagagg agcctcaaga |
| 25341 | tgaaacgagt gaagaagacg aagttgatga caaagaaaaa gaacaaactg aacaaccaac cgaagaagga |
| 25411 | gtcgacccag acgttcaagg ttaattgtga ccattgtgag cataagttcg accttacatc taaacagatt |
| 25481 | atttcgaaac atatcgaaaa gggcgtagag tggagattct tcgaatgtcc taagtgccat tatcggttca |
| 25551 | ccacttatgt aggaaacaag gaaattgaaa accttattcg atttagaaat acttgtcgag ctaaaatgaa |
| 25621 | gcaggaactt caaaaaggag ctgctgctaa tcaaaacact taccattcat atcgaattca ggatgagcaa |
| 25691 | gctgggcata aaatctcagg gcttatggcg aagctaaaga aggagataaa cattgaaaaa cgagaaaaag |
| 25761 | aatgggtatc tatatagctg ggaaaaggct attcatgaaa ataatattcg tctaaccctt gaacaggaac |
| 25831 | aagctgtact gaaagccttc agcgatgcag gaactgattt aattgcaaag attaaaaagt ctcgaaatgg |
| 25901 | atacttgcct aaaagaatct ataaagacta cgcttacgac ctgcacgctg ttcttgttca actaatgact |
| 25971 | gaatactctc ataaggcggc aatgaacgca gtagatggcc aggtagttca tattctacaa gtattagcag |
| 26041 | aagatggaaa tgctacggct gaaaagttcg aaaaggaagt cagggctgca tctttagtat tttcacgaag |
| 26111 | agcagccgag gcagttgtca aaggtgaaat ctataaggac ggcaaaaacc tctcgaaacg tgtttggtct |
| 26181 | tcagccgcac gcgcaggaaa tgatgttcaa caaatagtca cacaaggcct agcaagtgga atgtctgcta |
| 26251 | cagatatggc taaaatgctc gagaaatata tcgaccctaa ggttcgaaaa gattgggact ttgataagat |
| 26321 | agctgagaag ctagggaaac ctgctgctca taaatatcaa aatctcgaat acaatgccct tcgacttgct |
| 26391 | cgaactacca ttagccattc cgccacagct ggagtgagac aatggggcaa ggttaatcct tatgctcgaa |
| 26461 | aagttcaatg gcattctgtt cacgctccag gtcgaacgtg tcaagcgtgt atcgatttag atggtgaagt |
| 26531 | atttcctatc gaagaatgtc ctttcgacca tcctaatgga atgtgctacc aaactgtatg gtacgaaaac |
| 26601 | tcactcgaag aaatcgctga tgagttgaga ggctgggtag acggagaacc taatgatgta ttagacgaat |
| 26671 | ggtacgacga tttaagttca ggaaaagttg agaaatacag cgacctcgac tttgttaaaa gttattaggc |
| 26741 | tcggttcaat accgagtctt tttgtctata aattgtctaa tttcgagaac cttcgaaaag tagtaaaatg |
| 26811 | atattcagtt atgttataat ataagttgaa aaggaacctt gtcgccttaa tgactcgaaa ttggtttcac |
| 26881 | tgttccaatt aaataaaaac agcagattca gccggagggc ggaaaactca ggaggaaaat aaatggctta |
| 26951 | tcaattagaa gacttgttaa aaggtctaga tgaaccaact atcaaacagg tgaaggaaat tatttcgaaa |
| 27021 | acttcgaaag aactcgatgc taaaattttc attgacggcg acggtcaaca ttttgtacct cacgcacgtt |
| 27091 | tcgatgaagt tgttcaacag cgcgatgcag ctaacggctc aattaattct tataaagaac aagtcgcgac |
| 27161 | gctttctaaa caggtcaaag ataacggtga tgcgcagacc actatccaaa accttcaaga gcaactcgac |
| 27231 | aagcagtctc aacttgcaaa aggcgctgtg attacttcag ctcttcatcc gttgattagt gactccattg |
| 27301 | ctccagcagc agacattctt ggatttatga accttgacaa cattacggtc gaaagtgacg gtaaagttaa |
| 27371 | aggtcttgat gaagagttga aagctgttcg tgagtctcgt aaatacttat tcaaagaagt cgaagttccc |
| 27441 | gcagaacaag aggctcaagc taagtcgcca gccgggactg gaaatttagg aaatccaggt cgtgtcggtg |
| 27511 | gtggtgttcc cgaacctcgt gaaatcggct cttttggtaa gcaacttgct gctgctcaac aaacggcagg |
| 27581 | agcacaagaa caatcatcat tctttaaata ataggaggaa ctaactatgc ctaatgtgcg agttaagaaa |
| 27651 | actgatttta atcaaaccac tcgaagcatt gtcgcaattc ctgaccacta cgttgctttg gctgctcaaa |
| 27721 | ttccagctac cgcagcaact caagtaggga acaagaaata cattcttgcc ggaacttgcg tgaaaaatgc |
| 27791 | tactacattt gaaggacgca aaactggact cgaagtagta tctaccggtg aacaattcga cggagttatc |
| 27861 | ttcgctgacc aagaagtgtt tgaaggtgaa gaaaaagtaa ccgtgacagt attagttcac ggattcgtca |
| 27931 | aatatgcagc ccttcgaaaa gttggcgatg ctgtgcctga atctaaaaac gcaatgattc ttgtcgttaa |
| 28001 | ataggaggaa ttatagatga atatttatga ttatatcaac gcaggggaga ttgctagcta cattcaagca |
| 28071 | cttccttcaa acgctcttca ataccttgga ccaactcttt tccctaatgc tcaacaaaca gggacagaca |
| 28141 | tttcatggct caagggtgca aataatttgc cagtaactat ccagccatct aactacgacg cgaaagcaag |
| 28211 | tcttcgtgaa cgtgctggat ttagcaaaca agctactgag atggcattct tccgtgagtc tatgcgactt |
| 28281 | ggtgaaaaag accgtcaaaa cttgcaaatg ctattgaacc aaagttcagc tcttgcccaa ccacttatca |
| 28351 | ctcaactcta taatgatact aagaaccttg tagacggtgt tgaagcgcaa gcagaataca tgcgtatgca |
| 28421 | attgcttcaa tacggtaaat tcactgtcaa atcaactaac agcgaggctc aatacactta cgactacaac |
| 28491 | atggatgcta agcaacaata tgcagtcact aagaaatgga ctaacccagc tgaaagtgac cctatcgctg |
| 28561 | acattttagc agcaatggat gacatcgaaa atcgtacagg tgttcgccct actcgaatgg tcttgaaccg |
| 28631 | aaacacttat aaccaaatga ctaagagtga ctctatcaag aaagctcttg caattggtgt tcaaggttct |
| 28701 | tgggaaaact tcttgcttct tgcaagtgac gctgagaaat tcatcgctga aaaaacaggt cttcaaatcg |
| 28771 | ctgtctactc taagaaaatt gctcagttcg ctgacgctga caaacttcct gacgttggta acattcgtca |
| 28841 | gttcaacttg attgacgacg gtaaagtggt attgcttcca cctgacgcag ttggtcacac ttggtacggt |
| 28911 | actactccag aagcattcga cttggcttca ggcggaacag acgctcaagt tcaagttctt tcaggcggac |
| 28981 | ctaccgttac aacttatctt gaaaaacatc ctgtcaacat tgcaacagtt gtatcagctg ttatgattcc |
| 29051 | atcattcgaa ggaattgact atgtaggagt tctcacaact aattaggagg tcgctatatg gctacattga |
| 29121 | aagctcttag caccttaatc gtttccggag cagtagtgca ttcagggtcg gtattttctt gccctgaagc |
| 29191 | gcttgcttcg tctttaattg aacgcaattt tgcgttcgag attaaggcgg ctgaagatgg agaaacggta |
| 29261 | gaaactgttc ctcaaacaat tgaatcagtt gaagaaattg acgaagttga acaaatgcgc gaagagtatg |
| 29331 | cggctaaaac cgttcctgag ctcgttgaat tagcaagagc taatggaatt gacatttctt caatttctcg |
| 29401 | aaaaagcgaa tatatcgacg ctttaattaa gtacgaacta ggagagtaaa atggcagctc aaacggacat |
| 29471 | tgaattagtc aaaatcaata tcgataacga taattctccg tcaccaatga ctgaccaaag tatctcagct |
| 29541 | cttttagaca agcataaatc tgtcgcctat gttagttata tgatttgctt aatgaagacc cggaatgacg |
| 29611 | tggtaaccct tggacctatc agtctaaaag gtgacgcaga ctactggaaa caaatggcgc aattctatta |
| 29681 | tgaccaatat aagcaagaac agcttgaaac tgatgaaaag tcgaacgctg gttcgacaat cttaatgaaa |
| 29751 | agggctgatg ggacatgagt tatgacgtga attatgttaa gaatcaagtt cgtagagcca ttgaaaccgc |
| 29821 | tcctactaaa atcaaggtac ttcgaaactc ttgggtcagt gatggatatg gaggaaagaa aaaggataaa |
| 29891 | gcgaatgaag tcgtagcaga cgaccttgtt tgtttagttg ataattcaac tgttcctgac cttttagcca |
| 29961 | attctactga cgcgggaaaa atttttgccc aaaatggagt gaaaattttc attctatatg atgaaggcaa |
| 30031 | aatcattcaa cgagccgata ctatcgaaat taaaaactca ggaagacggt acagggtagt agaaacccac |
| 30101 | aatcttctcg agcaagacat tttgatagaa cttaaattgg aggtgaacga ctaatgtctc agcctgaatt |
| 30171 | agtatggaag cctgaagaat ttgttagtaa ctgtgaacgg tatcgaaaca agtttcaagt cgctgtcata |
| 30241 | acagtctgcg aagtcgctgc tactaagatg gaagaatacg caaagacgca tgctatttgg acagaccgta |
| 30311 | cagggaatgc tcgacagaaa ctcaaaggag aagctgcttg ggtaagcgca gaccaaatca tgatagctgt |
| 30381 | atcacatcac atggactacg ggttttggct agaactagct catggtcgaa aatacaaaat tctcgaacag |
| 30451 | gctgtagaag acaatgtcga agaacttttt agagcgttga gaaggttatt agactaggag tgaacatgac |
| 30521 | taaacgaacg acaatgatgg acagattgaa ggaaattctt cctacatttc agctctcgcc tgctcctatg |
| 30591 | cttccaggag ttgaatttga cgagcaagat acagataggc cggatgacta cattgttctt cgatatagtc |
| 30661 | atagaatgcc cagcgcaaca aatagcctag gaagttttgc ttattggaaa gttcaaatct acgtccattc |
| 30731 | aaactcaatt attggtatcg acgaatatag cagaaaggtt cgaaacatta tcaaggacat gggctacgaa |
| 30801 | gtaacctatg cagaaactgg tgactacttc gacacaatgc tttctagata ccgactagaa atcgaatata |
| 30871 | gaattccaca aggaggaaac taataatgag taaagacatt ctttacggaa tcaagctcgt gcaaatcgag |
| 30941 | gagcttgacc cattgactca gttgccaaaa gtcggcggag ctaactttgt cgtagatacg gcagaaacag |
| 31011 | cagaactcga agccgtgacc tcggagggaa ctgaagatgt gaaacgcaat gacacgcgca ttcttgctat |
| 31081 | cgtgcgtact ccagaccttt tatacggtta tgacttaaca ttcaaggaca acacgtttga ccctgaaatc |
| 31151 | atggccctaa ttgaaggtgg tacagtacgt caacaaggcg gaactattgc tggatacgac accccaatgc |
| 31221 | ttgcacaagg tgcttctaat atgaaaccat ttagaatgaa catctatgtg ccaaactatg taggtgactc |
| 31291 | aattgtcaac tacgtgaaaa tcactttgaa taactgtacc ggtaaagctc cagggctttc aatcgggaaa |
| 31361 | gagttctacg ctcctgagtt caacatcaag gcacgtgaag caaccaaagc aggtttgcca gttaagtcaa |
| 31431 | tggactatgt ggcacaactt ccagcggttc ttcgtcgcgt gacattcgat ttgaacggtg gaacaggaac |
| 31501 | cgccgacgca gttcgagttg aagcaggtaa gaagatttct ccaaaaccag ttgaccctac cttaacaggt |
| 31571 | aaggctttca aaggctggaa agttgaagga gaatcaacta tttgggactt cgacaaccac atgatgcctg |
| 31641 | accgagacgt caaactcgta gcacaatttg catagaaatt tagaaagaag ggtctgttat gactaatatt |
| 31711 | atcacagctg agcagtttaa gcaacttgca tttcaaatca tcgcacttcc aggattttca aaaggtagtg |
| 31781 | aacctatcca tgttaaaatt cgagcagcag gtgtcatgaa cctaatcgct aacgggaaaa tccctaatac |
| 31851 | gcttttaggt aaagtgacag aactgtttgg agaaacttcg acagtcacta aagacaatgc tagtctagca |
| 31921 | tcaattactg accaacagaa gaaagaagcg ctcgaccgat tgaacaaaac cgataccggt attcaagaca |
| 31991 | tggctgaact tcttcgagta ttcgcagaag cttcaatggt agagcctact tacgctgaag tcggcgagta |
| 32061 | tatgacagat gagcaactta tgacaatctt cagtgcaatg tacggtgaag tgactcaagc tgaaaccttt |
| 32131 | cgtacagacg aaggaaatgt ctaatgtcat agcagtcgct actgaatttc atattagacc tagcgaggtg |
| 32201 | gtcgggatgc aaactgattt aggcaaatac tgcttcgacg cagcagccgt tgcttatatt agatatttgc |
| 32271 | aggaagacaa gactcctagg tatcctggtg acgaaaagaa aaatccagga ttgcaaatgc ttatggagtg |
| 32341 | actattttca gtcgctcctc tttttgtata tagaaaggaa attacatgga ttttgggtca attgcagcaa |
| 32411 | aaatgacttt ggatatctca aacttcacaa gtcaattaaa tcttgctcaa agtcaagcgc aacggctcgc |
| 32481 | actagagtct tcgaagtcct ttcaaattgg ttctgcttta acaggattag ggaaaggact tacgactgcg |
| 32551 | gttacccttc ctcttatggg atttgcagcc gcctctatta aagtagggaa tgaattccaa gctcaaatgt |
| 32621 | cccgtgttca agctattgca ggagcgacag cggaagagct tggtagaatg aagactcaag caatcgacct |
| 32691 | tggtgctaaa actgctttta gtgcaaaaga ggcggctcaa ggtatggaaa atctagcttc agccggtttc |
| 32761 | caggtaaatg aaatcatgga cgctatgcca ggggtacttg acctggctgc cgtatctgga ggagatgtgg |
| 32831 | ccgcgagctc cgaggccatg gctagttcac ttcgagcctt tggattagag gcaaaccagg cgggtcacgt |
| 32901 | ggctgacgta tttgctcgag cagcagctga tacgaacgca gaaactagcg acatggcaga ggcgatgaaa |
| 32971 | tacgtcgcac ccgttgctca ctctatgggc ttgagccttg aagaaacggc tgcgtctatt gggattatgg |
| 33041 | ccgacgccgg tattaagggc tcgcaagccg gaaccacgct tagaggcgct ctctcgcgta ttgccaaacc |
| 33111 | tacgaaagcg atggtcaaat caatgcagga attaggagtt tcgttctacg acgcgaacgg aaacatgatt |
| 33181 | ccactaagag aacaaatcgc tcaactgaaa acagctactg caggactaac acaagaggaa cgaaatcgtc |
| 33251 | accttgttac cttgtatggc caaaactcgt tgtcaggtat gcttgcacta ttagacgcag gtcctgagaa |
| 33321 | attggataag atgaccaatg ctctcgtgaa ctcggacgga gctgctaagg aaatggcaga aactatgcag |
| 33391 | gacaaccttg ctagtaaaat cgagcaaatg ggaggagctt tcgagtctgt tgctattatt gttcaacaaa |
| 33461 | tccttgagcc tgcacttgct aaaatcgtgg gagcaatcac aaaagttctc gaagcattcg taaatatgtc |
| 33531 | acctatcggt caaaagatgg ttgtcatatt cgcaggaatg gttgcagccc ttggaccact gcttctaatt |
| 33601 | gcaggaatgg tgatgacaac tattgtcaag ttaagaattg ctattcagtt tttaggtcca gcatttatgg |
| 33671 | gaacgatggg aaccattgca ggagttatag caatattcta tgctctggtc gccgtgttca tgatagccta |
| 33741 | cacaaaatcg gagagattta gaaactttat caacagtctt gcgcctgcta ttaaagctgg gtttggagga |
| 33811 | gcgttggaat ggctacttcc acgactgaaa gagttaggag aatggttaca gaaggcaggc gagaaggcga |
| 33881 | aagagttcgg tcagtctgta gggtctaaag tgtcaaaact gctcgaacag tttggaataa gtatcggtca |
| 33951 | ggcaggaggc tcgattggtc agttcattgg aaatgttctc gaaaggctag gaggcgcatt tggaaaagta |
| 34021 | ggaggagtca tttcaattgc tgtttcactt gtaacaaaat tcggtctcgc atttctaggg attacaggac |
| 34091 | cactcgggat tgctattagt ctgttagttt catttttgac agcttgggct agaacaggtg agttcaacgc |
| 34161 | agacggaatt actcaagtat tcgaaaactt gacaaacaca attcagtcga cggctgattt catctctcaa |
| 34231 | taccttccag tctttgtcga aaaaggaact caaattttag ttaagattat tgaaggaatt gcatctgctg |
| 34301 | ttcctcaagt agttgaagtg atttcacaag tcattgaaaa tattgtgatg acaatttcga cagttatgcc |
| 34371 | tcaattagtc gaagcaggaa ttaagatact cgaagcgctt ataaatggtc ttgttcaatc tcttcctact |
| 34441 | atcattcaag cagctgttca aattatcact gctttattca atggtcttgt tcaggcactt cctacgctta |
| 34511 | ttcaagcagg tcttcaaatt ttgtcagctc tcataaacgg actagttcaa gcgcttccgg caattattca |
| 34581 | agcagctgtt caaattatca tgtcgcttgt tcaagcacta attgaaaact tgcctatgat aatcgaagca |
| 34651 | gcgatgcaga ttataatggg tctagtcaac gcactgattg aaaatatagg acctatctta gaagcaggga |
| 34721 | ttcaaattct aatggcttta atcgagggac ttattcaagt gcttcctgaa ctaattacag cagcgattca |
| 34791 | aatcattact tcactattag aagcaatctt gtcgaacctt cctcaacttc tagaagccgg agttaaattg |
| 34861 | cttttatcac ttcttcaagg gttgctaaat atgcttcctc aactaattgc aggggctttg caaatcatga |
| 34931 | tggcacttct taaagcagtt atcgacttcg tccctaaact tcttcaagca ggtgttcaac ttcttaaggc |
| 35001 | attgattcaa ggtattgctt cacttctcgg ctcactttta tcgacagctg gaaacatgct ttcatcatta |
| 35071 | gttagcaaga ttgctagctt tgtgggacag atggtttcag gaggtgcgaa cctgattcga aacttcatta |
| 35141 | gtggtattgg gtcaatgatt ggttcagctg tctctaaaat tggcagcatg ggaacttcaa ttgtttctaa |
| 35211 | ggttactgga ttcgctggac aaatggtaag cgcaggggtc aaccttgttc gaggatttat caatggtatc |
| 35281 | agttccatgg taagttctgc ggtaagtgcg gcggctaata tggctagcag tgcattaaat gccgttaagg |
| 35351 | gattcttagg tattcactct ccttcacgtg tcatggagca gatgggtatc tatacgggtc aagggttcgt |
| 35421 | aaatggtatt ggtaacatga ttcgaactac acgtgacaag gctaaagaaa tggctgaaac tgttactgaa |
| 35491 | gctctcagcg acgtgaagat ggatattcaa gaaaatggag ttatagaaaa ggttaaatca gtttacgaaa |
| 35561 | agatggctga ccaacttcct gaaactcttc cagctcctga tttcgaagat gttcgtaaag cagccggttc |
| 35631 | gcctcgagtg gacttgttca atacaggaag tgacaaccct aaccaacctc agtcacaatc taaaaacaat |
| 35701 | caaggcgagc aaaccgttgt caacattgga acaatcgtag ttcgaaacaa tgacgacgtt gacaaactgt |
| 35771 | cgagaggatt gtataataga agtaaagaaa ctctatcagg gtttggtaac attgtaacac cgtaaaggag |
| 35841 | aaatagatgg ctagcagaca gacgctattg gtcgacggaa ttgaccttgt cgacaaaggt gcaaccgtgc |
| 35911 | tagaatatgt aggactcact ttcgcaggat ttaaggactc aggatttaaa aaccctgaag gcatagacgg |
| 35981 | agtattagat tctccgtcta atgctatgtc cgctcttact ggaagcgtga ccttaatgtt ccacggagaa |
| 36051 | accgaaaagc aagttaatca aaaatacagg cagttcaaac aatttattcg ctcgaagtca ttttggagaa |
| 36121 | tttcgacact tgaagaccct ggatactatc gaacgggaaa atttttagga gaaaccgagc aaggaaaact |
| 36191 | tgtagacgtt caagccttta aagatacttc ccttgtagtt aaattaggga ttcagttcaa agatgcttac |
| 36261 | gagtacagcg actcaactgt tcgaaaggtt tataagtttc aacccgcttt gggaggcgat agcttaccta |
| 36331 | acccaggaag acctactcga caatttagag tagaaataag aactacttct caaatcaaag gatattttcg |
| 36401 | aattggcgaa aaaagttcag gacagtttgt tgagttcggt actaattcag tattgatgga aagtggctcg |
| 36471 | attattattc taaatcttgg aacttttgaa cttattaaaa ttagcagtgc aaatcaagcg actaacttat |
| 36541 | ttagatacat taaacgaggc gcattcttca agattcctaa tggaaattca acaattacca ttgaataccg |
| 36611 | agccgatgac gcagcagctt ggacctctac tcttcccgct caagttgaac tgtttctaaa tccgtcttac |
| 36681 | tattagaaag ggaatatatg attgacaata atttacctat gagtccaatt cctggcgaaa ttgttcaagt |
| 36751 | atatgaccaa aacttcaatc taattggagc aagtgatgaa atctttagca agcattacga agacgaaatt |
| 36821 | gtgactcgag ctcgaggaaa agaaactttc acttttgaaa gtattgaaac ctcatctatc tatcaacact |
| 36891 | taaaggttga aaacattatc cagtatggag gaagatggtt tcgaattaaa tatgctcagg acgtagaaga |
| 36961 | tgtcaaaggg cttaccaagt ttacctgcta cgcattatgg tatgaactag cagaaggctt gcctaggaag |
| 37031 | ttgaaacacg ttgcttcttc tgtaggcgct gtcgcgctag atattatcaa agacgcaggt gaatgggttc |
| 37101 | gactagtttg tcctcctgac ggtgctaaca aacaagttcg aagcataaca gccgcagaaa attcaatgct |
| 37171 | ttggcatctt cgatatcttg caaagcaata caatttagaa ttgacatttg gttatgaaga aattatcaag |
| 37241 | caagaggtta gaattgttca aaccgttgta tttcttcagc cttatgtcga gtctaaagta gactttcctc |
| 37311 | ttgtagttga agagaatttg aaatatgtca ctaggcagga agattctcga aacctgtgta cggcttacaa |
| 37381 | gttgacaggt aaaaaggaag aaggcagtca agagccttta acgtttgctt ctatcaacaa tggaagtgaa |
| 37451 | tatctcattg atgtttcgtg gtttactaca cgccacatga agcctcgata tattgctaaa tctaaaagcg |
| 37521 | acgaacattt tagaattaaa gaaaatttga tgagtgctgc gcgtgcttat cttgacatct acagtcgccc |
| 37591 | actaattgga tatgaggctt cagcggtcct ttataacaag gttcctgact tgcatcatac tcaactaatt |
| 37661 | gtcgacgacc attatgatgt tatcgagtgg cgaaagatat ctgctcgaaa aattgactac gacgaccttt |
| 37731 | caaactctac tatcattttc caagaccctc gaaaagactt gatggacttg ctaaatgagg acggcgaagg |
| 37801 | agtcctttca ggggaaactg taaatgagtc ccaagttgtt attagatacg cagatgacat tttagggact |
| 37871 | aattttaatg cagaatctgg gaaatacatt ggtgtcctta atactaataa gaaaccgagc gaattagttc |
| 37941 | ctgacgactt tacatggatt cgactagaag gtcctaaagg tgacgcaggt ttaccgggag ctcctgggcg |
| 38011 | tgatggagtc gacggtgtac ctggaaagag cggagtaggg atagcagata cagctatcac ttatgctgta |
| 38081 | tccgtttccg gaacgcaaga gcctgaaaat ggatggagcg aacaagttcc tgaactcata aaaggtcgat |
| 38151 | tcttgtggac taaaacattt tggagatata ctgacggctc acatgaaact ggatactccg ttgcctatat |
| 38221 | agggcaagac ggaaattccg gaaaagacgg aatcgcaggt aaggacggag taggtatagc cgcaactgaa |
| 38291 | gtcatgtatg caagttcgcc atctgctact gaagctccag ctggtggatg gtctacgcaa gttcctaccg |
| 38361 | tcccaggtgg tcagtattta tggactcgaa caagatggcg ctacactgac caaactgatg aaattggata |
| 38431 | ttcagtttca agaatgggcg agcagggtcc taaaggtgac gcaggtcgtg acggtattgc aggaaagaac |
| 38501 | ggaatagggt tgaagtcaac ttcagtttct tatggaatta gtcccactga ttctgcgatt cctggagtat |
| 38571 | gggcttcaca agttccttct ttaatcaaag gtcaatatct ttggactcga actatttgga cctataccga |
| 38641 | ttcaactacc gaaacgggct atcaaaaaac ctacattcca aaagacggga atgacggtaa aaatggaatt |
| 38711 | gctggtaagg atggggtagg aattaagtct acgaccatta cctacgcagg ctcaacctca ggaacagttg |
| 38781 | cgcctacttc aaattggact tctgctattc caaatgttca accgggattc ttcttgtgga cgaaaactgt |
| 38851 | ttggaactat actgatgaca ctagcgaaac aggttactca gtttccaaga taggtgaaac aggtcctaga |
| 38921 | ggagttcaag gtcttcaagg tcctcaaggg cttcaaggaa ttcctggacc tgcaggagct gacggacgtt |
| 38991 | cgcaatatac tcacctcgct ttctctaata gtccaaacgg tgagggattt agtcatactg acagcggacg |
| 39061 | agcatacgtc ggtcagtatc aagatttcaa tcccgtccat tcaaaagacc ctgcagccta tacatggacg |
| 39131 | aaatggaagg ggaatgacgg agctcaaggg atacccggga agccaggcgc agacggtaag actaattatt |
| 39201 | tccatatagc ttacgcttca agtgcagacg gatcacgtga gttcagtttg gaagataata atcaacaata |
| 39271 | tatgggttat tactccgatt atgagcaagc agatagcagg gatcgaacta agtatcgatg gtttgaccgc |
| 39341 | cttgccaatg ttcaagtggg aggtcgaaac gagttcctta attctttatt tgaatttggt ttaaaacctc |
| 39411 | gctattctag ttacaatcta atggacggac aagatcaaac gcaaggacag atatctgcta ctattgacga |
| 39481 | acgtcaacgg ttcaaaggtg ctaactcttt acgacttgac tcaacatgga acggtaaacc gcagaaccaa |
| 39551 | aaactgacct tttctttagg aggagatacg cgattaggta ctccaaccga gtggtctaat ttagaaggtc |
| 39621 | gtatcagttt ctgggctaag gcctctagga acggagtgag cttagctgca cggccgggtt atcgtagtaa |
| 39691 | cgtatttacc gcaaccttaa ccgatcaatg gaagttctac gattttaaat tctttgacaa agttaattca |
| 39761 | aattgtaccg ctgaagcaat tttccatgta ttcactcaaa gttgttcagt gtggctcaat catattaaaa |
| 39831 | tcgaacttgg taatatctct actcctttta gtgaagcaga ggaagacctt aaatatcgaa ttgactcaaa |
| 39901 | agccgatcaa aagctaacta accaacagtt gacggcactc acggaaaagg ctcaactaca tgacgcagaa |
| 39971 | ctgaaagcta aggctacaat ggagcagtta agtaacttag aaaaggctta tgaaggtaga atgaaagcta |
| 40041 | atgaagaagc tatcaaaaaa tcggaagccg acctaatctt agcggcaagt cgaattgaag ctactatcca |
| 40111 | agaacttggc gggctacggg aactgaagaa gttcgtcgac agttacatga gctcttctaa tgaaggtcta |
| 40181 | attatcggta agaacgacgg tagctctacc attaaggtat caagtgaccg aatttctatg ttctccgcag |
| 40251 | ggaatgaagt tatgtacctt acgcaagggt tcattcacat cgataacggg atctttaccc aatccattca |
| 40321 | agtcggccga tttagaacgg aacaatactc gtttaatcca gacatgaacg tgattcggta tgtaggataa |
| 40391 | ggagaataac atgacaaaat ttatcaactc atacggccct cttcacttga acctttacgt cgaacaagtt |
| 40461 | agtcaggacg taacgaacaa ctcctcgcga gttagttggc gagctactgt cgaccgcgat ggagcttatc |
| 40531 | gaacgtggac ttatggaaat attagtaacc tttccgtatg gttaaatggt tcaagtgttc atagcagtca |
| 40601 | cccagactac gacacgtccg gcgaagaggt aacgctcgca agtggagaag tgactgttcc tcacaatagt |
| 40671 | gacgggacaa agacaatgtc cgtttgggct tcgtttgacc ctaataacgg cgttcacgga aatatcacta |
| 40741 | tctctactaa ttacacttta gacagtattc caaggtctac acagatttct agttttgagg gaaatcgaaa |
| 40811 | tctaggatct ttacatacgg ttatctttaa ccgaaaagtg aactctttta cgcatcaagt ttggtaccga |
| 40881 | gttttcggta gcgactggat agatttaggt aagaaccata ctactagcgt atcctttacg ccgtcactgg |
| 40951 | acttagcaag gtacttacct aaatcaagtt ccggaacaat ggacatctgt attcgaacct ataacggaac |
| 41021 | tacgcaaatt ggtagtgacg tctattcaaa cggatggagg ttcaacatcc ccgattcagt acgtcctact |
| 41091 | ttttcgggca tttctttagt agacacgact tcagcggttc gacagatttt aacagggaac aacttcctcc |
| 41161 | aaatcatgtc gaacattcaa gtcaacttca acaatgcttc cggcgcttac ggatccacta tccaagcatt |
| 41231 | tcacgctgag ctcgtaggta aaaaccaagc tatcaacgaa aacggcggca aattgggtat gatgaacttt |
| 41301 | aatggctccg ctaccgtaag agcatgggtt acagacacgc gaggaaaaca atcgaacgtc caagacgtat |
| 41371 | ctatcaatgt tatagaatac tatggaccgt ctatcaattt ctccgttcaa cgtactcgtc aaaatcctgc |
| 41441 | aattatccaa gctcttcgaa atgctaaggt cgcacctata acggtaggag gtcaacagaa aaacatcatg |
| 41511 | caaattacct tctccgtggc gccgttgaac actactaatt tcacagaaga tagaggttcg gcgtcaggga |
| 41581 | cgttcactac tatttcccta atgactaact cgtccgcgaa cttagctggt aactacgggc cggacaagtc |
| 41651 | ttacatagtt aaggctaaaa tccaagacag gttcacttcg actgaattta gtgctacggt agctaccgaa |
| 41721 | tcagtagttc ttaactatga caaggacggt cgacttggag ttggtaaggt tgtagaacaa gggaaggcag |
| 41791 | ggtcaattga tgcagcaggt gatatatatg ctggaggtcg acaagttcaa cagtttcagc tcactgataa |
| 41861 | taatggagca ttgaacaggg gtcaatataa cgatgtttgg aataagcgtg aaacagagtt tacatggcga |
| 41931 | agtaacaaat acgaggacaa ccctacggga actcgaggtg aatggggact atttcaaaat ttctggttag |
| 42001 | atagctggaa aatggttcaa tccttcatta caatgtcagg aagaatgttc atcaggacag cgaacgatgg |
| 42071 | aaacagctgg agacctaaca agtggaaaga ggttctattt aagcaagact tcgaacagaa taattggcag |
| 42141 | aaacttgttc ttcaaagtgg gtggaaccat cactcaacct atggcgacgc attctattcg aaaactcttg |
| 42211 | acggcatagt atatttgaga ggaaatgtgc ataaaggact tatcgacaaa gaggctacta ttgcagtact |
| 42281 | tcctgaagga tttagaccga aagtttcaat gtatcttcag gctctcaata actcatatgg aaatgccatt |
| 42351 | ctatgtatat acactgacgg aagacttgtg gtgaaatcga atgtagataa ttcttggtta aatttagaca |
| 42421 | atgtctcatt tcgtatttaa tttgagctga aatcatgtta taatattttt tagaaaggag gtgagaacta |
| 42491 | tgttgaacct tacaaaatcg cgccaaattg tggcagagtt cactattgga caaggagctg aaaagaaact |
| 42561 | tgtcaaaaca acgattgtga acattgatgc aaacgcagta tcaaccgtct ctgaaactct tcatgaccca |
| 42631 | gacttgtatg ctgcgaaccg tcgagaactt cgagctgacg agcaaaaact tcgcgaaact cgttacgcaa |
| 42701 | tcgaagatga aattctagct gaacagtcaa agactgaaac agctctaaca gctgaataag gaggcgtcaa |
| 42771 | tctatgccaa tgtggctaaa cgacacagca gtcttgacga cgattattac agcgtgcagc ggagtgctta |
| 42841 | ctgtcctact aaataagtta ttcgaatgga aatcgaataa agccaagagc gttttagagg atatctctac |
| 42911 | aactcttagc actcttaaac agcaggtcga cgggattgac caaacgacag tagcaatcaa tcaccaaaat |
| 42981 | gacgtcattc aagacggaac tagaaaaatt caacgttacc gtctttatca cgacttaaaa agggaagtga |
| 43051 | taacaggcta tacaactctc gaccatttta gagagctctc tattttattc gaaagttata agaaccttgg |
| 43121 | cggaaatggt gaagttgaag ccttgtatga aaaatacaag aaattaccaa ttagggagga agatttagat |
| 43191 | gaaactatct aacgaacaat atgacgtagc aaagaacgtg gtaaccgtag tcgttccagc agcgattgca |
| 43261 | ctaattacag gtcttggagc gttgtatcaa tttgacacta ctgctatcac aggaaccatt gcacttcttg |
| 43331 | caacttttgc aggtactgtt ctaggagttt ctagccgaaa ctaccaaaag gaacaagaag ctcaaaacaa |
| 43401 | tgaggtggaa taatgggagt cgatattgaa aaaggcgttg cgtggatgca ggcccgaaag ggtcgagtat |
| 43471 | cttatagcat ggactttcga gacggtcctg atagctatga ctgctcaagt tctatgtact atgctctccg |
| 43541 | ctcagccgga gcttcaagtg ctggatgggc agtcaatact gagtacatgc acgcatggct tattgaaaac |
| 43611 | ggttatgaac taattagtga aaatgctccg tgggatgcta aacgaggcga catcttcatc tggggacgca |
| 43681 | aaggtgctag cgcaggcgct ggaggtcata cagggatgtt cattgacagt gataacatca ttcactgcaa |
| 43751 | ctacgcctac gacggaattt ccgtcaacga ccacgatgag cgttggtact atgcaggtca accttactac |
| 43821 | tacgtctatc gcttgactaa cgcaaatgct caaccggctg agaagaaact tggctggcag aaagatgcta |
| 43891 | ctggtttctg gtacgctcga gcaaacggaa cttatccaaa agatgagttc gagtatatcg aagaaaacaa |
| 43961 | gtcttggttc tactttgacg accaaggcta catgctcgct gagaaatggt tgaaacatac tgatggaaat |
| 44031 | tggtattggt tcgaccgtga cggatacatg gctacgtcat ggaaacggat tggcgagtca tggtactact |
| 44101 | tcaatcgcga tggttcaatg gtaaccggtt ggattaagta ttacgataat tggtattatt gtgatgctac |
| 44171 | caacggcgac atgaaatcga atgcgtttat ccgttataac gacggctggt atctactatt accggacgga |
| 44241 | cgtctggcag ataaacctca attcaccgta gagccggacg ggctcattac tgctaaagtt taaaatatag |
| 44311 | agaggaggaa gctcttttct taatattgtt tctcttaatc ccgcaaggtt tcgaccctgc ggggttttgt |
| 44381 | gtcgtatatt actctattta cttattcgaa gatttcaatt ataattaaat agtcaacatg attcatgatt |
| 44451 | gttgatatga ccctttccgc cctacataat ttgtggggcg tttatttttt ataaaaattt tttacaaaat |
| 44521 | gcttgacaac attcactcat tatcgtataa tacaattata aaaataaata aagccgaaag gcgaggagga |
| 44591 | cattatgtca aaaattaaat tcgaaaacct taaaaaaggc gatgttgtgc tacgagctaa atctcaaacg |
| 44661 | aaqtttaaaa tcgtttcaat tttagcagac gaaaagaaag cagaccttga atcattagaa gacggaggtg |
| 44731 | aacttcacct ttcagcttca actctcgaac gttggtacac aatggaagat gaaactgaac ctaaaaaaga |
| 44801 | agaagctgct aaacctgcta aaaaggctgc tcctgcagtt gctcgacctg ctcgaaaagg tagagtcgtt |
| 44871 | cccaaaccta aaaaagaagt ccttgaggaa gaaattcctg aagttaagga acagccggaa gaagttggtt |
| 44941 | cagttagtga gaaatctact gttcgaaaac ctgctcctaa aaaagaaagc gtgatggcga ttactaaggc |
| 45011 | tcttgaaagt cgaattgttg aagcctttcc tgcgtctact cgaatcgtca ctcagtctta catcgcctat |
| 45081 | cgctctaaga agaacttcgt tactatcgaa gaaactcgaa aaggtgtttc tattggagtt cgcgcaaaag |
| 45151 | ggttgacaga agaccaaaag aaacttcttg catctattgc tcctgcatct tacgaatggg cgattgacgg |
| 45221 | aatttttaaa ctcgtcaagg aagaagatat tgacaccgca atggaattga ttgaagcttc tcacctttct |
| 45291 | tcgctatgat tgaaatcgtt atagcacgtt cgaaagctag gcgaggtcga accctattta ttgaaacatg |
| 45361 | ggcaagcact gatgaagatg cagttaaaat ggcagaaaag atttccagct tgcccaatgt agtcgagacg |
| 45431 | tcttctaata acttcgaact accttataag tatttcaata atgttataga cgctctagat gaatgggagc |
| 45501 | ttcacatctt cggcgaactt gataaagatg ttcaagacta cattgactct cgaaaccgaa tagcttcttc |
| 45571 | aagcaatgag cagttttcgt tcaagactac tccattcgcg caccaggttg aatgtttcga atacgcacaa |
| 45641 | gagcatccat gtttcctttt aggcgatgag caaggtttag ggaaaactaa acaggcaatt gatattgcag |
| 45711 | ttagcaggaa ggcaagtttc aaacattgtt taatcgtatg ttgcatatca gggctcaaat ggaattgggc |
| 45781 | aaaagaagta ggtattcatt caaatgagtc agctcatatt ttaggaagtc gagtcactaa agatgggaaa |
| 45851 | ttagtgattg acggagtttc taaacgggca gaagacttgc ttggtggcca cgacgaattc ttccttatca |
| 45921 | ctaacattga aactcttcgc gatgctgtgt tcattaaata cttaaatgaa ctgacaaaaa gcggagaaat |
| 45991 | tggaatggtt attattgacg agattcacaa gtgtaagaac ccttcaagta agcaaggggc ttcaattcaa |
| 46061 | aagctccaaa gttattacaa gatgggactt acaggaactc ctctaatgaa taacccaatc gatgtattca |
| 46131 | atgttatgaa gtggctaggg gcggaacatc atacactgac tcagttcaaa gagcgatact gtatcgtcga |
| 46201 | ccagttcaat caaatcactg gatatcgaaa tctagctgaa cttcgcgagc ttgtcaacga ctacatgctt |
| 46271 | agaagaacga aggaagaagt tttagacctg cctgaaaaga ttcgagtcac agagtatgtc gacatgaact |
| 46341 | cgaaacagtc aaaaatctat aaggaagttt tgactaaact tgttcaagaa atagataaag tcaagctcat |
| 46411 | gcctaaccct ctagccgaaa cgattcgact tcgacaagcg actggaaatc cttcgatttt aactactcaa |
| 46481 | gatgtcaagt cttgcaagtt cgaaagatgt atcgaaattg tcgaggaatg tatccagcaa ggaaagtcct |
| 46551 | gcgtgatatt tagcaattgg gaaaaggtta ttgaacctct tgctaagata ctttcgaaga cagtcaaatg |
| 46621 | caacctggta acaggagaaa ccgcagataa gttcaacgaa attgaagaat ttatgaatca cagaaaggct |
| 46691 | tctgttattt taggaactat aggtgcgcta ggaacaggat ttactttgac gaaagcggat acggttattt |
| 46761 | tcttagatag tccgtggaca cgcgcagaaa aggaccaagc cgaagatagg tgtcatagaa ttggcgcaaa |
| 46831 | aagttctgtc actatctaca cgcttgtcgc caaaggtact gttgacgaac gtatagaaga ccttattgaa |
| 46901 | cggaaaggag aattagcaga ttatatcgta gatggtaagc ctatgaaatc taaaattggt aaccttttcg |
| 46971 | atatcctgct taaatagaat gaaaactatc tccatattaa ggaaagacac taaaaggaag ccggacagga |
| 47041 | acggaagaaa aactgcactc gaactagctc aagagattga tatgtcacct agtgagttag cagagctcct |
| 47111 | tcaaattcct gaaaggacgg caaccagaat tttaaaactc gacaaactgc tcaacaaaga gcaatgctca |
| 47181 | ataatagaaa ggtatataaa tgaaattcac tgaaggaaaa aattggtata aagttggaga gatatgtcaa |
| 47251 | atgttgaacc gctctctatc tacgattaat gtttggtatg aagcaaaaga cttcgctgaa gaaaataaca |
| 47321 | ttcacttccc gtttgttctt cctgaaccta gaacagacct tgaccatcgt ggttctcgat tctgggatga |
| 47391 | cgaaggcgtg aacaaactca aacgatttag ggacaaccta atgcgcggtg acttggcatt ctacactcga |
| 47461 | actcttgtag ggaaaactga aagggaagca attcaagaag atgctaaagc atttaaacgt gaacatggat |
| 47531 | tggagaatta aatgaaattt gaagatgaaa aacagttcat cgctgcaatt gaagaagccg gtgaattaaa |
| 47601 | tgctaccaaa ggcgacatgg agaaacaagt caaaagtctt cgtgatgctc taaaagagta catgaaagaa |
| 47671 | aatgacattg aatctgctca aggtaagcac ttttctgcta ccttctacac gacagagcgc tcaactatgg |
| 47741 | acgaagaacg cttgaaagaa attatcgaaa aattagttga cgaagccgag acggaagaaa tgtgtgaaaa |
| 47811 | actttcaggg cttatcgaat acaagcctgt catcaatacg aaacttctcg aggatatgat ttatcacggc |
| 47881 | gagattgacc aagaagcaat tcttccagca gttgtcattt ctgttacaga aggcattcgt tttggaaagg |
| 47951 | ctaaaattta gcgatatttt tggttctgcg acgtttttag ggttagcaga atccaatcac accacttgcg |
| 48021 | caggcaaccg ctgtctgcgt taattttaga aggttaatat tataccataa ggaggagata agtggcaagg |
| 48091 | caaagaatag gcaattcagg aaagcctaaa aatgaaattg aactaacatt caaagacaag cctaaaactc |
| 48161 | gttctacctt attcaagaag gacgtggcaa caggtctttc aaaagtcgag catgattatt ttcaaatagt |
| 48231 | tgaagcactt aacggaaaac aattcgaacc taatatgaag caggtgtcat ctttctttat agttcagtat |
| 48301 | gaatttattt tcaatattaa gtgcatcgat tataactggt tcaacttttc gagcactatg aaaaatgttc |
| 48371 | gaacttattt aaacattgag tcgaacattg aactttgtcg atttttagct gaaagttttg ttaaatatga |
| 48441 | aaatgttcga aaaagattga acctaagcga aaggttcata acggtctcga ctttcaaaag agcctggatt |
| 48511 | ttggacgaac tcgaaggaaa aacgggttca aaattcgaag gattttatta gtttagtaga ctatttttag |
| 48581 | attttttaaa atgtggttta caaaatgacc tcaataggcg tataatttat caatcttgat tctttcgggc |
| 48651 | cggtatatat acaccaataa tcgagaaata ataaattata gtatcgaaaa tataaaaagg agaaaagttg |
| 48721 | gaaaatttag ctgatagaat atggaagaaa aagttaaatg accttttcga gagaagtggg ctacctcaaa |
| 48791 | agtatttcga acctcaagtg ttagtcgaac gaaaagccga caaggaatgt tgggaatggc tagaagctgt |
| 48861 | tcgagcaaat atagtcgaag aagttcgaaa cggtcttagc attgttattg cttcgaatac tgtcgggaat |
| 48931 | gggaaaacta gctgggcggt tcgacttttg caacgctatt tagcagaaac tgcacttgac ggaagaattg |
| 49001 | ttgagaaagg aatgtttgta gtgtcagctc aactattgac tgagttcggc gactataatt attttcaaac |
| 49071 | catgcaagaa tttctcgaac gtttcgagcg ccttaagact tgtgagctat tagtcataga cgaaataggt |
| 49141 | ggaggttcct taaccaaggc ctcttatcct tatctgtatg acttggttaa ttatagggtt gacaataact |
| 49211 | tgtcgactat ttatacgact aattatactg acgatgaaat tattgacctt ttaggccaaa ggctttatag |
| 49281 | tcgtatatat gatacttcag tggttctaga ttttcaggca agcaatgtaa gaggattgga ggtaagcgaa |
| 49351 | attgaatcat agatatagta acatcacaac tatttttctt tggcagattg tctttctttg tatttgctgc |
| 49421 | gcggtgtcct attgtgcagg agtgcataat gagcgagagt ctcaagataa ggtgattcaa agttataagc |
| 49491 | agaaagaaaa gtcagccgtc tacttgacag tcgatagttc aggagcttgg ctaggaagtg ctccgggagc |
| 49561 | caaggaaagt cctctctaca atgaaaaggg acagcatgta ggaaaattga aagaggtggg agagtgatac |
| 49631 | agcttcaagt cttaaataaa gttctcgaag aaaagagctt atccatttta gaaaataatg gaattgacca |
| 49701 | agaatacttc acggattatt tagacgagta tcaatttatt caagaacact tttcgagata tggaagagtt |
| 49771 | ccggacgacg aaactattct cgaccatttt cctggattcg aatttttcga aattggcgaa actgatgaat |
| 49841 | accttatcga caagctaaaa gaggagcatc tatataattc acttgttcca attttaacgg aagcggctga |
| 49911 | ggacattcaa gtagatagta acattgcgat tgcgaatata attccaaaac tagaagaact tttcaatcgc |
| 49981 | tctaaattcg taggcggact agacattgct cgaaatgcta aacttcgact agactgggcg aatactatta |
| 50051 | gaaaccatga cggtgaaaga cttggaatat cgacagggtt tgaactattg gacgacgtgc ttggaggctt |
| 50121 | acttcctggt gaggatttga ttgtcataat ggctcgacct ggacaaggta agtcgtggac tattgataaa |
| 50191 | atgcttgcaa ctgcttggaa gaacgggcat gatgtccttc tatatagcgg ggaaatgagt gaaatgcaag |
| 50261 | ttggtgctcg tatagatact attctttcga atgttagcat caattcaatt accaaaggga tttggaacga |
| 50331 | ccatcagttc gaaaaatatg aggaccatat tcaagcaatg actgaggctg aaaattccct tgtggtagtc |
| 50401 | acgcccttta tgattggagg aaagaacctt acccctgcaa ttttagatag catgatatct aaatatagac |
| 50471 | catctgtggt ggggattgac cagctttcac tcatgagcga gtcttatcca agcagggagc agaagcgaat |
| 50541 | ccagtacgcc aacatcacca tggacctata taagatttct gctaaatatg gaattcctat tgtgcttaat |
| 50611 | gtccaagcag ggcgttcggc taaaactgaa ggcgctgaaa gtatggaact agaacatata gcagaaagtg |
| 50681 | atggagtagg tcaaaatgct agcagagtta tcgctatgaa gcgtgacgaa aaatccggca tacttgaact |
| 50751 | atctgtcgtt aaaaaccgat atggcgaaga ccgaaaaatc atcgaatata tgtgggacgt tgaaactgga |
| 50821 | acctatactc ttataggatt caaagaggaa ggcgaagaag gaactgaaaa aggcgaaagc tctccattga |
| 50891 | aagcaaaagc ctctaggtcg actgctcgtc ttcgaagtaa ggttacaagg gaaggagttg aagcattttg |
| 50961 | atgaaagtaa atggtcttca aattgaagcg actcctgaac aaataattga aaaactttcg agacaacttg |
| 51031 | aagacgaagg aacattcatt tttagacgaa ctaagtcgct tggaagcaac tatcaattct catgcccgtt |
| 51101 | tcatgcagga gggactgaaa agcatccctc ttgtggcatg agtagaaatc cttcttattc aggaagtaag |
| 51171 | gtgacggaag ctggaacggt tcactgtttc acttgcggct acacttcagg actaactgaa ttcgtctcga |
| 51241 | atgtattagg tcgaaacgat ggagggttct atggaaacca gtggctgaaa aggaattttg gaacatctag |
| 51311 | cgaagtagtt aggcaaggcg tcagccctga agcgtttcga agaaatggga gaactgaaaa agtcgagcat |
| 51381 | aaaatcattc ctgaagagga acttgataaa taccggttta ttcatcctta tatgtatgaa cggaaattga |
| 51451 | cggacgagct catcgagatg tttgatgtag gttatgacaa actgcatgat tgcatcacct ttccagtacg |
| 51521 | gaacctcaag ggcgaaacag tattcttcaa ccgtcgaagt gttcgttcta agtttcacca gtacggtgaa |
| 51591 | gatgacccta aaacggaatt tctttatggc caatatgagc ttgtagcatt tcgagactat tttgaaaaac |
| 51661 | ctattagtca agtattcgtg actgagtctg ttatcaactg cttgactctt tggtcaatga agattccagc |
| 51731 | agtcgctctt atgggagtag gtggaggaaa tcaaatcaat ttactaaaac gacttcctta tagaaatatt |
| 51801 | gttctagcac ttgaccctga taacgctggg cagacagcgc aggaaaaact ctaccgacag ttaaagcgaa |
| 51871 | gcaaggtcgt tagatttttg aactacccta aagagttcta tgataataag tgggatataa acgaccatcc |
| 51941 | ggaattatta aattttaatg atttagtctt gtagaaattc atttattatc gtataataaa gttagaaaat |
| 52011 | tttaaaaaga ggtcatatca atatgaaaga agcgaataga ctagtttcta gctatgtagg attcgaatgc |
| 52081 | tggactgacg aagaatgtat caggaacttt gaactagacc ctgatatgtc aattgcgtct gcttatcatc |
| 52151 | gttattttgg gatgctttat tcctatgcaa aaaggtttaa atgcttatct cgacatgaca ttgaaagcat |
| 52221 | tgcattcgag actatttcaa aatgtttggc aacgttcaaa tcaaaccaag gggccaagtt ttcaacttac |
| 52291 | cttacaagac tcttcaagaa tagaatagtc ttagaatata ggtacctaaa tgcaccttcc atgaatcgaa |
| 52361 | attggtatgt agaagtgacg ttcgatagcg tttcgacaaa tgaagaaggc gacgatttta gtatcctatc |
| 52431 | gacagttggc tattgtgaag actacggaaa aattgaaatt gaagcaagtc ttgacttcat gacgctttct |
| 52501 | aatacagagt atgcttatat ctcgtctgtc attcaaaacg gtccttcagt aagcgacgca gaaattgcgc |
| 52571 | gtgaaattgg agtaagcagg tctgctatta gtcagtctaa gaagtcacta aaaaataaat taaaagattt |
| 52641 | tatataactg gtttacaaat cacgtgaatt tcgtgtatat tatatatgaa aggacaaact ttgaaacctt |
| 52711 | aaaaacttca aaaatctttc aaccattaaa aacttataaa ggagaatcga tatgggaaaa gtatcaattc |
| 52781 | aaaaatcagg aacatttagc tcagggtcta ataacgagtt tttcacactc gctgaccacg gtgacagcgc |
| 52851 | aattgtcact ctattgtatg atgacccgga aggcgaagac atggattatt tcgtagtcca cgaagcagac |
| 52921 | gttgacggtc gtcgacgcta tatcaattgc aatgctattg gcgaagacgg ggaaacagtc catcctgata |
| 52991 | attgtccatt atgccaaaac ggattccctc gtattgaaaa actatttctt caactttaca accatgatac |
| 53061 | gggaaaagtt gaaacatggg accgaggccg ttcttatgtt caaaagattg ttacatttat caataaatat |
| 53131 | ggaagccttg tgactcagcc ttttgaaatt attcgttcag gagctaaagg tgaccaacga actacttatg |
| 53201 | aattccttcc agagcgtccg gaagacagtg ctactcttga agattttcca gaaaagagcg aacttcttgg |
| 53271 | aactctaatt ttagacctcg acgaagacca aatgtttgac gtggttgacg gcaagttcac tcttcaagaa |
| 53341 | gagcgttctt caagtcgttc aaattcacgt agaggagcat ctcctgcgcc tagacgaggt tccggtcgag |
| 53411 | aatcttcaca aggtcgaaca gctgaaagaa ctccttcagt tagtcgaaga actcctccaa cacgaggtcg |
| 53481 | aggattctaa catgagggcg cgagccctct ttattattga ttaagaaagg gaaaataatg gcacaaaaag |
| 53551 | gactctttgg tgcaaagcct cgttctagca agaagaacga tgctcagtta cttgctcaac ggaaaaacag |
| 53621 | gaagcctgca gttgaggtta cttacatttc aggaaacgct ctaaaggacg cagttgctag agctcgtact |
| 53691 | ctttcaacta ggattcttgg acacgttctt gatagacttg agttaatcac tgaggaagca aaactcgagc |
| 53761 | agtatgtaga caaaatgatt gaagacggaa taggttctat tgacgtagaa actgatggac tcgatactat |
| 53831 | tcacgatgag ctggcaggag tctgcttgta ctcacctagt caaaaaggaa tctatgctcc tgtcaatcat |
| 53901 | gttagcaata tgacgaagat gcgaattaag aatcaaattt ctcctgagtt catgaagaaa atgcttcaac |
| 53971 | ggattgtaga ttcaggaatt cctgtcatct atcataattc gaaatttgac atgaaatcga tttattggcg |
| 54041 | actcggcgtc aaaatgaatg agccagcgtg ggatacatat ttagccgcaa tgcttttaaa tgaaaacgag |
| 54111 | tctcacagct tgaaaagtct tcactctaaa tatgttagga acgaagaaaa cgcagaggtt gcaaaattta |
| 54181 | atgacttatt taaaggaatt ccttttagtt taattcctcc tgatgttgcc tatatgtatg cggcctatga |
| 54251 | ccctttgcaa actttcgaac tctatgaatt tcaagaacaa tacttgactc caggaactga acaatgtgaa |
| 54321 | gaatataacc tggaaaaagt ctcatgggtt cttcataata ttgagatgcc tctaattaaa gttctcttcg |
| 54391 | acatggaagt ctacggtgtc gacttagacc aagataagct ggcagaaatt agagaacagt ttactgccaa |
| 54461 | tatgaacgag gctgagcaag agtttcaaca gcttgtcagc gaatggcagc ctgaaattga agaacttcga |
| 54531 | caaactaatt tccagagcta tcaaaaactc gaaatggatg caagaggtcg agtgacggta agcatttcca |
| 54601 | gtcctactca attagcaatt ctgttttatg atatcatggg attgaaaagt cctgaaaggg ataaacctag |
| 54671 | aggaacaggc gaaagtattg tcgagcattt tgataacgat atctcaaaag cacttttgaa atatagaaaa |
| 54741 | tatgcaaaat tagtttcgac ctatacaaca cttgaccaac accttgcaaa gcctgacaat cgaattcaca |
| 54811 | ctacattcaa acagtacgga gctaagacag ggcgtatgtc aagtgagaat cctaacttac agaatattcc |
| 54881 | ttctcgcggt gagggtgcag tagttcgaca aatctttgca gccagtgaag ggcattacat tattggtagt |
| 54951 | gactactctc aacaagaacc tcgttcattg gcggaattaa gtggcgacga aagtatgcga catgcttacg |
| 55021 | aacaaaacct ggacctatat tcagttatcg gttcgaaact ttatggtgtt ccctatgaag agtgtttaga |
| 55091 | gttctatccc gacggaacga ctaacaagga aggaaaactt cgaagaaatt ctgtcaagtc cgttctttta |
| 55161 | ggtcttatgt acggccgcgg ggctaactca atcgctgagc agatgaatgt atctgtcaaa gaagcgaata |
| 55231 | aggttattga agatttcttc accgagttcc ctaaagtggc agactatatc atattcgttc aacagcaggc |
| 55301 | gcaggacttg ggatatgttc aaacagctac cggtcgaaga agaaggcttc ctgatatgag tcttcctgaa |
| 55371 | tacgagttcg agtatatcga cgctagcaag aacgaagatt tcgacccctt taactttgac gcagaccaac |
| 55441 | agatggacga tactgttcct gaacatatta tcgaaaaata ttgggcccag ctagatagag cctggggatt |
| 55511 | taagaagaag caagaaatta aagaccaggc aaaagccgaa ggaattctta ttaaggataa cggaggcaag |
| 55581 | atagctgatg ctcagcgcca atgtttgaac tcagttattc aaggaacggc agccgacatg actaagtacg |
| 55651 | caatgattaa ggtacacaat gacgctgaat tgaaagaatt aggattccat ttaatgattc cagttcacga |
| 55721 | tgagttacta ggtgaggttc ctatcaagaa cgcaaaacgg ggagcagaaa ggttgacaga agttatgatt |
| 55791 | gaagcagcca aggacattat tagtcttcca atgaaatgtg accccagtat agtagaaaga tggtatggtg |
| 55861 | aagaaattga aatctaaaat ctattcagtt gcatatataa ttctagtagt tattgcgaac cttgtgacaa |
| 55931 | tttatttcga acctttaaat gtgaaaggaa ttttaattcc tccaagcagt tggtttatgg gattcacttt |
| 56001 | cctgcttata aatctaataa gcaagtacga gaagccaaaa tttgcaggtt ctttgatatg ggtagggtta |
| 56071 | ttccttacct cgttgatttg ctttatgcaa aacctaccac aatcgcttgt cgtggcttca ggagttgcat |
| 56141 | tttggataag tcaaaaagca agtgtcttta tattcgacaa gctctcgaat aaattagact cgaagattgc |
| 56211 | aaatgctttg tctagcaaca tcggttctat tatagacgca accatatgga tttcattagg actgagtcct |
| 56281 | cttggaattg gaacggttgc atatatagat attccgtcag ccgtactagg ccaagttcta gttcagttta |
| 56351 | tcttgcagtc aattgcttcg agatatttga aaaagtagtc aggaaaattc ctgattatct tgcagtcaat |
| 56421 | tgcttcgaga tatttgaaaa agtagtcagg aaaattcctg attatttttt ttacaaaaac gcttgacttt |
| 56491 | attcattcat tattat |

TABLE 5

| | |
|---|---|
| >dp1ORF001 DNA sequence | (SEQ ID NO. 11) |

atgattgacaataatttacctatgagtccaattcctggcgaaattgttcaagtatatgac
caaaacttcaatctaattggagcaagtgatgaaatctttagcaagcattacgaagacgaa
attgtgactcgagctcgaggaaaagaaactttcacttttgaaagtattgaaacctcatct
atctatcaacacttaaaggttgaaaacattatccagtatggaggaagatggtttcgaatt
aaatatgctcaggacgtagaagatgtcaaagggcttaccaagtttacctgctacgcatta
tggtatgaactagcagaaggcttgcctaggaagttgaaacacgttgcttcttctgtaggc
gctgtcgcgctagatattatcaaagacgcaggtgaatgggttcgactagtttgtcctcct
gacggtgctaacaaacaagttcgaagcataacagccgcagaaaattcaatgctttggcat
cttcgatatcttgcaaagcaatacaatttagaattgacatttggttatgaagaaattatc
aagcaagaggttagaattgttcaaaccgttgtatttcttcagccttatgtcgagtctaaa
gtagactttcctcttgtagttgaagagaatttgaaatatgtcactaggcaggaagattct
cgaaacctgtgtacggcttacaagttgacaggtaaaaaggaagaaggcagtcaagagcct
ttaacgtttgcttctatcaacaatggaagtgaatatctcattgatgtttcgtggtttact
acacgccacatgaagcctcgatatattgctaaatctaaaagcgacgaacattttagaatt
aaagaaaatttgatgagtgctgcgcgtgcttatcttgacatctacagtcgcccactaatt
ggatatgaggcttcagcggtcctttataacaaggttcctgacttgcatcatactcaacta
attgtcgacgaccattatgatgttatcgagtggcgaaagatatctgctcgaaaaattgac
tacgacgacctttcaaactctactatcattttccaagaccctcgaaaagacttgatggac
ttgctaaatgaggacggcgaaggagtcctttcaggggaaactgtaaatgagtcccaagtt
gttattagatacgcagatgacattttagggactaattttaatgcagaatctgggaaatac
attggtgtccttaatactaataagaaaccgagcgaattagttcctgacgactttacatgg
attcgactagaaggtcctaaaggtgacgcaggtttaccgggagctcctgggcgtgatgga
gtcgacggtgtacctggaaagagcggagtagggatagcagatacagctatcacttatgct
gtatccgtttccggaacgcaagagcctgaaaatggatggagcgaacaagttcctgaactc
ataaaaggtcgattcttgtggactaaaacattttggagatatactgacggctcacatgaa
actggatactccgttgcctatatagggcaagacggaaattccggaaaagacggaatcgca
ggtaaggacggagtaggtatagccgcaactgaagtcatgtatgcaagttcgccatctgct
actgaagctccagctggtggatggtctacgcaagttcctaccgtcccaggtggtcagtat
ttatggactcgaacaagatggcgctacactgaccaaactgatgaaattggatattcagtt
tcaagaatgggcgagcagggtcctaaaggtgacgcaggtcgtgacggtattgcaggaaag
aacggaatagggttgaagtcaacttcagtttcttatggaattagtcccactgattctgcg
attcctggagtatgggcttcacaagttccttctttaatcaaaggtcaatatctttggact
cgaactatttggacctataccgattcaactaccgaaacgggctatcaaaaaacctacatt
ccaaaagacgggaatgacggtaaaaatggaattgctggtaaggatggggtaggaattaag
tctacgaccattacctacgcaggctcaacctcaggaacagttgcgcctacttcaaattgg
acttctgctattccaaatgttcaaccgggattcttcttgtggacgaaaactgtttggaac
tatactgatgacactagcgaaacaggttactcagtttccaagataggtgaaacaggtcct
agaggagttcaaggtcttcaaggtcctcaagggcttcaaggaattcctggacctgcagga
gctgacggacgttcgcaatatactcacctcgctttctctaatagtccaaacggtgaggga
tttagtcatactgacagcggacgagcatacgtcggtcagtatcaagatttcaatcccgtc
cattcaaaagaccctgcagcctatacatggacgaaatggaaggggaatgacggagctcaa
gggatacccgggaagccaggcgcagacggtaagactaattatttccatatagcttacgct
tcaagtgcagacggatcacgtgagttcagtttggaagataataatcaacaatatatgggt
tattactccgattatgagcaagcagatagcagggatcgaactaagtatcgatggtttgac
cgccttgccaatgttcaagtgggaggtcgaaacgagttccttaattctttatttgaattt
ggtttaaaacctcgctattctagttacaatctaatggacggacaagatcaaacgcaagga
cagatatctgctactattgacgaacgtcaacggttcaaaggtgctaactctttacgactt
gactcaacatggaacggtaaaccgcagaaccaaaaactgaccttttctttaggaggagat
acgcgattaggtactccaaccgagtggtctaatttagaaggtcgtatcagtttctgggct
aaggcctctaggaacggagtgagcttagctgcacggccgggttatcgtagtaacgtattt
accgcaaccttaaccgatcaatggaagttctacgattttaaattctttgacaaagttaat
tcaaattgtaccgctgaagcaattttccatgtattcactcaaagttgttcagtgtggctc
aatcatattaaaatcgaacttggtaatatctctactccttttagtgaagcagaggaagac
cttaaatatcgaattgactcaaaagccgatcaaaagctaactaaccaacagttgacggca
ctcacggaaaaggctcaactacatgacgcagaactgaaagctaaggctacaatggagcag
ttaagtaacttagaaaaggcttatgaaggtagaatgaaagctaatgaagaagctatcaaa
aaatcggaagccgacctaatcttagcggcaagtcgaattgaagctactatccaagaactt
ggcgggctacgggaactgaagaagttcgtcgacagttacatgagctcttctaatgaaggt
ctaattatcggtaagaacgacggtagctctaccattaaggtatcaagtgaccgaatttct
atgttctccgcagggaatgaagttatgtaccttacgcaagggttcattcacatcgataac
gggatctttacccaatccattcaagtcggccgatttagaacggaacaatactcgtttaat
ccagacatgaacgtgattcggtatgtaggataa

| | |
|---|---|
| >dp1ORF002 DNA sequence | (SEQ ID NO. 12) |

atggattttgggtcaattgcagcaaaaatgactttggatatctcaaacttcacaagtcaa
ttaaatcttgctcaaagtcaagcgcaacggctcgcactagagtcttcgaagtcctttcaa
attggttctgctttaacaggattagggaaaggacttacgactgcggttacccttcctctt
atgggatttgcagccgcctctattaaagtagggaatgaattccaagctcaaatgtcccgt
gttcaagctattgcaggagcgacagcggaagagcttggtagaatgaagactcaagcaatc
gaccttggtgctaaaactgcttttagtgcaaaagaggcggctcaaggtatggaaaatcta
gcttcagccggtttccaggtaaatgaaatcatggacgctatgccaggggtacttgacctg
gctgccgtatctggaggagatgtggccgcgagctccgaggccatggctagttcacttcga
gcctttggattagaggcaaaccaggcgggtcacgtggctgacgtatttgctcgagcagca
gctgatacgaacgcagaaactagcgacatggcagaggcgatgaaatacgtcgcacccgtt
gctcactctatgggcttgagccttgaagaaacggctgcgtctattgggattatggccgac
gccggtattaagggctcgcaagccggaaccacgcttagaggcgctctctcgcgtattgcc
aaacctacgaaagcgatggtcaaatcaatgcaggaattaggagtttcgttctacgacgcg
aacggaaacatgattccactaagagaacaaatcgctcaactgaaaacagctactgcagga
ctaacacaagaggaacgaaatcgtcaccttgttaccttgtatggccaaaactcgttgtca
ggtatgcttgcactattagacgcaggtcctgagaaattggataagatgaccaatgctctc
gtgaactcggacggagctgctaaggaaatggcagaaactatgcaggacaaccttgctagt
aaaatcgagcaaatgggaggagctttcgagtctgttgctattattgttcaacaaatcctt
gagcctgcacttgctaaaatcgtgggagcaatcacaaaagttctcgaagcattcgtaaat
atgtcacctatcggtcaaaagatggttgtcatattcgcaggaatggttgcagcccttgga
ccactgcttctaattgcaggaatggtgatgacaactattgtcaagttaagaattgctatt
cagtttttaggtccagcatttatgggaacgatgggaaccattgcaggagttatagcaata
ttctatgctctggtcgccgtgttcatgatagcctacacaaaatcggagagatttagaaac
tttatcaacagtcttgcgcctgctattaaagctgggtttggaggagcgttggaatggcta
cttccacgactgaaagagttaggagaatggttacagaaggcaggcgagaaggcgaaagag
ttcggtcagtctgtagggtctaaagtgtcaaaactgctcgaacagtttggaataagtatc
ggtcaggcaggaggctcgattggtcagttcattggaaatgttctcgaaaggctaggaggc
gcatttggaaaagtaggaggagtcatttcaattgctgtttcacttgtaacaaaattcggt
ctcgcatttctagggattacaggaccactcgggattgctattagtctgttagtttcattt
ttgacagcttgggctagaacaggtgagttcaacgcagacggaattactcaagtattcgaa
aacttgacaaacacaattcagtcgacggctgatttcatctctcaataccttccagtcttt
gtcgaaaaaggaactcaaattttagttaagattattgaaggaattgcatctgctgttcct
caagtagttgaagtgatttcacaagtcattgaaaatattgtgatgacaatttcgacagtt
atgcctcaattagtcgaagcaggaattaagatactcgaagcgcttataaatggtcttgtt
caatctcttcctactatcattcaagcagctgttcaaattatcactgctttattcaatggt
cttgttcaggcacttcctacgcttattcaagcaggtcttcaaattttgtcagctctcata
aacggactagttcaagcgcttccggcaattattcaagcagctgttcaaattatcatgtcg
cttgttcaagcactaattgaaaacttgcctatgataatcgaagcagcgatgcagattata
atgggtctagtcaacgcactgattgaaaatataggacctatcttagaagcagggattcaa
attctaatggctttaatcgagggacttattcaagtgcttcctgaactaattacagcagcg
attcaaatcattacttcactattagaagcaatcttgtcgaaccttcctcaacttctagaa
gccggagttaaattgcttttatcacttcttcaagggttgctaaatatgcttcctcaacta
attgcaggggctttgcaaatcatgatggcacttcttaaagcagttatcgacttcgtccct
aaacttcttcaagcaggtgttcaacttcttaaggcattgattcaaggtattgcttcactt
ctcggctcacttttatcgacagctggaaacatgctttcatcattagttagcaagattgct
agctttgtgggacagatggtttcaggaggtgcgaacctgattcgaaacttcattagtggt
attgggtcaatgattggttcagctgtctctaaaattggcagcatgggaacttcaattgtt
tctaaggttactggattcgctggacaaatggtaagcgcaggggtcaaccttgttcgagga
tttatcaatggtatcagttccatggtaagttctgcggtaagtgcggcggctaatatggct
agcagtgcattaaatgccgttaagggattcttaggtattcactctccttcacgtgtcatg
gagcagatgggtatctatacgggtcaagggttcgtaaatggtattggtaacatgattcga
actacacgtgacaaggctaaagaaatggctgaaactgttactgaagctctcagcgacgtg
aagatggatattcaagaaaatggagttatagaaaaggttaaatcagtttacgaaaagatg
gctgaccaacttcctgaaactcttccagctcctgatttcgaagatgttcgtaaagcagcc
ggttcgcctcgagtggacttgttcaatacaggaagtgacaaccctaaccaacctcagtca
caatctaaaaacaatcaaggcgagcaaaccgttgtcaacattggaacaatcgtagttcga
aacaatgacgacgttgacaaactgtcgagaggattgtataatagaagtaaagaaactcta
tcagggtttggtaacattgtaacaccgtaa

| | |
|---|---|
| >dp1ORF003 DNA sequence | (SEQ ID NO. 13) |

atggcacaaaaaggactctttggtgcaaagcctcgttctagcaagaagaacgatgctcag
ttacttgctcaacggaaaaacaggaagcctgcagttgaggttacttacatttcaggaaac
gctctaaaggacgcagttgctagagctcgtactctttcaactaggattcttggacacgtt
cttgatagacttgagttaatcactgaggaagcaaaactcgagcagtatgtagacaaaatg
attgaagacggaataggttctattgacgtagaaactgatggactcgatactattcacgat
gagctggcaggagtctgcttgtactcacctagtcaaaaaggaatctatgctcctgtcaat
catgttagcaatatgacgaagatgcgaattaagaatcaaatttctcctgagttcatgaag
aaaatgcttcaacggattgtagattcaggaattcctgtcatctatcataattcgaaattt
gacatgaaatcgatttattggcgactcggcgtcaaaatgaatgagccagcgtgggataca
tatttagccgcaatgcttttaaatgaaaacgagtctcacagcttgaaaagtcttcactct
aaatatgttaggaacgaagaaaacgcagaggttgcaaaatttaatgacttatttaaagga
attccttttagtttaattcctcctgatgttgcctatatgtatgcggcctatgaccctttg
caaactttcgaactctatgaatttcaagaacaatacttgactccaggaactgaacaatgt
gaagaatataacctggaaaaagtctcatgggttcttcataatattgagatgcctctaatt
aaagttctcttcgacatggaagtctacggtgtcgacttagaccaagataagctggcagaa
attagagaacagtttactgccaatatgaacgaggctgagcaagagtttcaacagcttgtc
agcgaatggcagcctgaaattgaagaacttcgacaaactaatttccagagctatcaaaaa
ctcgaaatggatgcaagaggtcgagtgacggtaagcatttccagtcctactcaattagca
attctgttttatgatatcatgggattgaaaagtcctgaaagggataaacctagaggaaca
ggcgaaagtattgtcgagcattttgataacgatatctcaaaagcacttttgaaatataga
aaatatgcaaaattagtttcgacctatacaacacttgaccaacaccttgcaaagcctgac
aatcgaattcacactacattcaaacagtacggagctaagacagggcgtatgtcaagtgag
aatcctaacttacagaatattccttctcgcggtgagggtgcagtagttcgacaaatcttt
gcagccagtgaagggcattacattattggtagtgactactctcaacaagaacctcgttca
ttggcggaattaagtggcgacgaaagtatgcgacatgcttacgaacaaaacctggaccta
tattcagttatcggttcgaaactttatggtgttccctatgaagagtgtttagagttctat
cccgacggaacgactaacaaggaaggaaaacttcgaagaaattctgtcaagtccgttctt
ttaggtcttatgtacggccgcggggctaactcaatcgctgagcagatgaatgtatctgtc
aaagaagcgaataaggttattgaagatttcttcaccgagttccctaaagtggcagactat
atcatattcgttcaacagcaggcgcaggacttgggatatgttcaaacagctaccggtcga
agaagaaggcttcctgatatgagtcttcctgaatacgagttcgagtatatcgacgctagc
aagaacgaagatttcgacccctttaactttgacgcagaccaacagatggacgatactgtt
cctgaacatattatcgaaaaatattgggcccagctagatagagcctggggatttaagaag
aagcaagaaattaaagaccaggcaaaagccgaaggaattcttattaaggataacggaggc
aagatagctgatgctcagcgccaatgtttgaactcagttattcaaggaacggcagccgac
atgactaagtacgcaatgattaaggtacacaatgacgctgaattgaaagaattaggattc
catttaatgattccagttcacgatgagttactaggtgaggttcctatcaagaacgcaaaa
cggggagcagaaaggttgacagaagttatgattgaagcagccaaggacattattagtctt
ccaatgaaatgtgaccccagtatagtagaaagatggtatggtgaagaaattgaaatctaa

| | |
|---|---|
| >dp1ORF004 DNA sequence | (SEQ ID NO. 14) |

atgacaaaatttatcaactcatacggccctcttcacttgaacctttacgtcgaacaagtt
agtcaggacgtaacgaacaactcctcgcgagttagttggcgagctactgtcgaccgcgat
ggagcttatcgaacgtggacttatggaaatattagtaacctttccgtatggttaaatggt
tcaagtgttcatagcagtcacccagactacgacacgtccggcgaagaggtaacgctcgca
agtggagaagtgactgttcctcacaatagtgacgggacaaagacaatgtccgtttgggct
tcgtttgaccctaataacggcgttcacggaaatatcactatctctactaattacacttta
gacagtattccaaggtctacacagatttctagttttgagggaaatcgaaatctaggatct
ttacatacggttatctttaaccgaaaagtgaactcttttacgcatcaagtttggtaccga
gttttcggtagcgactggatagatttaggtaagaaccatactactagcgtatcctttacg
ccgtcactggacttagcaaggtacttacctaaatcaagttccggaacaatggacatctgt
attcgaacctataacggaactacgcaaattggtagtgacgtctattcaaacggatggagg
ttcaacatccccgattcagtacgtcctactttttcgggcatttctttagtagacacgact
tcagcggttcgacagattttaacagggaacaacttcctccaaatcatgtcgaacattcaa
gtcaacttcaacaatgcttccggcgcttacggatccactatccaagcatttcacgctgag
ctcgtaggtaaaaaccaagctatcaacgaaaacggcggcaaattgggtatgatgaacttt
aatggctccgctaccgtaagagcatgggttacagacacgcgaggaaaacaatcgaacgtc
caagacgtatctatcaatgttatagaatactatggaccgtctatcaatttctccgttcaa
cgtactcgtcaaaatcctgcaattatccaagctcttcgaaatgctaaggtcgcacctata
acggtaggaggtcaacagaaaaacatcatgcaaattaccttctccgtggcgccgttgaac
actactaatttcacagaagatagaggttcggcgtcagggacgttcactactatttcccta
atgactaactcgtccgcgaacttagctggtaactacgggccggacaagtcttacatagtt
aaggctaaaatccaagacaggttcacttcgactgaatttagtgctacggtagctaccgaa
tcagtagttcttaactatgacaaggacggtcgacttggagttggtaaggttgtagaacaa
gggaaggcagggtcaattgatgcagcaggtgatatatatgctggaggtcgacaagttcaa
cagtttcagctcactgataataatggagcattgaacaggggtcaatataacgatgtttgg
aataagcgtgaaacagagtttacatggcgaagtaacaaatacgaggacaaccctacggga
actcgaggtgaatggggactatttcaaaatttctggttagatagctggaaaatggttcaa
tccttcattacaatgtcaggaagaatgttcatcaggacagcgaacgatggaaacagctgg
agacctaacaagtggaaagaggttctatttaagcaagacttcgaacagaataattggcag
aaacttgttcttcaaagtgggtggaaccatcactcaacctatggcgacgcattctattcg
aaaactcttgacggcatagtatatttgagaggaaatgtgcataaaggacttatcgacaaa
gaggctactattgcagtacttcctgaaggatttagaccgaaagtttcaatgtatcttcag
gctctcaataactcatatggaaatgccattctatgtatatacactgacggaagacttgtg
gtgaaatcgaatgtagataattcttggttaaatttagacaatgtctcatttcgtatttaa

| | |
|---|---|
| >dp1ORF005 DNA sequence | (SEQ ID NO. 15) |

atggctaaaaaatcaaaagctatctcacacacagacgaactgattagtcagtcgtttgac
agccccttggcaaagaatcaaaagttcaagaaagagcttcaggaagttgaaaagtattat
caatacttcgacggatttgatgtcacggacttgaatactgactatgggcaaacatggaag
attgacgaagactcagtcgactataaacctactcgagaaattcgaaactatattcgacaa
cttatcaaaaagcaatcacgctttatgatgggtaaagagccagagcttatctttagtcca
gttcaagacaatcaagatgaacaggctgagaacaagcgtattctattcgactctatttta
aggaattgtaaattctggagcaaaagtacaaatgcattagtcgacgccacagtaggtaag
cgggtattgatgacagtagtagcaaatgccgctcaacaaattgacgtccagttttattca
atgcctcagttcacctatacagttgaccctagaaacccttccagcttgctttctgttgac
attgtttatcaggacgagcgtacaaaaggaatgagcactgaaaaacaactttggcatcat
tatagatatgaaatgaaagctggaacaagtcaatcaggaattgcaacagctttagaagac
attgaagaacaatgttggctcacttatgccttaacggatggagagtcgaaccaaatctat
atgacagaaagtggccaaactactatcaaggagacagaggctaaacttgtagaaattgaa
gacaacctaggaaacaagattgaagttcctttaaaagttcaagaatccgccccaaccggc
ttgaagcaaattccttgtcgagttattcttaatgaaccattgactaatgacatatacggg
acaagcgatgtcaaagaccttatcacagtagcagataacttgaacaaaactattagtgac
ttacgagattcacttcgatttaaaatgttcgagcagcctgttatcattgatggctcttct
aagtcaattcaaggaatgaagattgcgccaaacgctttggtcgaccttaagagtgaccct
acttcctcaatcggcggtactggaggcaagcaagctcaagtcacttccatttcaggaaac
ttcaacttccttccagcggctgaatattatttagagggcgctaagaaagccatgtatgaa
ctaatggaccagccaatgcctgaaaaggtacaggaggcgccatcaggaattgcaatgcag
ttcttattctacgacctaatttctcgatgtgacggaaaatggattgagtgggatgatgct
attcaatggctcattcaaatgctggaagaaattttagcaacagtgaatgttgacttggga
aatattcctcaagatattcaatcaagttatcaaacacttacgacaatgactatcgaacac
cactatccaattcctagcgatgaactttctgctaagcaacttgcgctcactgaagttcaa
actaatgtacgcagccaccaatcttacattgaagaattcagtaagaaggaaaaggcggac
aaggaatgggaacgcattttggaagaacttgctcagcttgacgaaatctcagctggagca
ttgcctgtattagcaaacgaattaaacgaacaagaggagcctcaagatgaaacgagtgaa
gaagacgaagttgatgacaaagaaaaagaacaaactgaacaaccaaccgaagaaggagtc
gacccagacgttcaaggttaa

| | |
|---|---|
| >dp1ORF006 DNA sequence | (SEQ ID NO. 16) |

atgattgaaatcgttatagcacgttcgaaagctaggcgaggtcgaaccctatttattgaa
acatgggcaagcactgatgaagatgcagttaaaatggcagaaaagatttccagcttgccc
aatgtagtcgagacgtcttctaataacttcgaactaccttataagtatttcaataatgtt
atagacgctctagatgaatgggagcttcacatcttcggcgaacttgataaagatgttcaa
gactacattgactctcgaaaccgaatagcttcttcaagcaatgagcagttttcgttcaag
actactccattcgcgcaccaggttgaatgtttcgaatacgcacaagagcatccatgtttc
cttttaggcgatgagcaaggtttagggaaaactaaacaggcaattgatattgcagttagc
aggaaggcaagtttcaaacattgtttaatcgtatgttgcatatcagggctcaaatggaat
tgggcaaaagaagtaggtattcattcaaatgagtcagctcatattttaggaagtcgagtc
actaaagatgggaaattagtgattgacggagtttctaaacgggcagaagacttgcttggt
ggccacgacgaattcttccttatcactaacattgaaactcttcgcgatgctgtgttcatt
aaatacttaaatgaactgacaaaaagcggagaaattggaatggttattattgacgagatt
cacaagtgtaagaacccttcaagtaagcaaggggcttcaattcaaaagctccaaagttat
tacaagatgggacttacaggaactcctctaatgaataacccaatcgatgtattcaatgtt
atgaagtggctaggggcggaacatcatacactgactcagttcaaagagcgatactgtatc
gtcgaccagttcaatcaaatcactggatatcgaaatctagctgaacttcgcgagcttgtc
aacgactacatgcttagaagaacgaaggaagaagttttagacctgcctgaaaagattcga
gtcacagagtatgtcgacatgaactcgaaacagtcaaaaatctataaggaagttttgact
aaacttgttcaagaaatagataaagtcaagctcatgcctaaccctctagccgaaacgatt
cgacttcgacaagcgactggaaatccttcgattttaactactcaagatgtcaagtcttgc
aagttcgaaagatgtatcgaaattgtcgaggaatgtatccagcaaggaaagtcctgcgtg
atatttagcaattgggaaaaggttattgaacctcttgctaagatactttcgaagacagtc
aaatgcaacctggtaacaggagaaaccgcagataagttcaacgaaattgaagaatttatg
aatcacagaaaggcttctgttattttaggaactataggtgcgctaggaacaggatttact
ttgacgaaagcggatacggttattttcttagatagtccgtggacacgcgcagaaaaggac
caagccgaagataggtgtcatagaattggcgcaaaaagttctgtcactatctacacgctt
gtcgccaaaggtactgttgacgaacgtatagaagaccttattgaacggaaaggagaatta
gcagattatatcgtagatggtaagcctatgaaatctaaaattggtaaccttttcgatatc
ctgcttaaatag

| | |
|---|---|
| >dp1ORF007 DNA sequence | (SEQ ID NO. 17) |

atgacaataagcctgagaaataaactacctaagttcaacttcgtcccttttagtaagaaa
caactccagctcctaacatggtggacaaagggctcaccttttcgaactttcgatatcgtc
atagcagacggttccattcgttcaggaaaaacagtatcgatggctctttcattttccctt
tgggccatgacggaattcaacggacaaaactttgccatctgtggtaagacaattcactca
gctcgacgaaatgttattcagcctctaaagcaaatgctcacaagtcgcgggtatgaaatt
cgagatgttcgaaatgaaaatctacttattattagacactttagaaatggcgaagaaatt
gtcaactacttctatatatttggaggaaaagatgagtcgagtcaagaccttatacagggg
gtaacattagcaggtatcttctgtgatgaggtggcactgatgcctgaatcgtttgtcaac
caagcgacagggcgctgttccgtaacaggttcgaaaatgtggttctcttgtaacccggcc
aatcctaatcactacttcaagaagaactggattgacaaacaggtcgaaaagcgtatctta
tatcttcactttacaatggacgacaaccctagcttgacggatagcattaaaaggcgctat
gagaaaatgtatgctggagtcttcaggaaaagatttattctcggcctttgggtaacagca
gatggtctagtttattcaatgttcaatgaagagcagcatgtcaaaaagctcaatatagaa
ttcgaccgtttattcgtagcaggcgactttggtatctataatgcaacaaccttcggcctt
tatggattctcgaaacgtcataagcgctaccatctaattgagtcatactaccactcaggg
cgcgaggcggaagagcaactaactgaggcggatgttaattcgaatattcaatttagttca
gttctacaaaagactactaaagagtacgcaaatgatttagtcgatatgatacgaggaaag
caaatcgaatatataattctcgacccgtctgcttctgctatgattgttgaacttcaaaag
catccttatatagctagaaagaatatccctatcattcctgctcgaaatgacgtgacgctt
ggcatttcatttcacgctgaactcttggctgagaatagatttacactcgaccctagcaac
acgcacgacattgatgaatactatgcttacagctgggacagtaaagcgagccaaacggga
gaagatagagtcattaaagagcatgaccactgcatggataggaacagatatgcctgtctc
actgacgctctaatcaacgatgacttcggtttcgaaatacaaatattatccggaaaaggc
gctagaaactaa

| | |
|---|---|
| >dp1ORF008 DNA sequence | (SEQ ID NO. 18) |

gtgatacagcttcaagtcttaaataaagttctcgaagaaaagagcttatccattttagaa
aataatggaattgaccaagaatacttcacggattatttagacgagtatcaatttattcaa
gaacacttttcgagatatggaagagttccggacgacgaaactattctcgaccattttcct
ggattcgaatttttcgaaattggcgaaactgatgaataccttatcgacaagctaaaagag
gagcatctatataattcacttgttccaattttaacggaagcggctgaggacattcaagta
gatagtaacattgcgattgcgaatataattccaaaactagaagaacttttcaatcgctct
aaattcgtaggcggactagacattgctcgaaatgctaaacttcgactagactgggcgaat
actattagaaaccatgacggtgaaagacttggaatatcgacagggtttgaactattggac
gacgtgcttggaggcttacttcctggtgaggatttgattgtcataatggctcgacctgga
caaggtaagtcgtggactattgataaaatgcttgcaactgcttggaagaacgggcatgat
gtccttctatatagcggggaaatgagtgaaatgcaagttggtgctcgtatagatactatt
ctttcgaatgttagcatcaattcaattaccaaagggatttggaacgaccatcagttcgaa
aaatatgaggaccatattcaagcaatgactgaggctgaaaattcccttgtggtagtcacg
ccctttatgattggaggaaagaaccttacccctgcaattttagatagcatgatatctaaa
tatagaccatctgtggtggggattgaccagctttcactcatgagcgagtcttatccaagc
agggagcagaagcgaatccagtacgccaacatcaccatggacctatataagatttctgct
aaatatggaattcctattgtgcttaatgtccaagcagggcgttcggctaaaactgaaggc
gctgaaagtatggaactagaacatatagcagaaagtgatggagtaggtcaaaatgctagc
agagttatcgctatgaagcgtgacgaaaaatccggcatacttgaactatctgtcgttaaa
aaccgatatggcgaagaccgaaaaatcatcgaatatatgtgggacgttgaaactggaacc
tatactcttataggattcaaagaggaaggcgaagaaggaactgaaaaaggcgaaagctct
ccattgaaagcaaaagcctctaggtcgactgctcgtcttcgaagtaaggttacaagggaa
ggagttgaagcattttga

| | |
|---|---|
| >dp1ORF009 DNA sequence | (SEQ ID NO. 19) |

atgacagactttaaaaaacgcttcaagaaagcagtaacagaaacaatcaatcgtgacggt
atcgagaaccttatggattggctcgaaaatgataccaatttcttctcaagtccagcaagc
actcgataccatggaagctatgaaggtggacttgtcgagcactcattaaacgtgttcaat
caactacttttcgaaatggataccatggtaggcaaaggctgggaagacatttacccaatg
gaaacagttgcaatcgtagcactatttcacgacctttgcaaagttggtcagtatcgtgaa
actgaaaaatggcgcaagaacagcgacggtgaatgggaaagctatttagcatatgaatac
gaccctgagcaacttacaatgggacatggtgcaaaatctaatttccttcttcaacgtttc
attcaactcacgccagttgaagctcaagcaattttctggcatatgggagcctatgatatt
agtccttatgcaaatttgaatggatgtggagcagccttcgaaactaatccacttgcattc
ttaatccatcgcgcagatatggccgcaacttatgtagtcgaaaatgaaaacttcgaatac
tctcaaggtccagttgaacaagaggctgaggttgaagaagtagttgaagaaaaacctaag
agttcaactcgtaagaaacctgcgcctaaggaagaaaaagttgaagaggctgaagaaaaa
ccaaaagctggaatcactcgacgtcgcaaacctgcgccaaaagaggaagaggtagaagag
cctaaagaagagcctaagaaagcatcttctaaaattcgaatgcctaaaaagactgaaaag
gtcgaagaggtagaaagcgcagacgagccgaaagttgaagaagcagaggacgacaatgtg
gtggtacctgctggatatgttcgagatgtctactacttctacagtgaagtcgctgacgtt
tactacaagaaagatgtcgacgagcctgacgatgacagcgacattcttgtagacgaagaa
gagtacatggacgcaatgtgtcctgtattagaagaagacttcttctacgaacttgacggc
aaggttcacaaattagcaaaaggtgaacgcttgccggaagaatacgacgaagaaacttgg
gaacctatcactgaagcagaatacatcaagcgaacagaaaaacctaaagcagttgcaaaa
cctactcgaaaaactccagcgccttctcgtcgccctcgcccttaa

| | |
|---|---|
| >dp1ORF010 DNA sequence | (SEQ ID NO. 20) |

atgaaattggaacagttgatgaaggactggaataaggattcgaaagctcttgtagcagtt
caaggacttgaacgtgaagcgcttccaagaatccctttttctgcgccttctatgaattat
caaacctacggcgggctccctcgaaaaagggtagttgaattcttcggtcctgagtcaagt
gggaaaactacttcagctctcgacattgtcaagaatgcgcaaatggtatttgagcaggaa
tgggaacagaagactgaagaactcaaggaaaagctggaaaatgcgcgtgcatccaaagct
agcaagactgctgtcaaggaacttgaaatgcaactcgatagtcttcaagagcctcttaag
attgtatatcttgaccttgagaatacattagacactgagtgggctaaaaagattggagtc
gatgttgacaatatttggatagttcgccctgaaatgaacagcgctgaagaaatacttcaa
tatgttttagacattttcgaaacaggtgaagttggcctagtagttctagattccttgcct
tacatggtcagtcaaaaccttattgatgaagagttgactaaaaaggcctatgcaggaatc
tcagcgcctttgactgaatttagtcgaaaggttactcctcttcttactcgctacaatgca
atattcctaggcatcaatcaaattcgagaagatatgaatagtcagtacaatgcctattca
actccaggcggaaagatgtggaagcatgcttgtgcagttcgacttaaatttagaaaaggt
gactaccttgacgaaaacggtgcatcattgacccgtactgctcgaaaccctgcagggaat
gtagtagagtcattcgtcgagaagaccaaagcatttaagccggacagaaaattagtttcc
tatacgctttcctatcatgatggaattcaaattgaaaatgaccttgtagatgtcgctgtc
gaatttggagtcattcaaaaggcaggggcatggttcagtatcgtcgaccttgaaactgga
gaaattatgacagatgaagacgaagaaccattgaagttccaaggcaaggcaaatctagtt
cgacgcttcaaggaggatgactacttattcgacatggtgatgactgcggttcacgaaatt
atcactcgagaagaaggctaa

| | |
|---|---|
| >dp1ORF011 DNA sequence | (SEQ ID NO. 21) |

atgaatatttatgattatatcaacgcaggggagattgctagctacattcaagcacttcct
tcaaacgctcttcaataccttggaccaactcttttccctaatgctcaacaaacagggaca
gacatttcatggctcaagggtgcaaataatttgccagtaactatccagccatctaactac
gacgcgaaagcaagtcttcgtgaacgtgctggatttagcaaacaagctactgagatggca
ttcttccgtgagtctatgcgacttggtgaaaaagaccgtcaaaacttgcaaatgctattg
aaccaaagttcagctcttgcccaaccacttatcactcaactctataatgatactaagaac
cttgtagacggtgttgaagcgcaagcagaatacatgcgtatgcaattgcttcaatacggt
aaattcactgtcaaatcaactaacagcgaggctcaatacacttacgactacaacatggat
gctaagcaacaatatgcagtcactaagaaatggactaacccagctgaaagtgaccctatc
gctgacattttagcagcaatggatgacatcgaaaatcgtacaggtgttcgccctactcga
atggtcttgaaccgaaacacttataaccaaatgactaagagtgactctatcaagaaagct
cttgcaattggtgttcaaggttcttgggaaaacttcttgcttcttgcaagtgacgctgag
aaattcatcgctgaaaaaacaggtcttcaaatcgctgtctactctaagaaaattgctcag
ttcgctgacgctgacaaacttcctgacgttggtaacattcgtcagttcaacttgattgac
gacggtaaagtggtattgcttccacctgacgcagttggtcacacttggtacggtactact
ccagaagcattcgacttggcttcaggcggaacagacgctcaagttcaagttctttcaggc
ggacctaccgttacaacttatcttgaaaaacatcctgtcaacattgcaacagttgtatca
gctgttatgattccatcattcgaaggaattgactatgtaggagttctcacaactaattag

| | |
|---|---|
| >dp1ORF012 DNA sequence | (SEQ ID NO. 22) |

atgagtattaagttcaaaaccgaagaactttcaaaaattgtttctcagctcaataagttg
aagcctagcaagttgctagaaatcacaaactattggcatatttttggtgacggcgaatgc
gtcatgtttacagcgtatgatggctcaaacttccttcgatgcattatcgacagcgatgtt
gaaattgacgtgattgtgaaagcagagcagtttggaaaacttgtagaaaagaccacggcc
gcaaccgtcacattagttcctgaagaatcttcgctaaaagttattgggaatggtgagtac
aatattgatattgttacagaagatgaagagtaccctacattcgaccacttgctcgaagac
gtgagtgaagaaaatgctctcactttgaaaagctcgctgttctacggaatcgccaatatc
aacgattctgcggtatctaaatcaggagcagatggaatttataccggcttcctgttaaaa
ggcggaaaagcaattactacagacatcattcgcgtatgtatcaaccctatcaaggaaaag
ggactagaaatgctcattccttacaacctaatgagtattttagcaagtattcctgatgag
aagatgtacttctggcaaattgacgatactactgtctatatttcatcggcttcagtcgaa
atttatggaaaattgatggaaggtatggaagattatgaagacgtttcacagcttgactca
attgagtttgaagatgatgcggctatccctacagcagaaatcctgagcgtattagaccgc
cttgtactattcacttcagcctttgacaaaggaaccgtcgaattcttattcttgaaagac
cgacttcgaattaaaacttctactagcagttatgaagacatcatgtacgcatctgctggc
aagaaagtttcgaagaaagaattcacttgccaccttaacagcttactcttgaaggaaatt
gtatcaaccgtcaccgaagaaaacttcactgtctcttatggaagcgaaaccgcaattaag
atttcatcgaatggtgtcgtttacttcctagcacttcaagagccggaagaataa

| | |
|---|---|
| >dp1ORF013 DNA sequence | (SEQ ID NO. 23) |

atgaatttagcttctaaataccgtcctcaaactttcgaggaagtggtagctcaagaatat
gtcaaagaaattcttttgaatcaattacaaaatggcgctatcaaacacggctatctattc
tgtggtggcgctggaactggtaaaaccactactgctcgaattttcgcgaaggatgtgaac
aaaggacttggctctcctattgaaattgatgctgcttctaataatggggtagaaaatgtt
cgaaacattattgaagattctagatacaagtctatggacagcgagttcaaagtttacatc
attgacgaggttcatatgctttcaaccggagcatttaatgcgctgttgaaaacattagaa
gagccctcatcgggaaccgtgttcattctatgtactactgaccctcaaaagattcctgac
actattctcagtcgagttcaacggtttgactttactcgaattgataatgacgacatcgtt
aatcaacttcaatttattatcgaaagtgaaaatgaagaaggagctggttatagttatgag
cgtgacgccctttcgtttattgggaaacttgcaaatggaggaatgcgtgacagtatcaca
aggctcgaaaaagtccttgattatagtcatcacgttgacatggaagccgtttctaatgca
ctaggagttccggactacgaaacattcgcttcacttgttgaagctattgccaactatgac
ggctcaaagtgtttagaaattgtaaatgacttccactactcaggaaaagacttgaaatta
gtgactcgaaactttacagacttccttttagaggtttgtaagtattggctagttcgagat
atttcaatcactcaacttcctgctcattttgaaagtaagctagagcaattctgtgaggct
tttcaatatcctactctattgtggatgctagaagaaatgaatgaacttgctggagttgtt
aaatgggagcctaatgctaaaccgataattgaaaccaaacttcttttgatgagcaaggag
gagtga

| | |
|---|---|
| >dp1ORF014 DNA sequence | (SEQ ID NO. 24) |

atgaaagtaaatggtcttcaaattgaagcgactcctgaacaaataattgaaaaactttcg
agacaacttgaagacgaaggaacattcatttttagacgaactaagtcgcttggaagcaac
tatcaattctcatgcccgtttcatgcaggagggactgaaaagcatccctcttgtggcatg
agtagaaatccttcttattcaggaagtaaggtgacggaagctggaacggttcactgtttc
acttgcggctacacttcaggactaactgaattcgtctcgaatgtattaggtcgaaacgat
ggagggttctatggaaaccagtggctgaaaaggaattttggaacatctagcgaagtagtt
aggcaaggcgtcagccctgaagcgtttcgaagaaatgggagaactgaaaaagtcgagcat
aaaatcattcctgaagaggaacttgataaataccggtttattcatccttatatgtatgaa
cggaaattgacggacgagctcatcgagatgtttgatgtaggttatgacaaactgcatgat
tgcatcacctttccagtacggaacctcaagggcgaaacagtattcttcaaccgtcgaagt
gttcgttctaagtttcaccagtacggtgaagatgaccctaaaacggaatttctttatggc
caatatgagcttgtagcatttcgagactattttgaaaaacctattagtcaagtattcgtg
actgagtctgttatcaactgcttgactctttggtcaatgaagattccagcagtcgctctt
atgggagtaggtggaggaaatcaaatcaatttactaaaacgacttccttatagaaatatt
gttctagcacttgaccctgataacgctgggcagacagcgcaggaaaaactctaccgacag
ttaaagcgaagcaaggtcgttagatttttgaactaccctaaagagttctatgataataag
tgggatataaacgaccatccggaattattaaattttaatgatttagtcttgtag

| | |
|---|---|
| >dp1ORF015 DNA sequence | (SEQ ID NO. 25) |

atgggatttaatctatacttcgcaggaggtcacgctattagcactgacgattatttgaag
gaaagaggagccaatcgcctattcaatcaactgtacgaaagaaacgggattggcaaaagg
tggattgagcataagaaaaccaatccaagcactacttcaaaactattcgtcgactctagt
gcatattctgctcataccaaaggggctgaagttgacattgacgcctatatcgaatacgtg
aatgataacgtgggaatgtttgactgtatcgccgaactcgataaaattcctggtgtattt
agacagcctaagacacgtgaacagcttttggaagcaccacaaatttcttgggataattat
ctatacatgcgcgagcgaatggttgagaaagacaagctcttacctattttccatatggga
gaagactttaaatggctcaacttgatgctcgaaactacattcgaaggcggaaagcatatt
ccttacattggaatttcaccagccaatgactcgactacgaagcataaagacaagtggatg
gaaagagtattcgaagttattcgaaacagttctaatccagacgttaagactcacgcattt
gggatgacagttactagccaattagagcgtcacccattctatagcgccgactctacttct
gtactgctcacaggagcgatgggaaacattatgacgtcaaaaggattagttgacttgtca
cagaagaatggaggaattgatgctgtccgtaggctgccaaaaccggttcaagttgaaatt
gaatccattatcgaagaaactggagcgcattttagcctagagcaattagttgaggactat
aaacttcgagcattgttcaatgttcaatacatgctgaattgggcagagaactatgaattc
aagggaattaaaaatcgtcaacgtcgactattttag

| | |
|---|---|
| >dp1ORF016 DNA sequence | (SEQ ID NO. 26) |

atgggagtcgatattgaaaaaggcgttgcgtggatgcaggcccgaaagggtcgagtatct
tatagcatggactttcgagacggtcctgatagctatgactgctcaagttctatgtactat
gctctccgctcagccggagcttcaagtgctggatgggcagtcaatactgagtacatgcac
gcatggcttattgaaaacggttatgaactaattagtgaaaatgctccgtgggatgctaaa
cgaggcgacatcttcatctggggacgcaaaggtgctagcgcaggcgctggaggtcataca
gggatgttcattgacagtgataacatcattcactgcaactacgcctacgacggaatttcc
gtcaacgaccacgatgagcgttggtactatgcaggtcaaccttactactacgtctatcgc
ttgactaacgcaaatgctcaaccggctgagaagaaacttggctggcagaaagatgctact
ggtttctggtacgctcgagcaaacggaacttatccaaaagatgagttcgagtatatcgaa
gaaaacaagtcttggttctactttgacgaccaaggctacatgctcgctgagaaatggttg
aaacatactgatggaaattggtattggttcgaccgtgacggatacatggctacgtcatgg
aaacggattggcgagtcatggtactacttcaatcgcgatggttcaatggtaaccggttgg
attaagtattacgataattggtattattgtgatgctaccaacggcgacatgaaatcgaat
gcgtttatccgttataacgacggctggtatctactattaccggacggacgtctggcagat
aaacctcaattcaccgtagagccggacgggctcattactgctaaagtttaa

| | |
|---|---|
| >dp1ORF017 DNA sequence | (SEQ ID NO. 1) |

atgattggacagggacttgttaaatctaccatttcgaaatggaaacaacttccaaaatat
ataatcgtcgaaggtgaagtaggttcaggacggaagaccttaatccgttatattgcttcg
aaatttgacgctgattctattgtagtaggaacgagtgtagatgacattcgaaacatcatt
caggatgcacagactattttcaaggcgagaatctacgtgatagacggaaatagcctgtca
atgtcagctcttaactcgcttttgaagatagcggaagagccacctttaaactgtcatata
gccatgactgttgatagcatcaataatgctttacctacgcttgcaagtagagcaaaagtt
ctaaccatgctaccttatactaatgaagagaaaatgcagtttgtcaagtcctacaagaag
gtagatacttcaggaattgacgaccgagcgattgtagactattgcaatcttgccagcaat
cttcaaatgcttgaagacatattagaatatggcgcagaagagctatttgaaaaggttaca
acattttatgacttaatatgggaggcaagtgctagcaattcgctaaaggttactaattgg
ctcaaatttaaggaaactgatgaaggaaaaattgagcctaaacttttcctcaactgtctt
ttaaattggtcgacagttgtcatcaggaagcactatgtagaaatgtctttcgaagaactt
gaggcccatgaccttttagtgagggaagcatctaggtgtttgcgaaaggtatctaaaaag
ggctcaaatgcgcgtgtctgcgtgaacgaatttatcaggagggtcaaacaagttgagtga

| | |
|---|---|
| >dp1ORF018 DNA sequence | (SEQ ID NO. 27) |

atggctagcagacagacgctattggtcgacggaattgaccttgtcgacaaaggtgcaacc
gtgctagaatatgtaggactcactttcgcaggatttaaggactcaggatttaaaaaccct
gaaggcatagacggagtattagattctccgtctaatgctatgtccgctcttactggaagc
gtgaccttaatgttccacggagaaaccgaaaagcaagttaatcaaaaatacaggcagttc
aaacaatttattcgctcgaagtcattttggagaatttcgacacttgaagaccctggatac
tatcgaacgggaaaatttttaggagaaaccgagcaaggaaaacttgtagacgttcaagcc
tttaaagatacttcccttgtagttaaattagggattcagttcaaagatgcttacgagtac
agcgactcaactgttcgaaaggtttataagtttcaacccgctttgggaggcgatagctta
cctaacccaggaagacctactcgacaatttagagtagaaataagaactacttctcaaatc
aaaggatattttcgaattggcgaaaaaagttcaggacagtttgttgagttcggtactaat
tcagtattgatggaaagtggctcgattattattctaaatcttggaacttttgaacttatt
aaaattagcagtgcaaatcaagcgactaacttatttagatacattaaacgaggcgcattc
ttcaagattcctaatggaaattcaacaattaccattgaataccgagccgatgacgcagca
gcttggacctctactcttcccgctcaagttgaactgtttctaaatccgtcttactattag

| | |
|---|---|
| >dp1ORF019 DNA sequence | (SEQ ID NO. 28) |

atgaatgtttatctcaatcaaatgggaaatgtagttcgagaaacttcggtttcaacagtc
tggaaaaccctcactcaaaaagggctcgtttctaatcatcgaatattcgctgttcgagat
gataaggagtttctgtctaatgagtcgaggtggaaaaggcttccggatgttagatatggg
acacttgttttgatggttactaaaattgacaagcgaagcaagttgctaaaggcctttcct
gataattgtgttgagtttgagaaaatgactgacgcgcagttgaaaaggcattttgtgtct
aaatactcgactattgatagcgacatgattgacatggttatccagttctgtctaaacgat
tactctagaattgacaatgaattggacaagctgtcgcgattgaaaaaggttgacgcatca
gtagttgaatccattgtcaagcacaagaccgaaattgacattttcagcctagttgatgat
gtattggaatataggccggagcaggcaattatgaaagtgactgaacttttagccaaagga
gaaagtcctattggattgcttaccttgctttatcaaaattttaataacgcttgtcttgtg
ctaggagccgatgagcctaaagaagccaatctaggcattaagcagttcttaatcaataag
attgtctataactttcaatacgagctggactcagcctttgaaggcatggctattttaggt
caagctatcgagggcataaagaatggtcgctatacagaaagttcagtggtctatatttct
ttgtataaaattttttcacttacttaa

| | |
|---|---|
| >dp1ORF020 DNA sequence | (SEQ ID NO. 29) |

atggttaatcaatacaatcagcctgaaagaggcaagattcgaatcaatgttcgcgaccct
gagaaaatgcctatcatggaaattttcggtcctacaattcaaggtgaaggaatggttata
ggtcaaaagactattttcattcgaactggtggatgcgactatcattgcaactggtgtgac
tcagcctttacctggaacggtactactgagccggaatatatcacaggcaaagaagctgct
agtcgaatcttgaaactagctttcaatgataaaggtgaacagatttgtaaccacgtgaca
ttgactggaggaaatcctgccttaatcaacgagcctatggctaagatgatttcgattcta
aaagaacatggattcaagtttggtctcgaaactcaaggaactcgattccaagaatggttc
aaagaagtaagcgatatcactattagtcctaaaccgccttcaagtggaatgagaactaat
atgaaaattcttgaagctattgtagatagaatgaatgatgaaaaccttgactggtcattt
aaaatcgttatctttgacgaaaatgacctagcttatgcgcgtgatatgtttaaaactttc
gaaggcaagttacgtccagtgaactacctttcagttgggaatgcaaacgcatacgaagaa
ggaaaaatcagtgataggcttcttgaaaagttgggatggctttgggataaagtgtatgaa
gacccagctttcaacaatgttcgacctttaccgcaacttcatacacttgtttatgataat
aaaagaggagtataa

| | |
|---|---|
| >dp1ORF021 DNA sequence | (SEQ ID NO. 30) |

atgcaaacgcatacgaagaaggaaaaatcagtgataggcttcttgaaaagttgggatggc
tttgggataaagtgtatgaagacccagctttcaacaatgttcgacctttaccgcaacttc
atacacttgtttatgataataaaagaggagtataaaatgaaaattgagcatctagataaa
atcggtaacgtattagggagagagaacggatgggcttcccttaagccggatgaaattgta
accttggacaatactgaggcagccgttcaaagactttttggtctattaggcgaggacgca
gaacgtgacgggttgcaagatactccattccgttttgttaaagcactcgctgaacatacc
gtagggtatcgagaagaccctaaacttcatctcgaaaaaacattcgacgtcgaccatgaa
gaccttgttcttgtgaaagacattccattcaattctttatgtgagcatcatttagctccg
ttcgtagggaaggtgcatattgcatacattcctaaggataagattacaggtctttcaaaa
ttcggtcgagtggttgaaggatacgctaaacgacttcaagtacaagagcgcttgactcaa
caaatcgctgacgctattcaggaagttctaaatcctcaagcagttgcggtcatcgtagag
gctgagcatacttgcatgagcggacgcggtattaagaagcacggggcaacgacagtgact
tcaactatgcgaggtcttttccaagatgacgcatctgctcgagcagaattgcttcagttg
attaaaaagtag

| | |
|---|---|
| >dp1ORF022 DNA sequence | (SEQ ID NO. 31) |

atgagtaaagacattctttacggaatcaagctcgtgcaaatcgaggagcttgacccattg
actcagttgccaaaagtcggcggagctaactttgtcgtagatacggcagaaacagcagaa
ctcgaagccgtgacctcggagggaactgaagatgtgaaacgcaatgacacgcgcattctt
gctatcgtgcgtactccagaccttttatacggttatgacttaacattcaaggacaacacg
tttgaccctgaaatcatggccctaattgaaggtggtacagtacgtcaacaaggcggaact
attgctggatacgacaccccaatgcttgcacaaggtgcttctaatatgaaaccatttaga
atgaacatctatgtgccaaactatgtaggtgactcaattgtcaactacgtgaaaatcact
ttgaataactgtaccggtaaagctccagggctttcaatcgggaaagagttctacgctcct
gagttcaacatcaaggcacgtgaagcaaccaaagcaggtttgccagttaagtcaatggac
tatgtggcacaacttccagcggttcttcgtcgcgtgacattcgatttgaacggtggaaca
ggaaccgccgacgcagttcgagttgaagcaggtaagaagatttctccaaaaccagttgac
cctaccttaacaggtaaggctttcaaaggctggaaagttgaaggagaatcaactatttgg
gacttcgacaaccacatgatgcctgaccgagacgtcaaactcgtagcacaatttgcatag

| | |
|---|---|
| >dp1ORF023 DNA sequence | (SEQ ID NO. 32) |

atggccaagtccaatttaactagaattgcaaagatggttagagcaggaaacagtgaaggt
cctgcttcatcttttgtcaattcgctgacccgggttattgaacgaactcagcctgaatat
aatccttcgacatattataagcccagcggggttggtggatgtattcgaaaaatgtatttc
gaaagaatcggtgagtctattatagataacgcagattctaacctaattgcaatgggcgaa
gctggaacatttaggcacgaagttctccaagagtacatggttaaaatggctgaaatcgat
gaggactttgaatggttgaatgtagcagagttcttgaaagaaaatccagttgaaggaact
atcgtcgacgagcgtttcaagaaaaacgattatgaaacgaagtgtaagaacgaacttctt
caactttcattcttgtgtgacggactagttcgatataaaggcaagctctacattttagag
attaagactgaaaccatgttcaagttcactaaacatactgagccctatgaagaacacaag
atgcaagcaacttgctacggaatgtgtctaggagtcgatgatgtcattttcctttatgaa
aatcgagataacttcgaaaagaaagcctacacgtttcacatcacagacgagatgaaaaat
caagtccttggaaaaattatgacctgcgaagagtatgtagagaaaggcgaaagtcctaaa
atctattgctcttcagcctattgcccatattgtagaaaggaaggtcgaaatctgtga

| | |
|---|---|
| >dp1ORF024 DNA sequence | (SEQ ID NO. 33) |

atgaacgcagtagatggccaggtagttcatattctacaagtattagcagaagatggaaat
gctacggctgaaaagttcgaaaaggaagtcagggctgcatctttagtattttcacgaaga
gcagccgaggcagttgtcaaaggtgaaatctataaggacggcaaaaacctctcgaaacgt
gtttggtcttcagccgcacgcgcaggaaatgatgttcaacaaatagtcacacaaggccta
gcaagtggaatgtctgctacagatatggctaaaatgctcgagaaatatatcgaccctaag
gttcgaaaagattgggactttgataagatagctgagaagctagggaaacctgctgctcat
aaatatcaaaatctcgaatacaatgcccttcgacttgctcgaactaccattagccattcc
gccacagctggagtgagacaatggggcaaggttaatccttatgctcgaaaagttcaatgg
cattctgttcacgctccaggtcgaacgtgtcaagcgtgtatcgatttagatggtgaagta
tttcctatcgaagaatgtcctttcgaccatcctaatggaatgtgctaccaaactgtatgg
tacgaaaactcactcgaagaaatcgctgatgagttgagaggctgggtagacggagaacct
aatgatgtattagacgaatggtacgacgatttaagttcaggaaaagttgagaaatacagc
gacctcgactttgttaaaagttattag

| | |
|---|---|
| >dp1ORF025 DNA sequence | (SEQ ID NO. 34) |

atggcaaagaacaaaaagcgaaaaaaagtaaatgtcaaaaggaaaatgcttatccctaca
aatctctcgaaaaaagtaaatgtaaaagcaatcgcttatagaaaagtcactgttaagtgg
ctgcctaatacagatgaaattcaagtatatttcgacctttatataaataaaaacaggctg
acaatgttaggcactattgacccggacaagagctattttgaaggaattaggattgtttgt
aagaaacctcagccttggatgactgttaaggagctccaggttgcgcgtgcagacgcccca
ggtttttttgcagttcttaaagcctattgtcacacggttggcgatgtactagatagcgga
gcagagcctactgaaattgttcaaggtattatgtataaagacggtgaactatttaaggac
agtgaaattgtcagccttttcaaatacgatgtcaaagagccttatgagtttccaaaggac
cttcctataaccttggacaactttttagagttcattatgtctagccagcatactagagca
cttgttttgcgttgtgctaatataggtgagttttccaagaattggcggaaatggcaaaaa
gctatccagctcctgctcgactatgccaaggcggatgactttaaagtagacgaaactgtt
tgggacttttcacccggctctaaagctggaaaggtagcacgtcgtaaaggctatgaggca
attcaacaagcccttgagcagataaataaataa

| | |
|---|---|
| >dp1ORF026 DNA sequence | (SEQ ID NO. 35) |

atggcgaaagctactggaccaaaagttcgaagaggaaaaactcctccacggccaaaagac
aaaaaaggaatcaaagcaaatgcgcgtgtcaataaagaccagttcgtagagtatgactat
aaaggcatcaagatgacaattaaggaacgtgatgctagaatgaaattggaatttattaga
ggcatgactattcaggaaattgcagcccgctatggattaaatgaaaagcgtgttggcgaa
atacgggctcgcgataaatgggtgaaggctaagaaagagttcgagaatgaaaaggctctt
gttactaatgatacattgactcaaatgtatgcagggtttaaagtctcagtcaatattaaa
tatcacgccgcctgggagaaactaatgaacatcgtcgaaatgtgtttagataatcctgac
agatatttatttactaaagaaggaaatattagatggggcgcattagatgtcctttcgaac
cttatagatagagctcaaaaaggacaagaaagagcgaatggaatgcttccggaagaggtt
cgatatagactacaaattgagcgcgagaaaattacattgctccgggccaaaatgggcgac
caggaaattgaaggcgaggttaaagataacttcgtagaagcactagataaagcagctcaa
gccgtttggcaagaatttagtgacgcaacaggttcctacattaaaggagtgactgataat
gacaataagcctgagaaataa

| | |
|---|---|
| >dp1ORF027 DNA sequence | (SEQ ID NO. 36) |

atgggaaaagtatcaattcaaaaatcaggaacatttagctcagggtctaataacgagttt
ttcacactcgctgaccacggtgacagcgcaattgtcactctattgtatgatgacccggaa
ggcgaagacatggattatttcgtagtccacgaagcagacgttgacggtcgtcgacgctat
atcaattgcaatgctattggcgaagacggggaaacagtccatcctgataattgtccatta
tgccaaaacggattccctcgtattgaaaaactatttcttcaactttacaaccatgatacg
ggaaaagttgaaacatgggaccgaggccgttcttatgttcaaaagattgttacatttatc
aataaatatggaagccttgtgactcagccttttgaaattattcgttcaggagctaaaggt
gaccaacgaactacttatgaattccttccagagcgtccggaagacagtgctactcttgaa
gattttccagaaaagagcgaacttcttggaactctaattttagacctcgacgaagaccaa
atgtttgacgtggttgacggcaagttcactcttcaagaagagcgttcttcaagtcgttca
aattcacgtagaggagcatctcctgcgcctagacgaggttccggtcgagaatcttcacaa
ggtcgaacagctgaaagaactccttcagttagtcgaagaactcctccaacacgaggtcga
ggattctaa

| | |
|---|---|
| >dp1ORF028 DNA sequence | (SEQ ID NO. 37) |

atgtcaaaaattaaattcgaaaaccttaaaaaaggcgatgttgtgctacgagctaaatct
caaacgaagtttaaaatcgtttcaattttagcagacgaaaagaaagcagaccttgaatca
ttagaagacggaggtgaacttcacctttcagcttcaactctcgaacgttggtacacaatg
gaagatgaaactgaacctaaaaaagaagaagctgctaaacctgctaaaaaggctgctcct
gcagttgctcgacctgctcgaaaaggtagagtcgttcccaaacctaaaaaagaagtcctt
gaggaagaaattcctgaagttaaggaacagccggaagaagttggttcagttagtgagaaa
tctactgttcgaaaacctgctcctaaaaaagaaagcgtgatggcgattactaaggctctt
gaaagtcgaattgttgaagcctttcctgcgtctactcgaatcgtcactcagtcttacatc
gcctatcgctctaagaagaacttcgttactatcgaagaaactcgaaaaggtgtttctatt
ggagttcgcgcaaaagggttgacagaagaccaaaagaaacttcttgcatctattgctcct
gcatcttacgaatgggcgattgacggaatttttaaactcgtcaaggaagaagatattgac
accgcaatggaattgattgaagcttctcacctttcttcgctatga

| | |
|---|---|
| >dp1ORF029 DNA sequence | (SEQ ID NO. 38) |

atgaaatcagtagttttattatccggcggagtcgactcagccacttgtttagcaattgaa
gttgacaagtggggttctaaaaatgttcatgctatagcattcaattacggacaaaagcat
gaagcagaacttgaaaatgctgctaatgttgcaatgttctacggagtcaagttcaccatt
cttgaaattgactcgaaaatctactcaagctctagctcttccttattacaaggaaaaggc
gaaatttcacatggaaaatcttacgctgaaatcctagcagagaaggaagtagttgacacc
tatgttccatttagaaatggactaatgctttcacaggctgcggcttatgcttattcggtt
ggagcttcttacgtcgtatatggtgctcacgcagacgatgcggctggaggtgcttaccct
gattgcactcctgagttctataattcaatgtcaaatgcaatggaatatggaactggaggc
aaggtaacccttgtcgctcctctacttactctaaccaaggcgcaagtcgttaaatgggga
attgatttagatgttccttatttcttgactcgttcatgttatgaaagtgacgctgaaagt
tgtggaacttgcgcaacttgtatcgaccgcaaaaaggcattcgaagaaaatggaatgact
gaccctattcattataaggagaattga

| | |
|---|---|
| >dp1ORF030 DNA sequence | (SEQ ID NO. 39) |

atgaataacgaaaaaattattgaaaaaattaaaaatcttattcaattagcaaatgacaac
ccgagtgacgaagaggggcaaactgcccttcttatggctcaaaagttgatgctaaagaat
aatatcgcacttgctcaagttgaacaatttgatgaacctaaacagttcgagacttctcaa
gctgttgggaaagaagcaggtcgaatattttggtgggaacgtgaacttggtcatattctc
gcgactaattttaggtgcttttgtattaatcagcgtgatatgcgcttgaataaaagtcga
ataattttcttcggcgaaaaacaagacgctgaattagtgtctaaaatatatgaggctgct
ttgctttatcttcgttaccgtattgaccgacttcctactcgcgaaccttcctacaagaat
tcatacctcaaaggctttttgtcagccttagccattcgatttaaaaagcaggtggaagaa
tattcacttatggtcctacctagcgagcaaacaaaaaatgcgcttcaggacacatttcga
aatttaaagaaggaaggaattgacagacctcaacatgacttcaatcttgaagcgtatatt
gaagggcggtttcatggcgagaatgcaaagattatgcccgatgaaattttggaaggcggt
aactaa

| | |
|---|---|
| >dp1ORF031 DNA sequence | (SEQ ID NO. 40) |

atggcttatcaattagaagacttgttaaaaggtctagatgaaccaactatcaaacaggtg
aaggaaattatttcgaaaacttcgaaagaactcgatgctaaaattttcattgacggcgac
ggtcaacattttgtacctcacgcacgtttcgatgaagttgttcaacagcgcgatgcagct
aacggctcaattaattcttataaagaacaagtcgcgacgctttctaaacaggtcaaagat
aacggtgatgcgcagaccactatccaaaaccttcaagagcaactcgacaagcagtctcaa
cttgcaaaaggcgctgtgattacttcagctcttcatccgttgattagtgactccattgct
ccagcagcagacattcttggatttatgaaccttgacaacattacggtcgaaagtgacggt
aaagttaaaggtcttgatgaagagttgaaagctgttcgtgagtctcgtaaatacttattc
aaagaagtcgaagttcccgcagaacaagaggctcaagctaagtcgccagccgggactgga
aatttaggaaatccaggtcgtgtcggtggtggtgttcccgaacctcgtgaaatcggctct
tttggtaagcaacttgctgctgctcaacaaacggcaggagcacaagaacaatcatcattc
tttaaataa

| | |
|---|---|
| >dp1ORF032 DNA sequence | (SEQ ID NO. 41) |

atgaaagaagcgaatagactagtttctagctatgtaggattcgaatgctggactgacgaa
gaatgtatcaggaactttgaactagaccctgatatgtcaattgcgtctgcttatcatcgt
tattttgggatgctttattcctatgcaaaaaggtttaaatgcttatctcgacatgacatt
gaaagcattgcattcgagactatttcaaaatgtttggcaacgttcaaatcaaaccaaggg
gccaagttttcaacttaccttacaagactcttcaagaatagaatagtcttagaatatagg
tacctaaatgcaccttccatgaatcgaaattggtatgtagaagtgacgttcgatagcgtt
tcgacaaatgaagaaggcgacgattttagtatcctatcgacagttggctattgtgaagac
tacggaaaaattgaaattgaagcaagtcttgacttcatgacgctttctaatacagagtat
gcttatatctcgtctgtcattcaaaacggtccttcagtaagcgacgcagaaattgcgcgt
gaaattggagtaagcaggtctgctattagtcagtctaagaagtcactaaaaaataaatta
aaagattttatataa

| | |
|---|---|
| >dp1ORF033 DNA sequence | (SEQ ID NO. 42) |

atggcaagacctaagttacctcaaattgatattcgagaagaagaaatacgagatgctcaa
gacgtagcagactcgtatggtgcgattatcaataaagtagtcgacgaaattgttgaagca
gcttgcggttcacttgaccaggcaatggaagaaattcaaatagttgtaagccaaaatcct
gtcattatggaagaccttaactactacattggctatcttcccactcttctttatttcgcc
gcagatagggcggaaatggtgggaatacaaatggattcaagttctgctatcaggaaagaa
aaatacgataatctatacattttagccgccgggaaaactattcctgacaagcaagcagaa
actcgaaaacttgtcatgaatgaagaagtcatcgaaaatgcttacaagcgagcctacaag
aaagttcaattaaagctagaacaggccgataaggtattagcatctttaaaacgaattcaa
acctggcaactagcagagttagaaactcagtcaaataattcaaaaggagtattattaaat
gcaaaaagacgtagacgtgaaaatgattga

| | |
|---|---|
| >dp1ORF034 DNA sequence | (SEQ ID NO. 43) |

atgagtcaaaacactacacgcactgacgctgaattgacaggcgttactcttttaggaaac
caagacaccaaatacgattatgactataatccagacgtccttgaaactttccctaacaaa
catcctgaaaataattacctagtaacatttgacggatatgaattcacttccctttgccct
aaaacaggacagcctgacttcgcgaatgttttcattagttacattccaaacgaaaagatg
gttgaatctaaatcattgaaattgtacttattcagtttccgtaaccacggtgacttccac
gaagattgcatgaacattattttgaatgacttgtatgaattgatggaacctaagtacatt
gaagtcatgggcctattcactcctcgtggtggaatttcaatttacccattcgtcaacaaa
gtgaatcctcaatttgcaactcctgaacttgaacagcttcaacttcaacgcaaattgaac
ttccttggaaatgttcaaggtcttggacgagctattcgatag

| | |
|---|---|
| >dp1ORF035 DNA sequence | (SEQ ID NO. 44) |

atgcacctaatgaaggattcgaagatgttgaggacatggaagtccttagcattcgagttc
gaaacgaaggtgaggacgacgagtgggttgaagttatcgcctgctatgaaaacgatgacg
aggacgaagatttggaagggttataaaatgaaggtatttatcaacaatcatactgaagct
gatattgactacaaagatattctaaattttgtagcttatcgaaactctcctaaccctcaa
attcaaatcactagctggaacgctttgctttcctgctatacacggaatgagctttcttat
aaaggagtttcaataacggacttttttgaagccattcaaactattgcaagttccttcact
cacctagactcgaaaacaattgatacacaaaatgaaaagcgactcgaaaggattgaggaa
cttcagtcaagaataggtcattgtaactgtactatcgacgaacttaaaaaaggagtccac
gaaatgccggatattgaatcagctatttcttaccagtacggacagattcttgcttatgaa
gatgaacttaattttctgctaaactaa

| | |
|---|---|
| >dp1ORF036 DNA sequence | (SEQ ID NO. 45) |

gtgttagtcgaacgaaaagccgacaaggaatgttgggaatggctagaagctgttcgagca
aatatagtcgaagaagttcgaaacggtcttagcattgttattgcttcgaatactgtcggg
aatgggaaaactagctgggcggttcgacttttgcaacgctatttagcagaaactgcactt
gacggaagaattgttgagaaaggaatgtttgtagtgtcagctcaactattgactgagttc
ggcgactataattattttcaaaccatgcaagaatttctcgaacgtttcgagcgccttaag
acttgtgagctattagtcatagacgaaataggtggaggttccttaaccaaggcctcttat
ccttatctgtatgacttggttaattatagggttgacaataacttgtcgactatttatacg
actaattatactgacgatgaaattattgaccttttaggccaaaggctttatagtcgtata
tatgatacttcagtggttctagattttcaggcaagcaatgtaagaggattggaggtaagc
gaaattgaatcatag

| | |
|---|---|
| >dp1ORF037 DNA sequence | (SEQ ID NO. 46) |

atggtgaagaaattgaaatctaaaatctattcagttgcatatataattctagtagttatt
gcgaaccttgtgacaatttatttcgaacctttaaatgtgaaaggaattttaattcctcca
agcagttggtttatgggattcactttcctgcttataaatctaataagcaagtacgagaag
ccaaaatttgcaggttctttgatatgggtagggttattccttacctcgttgatttgcttt
atgcaaaacctaccacaatcgcttgtcgtggcttcaggagttgcattttggataagtcaa
aaagcaagtgtctttatattcgacaagctctcgaataaattagactcgaagattgcaaat
gctttgtctagcaacatcggttctattatagacgcaaccatatggatttcattaggactg
agtcctcttggaattggaacggttgcatatatagatattccgtcagccgtactaggccaa
gttctagttcagtttatcttgcagtcaattgcttcgagatatttgaaaaagtag

| | |
|---|---|
| >dp1ORF038 DNA sequence | (SEQ ID NO. 47) |

atgagagtttctaaaaccttaacattcgacgcagctcatcaactagttggacattttgga
aaatgcgcaaatttgcacgggcatacttacaaagtcgaaatttcattagcaggcggaact
tatgaccacggttcgagtcaagggatggttgttgacttttatcacgtcaagaaaatcgca
ggtacattcattgacagacttgaccacgctgttcttcttcaagggaatgaaccaatcgct
ttagcaaatgcagttgacaccaagcgagttctatttggatttagaactacggctgagaat
atgtcaagattccttacctggactctcacggagcttatgtggaagcatgctcgtatcgac
tctatcaaactatgggaaactcctacaggttgcgcagaatgtacttactacgagattttc
acagaagacgagattgaaatgttcaagaacgtaacctttatcgacaaagacgaaaagatt
actgtccgcgaaattttagagcaggagcaggataatggttaa

| | |
|---|---|
| >dp1ORF039 DNA sequence | (SEQ ID NO. 48) |

atgaataaaagtgcaaccttttggcttgttcgaacagctcttattgcggctctatatgtg
acattgaccgttgcattttctgctattagttatggacctattcaatttagagtcagtgaa
gccttgattcttctacctttatggaaccatagatggactccggggattgtattaggaaca
attattgcaaacttcttttcacctcttggactgattgacgttttattcggttcacttgct
accttccttggagtagtggcaatggtgaaagttgctaagatggcaagtcctctatattca
cttatctgtccagttcttgctaatgcttaccttattgcgctggaacttcgaatagtttac
tctttacctttttgggaatctgtcatctatgtaggaattagtgaagcgattatcgtttta
atttcatacttccttatttccacgctggcgaagaacaatcattttagaacactgatagga
gcgaaaaatgggatttaa

| | |
|---|---|
| >dp1ORF040 DNA sequence | (SEQ ID NO. 49) |

gtgagctatactggaaaaatgttcgaggaagactttttcgaaggtgcaaaagactttgag
aaagatgctttcacggtccgtctatatgataccactaatggatttcgaggagttgcaaat
ccctgcgattatatagccgcaactaactttgggaccttgtttattgaactgaaaactact
aaagaagcttctttgagctttaataacatcactgataatcaatggttccagctatcacgc
gcagatggatgcaaatttattctcgccggaattttagtgtatttccaaaagcatgaaaag
attatatggtatccaatttcaagccttgaaaaaattaaacggtctggagttaaaagcgtc
aacccaaacttcatcgatgcagggtatgaagtttcttacaagaagcgtcgaactagattg
accattcctttccaaaatgttctagatgcagttgagcttcattacaaggagaaaagcaat
ggcaagacctaa

| | |
|---|---|
| >dp1ORF041 DNA sequence | (SEQ ID NO. 50) |

atgcaaaaagacgtagacgtgaaaatgattgaccctaaacttgaccgattaaaatacaca
ggtgattgggttgatgtacgaattagttctatcactaaaattgacgccgacagcgccgat
gtctcaagatgtcgaaaagtgcttcaaaaggctcaagtatattcagtggcggcaggtgaa
tgcattaaaattgcacacggatttgctcttgaacttcctaagggatatgaagcaatcttg
catcctcgttccagtctttttaagaaaactggtctaatcttcgtttctagcggagtgatt
gacgaaggttacaaaggtgacactgatgaatggttctcagtttggtatgctactcgtgac
gcagatatcttctacgaccaaagaattgcccaatttagaattcaggaaaagcaacctgct
atcaagttcaatttcgtagaatctttaggaaatgcggctcgtggaggccatggaagtaca
ggtgatttctaa

| | |
|---|---|
| >dp1ORF042 DNA sequence | (SEQ ID NO. 51) |

gtggcaaggcaaagaataggcaattcaggaaagcctaaaaatgaaattgaactaacattc
aaagacaagcctaaaactcgttctaccttattcaagaaggacgtggcaacaggtctttca
aaagtcgagcatgattattttcaaatagttgaagcacttaacggaaaacaattcgaacct
aatatgaagcaggtgtcatctttctttatagttcagtatgaatttattttcaatattaag
tgcatcgattataactggttcaacttttcgagcactatgaaaaatgttcgaacttattta
aacattgagtcgaacattgaactttgtcgatttttagctgaaagttttgttaaatatgaa
aatgttcgaaaaagattgaacctaagcgaaaggttcataacggtctcgactttcaaaaga
gcctggattttggacgaactcgaaggaaaaacgggttcaaaattcgaaggattttattag

| | |
|---|---|
| >dp1ORF043 DNA sequence | (SEQ ID NO. 52) |

atgactaatattatcacagctgagcagtttaagcaacttgcatttcaaatcatcgcactt
ccaggattttcaaaaggtagtgaacctatccatgttaaaattcgagcagcaggtgtcatg
aacctaatcgctaacgggaaaatccctaatacgcttttaggtaaagtgacagaactgttt
ggagaaacttcgacagtcactaaagacaatgctagtctagcatcaattactgaccaacag
aagaaagaagcgctcgaccgattgaacaaaaccgataccggtattcaagacatggctgaa
cttcttcgagtattcgcagaagcttcaatggtagagcctacttacgctgaagtcggcgag
tatatgacagatgagcaacttatgacaatcttcagtgcaatgtacggtgaagtgactcaa
gctgaaacctttcgtacagacgaaggaaatgtctaa

| | |
|---|---|
| >dp1ORF044 DNA sequence | (SEQ ID NO. 53) |

atggtaagtgttttgattagcagcagctcctttttgaagttcctgcttcattttagctcg
acaagtatttctaaatcgaataaggttttcaatttccttgtttcctacataagtggtgaa
ccgataatggcacttaggacattcgaagaatctccactctacgcccttttcgatatgttt
cgaaataatctgtttagatgtaaggtcgaacttatgctcacaatggtcacaattaacctt
gaacgtctgggtcgactccttcttcggttggttgttcagtttgttctttttctttgtcat
caacttcgtcttcttcactcgtttcatcttgaggctcctcttgttcgtttaattcgtttg
ctaatacaggcaatgctccagctgagatttcgtcaagctgagcaagttcttccaaaatgc
gttcccattccttgtccgccttttccttcttactga

| | |
|---|---|
| >dp1ORF045 DNA sequence | (SEQ ID NO. 54) |

atgaaacgagtgaagaagacgaagttgatgacaaagaaaaagaacaaactgaacaaccaa
ccgaagaaggagtcgacccagacgttcaaggttaattgtgaccattgtgagcataagttc
gaccttacatctaaacagattatttcgaaacatatcgaaaagggcgtagagtggagattc
ttcgaatgtcctaagtgccattatcggttcaccacttatgtaggaaacaaggaaattgaa
aaccttattcgatttagaaatacttgtcgagctaaaatgaagcaggaacttcaaaaagga
gctgctgctaatcaaaacacttaccattcatatcgaattcaggatgagcaagctgggcat
aaaatctcagggcttatggcgaagctaaagaaggagataaacattgaaaaacgagaaaaa
gaatgggtatctatatag

| | |
|---|---|
| >dp1ORF046 DNA sequence | (SEQ ID NO. 55) |

atgccaatgtggctaaacgacacagcagtcttgacgacgattattacagcgtgcagcgga
gtgcttactgtcctactaaataagttattcgaatggaaatcgaataaagccaagagcgtt
ttagaggatatctctacaactcttagcactcttaaacagcaggtcgacgggattgaccaa
acgacagtagcaatcaatcaccaaaatgacgtcattcaagacggaactagaaaaattcaa
cgttaccgtctttatcacgacttaaaaagggaagtgataacaggctatacaactctcgac
cattttagagagctctctattttattcgaaagttataagaaccttggcggaaatggtgaa
gttgaagccttgtatgaaaaatacaagaaattaccaattagggaggaagatttagatgaa
actatctaa

| | |
|---|---|
| >dp1ORF047 DNA sequence | (SEQ ID NO. 56) |

atgaaatttgaagatgaaaaacagttcatcgctgcaattgaagaagccggtgaattaaat
gctaccaaaggcgacatggagaaacaagtcaaaagtcttcgtgatgctctaaaagagtac
atgaaagaaaatgacattgaatctgctcaaggtaagcacttttctgctaccttctacacg
acagagcgctcaactatggacgaagaacgcttgaaagaaattatcgaaaaattagttgac
gaagccgagacggaagaaatgtgtgaaaaactttcagggcttatcgaatacaagcctgtc
atcaatacgaaacttctcgaggatatgatttatcacggcgagattgaccaagaagcaatt
cttccagcagttgtcatttctgttacagaaggcattcgttttggaaaggctaaaatttag

| | |
|---|---|
| >dp1ORF048 DNA sequence | (SEQ ID NO. 57) |

atggaaacaacactttatttcggttatcttacagcagattggaaagacggtcacaagaac
tacactttccactatgaaagcattcctgtaaaagaaactgagaaacaatataaggtcact
ggaatcaatcctaacttgtacttagacctaggctcagttattagaaagagcgaacttgac
attgcagtattcaaagcatgtcctgtcgctgaaactggagtcacacttactcgcgacatg
gaagttgatgctagaattgaaatcatcaagaaattaactacaagaatcgaacgccttaac
gaaagaattaaagcaagaaatgaacaaggtaaacaagaaagccgccacctagtatctgcg
ctagaagattgcgctcgtcaaattgctggaatttatcaataa

| | |
|---|---|
| >dp1ORF049 DNA sequence | (SEQ ID NO. 58) |

atgtttcaaccatttctcagcgagcatgtagccttggtcgtcaaagtagaaccaagactt
gttttcttcgatatactcgaactcatcttttggataagttccgtttgctcgagcgtacca
gaaaccagtagcatctttctgccagccaagtttcttctcagccggttgagcatttgcgtt
agtcaagcgatagacgtagtagtaaggttgacctgcatagtaccaacgctcatcgtggtc
gttgacggaaattccgtcgtaggcgtagttgcagtgaatgatgttatcactgtcaatgaa
catccctgtatgacctccagcgcctgcgctagcacctttgcgtccccagatgaagatgtc
gcctcgtttagcatcccacggagcattttcactaattag

| | |
|---|---|
| >dp1ORF050 DNA sequence | (SEQ ID NO. 59) |

atgaacaatcagcgaaagcaaatgaacaaacgaatcgtcgaacttcgcgaagactatcaa
cgtgcaagaggtcgaataaacttccttcttgctgtaaaggaccacggcgaagaactcgaa
aaccttgaagcctttgtgggatacattgacaatctagtcgaatgttttcctgaaagccaa
cgaaatgtcttgaggctatgtgtattagatgaccttccagtcactaatgcggccgctgaa
attggataccactatacatgggttcaccaacttcgagacaaagcagttgaaacacttgaa
gaaattttagatggggataacattattcgctctaaacacggaatcgaaattaaggagaaa
cttgatgaattatatggtaaaagtcattctagttag

| | |
|---|---|
| >dp1ORF051 DNA sequence | (SEQ ID NO. 60) |

atgagttatgacgtgaattatgttaagaatcaagttcgtagagccattgaaaccgctcct
actaaaatcaaggtacttcgaaactcttgggtcagtgatggatatggaggaaagaaaaag
gataaagcgaatgaagtcgtagcagacgaccttgtttgtttagttgataattcaactgtt
cctgaccttttagccaattctactgacgcgggaaaaatttttgcccaaaatggagtgaaa
attttcattctatatgatgaaggcaaaatcattcaacgagccgatactatcgaaattaaa
aactcaggaagacggtacagggtagtagaaacccacaatcttctcgagcaagacattttg
atagaacttaaattggaggtgaacgactaa

| | |
|---|---|
| >dp1ORF052 DNA sequence | (SEQ ID NO. 61) |

atgactaaacgaacgacaatgatggacagattgaaggaaattcttcctacatttcagctc
tcgcctgctcctatgcttccaggagttgaatttgacgagcaagatacagataggccggat
gactacattgttcttcgatatagtcatagaatgcccagcgcaacaaatagcctaggaagt
tttgcttattggaaagttcaaatctacgtccattcaaactcaattattggtatcgacgaa
tatagcagaaaggttcgaaacattatcaaggacatgggctacgaagtaacctatgcagaa
actggtgactacttcgacacaatgctttctagataccgactagaaatcgaatatagaatt
ccacaaggaggaaactaa

| | |
|---|---|
| >dp1ORF053 DNA sequence | (SEQ ID NO. 62) |

atgctaacattcgaaagaatagtatctatacgagcaccaacttgcatttcactcatttcc
ccgctatatagaaggacatcatgcccgttcttccaagcagttgcaagcattttatcaata
gtccacgacttaccttgtccaggtcgagccattatgacaatcaaatcctcaccaggaagt
aagcctccaagcacgtcgtccaatagttcaaaccctgtcgatattccaagtctttcaccg
tcatggtttctaatagtattcgcccagtctagtcgaagtttagcatttcgagcaatgtct
agtccgcctacgaatttagagcgattgaaaagttcttctagttttggaattatattcgca
atcgcaatgttactatctacttga

| | |
|---|---|
| >dp1ORF054 DNA sequence | (SEQ ID NO. 63) |

atgtgtgaaaattgtcaaaacgaaacattcaatactagaattttcaatgaagatgaaagt
ggctatgtcgacgcctcattcacttacaaggagattcgcgacaccgcagcagctattagc
aatcgagcggtagaaaagaaagaccgtgacagccttttagtcgctacagttatggctctt
cccgtttctcacgcagaagatttaggcaagagactttgtattgcaaattctcgattggaa
gcatttcgtgaagctgttcaagaggctctcgagaatgaaaaggctgaagatttaaaggac
gttatcttaggtcttatcgacgttgacaaaaaaattggcaaccttgcattgcaattagtt
gaatcaggagcattataa

| | |
|---|---|
| >dp1ORF055 DNA sequence | (SEQ ID NO. 64) |

atgcctaatgtgcgagttaagaaaactgattttaatcaaaccactcgaagcattgtcgca
attcctgaccactacgttgctttggctgctcaaattccagctaccgcagcaactcaagta
gggaacaagaaatacattcttgccggaacttgcgtgaaaaatgctactacatttgaagga
cgcaaaactggactcgaagtagtatctaccggtgaacaattcgacggagttatcttcgct
gaccaagaagtgtttgaaggtgaagaaaaagtaaccgtgacagtattagttcacggattc
gtcaaatatgcagcccttcgaaaagttggcgatgctgtgcctgaatctaaaaacgcaatg
attcttgtcgttaaatag

| | |
|---|---|
| >dp1ORF056 DNA sequence | (SEQ ID NO. 65) |

atggaaaataaatggaaagttatccattttcaaaactcatgtattaaacaagtagacgat
gaaaaaaggaggctcctgttcgaagttccaggaactccttatcgtctacaagtttgggtg
aaaatgagcttagttaaaattgaaacacgcgcaggaaatggctattataaaaggctagta
tgccaagacgattttgtattttatggtaaggagtcaatagatggttacttaattgacgcc
accataactggcaaatctttggcggaatattgtgagcctatgaacaggcatattctcgaa
actattgcatcgcgagaagcagctgaactgaacagagctaaaaagcaagaccaacagaaa
tggagatactag

| | |
|---|---|
| >dp1ORF057 DNA sequence | (SEQ ID NO. 66) |

atgcaaaaatctctatttggacctaagctagtgcctgctagttcaaggcgcaagaaaaga
acggttccaaaacctaaacctaaaatcgatgagcaagtggttgagcttatgaaccgcaga
gagcgtcaagtgcttgttcatagttgcatctattattattttaatgactcaattatagca
gacgggcagtatgacaaatggagccacgaactatattctcttatagtttcgcaccctgat
gagtttcgacagactgttctctataacgagtttaaacagtttgacggaaatactggaatg
ggtcttccatacgactgtcagtttgctgtaagggtcgcagaaaggcttttaagaaaatga

| | |
|---|---|
| >dp1ORF058 DNA sequence | (SEQ ID NO. 67) |

atgacatcacgcgcatacaaaccaattcccacgcgcagagctagtgctaaacaagagaag
gcagttgctaagcagttgggaggaaaagtacagcctaattcaggagccactgactactac
aaaggtgacgtcgtaacagactcaatgcttatagaatgcaagacagttatgaagccacaa
agttcagtcagcttgaaaaaggaatggttcctaaaaaatgaacaggaaaggttcgctcaa
aaactcgactattctgctatcgctttcgactttggtgacggaggcgaacagtatatagca
atgtctataagtcagttcaagcgaatattagaggatagaaatgataaccttatttaa

| | |
|---|---|
| >dp1ORF059 DNA sequence | (SEQ ID NO. 68) |

atgtctcagcctgaattagtatggaagcctgaagaatttgttagtaactgtgaacggtat
cgaaacaagtttcaagtcgctgtcataacagtctgcgaagtcgctgctactaagatggaa
gaatacgcaaagacgcatgctatttggacagaccgtacagggaatgctcgacagaaactc
aaaggagaagctgcttgggtaagcgcagaccaaatcatgatagctgtatcacatcacatg
gactacgggttttggctagaactagctcatggtcgaaaatacaaaattctcgaacaggct
gtagaagacaatgtcgaagaactttttagagcgttgagaaggttattagactag

| | |
|---|---|
| >dp1ORF060 DNA sequence | (SEQ ID NO. 69) |

gtgatagctgtatctgctatccctactccgctctttccaggtacaccgtcgactccatca
cgcccaggagctcccggtaaacctgcgtcacctttaggaccttctagtcgaatccatgta
aagtcgtcaggaactaattcgctcggtttcttattagtattaaggacaccaatgtatttc
ccagattctgcattaaaattagtccctaaaatgtcatctgcgtatctaataacaacttgg
gactcatttacagtttcccctgaaaggactccttcgccgtcctcatttagcaagtccatc
aagtcttttcgagggtcttggaaaatgatagtagagtttgaaaggtcgtcgtag

| | |
|---|---|
| >dp1ORF061 DNA sequence | (SEQ ID NO. 70) |

atggcgagaatgcaaagattatgcccgatgaaattttggaaggcggtaactaaaatgaaa
ttcgaagtttattctgcgcgactatttgacgaagaggcgacatatgataggtatcgtgaa
gcactagagaaagttggaaatgtcgcttacttttgtgaaattgatactggcaaccttgta
atcgaactcgagctagacagcctagatgacctaatcgcgctttcaaatgtagtgggaact
ggactaaaattatcacggccttatagagaagataagccttttcaattatggattgttgac
gggtacatggaataa

| | |
|---|---|
| >dp1ORF062 DNA sequence | (SEQ ID NO. 71) |

gtgagaagcttcaatcaattccattgcggtgtcaatatcttcttccttgacgagtttaaa
aattccgtcaatcgcccattcgtaagatgcaggagcaatagatgcaagaagtttcttttg
gtcttctgtcaacccttttgcgcgaactccaatagaaacaccttttcgagtttcttcgat
agtaacgaagttcttcttagagcgataggcgatgtaagactgagtgacgattcgagtaga
cgcaggaaaggcttcaacaattcgactttcaagagccttagtaatcgccatcacgctttc
ttttttaggagcaggttttcgaacagtagatttctcactaactga

| | |
|---|---|
| >dp1ORF063 DNA sequence | (SEQ ID NO. 72) |

atgaaattcactgaaggaaaaaattggtataaagttggagagatatgtcaaatgttgaac
cgctctctatctacgattaatgtttggtatgaagcaaaagacttcgctgaagaaaataac
attcacttcccgtttgttcttcctgaacctagaacagaccttgaccatcgtggttctcga
ttctgggatgacgaaggcgtgaacaaactcaaacgatttagggacaacctaatgcgcggt
gacttggcattctacactcgaactcttgtagggaaaactgaaagggaagcaattcaagaa
gatgctaaagcatttaaacgtgaacatggattggagaattaa

| | |
|---|---|
| >dp1ORF064 DNA sequence | (SEQ ID NO. 73) |

atggctacattgaaagctcttagcaccttaatcgtttccggagcagtagtgcattcaggg
tcggtattttcttgccctgaagcgcttgcttcgtctttaattgaacgcaattttgcgttc
gagattaaggcggctgaagatggagaaacggtagaaactgttcctcaaacaattgaatca
gttgaagaaattgacgaagttgaacaaatgcgcgaagagtatgcggctaaaaccgttcct
gagctcgttgaattagcaagagctaatggaattgacatttcttcaatttctcgaaaaagc
gaatatatcgacgctttaattaagtacgaactaggagagtaa

| | |
|---|---|
| >dp1ORF065 DNA sequence | (SEQ ID NO. 74) |

atgcagtttgtcataacctacatcaaacatctcgatgagctcgtccgtcaatttccgttc
atacatataaggatgaataaaccggtatttatcaagttcctcttcaggaatgattttatg
ctcgactttttcagttctcccatttcttcgaaacgcttcagggctgacgccttgcctaac
tacttcgctagatgttccaaaattccttttcagccactggtttccatagaaccctccatc
gtttcgacctaa

| | |
|---|---|
| >dp1ORF066 DNA sequence | (SEQ ID NO. 75) |

gtgaccaactgcgtcaggtggaagcaataccactttaccgtcgtcaatcaagttgaactg
acgaatgttaccaacgtcaggaagtttgtcagcgtcagcgaactgagcaattttcttaga
gtagacagcgatttgaagacctgttttttcagcgatgaatttctcagcgtcacttgcaag
aagcaagaagttttcccaagaaccttgaacaccaattgcaagagctttcttgatagagtc
actcttagtcatttggttataagtgtttcggttcaagaccattcgagtagggcgaacacc
tgtacgattttcgatgtcatccattgctgctaa

| | |
|---|---|
| >dp1ORF067 DNA sequence | (SEQ ID NO. 76) |

gtgacgattcgagtagacgcaggaaaggcttcaacaattcgactttcaagagccttagta
atcgccatcacgctttcttttttaggagcaggttttcgaacagtagatttctcactaact
gaaccaacttcttccggctgttccttaacttcaggaatttcttcctcaaggacttctttt
ttaggtttgggaacgactctaccttttcgagcaggtcgagcaactgcaggagcagccttt
ttagcaggtttagcagcttcttcttttttaggttcagtttcatcttccattgtgtaccaa
cgttcgagagttgaagctgaaaggtga

| | |
|---|---|
| >dp1ORF068 DNA sequence | (SEQ ID NO. 77) |

atggcagctcaaacggacattgaattagtcaaaatcaatatcgataacgataattctccg
tcaccaatgactgaccaaagtatctcagctcttttagacaagcataaatctgtcgcctat
gttagttatatgatttgcttaatgaagacccggaatgacgtggtaacccttggacctatc
agtctaaaaggtgacgcagactactggaaacaaatggcgcaattctattatgaccaatat
aagcaagaacagcttgaaactgatgaaaagtcgaacgctggttcgacaatcttaatgaaa
agggctgatgggacatga

| | |
|---|---|
| >dp1ORF069 DNA sequence | (SEQ ID NO. 78) |

atgaaactttatcacgccactgattttgataatcttggtaaaattctagctgaaggattg
aagccttcagctggagttatttacctagcagaaagttatgaaaaggctctagccttttta
tcgcttcgaaatgttgatactattgtcgttctcgaacttgaagtagatattgaaaaatgt
actgaaagtttcgaccataatgaaaagatgttttgtagcctatttcatttcgacacttgt
cgcgcttggacttatgacaagacaattgaagtagacgacattgacttttcgaaagctcga
aaatatgatagaaagtga

| | |
|---|---|
| >dp1ORF070 DNA sequence | (SEQ ID NO. 79) |

atgataaccttatttaaaataaacagtgaaggaacagttactccaattaaagggtcagcc
atgcaactgtacgcagaccttattcctatacaagaggacgatatacagttcgttgatata
actggacttgaccctattgttcgagaaaacgtacttgagctcatttcacggagccgtgta
ggagtttcaaaatatggtacaaacctcgaccagaatgatgtcgacgatttcctacagcac
gccaaagaagaagcgctcgactttgctaactacctaaccaagctacaaagtcaacaaaag
caaaataaatag

| | |
|---|---|
| >dp1ORF071 DNA sequence | (SEQ ID NO. 80) |

gtgaaacaggtcctagaggagttcaaggtcttcaaggtcctcaagggcttcaaggaattc
ctggacctgcaggagctgacggacgttcgcaatatactcacctcgctttctctaatagtc
caaacggtgagggatttagtcatactgacagcggacgagcatacgtcggtcagtatcaag
atttcaatcccgtccattcaaaagaccctgcagcctatacatggacgaaatggaagggga
atgacggagctcaagggatacccgggaagccaggcgcagacggtaagactaattatttcc
atatag

| | |
|---|---|
| >dp1ORF072 DNA sequence | (SEQ ID NO. 81) |

atgttccttcgtcttcaagttgtctcgaaagtttttcaattatttgttcaggagtcgctt
caatttgaagaccatttactttcatcaaaatgcttcaactccttcccttgtaaccttact
tcgaagacgagcagtcgacctagaggcttttgctttcaatggagagctttcgcctttttc
agttccttcttcgccttcctctttgaatcctataagagtataggttccagtttcaacgtc
ccacatatattcgatgatttttcggtcttcgccatatcggtttttaacgacagatag

| | |
|---|---|
| >dp1ORF073 DNA sequence | (SEQ ID NO. 82) |

gtgaacgcttgccggaagaatacgacgaagaaacttgggaacctatcactgaagcagaat
acatcaagcgaacagaaaaacctaaagcagttgcaaaacctactcgaaaaactccagcgc
cttctcgtcgccctcgcccttaaaagaaaggttgaaataaaatgtgtgaaaattgtcaaa
acgaaacattcaatactagaattttcaatgaagatgaaagtggctatgtcgacgcctcat
tcacttacaaggagattcgcgacaccgcagcagctattagcaatcgagcggtag

| | |
|---|---|
| >dp1ORF074 DNA sequence | (SEQ ID NO. 83) |

gtgacgaaaagaaaaatccaggattgcaaatgcttatggagtgactattttcagtcgctc
ctctttttgtatatagaaaggaaattacatggattttgggtcaattgcagcaaaaatgac
tttggatatctcaaacttcacaagtcaattaaatcttgctcaaagtcaagcgcaacggct
cgcactagagtcttcgaagtcctttcaaattggttctgctttaacaggattagggaaagg
acttacgactgcggttacccttcctcttatgggatttgcagccgcctctattaa

| | |
|---|---|
| >dp1ORF075 DNA sequence | (SEQ ID NO. 84) |

atggcaaagttttgtccgttgaattccgtcatggcccaaagggaaaatgaaagagccatc
gatactgtttttcctgaacgaatggaaccgtctgctatgacgatatcgaaagttcgaaaa
ggtgagccctttgtccaccatgttaggagctggagttgtttcttactaaaagggacgaag
ttgaacttaggtagtttatttctcaggcttattgtcattatcagtcactcctttaatgta
ggaacctgttgcgtcactaaattcttgccaaacggcttgagctgctttatctag

| | |
|---|---|
| >dp1ORF076 DNA sequence | (SEQ ID NO. 85) |

gtgagagcattttcttcactcacgtcttcgagcaagtggtcgaatgtagggtactcttca
tcttctgtaacaatatcaatattgtactcaccattcccaataacttttagcgaagattct
tcaggaactaatgtgacggttgcggccgtggtcttttctacaagttttccaaactgctct
gctttcacaatcacgtcaatttcaacatcgctgtcgataatgcatcgaaggaagtttgag
ccatcatacgctgtaaacatgacgcattcgccgtcaccaaaaatatgccaatag

| | |
|---|---|
| >dp1ORF077 DNA sequence | (SEQ ID NO. 86) |

atggaacgaataaagacgctatttcacgtgatttatgctaacggcactcatttagaagta
gcagctttgttcgataccgttgatgattatgatgacgttatagaggacatccaggggtat
attgatacccctgacctttataatcaaaggagcattagaatggcgccttacaatcctgac
atcaatggtgacgctattgctactgacattttactacgactagatgatattatctacgtc
gacgcaacttgtgaaactattaaatacgaggagcctattgcatga

| | |
|---|---|
| >dp1ORF078 DNA sequence | (SEQ ID NO. 87) |

atggcaacagtaaaggaaacagtaaaatttgacggacgtcttgtaactatcttcgactac
gacgatttagagtgggaaggatatgcacctaatgaaggattcgaagatgttgaggacatg
gaagtccttagcattcgagttcgaaacgaaggtgaggacgacgagtgggttgaagttatc
gcctgctatgaaaacgatgacgaggacgaagatttggaagggttataa

| | |
|---|---|
| >dp1ORF079 DNA sequence | (SEQ ID NO. 88) |

atggaactgataccattgataaatcctcgaacaaggttgacccctgcgcttaccatttgt
ccagcgaatccagtaaccttagaaacaattgaagttcccatgctgccaattttagagaca
gctgaaccaatcattgacccaataccactaatgaagtttcgaatcaggttcgcacctcct
gaaaccatctgtcccacaaagctagcaatcttgctaactaatgatgaaagcatgtttcca
gctgtcgataaaagtgagccgagaagtgaagcaataccttga

| | |
|---|---|
| >dp1ORF080 DNA sequence | (SEQ ID NO. 89) |

atgttgaaccttacaaaatcgcgccaaattgtggcagagttcactattggacaaggagct
gaaaagaaacttgtcaaaacaacgattgtgaacattgatgcaaacgcagtatcaaccgtc
tctgaaactcttcatgacccagacttgtatgctgcgaaccgtcgagaacttcgagctgac
gagcaaaaacttcgcgaaactcgttacgcaatcgaagatgaaattctagctgaacagtca
aagactgaaacagctctaacagctgaataa

| | |
|---|---|
| >dp1ORF081 DNA sequence | (SEQ ID NO. 90) |

atgttcaggaacagtatcgtccatctgttggtctgcgtcaaagttaaaggggtcgaaatc
ttcgttcttgctagcgtcgatatactcgaactcgtattcaggaagactcatatcaggaag
ccttcttcttcgaccggtagctgtttgaacatatcccaagtcctgcgcctgctgttgaac
gaatatgatatagtctgccactttagggaactcggtgaagaaatcttcaataaccttatt
cgcttctttgacagatacattcatctgctcagcgattga

| | |
|---|---|
| >dp1ORF082 DNA sequence | (SEQ ID NO. 91) |

gtgaacttcacctttcagcttcaactctcgaacgttggtacacaatggaagatgaaactg
aacctaaaaaagaagaagctgctaaacctgctaaaaaggctgctcctgcagttgctcgac
ctgctcgaaaaggtagagtcgttcccaaacctaaaaaagaagtccttgaggaagaaattc
ctgaagttaaggaacagccggaagaagttggttcagttagtgagaaatctactgttcgaa
aacctgctcctaaaaaagaaagcgtga

| | |
|---|---|
| >dp1ORF083 DNA sequence | (SEQ ID NO. 92) |

atgccttcagggtttttaaatcctgagtccttaaatcctgcgaaagtgagtcctacatat
tctagcacggttgcacctttgtcgacaaggtcaattccgtcgaccaatagcgtctgtctg
ctagccatctatttctcctttacggtgttacaatgttaccaaaccctgatagagtttctt
tacttctattatacaatcctctcgacagtttgtcaacgtcgtcattgtttcgaactacga
ttgttccaatgttga

| | |
|---|---|
| >dp1ORF084 DNA sequence | (SEQ ID NO. 93) |

atgaattatatggtaaaagtcattctagttagtgtctttgtactgtcagccttttgcatg
acttgctcaatggtttatttggttacaggtaagcaagaggaccaccgtagtaccgtcgcc
cttgtatttggcgctctcgtaagctctgcggcgttctattcgacactctttatcctcgcc
tatctgccatga

| | |
|---|---|
| >dp1ORF085 DNA sequence | (SEQ ID NO. 94) |

gtgatgactataatcaaggactttttcgagccttgtgatactgtcacgcattcctccatt
tgcaagtttcccaataaacgaaagggcgtcacgctcataactataaccagctccttcttc
attttcactttcgataataaattgaagttgattaacgatgtcgtcattatcaattcgagt
aaagtcaaaccgttgaactcgactgagaatagtgtcaggaatcttttgagggtcagtagt
acatag

| | |
|---|---|
| >dp1ORF086 DNA sequence | (SEQ ID NO. 95) |

atatgggaaaagtatcaattcaaaaatcaggaacatttagctcagggtctaataacgagt
ttttcacactcgctgaccacggtgacagcgcaattgtcactctattgtatgatgacccgg
aaggcgaagacatggattatttcgtag

| | |
|---|---|
| >dp1ORF087 DNA sequence | (SEQ ID NO. 96) |

atgattttgccttcatcatatagaatgaaaattttcactccattttgggcaaaaattttt
cccgcgtcagtagaattggctaaaaggtcaggaacagttgaattatcaactaaacaaaca
aggtcgtctgctacgacttcattcgctttatcctttttctttcctccatatccatcactg
acccaagagtttcgaagtaccttgattttagtaggagcggtttcaatggctctacgaact
tga

| | |
|---|---|
| >dp1ORF088 DNA sequence | (SEQ ID NO. 2) |

atgaaaaaagttcaaacttatcaagaatatctaaaactagttgagttcaaacgtcaactt
tctttaaatcttcgagaaggaaaaataggagtcgatgaagcggttattcaattattcacc
ttctatagtttcaacaatatcgaggaacctcctttcattgtactcaaaatgcaagaggct
gccgtgaacgggacttatgaagcaaaactcaatatgcttaaaagatttaaaattatttag

| | |
|---|---|
| >dp1ORF089 DNA sequence | (SEQ ID NO. 97) |

atgtcaatcatgtcgctatcaatagtcgagtatttagacacaaaatgccttttcaactgc
gcgtcagtcattttctcaaactcaacacaattatcaggaaaggcctttagcaacttgctt
cgcttgtcaattttagtaaccatcaaaacaagtgtcccatatctaacatccggaagcctt
ttccacctcgactcattagacagaaactccttatcatctcgaacagcgaatattcgatga

| | |
|---|---|
| >dp1ORF090 DNA sequence | (SEQ ID NO. 98) |

atgctaaaattttcattgacggcgacggtcaacattttgtacctcacgcacgtttcgatg
aagttgttcaacagcgcgatgcagctaacggctcaattaattcttataaagaacaagtcg
cgacgctttctaaacaggtcaaagataacggtgatgcgcagaccactatccaaaaccttc
aagagcaactcgacaagcagtctcaacttgcaaaaggcgctgtga

| | |
|---|---|
| >dp1ORF091 DNA sequence | (SEQ ID NO. 99) |

atgaaactatctaacgaacaatatgacgtagcaaagaacgtggtaaccgtagtcgttcca
gcagcgattgcactaattacaggtcttggagcgttgtatcaatttgacactactgctatc
acaggaaccattgcacttcttgcaacttttgcaggtactgttctaggagtttctagccga
aactaccaaaaggaacaagaagctcaaaacaatgaggtggaataa

| | |
|---|---|
| >dp1ORF092 DNA sequence | (SEQ ID NO. 100) |

atgaaaactatctccatattaaggaaagacactaaaaggaagccggacaggaacggaaga
aaaactgcactcgaactagctcaagagattgatatgtcacctagtgagttagcagagctc
cttcaaattcctgaaaggacggcaaccagaattttaaaactcgacaaactgctcaacaaa
gagcaatgctcaataatagaaaggtatataaatgaaattcactga

| | |
|---|---|
| >dp1ORF093 DNA sequence | (SEQ ID NO. 101) |

atgcaacatacgattaaacaatgtttgaaacttgccttcctgctaactgcaatatcaatt
gcctgtttagttttccctaaaccttgctcatcgcctaaaaggaaacatggatgctcttgt
gcgtattcgaaacattcaacctggtgcgcgaatggagtagtcttgaacgaaaactgctca
ttgcttgaagaagctattcggtttcgagagtcaatgtag

| | |
|---|---|
| >dp1ORF094 DNA sequence | (SEQ ID NO. 102) |

atgtacgaattagttctatcactaaaattgacgccgacagcgccgatgtctcaagatgtc
gaaaagtgcttcaaaaggctcaagtatattcagtggcggcaggtgaatgcattaaaattg
cacacggatttgctcttgaacttcctaagggatatgaagcaatcttgcatcctcgttcca
gtctttttaagaaaactggtctaa

| | |
|---|---|
| >dp1ORF095 DNA sequence | (SEQ ID NO. 103) |

gtgggaaaactacttcagctctcgacattgtcaagaatgcgcaaatggtatttgagcagg
aatgggaacagaagactgaagaactcaaggaaaagctggaaaatgcgcgtgcatccaaag
ctagcaagactgctgtcaaggaacttgaaatgcaactcgatagtcttcaagagcctctta
agattgtatatcttgaccttgagaatacattag

| | |
|---|---|
| >dp1ORF096 DNA sequence | (SEQ ID NO. 104) |

gtgattcataaattcttcaatttcgttgaacttatctgcggtttctcctgttaccaggtt
gcatttgactgtcttcgaaagtatcttagcaagaggttcaataaccttttcccaattgct
aaatatcacgcaggactttccttgctggatacattcctcgacaatttcgatacatctttc
gaacttgcaagacttgacatcttgagtagttaa

| | |
|---|---|
| >dp1ORF097 DNA sequence | (SEQ ID NO. 105) |

atggacgggattgaaatcttgatactgaccgacgtatgctcgtccgctgtcagtatgact
aaatccctcaccgtttggactattagagaaagcgaggtgagtatattgcgaacgtccgtc
agctcctgcaggtccaggaattccttgaagcccttgaggaccttgaagaccttgaactcc
tctaggacctgtttcacctatcttggaaactga

| | |
|---|---|
| >dp1ORF098 DNA sequence | (SEQ ID NO. 106) |

gtgaaaatgctccgtgggatgctaaacgaggcgacatcttcatctggggacgcaaaggtg
ctagcgcaggcgctggaggtcatacagggatgttcattgacagtgataacatcattcact
gcaactacgcctacgacggaatttccgtcaacgaccacgatgagcgttggtactatgcag
gtcaaccttactactacgtctatcgcttga

| | |
|---|---|
| >dp1ORF099 DNA sequence | (SEQ ID NO. 107) |

atgcaagttcgccatctgctactgaagctccagctggtggatggtctacgcaagttccta
ccgtcccaggtggtcagtatttatggactcgaacaagatggcgctacactgaccaaactg
atgaaattggatattcagtttcaagaatgggcgagcagggtcctaaaggtgacgcaggtc
gtgacggtattgcaggaaagaacggaatag

| | |
|---|---|
| >dp1ORF100 DNA sequence | (SEQ ID NO. 108) |

atgcagttgacaccaagcgagttctatttggatttagaactacggctgagaatatgtcaa
gattccttacctggactctcacggagcttatgtggaagcatgctcgtatcgactctatca
aactatgggaaactcctacaggttgcgcagaatgtacttactacgagattttcacagaag
acgagattgaaatgttcaagaacgtaa

| | |
|---|---|
| >dp1ORF101 DNA sequence | (SEQ ID NO. 109) |

gtgataattttagtccagttcccactacatttgaaagcgcgattaggtcatctaggctgt
ctagctcgagttcgattacaaggttgccagtatcaatttcacaaaagtaagcgacatttc
caactttctctagtgcttcacgatacctatcatatgtcgcctcttcgtcaaatagtcgcg
cagaataaacttcgaatttcattttag

| | |
|---|---|
| >dp1ORF102 DNA sequence | (SEQ ID NO. 110) |

atgataacgtgggaatgtttgactgtatcgccgaactcgataaaattcctggtgtattta
gacagcctaagacacgtgaacagcttttggaagcaccacaaatttcttgggataattatc
tatacatgcgcgagcgaatggttgagaaagacaagctcttacctattttccatatgggag
aagactttaaatggctcaacttga

| | |
|---|---|
| >dp1ORF103 DNA sequence | (SEQ ID NO. 111) |

ttgaatcatagatatagtaacatcacaactatttttctttggcagattgtctttctttgt
atttgctgcgcggtgtcctattgtgcaggagtgcataatgagcgagagtctcaagataag
gtgattcaaagttataagcagaaagaaaagtcagccgtctacttgacagtcgatagttca
ggagcttggctaggaagtgctccgggagccaaggaaagtcctctctacaatgaaaaggga
cagcatgtaggaaaattgaaagaggtgggagagtga

| | |
|---|---|
| >dp1ORF104 DNA sequence | (SEQ ID NO. 112) |

atgagaaaaagagtgattttgaagctaaaaaggttgaactggtatgtccttaattcctac
tctcgaatggttgagtttttcgaacttttgaacttttcgaatggttcgacttttcgaagg
attgaggttttcgaaccggttgagtttttcgagcattctcgacttttcgacccctttcta
tgctcgacttttcgagtgttttga

| | |
|---|---|
| >dp1ORF105 DNA sequence | (SEQ ID NO. 113) |

atgatagtcgcatccaccagttcgaatgaaaatagtcttttgacctataaccattccttc
accttgaattgtaggaccgaaaatttccatgataggcattttctcagggtcgcgaacatt
gattcgaatcttgcctctttcaggctgattgtattgattaaccattatcctgctcctgct
ctaaaatttcgcggacagtaa

| | |
|---|---|
| >dp1ORF106 DNA sequence | (SEQ ID NO. 114) |

atgaacctcgtcaatgatgtaaactttgaactcgctgtccatagacttgtatctagaatc
ttcaataatgtttcgaacattttctaccccattattagaagcagcatcaatttcaatagg
agagccaagtcctttgttcacatccttcgcgaaaattcgagcagtagtggttttaccagt
tccagcgccaccacagaatag

| | |
|---|---|
| >dp1ORF107 DNA sequence | (SEQ ID NO. 115) |

atgagcgtgacgccctttcgtttattgggaaacttgcaaatggaggaatgcgtgacagta
tcacaaggctcgaaaaagtccttgattatagtcatcacgttgacatggaagccgtttcta
atgcactag

| | |
|---|---|
| >dp1ORF108 DNA sequence | (SEQ ID NO. 116) |

atgcactcctgcacaataggacaccgcgcagcaaatacaaagaaagacaatctgccaaag
aaaaatagttgtgatgttactatatctatgattcaatttcgcttacctccaatcctctta
cattgcttgcctgaaaatctagaaccactgaagtatcatatatacgactataaagccttt
ggcctaaaaggtcaataa

| | |
|---|---|
| >dp1ORF109 DNA sequence | (SEQ ID NO. 117) |

atgtggttgtcgaagtcccaaatagttgattctccttcaactttccagcctttgaaagcc
ttacctgttaaggtagggtcaactggttttggagaaatcttcttacctgcttcaactcga
actgcgtcggcggttcctgttccaccgttcaaatcgaatgtcacgcgacgaagaaccgct
ggaagttgtgccacatag

| | |
|---|---|
| >dp1ORF110 DNA sequence | (SEQ ID NO. 118) |

atgatttcaattctagcatcaacttccatgtcgcgagtaagtgtgactccagtttcagcg
acaggacatgctttgaatactgcaatgtcaagttcgctctttctaataactgagcctagg
tctaagtacaagttaggattgattccagtgaccttatattgtttctcagtttcttttaca
ggaatgctttcatag

| | |
|---|---|
| >dp1ORF111 DNA sequence | (SEQ ID NO. 119) |

gtgactctatcaagaaagctcttgcaattggtgttcaaggttcttgggaaaacttcttgc
ttcttgcaagtgacgctgagaaattcatcgctgaaaaaacaggtcttcaaatcgctgtct
actctaagaaaattgctcagttcgctgacgctgacaaacttcctgacgttggtaacattc
gtcagttcaacttga

| | |
|---|---|
| >dp1ORF112 DNA sequence | (SEQ ID NO. 120) |

atgcaaactgatttaggcaaatactgcttcgacgcagcagccgttgcttatattagatat
ttgcaggaagacaagactcctaggtatcctggtgacgaaaagaaaaatccaggattgcaa
atgcttatggagtga

| | |
|---|---|
| >dp1ORF113 DNA sequence | (SEQ ID NO. 121) |

atgaaaacagttaaagaagcaatcaaacaattcggtgatgaatggtggtacgaaattatc
aacgaaaacggccaaatgattcaagacggaagaatcgaagacatgggcgaatacatggaa
gaaacggtcgaccaagttaagttcatcaactatggtgacatcgaatctcaaattatcaaa
ctatatatcgcataa

| | |
|---|---|
| >dp1ORF114 DNA sequence | (SEQ ID NO. 122) |

atgctattggcgaagacggggaaacagtccatcctgataattgtccattatgccaaaacg
gattccctcgtattgaaaaactatttcttcaactttacaaccatgatacgggaaaagttg
aaacatgggaccgaggccgttcttatgttcaaaagattgttacatttatcaataaatatg
gaagccttgtga

| | |
|---|---|
| >dp1ORF115 DNA sequence | (SEQ ID NO. 123) |

atgagcctcctttttttgatatatataatatacacgaattatcgcgagtttgtaaagccg
tttctaaataattttaaatcttttaagcatattgagttttgcttcataagtcccgttcac
ggcagcctcttgcattttgagtacaatgaaaggaggttcctcgatattgttgaaactata
gaaggtgaataa

| | |
|---|---|
| >dp1ORF116 DNA sequence | (SEQ ID NO. 124) |

atgaaattttcaaactttgctaaagcacttactaatgaatacctaatggtagtgaacaat
gaccaagctgaagtcttaggcgcaggaaatatcgaaaacattctcaacggttcgaacttt
gctaatgttgtagctgaagcgacagttttaaaactcgaaaaactcagcgaagaggaagct
attgagtag

| | |
|---|---|
| >dp1ORF117 DNA sequence | (SEQ ID NO. 125) |

atgataacaggctgctcgaacattttaaatcgaagtgaatctcgtaagtcactaatagtt
ttgttcaagttatctgctactgtgataaggtctttgacatcgcttgtcccgtatatgtca
ttagtcaatggttcattaagaataactcgacaaggaatttgcttcaagccggttggggcg
gattcttga

| | |
|---|---|
| >dp1ORF118 DNA sequence | (SEQ ID NO. 126) |

atgatattatctacgtcgacgcaacttgtgaaactattaaatacgaggagcctattgcat
gaacaatcagcgaaagcaaatgaacaaacgaatcgtcgaacttcgcgaagactatcaacg
tgcaagaggtcgaataaacttccttcttgctgtaaaggaccacggcgaagaactcgaaaa
ccttga

| | |
|---|---|
| >dp1ORF119 DNA sequence | (SEQ ID NO. 127) |

atggaggttcaacatccccgattcagtacgtcctactttttcgggcatttctttagtaga
cacgacttcagcggttcgacagattttaacagggaacaacttcctccaaatcatgtcgaa
cattcaagtcaacttcaacaatgcttccggcgcttacggatccactatccaagcatttca
cgctga

| | |
|---|---|
| >dp1ORF120 DNA sequence | (SEQ ID NO. 128) |

gtgttgaagcgcaagcagaatacatgcgtatgcaattgcttcaatacggtaaattcactg
tcaaatcaactaacagcgaggctcaatacacttacgactacaacatggatgctaagcaac
aatatgcagtcactaagaaatggactaacccagctgaaagtgaccctatcgctgacattt
tag

| | |
|---|---|
| >dp1ORF121 DNA sequence | (SEQ ID NO. 129) |

gtgcagacggatcacgtgagttcagtttggaagataataatcaacaatatatgggttatt
actccgattatgagcaagcagatagcagggatcgaactaagtatcgatggtttgaccgcc
ttgccaatgttcaagtgggaggtcgaaacgagttccttaattctttatttgaatttggtt
taa

| | |
|---|---|
| >dp1ORF122 DNA sequence | (SEQ ID NO. 130) |

atgttattctccttatcctacataccgaatcacgttcatgtctggattaaacgagtattg
ttccgttctaaatcggccgacttgaatggattgggtaaagatcccgttatcgatgtgaat
gaacccttgcgtaaggtacataacttcattccctgcggagaacatagaaattcggtcact
tga

| | |
|---|---|
| >dp1ORF123 DNA sequence | (SEQ ID NO. 131) |

atggttcgacttttcgaaggattgaggttttcgaaccggttgagtttttcgagcattctc
gacttttcgacccctttctatgctcgacttttcgagtgttttgaggttttcgagcaggtt
cgacttttcgagaaattgagtttttcgacctctaaattaggctcgattattcgaaaagtt
tag

| | |
|---|---|
| >dp1ORF124 DNA sequence | (SEQ ID NO. 132) |

atggtaaaagttaaagatttgcaagtaggaatgaaagttgtaaatgcaaaaggtactgaa
tttaaagtaactgaccgtcaaggtcgtaaatgggtaagcctagaacgtcttagtgatgga
cgtattcggttctatgataacgaatcactaatggacgaaaaagtggaggtagtaaaatga

| | |
|---|---|
| >dp1ORF125 DNA sequence | (SEQ ID NO. 133) |

atgtcctcagccgcttccgttaaaattggaacaagtgaattatatagatgctcctctttt
agcttgtcgataaggtattcatcagtttcgccaatttcgaaaaattcgaatccaggaaaa
tggtcgagaatagtttcgtcgtccggaactcttccatatctcgaaaagtgttcttga

| | |
|---|---|
| >dp1ORF126 DNA sequence | (SEQ ID NO. 134) |

atgagctcaagtacgttttctcgaacaatagggtcaagtccagttatatcaacgaactgt
atatcgtcctcttgtataggaataaggtctgcgtacagttgcatggctgaccctttaatt
ggagtaactgttccttcactgtttattttaaataaggttatcatttctatcctctaa

| | |
|---|---|
| >dp1ORF127 DNA sequence | (SEQ ID NO. 135) |

atgctaaatagctttcccattcaccgtcgctgttcttgcgccatttttcagtttcacgat
actgaccaactttgcaaaggtcgtgaaatagtgctacgattgcaactgtttccattgggt
aaatgtcttcccagcctttgcctaccatggtatccatttcgaaaagtagttgattga

| | |
|---|---|
| >dp1ORF128 DNA sequence | (SEQ ID NO. 136) |

atgacagcagttcaacaagttaagttctacttagaagaagccggcgctcactttctaaaa
gatgttgagtacagtgacaacttagagcaagcaattatgaaagatattcttaaatggaat
ggcgctcatagagatgagcacgatatgaaaataacttcatacgaagtattatag

| | |
|---|---|
| >dp1ORF129 DNA sequence | (SEQ ID NO. 137) |

atgaactttctgctaagcaacttgcgctcactgaagttcaaactaatgtacgcagccacc
aatcttacattgaagaattcagtaagaaggaaaaggcggacaaggaatgggaacgcattt
tggaagaacttgctcagcttgacgaaatctcagctggagcattgcctgtattag

| | |
|---|---|
| >dp1ORF130 DNA sequence | (SEQ ID NO. 138) |

gtgcttgactttattcctttattatcgtataatcataatataaataaaacaagcgtcaag
gacgcagaaagaggtcaattatggaaacaacactttatttcggttatcttacagcagatt
ggaaagacggtcacaagaactacactttccactatgaaagcattcctgtaa

| | |
|---|---|
| >dp1ORF131 DNA sequence | (SEQ ID NO. 139) |

atgctcaaccggctgagaagaaacttggctggcagaaagatgctactggtttctggtacg
ctcgagcaaacggaacttatccaaaagatgagttcgagtatatcgaagaaaacaagtctt
ggttctactttgacgaccaaggctacatgctcgctgagaaatggttga

| | |
|---|---|
| >dp1ORF132 DNA sequence | (SEQ ID NO. 140) |

gtgactggaaggtcatctaatacacatagcctcaagacatttcgttggctttcaggaaaa
cattcgactagattgtcaatgtatcccacaaaggcttcaaggttttcgagttcttcgccg
tggtcctttacagcaagaaggaagtttattcgacctcttgcacgttga

| | |
|---|---|
| >dp1ORF133 DNA sequence | (SEQ ID NO. 141) |

atgacttcttcattcatgacaagttttcgagtttctgcttgcttgtcaggaatagttttc
ccggcggctaaaatgtatagattatcgtatttttctttcctgatagcagaacttgaatcc
atttgtattcccaccatttccgccctatctgcggcgaaataa

| | |
|---|---|
| >dp1ORF134 DNA sequence | (SEQ ID NO. 142) |

atgacttcaatgtacttaggttccatcaattcatacaagtcattcaaaataatgttcatg
caatcttcgtggaagtcaccgtggttacggaaactgaataagtacaatttcaatgattta
gattcaaccatcttttcgtttggaatgtaa

| | |
|---|---|
| >dp1ORF135 DNA sequence | (SEQ ID NO. 143) |

atgaagcagaacttgaaaatgctgctaatgttgcaatgttctacggagtcaagttcacca
ttcttgaaattgactcgaaaatctactcaagctctagctcttccttattacaaggaaaag
gcgaaatttcacatggaaaatcttacgctgaaatcctag

| | |
|---|---|
| >dp1ORF136 DNA sequence | (SEQ ID NO. 144) |

gtgaagaaatcttcaataaccttattcgcttctttgacagatacattcatctgctcagcg
attgagttagccccgcggccgtacataagacctaaaagaacggacttgacagaatttctt
cgaagttttccttccttgttagtcgttccgtcgggatag

| | |
|---|---|
| >dp1ORF137 DNA sequence | (SEQ ID NO. 145) |

atgcttcgaacttgtttgttagcaccgtcaggaggacaaactagtcgaacccattcacct
gcgtctttgataatatctagcgcgacagcgcctacagaagaagcaacgtgtttcaacttc
ctaggcaagccttctgctagttcataccataatgcgtag

| | |
|---|---|
| >dp1ORF138 DNA sequence | (SEQ ID NO. 146) |

atgactatatcgaagaacaatgtagtcatccggcctatctgtatcttgctcgtcaaattc
aactcctggaagcataggagcaggcgagagctgaaatgtaggaagaatttccttcaatct
gtccatcattgtcgttcgtttagtcatgttcactcctag

| | |
|---|---|
| >dp1ORF139 DNA sequence | (SEQ ID NO. 147) |

atgatactaaatcactcaacttgtttgaccctcctgataaattcgttcacgcagacacgc
gcatttgagccctttttagatacctttcgcaaacacctagatgcttccctcactaaaagg
tcatgggcctcaagttcttcgaaagacatttctacatag

| | |
|---|---|
| >dp1ORF140 DNA sequence | (SEQ ID NO. 148) |

atgttttcgatatttcctgcgcctaagacttcagcttggtcattgttcactaccattagg
tattcattagtaagtgctttagcaaagtttgaaaatttcattttattttccctttatttg
tttttctttatactattattatacaataatgattga

| | |
|---|---|
| >dp1ORF141 DNA sequence | (SEQ ID NO. 149) |

gtgctaagagttgtagagatatcctctaaaacgctcttggctttattcgatttccattcg
aataacttatttagtaggacagtaagcactccgctgcacgctgtaataatcgtcgtcaag
actgctgtgtcgtttagccacattggcatagattga

| | |
|---|---|
| >dp1ORF142 DNA sequence | (SEQ ID NO. 150) |

gtgactgtcgaagtttctccaaacagttctgtcactttacctaaaagcgtattagggatt
ttcccgttagcgattaggttcatgacacctgctgctcgaattttaacatggataggttca
ctaccttttgaaaatcctggaagtgcgatgatttga

| | |
|---|---|
| >dp1ORF143 DNA sequence | (SEQ ID NO. 151) |

atgaagtttgggttgacgcttttaactccagaccgtttaattttttcaaggcttgaaatt
ggataccatataatcttttcatgcttttggaaatacactaaaattccggcgagaataaat
ttgcatccatctgcgcgtgatagctggaaccattga

| | |
|---|---|
| >dp1ORF144 DNA sequence | (SEQ ID NO. 152) |

gtgcaaatcaagcgactaacttatttagatacattaaacgaggcgcattcttcaagattc
ctaatggaaattcaacaattaccattgaataccgagccgatgacgcagcagcttggacct
ctactcttcccgctcaagttgaactgtttctaa

| | |
|---|---|
| >dp1ORF145 DNA sequence | (SEQ ID NO. 153) |

atggaaacagctggagacctaacaagtggaaagaggttctatttaagcaagacttcgaac
agaataattggcagaaacttgttcttcaaagtgggtggaaccatcactcaacctatggcg
acgcattctattcgaaaactcttgacggcatag

| | |
|---|---|
| >dp1ORF146 DNA sequence | (SEQ ID NO. 154) |

atgacaaactgcatgattgcatcacctttccagtacggaacctcaagggcgaaacagtat
tcttcaaccgtcgaagtgttcgttctaagtttcaccagtacggtgaagatgaccctaaaa
cggaatttctttatggccaatatgagcttgtag

| | |
|---|---|
| >dp1ORF147 DNA sequence | (SEQ ID NO. 155) |

atgtatctgtcaaagaagcgaataaggttattgaagatttcttcaccgagttccctaaag
tggcagactatatcatattcgttcaacagcaggcgcaggacttgggatatgttcaaacag
ctaccggtcgaagaagaaggcttcctgatatga

| | |
|---|---|
| >dp1ORF148 DNA sequence | (SEQ ID NO. 156) |

gtgtttcggttcaagaccattcgagtagggcgaacacctgtacgattttcgatgtcatcc
attgctgctaaaatgtcagcgatagggtcactttcagctgggttagtccatttcttagtg
actgcatattgttgcttagcatccatgttgtag

| | |
|---|---|
| >dp1ORF149 DNA sequence | (SEQ ID NO. 157) |

atgccattgaacttttcgagcataaggattaaccttgccccattgtctcactccagctgt
ggcggaatggctaatggtagttcgagcaagtcgaagggcattgtattcgagattttgata
tttatgagcagcaggtttccctag

| | |
|---|---|
| >dp1ORF150 DNA sequence | (SEQ ID NO. 158) |

gtggtcctttacagcaagaaggaagtttattcgacctcttgcacgttgatagtcttcgcg
aagttcgacgattcgtttgttcatttgctttcgctgattgttcatgcaataggctcctcg
tatttaatagtttcacaagttgcgtcgacgtag

| | |
|---|---|
| >dp1ORF151 DNA sequence | (SEQ ID NO. 159) |

atgattatatcaacgcaggggagattgctagctacattcaagcacttccttcaaacgctc
ttcaataccttggaccaactcttttccctaatgctcaacaaacagggacagacatttcat
ggctcaagggtgcaaataatttgccagtaa

| | |
|---|---|
| >dp1ORF152 DNA sequence | (SEQ ID NO. 160) |

atgtgcataaaggacttatcgacaaagaggctactattgcagtacttcctgaaggattta
gaccgaaagtttcaatgtatcttcaggctctcaataactcatatggaaatgccattctat
gtatatacactgacggaagacttgtggtga

| | |
|---|---|
| >dp1ORF153 DNA sequence | (SEQ ID NO. 161) |

atggtggacaaagggctcaccttttcgaactttcgatatcgtcatagcagacggttccat
tcgttcaggaaaaacagtatcgatggctctttcattttccctttgggccatgacggaatt
caacggacaaaactttgccatctgtggtaa

| | |
|---|---|
| >dp1ORF154 DNA sequence | (SEQ ID NO. 162) |

gtgacaataggctttaagaactgcaaaaaaacctggggcgtctgcacgcgcaacctggag
ctccttaacagtcatccaaggctgaggtttcttacaaacaatcctaattccttcaaaata
gctcttgtccgggtcaatagtgcctaa

| | |
|---|---|
| >dp1ORF155 DNA sequence | (SEQ ID NO. 163) |

atgaatacgaccctgagcaacttacaatgggacatggtgcaaaatctaatttccttcttc
aacgtttcattcaactcacgccagttgaagctcaagcaattttctggcatatgggagcct
atgatattagtccttatgcaaatttga

| | |
|---|---|
| >dp1ORF156 DNA sequence | (SEQ ID NO. 164) |

atgctagtatctccatttctgttggtcttgctttttagctctgttcagttcagctgcttc
tcgcgatgcaatagtttcgagaatatgcctgttcataggctcacaatattccgccaaaga
tttgccagttatggtggcgtcaattaa

| | |
|---|---|
| >dp1ORF157 DNA sequence | (SEQ ID NO. 165) |

gtgcttgctggacttgagaagaaattggtatcattttcgagccaatccataaggttctcg
ataccgtcacgattgattgtttctgttactgctttcttgaagcgttttttaaagtctgtc
atattagacccctttcattttctataa

| | |
|---|---|
| >dp1ORF158 DNA sequence | (SEQ ID NO. 166) |

gtgaacgccgttattagggtcaaacgaagcccaaacggacattgtctttgtcccgtcact
attgtgaggaacagtcacttctccacttgcgagcgttacctcttcgccggacgtgtcgta
gtctgggtgactgctatgaacacttga

| | |
|---|---|
| >dp1ORF159 DNA sequence | (SEQ ID NO. 167) |

atgatttggtctgcgcttacccaagcagcttctcctttgagtttctgtcgagcattccct
gtacggtctgtccaaatagcatgcgtctttgcgtattcttccatcttagtagcagcgact
tcgcagactgttatgacagcgacttga

| | |
|---|---|
| >dp1ORF160 DNA sequence | (SEQ ID NO. 168) |

atgggttacagacacgcgaggaaaacaatcgaacgtccaagacgtatctatcaatgttat
agaatactatggaccgtctatcaatttctccgttcaacgtactcgtcaaaatcctgcaat
tatccaagctcttcgaaatgctaa

| | |
|---|---|
| >dp1ORF161 DNA sequence | (SEQ ID NO. 169) |

atgcaaaaaggtttaaatgcttatctcgacatgacattgaaagcattgcattcgagacta
tttcaaaatgtttggcaacgttcaaatcaaaccaaggggccaagttttcaacttacctta
caagactcttcaagaatagaatag

| | |
|---|---|
| >dp1ORF162 DNA sequence | (SEQ ID NO. 170) |

atgacagaagttgcggtaaatagcccgcaaaaggtgagagtagttatggtcgggaatatt
gaatttctcgaatatttaaaaaggaagtacggaacagaaacttccatcagttatattata
gaaaatgaaaggggtctaatatga

| | |
|---|---|
| >dp1ORF163 DNA sequence | (SEQ ID NO. 171) |

gtgaccgaatttctatgttctccgcagggaatgaagttatgtaccttacgcaagggttca
ttcacatcgataacgggatctttacccaatccattcaagtcggccgatttagaacggaac
aatactcgtttaatccagacatga

| | |
|---|---|
| >dp1ORF164 DNA sequence | (SEQ ID NO. 172) |

atgtactcttggagaacttcgtgcctaaatgttccagcttcgcccattgcaattaggtta
gaatctgcgttatctataatagactcaccgattctttcgaaatacatttttcgaatacat
ccaccaaccccgctgggcttataa

| | |
|---|---|
| >dp1ORF165 DNA sequence | (SEQ ID NO. 173) |

atgagtgaaagctggtcaatccccaccacagatggtctatatttagatatcatgctatct
aaaattgcaggggtaaggttctttcctccaatcataaagggcgtgactaccacaagggaa
ttttcagcctcagtcattgcttga

| | |
|---|---|
| >dp1ORF166 DNA sequence | (SEQ ID NO. 174) |

gtggtcatgctctttaatgactctatcttctcccgtttggctcgctttactgtcccagct
gtaagcatagtattcatcaatgtcgtgcgtgttgctagggtcgagtgtaaatctattctc
agccaagagttcagcgtgaaatga

| | |
|---|---|
| >dp1ORF167 DNA sequence | (SEQ ID NO. 175) |

atgcttattcggttggagcttcttacgtcgtatatggtgctcacgcagacgatgcggctg
gaggtgcttaccctgattgcactcctgagttctataattcaatgtcaaatgcaatggaat
atggaactggaggcaaggtaa

| | |
|---|---|
| >dp1ORF168 DNA sequence | (SEQ ID NO. 176) |

atgagactttttccaggttatattcttcacattgttcagttcctggagtcaagtattgtt
cttgaaattcatagagttcgaaagtttgcaaagggtcataggccgcatacatataggcaa
catcaggaggaattaaactaa

| | |
|---|---|
| >dp1ORF169 DNA sequence | (SEQ ID NO. 177) |

atgaacacagcatcgcgaagagtttcaatgttagtgataaggaagaattcgtcgtggcca
ccaagcaagtcttctgcccgtttagaaactccgtcaatcactaatttcccatctttagtg
actcgacttcctaaaatatga

| | |
|---|---|
| >dp1ORF170 DNA sequence | (SEQ ID NO. 178) |

atgatgattgttcttgtgctcctgccgtttgttgagcagcagcaagttgcttaccaaaag
agccgatttcacgaggttcgggaacaccaccaccgacacgacctggatttcctaaatttc
cagtcccggctggcgacttag

| | |
|---|---|
| >dp1ORF171 DNA sequence | (SEQ ID NO. 179) |

atgtcattttctttcatgtactcttttagagcatcacgaagacttttgacttgtttctcc
atgtcgcctttggtagcatttaattcaccggcttcttcaattgcagcgatgaactgtttt
tcatcttcaaatttcatttaa

| | |
|---|---|
| >dp1ORF172 DNA sequence | (SEQ ID NO. 180) |

atgtttcgaacattttctaccccattattagaagcagcatcaatttcaataggagagcca
agtcctttgttcacatccttcgcgaaaattcgagcagtagtggttttaccagttccagcg
ccaccacagaatagatag

| | |
|---|---|
| >dp1ORF173 DNA sequence | (SEQ ID NO. 181) |

atgacattagacatttccttcgtctgtacgaaaggtttcagcttgagtcacttcaccgta
cattgcactgaagattgtcataagttgctcatctgtcatatactcgccgacttcagcgta
agtaggctctaccattga

| | |
|---|---|
| >dp1ORF174 DNA sequence | (SEQ ID NO. 182) |

atgtcccatcagcccttttcattaagattgtcgaaccagcgttcgacttttcatcagttt
caagctgttcttgcttatattggtcataatagaattgcgccatttgtttccagtagtctg
cgtcaccttttagactga

| | |
|---|---|
| >dp1ORF175 DNA sequence | (SEQ ID NO. 183) |

atgcgcgtgatgtcatggcagataggcgaggataaagagtgtcgaatagaacgccgcaga
gcttacgagagcgccaaatacaagggcgacggtactacggtggtcctcttgcttacctgt
aaccaaataaaccattga

| | |
|---|---|
| >dp1ORF176 DNA sequence | (SEQ ID NO. 184) |

gtgataaagacggtaacgttgaatttttctagttccgtcttgaatgacgtcattttggtg
attgattgctactgtcgtttggtcaatcccgtcgacctgctgtttaagagtgctaagagt
tgtagagatatcctctaa

| | |
|---|---|
| >dp1ORF177 DNA sequence | (SEQ ID NO. 185) |

atgaacctaaacagttcgagacttctcaagctgttgggaaagaagcaggtcgaatatttt
ggtgggaacgtgaacttggtcatattctcgcgactaattttaggtgcttttgtattaatc
agcgtgatatgcgcttga

| | |
|---|---|
| >dp1ORF178 DNA sequence | (SEQ ID NO. 186) |

atgacaactgtcgaccaatttaaaagacagttgaggaaaagtttaggctcaatttttcct
tcatcagtttccttaaatttgagccaattagtaacctttagcgaattgctagcacttgcc
tcccatattaagtcataa

| | |
|---|---|
| >dp1ORF179 DNA sequence | (SEQ ID NO. 187) |

atgggtagggttattccttacctcgttgatttgctttatgcaaaacctaccacaatcgct
tgtcgtggcttcaggagttgcattttggataagtcaaaaagcaagtgtctttatattcga
caagctctcgaataa

| | |
|---|---|
| >dp1ORF180 DNA sequence | (SEQ ID NO. 188) |

atgttcgacatgatttggaggaagttgttccctgttaaaatctgtcgaaccgctgaagtc
gtgtctactaaagaaatgcccgaaaaagtaggacgtactgaatcggggatgttgaacctc
catccgtttgaatag

| | |
|---|---|
| >dp1ORF181 DNA sequence | (SEQ ID NO. 189) |

atggaagtttctgttccgtacttcctttttaaatattcgagaaattcaatattcccgacc
ataactactctcaccttttgcgggctatttaccgcaacttctgtcataggctgtcctcct
ttgcttatactgtaa

| | |
|---|---|
| >dp1ORF182 DNA sequence | (SEQ ID NO. 190) |

gtgcttgcccatgtttcaataaatagggttcgacctcgcctagctttcgaacgtgctata
acgatttcaatcatagcgaagaaaggtgagaagcttcaatcaattccattgcggtgtcaa
tatcttcttccttga

| | |
|---|---|
| >dp1ORF183 DNA sequence | (SEQ ID NO. 191) |

gtgattccagcttttggtttttcttcagcctcttcaactttttcttccttaggcgcaggt
ttcttacgagttgaactcttaggtttttcttcaactacttcttcaacctcagcctcttgt
tcaactggaccttga

| | |
|---|---|
| >dp1ORF184 DNA sequence | (SEQ ID NO. 192) |

gtgaacttgccgtcaaccacgtcaaacatttggtcttcgtcgaggtctaaaattagagtt
ccaagaagttcgctcttttctggaaaatcttcaagagtagcactgtcttccggacgctct
ggaaggaattcataa

| | |
|---|---|
| >dp1ORF185 DNA sequence | (SEQ ID NO. 193) |

atgaaattcgagatgttcgaaatgaaaatctacttattattagacactttagaaatggcg
aagaaattgtcaactacttctatatatttggaggaaaagatgagtcgagtcaagacctta
tacagggggtaa

| | |
|---|---|
| >dp1ORF18G DNA sequence | (SEQ ID NO. 194) |

atgctcgaaaaactcaaccggttcgaaaacctcaatccttcgaaaagtcgaaccattcga
aaagttcaaaagttcgaaaaactcaaccattcgagagtaggaattaaggacataccagtt
caacctttttag

| | |
|---|---|
| >dp1ORF187 DNA sequence | (SEQ ID NO. 195) |

atggtcttgttcaatctcttcctactatcattcaagcagctgttcaaattatcactgctt
tattcaatggtcttgttcaggcacttcctacgcttattcaagcaggtcttcaaattttgt
cagctctcataa

| | |
|---|---|
| >dp1ORF188 DNA sequence | (SEQ ID NO. 196) |

atgttcgtaaagcagccggttcgcctcgagtggacttgttcaatacaggaagtgacaacc
ctaaccaacctcagtcacaatctaaaaacaatcaaggcgagcaaaccgttgtcaacattg
gaacaatcgtag

| | |
|---|---|
| >dp1ORF189 DNA sequence | (SEQ ID NO. 197) |

atgcaaacgcagtatcaaccgtctctgaaactcttcatgacccagacttgtatgctgcga
accgtcgagaacttcgagctgacgagcaaaaacttcgcgaaactcgttacgcaatcgaag
atgaaattctag

| | |
|---|---|
| >dp1ORF190 DNA sequence | (SEQ ID NO. 198) |

atgtattcactcaaagttgttcagtgtggctcaatcatattaaaatcgaacttggtaata
tctctactccttttagtgaagcagaggaagaccttaaatatcgaattgactcaaaagccg
atcaaaagctaa

| | |
|---|---|
| >dp1ORF191 DNA sequence | (SEQ ID NO. 199) |

atgtccattgttccggaacttgatttaggtaagtaccttgctaagtccagtgacggcgta
aaggatacgctagtagtatggttcttacctaaatctatccagtcgctaccgaaaactcgg
taccaaacttga

| | |
|---|---|
| >dp1ORF192 DNA sequence | (SEQ ID NO. 200) |

atggtcgacgtcgaatgttttttcgagatgaagtttagggtcttctcgataccctacggt
atgttcagcgagtgctttaacaaaacggaatggagtatcttgcaacccgtcacgttctgc
gtcctcgcctaa

| | |
|---|---|
| >dp1ORF193 DNA sequence | (SEQ ID NO. 201) |

atgatttcagctcaaattaaatacgaaatgagacattgtctaaatttaaccaagaattat
ctacattcgatttcaccacaagtcttccgtcagtgtatatacatagaatggcatttccat
atgagttattga

| | |
|---|---|
| >dp1ORF194 DNA sequence | (SEQ ID NO. 202) |

atgaacccttgcgtaaggtacataacttcattccctgcggagaacatagaaattcggtca
cttgataccttaatggtagagctaccgtcgttcttaccgataattagaccttcattagaa
gagctcatgtaa

| | |
|---|---|
| >dp1ORF195 DNA sequence | (SEQ ID NO. 203) |

atgttcacaatcgttgttttgacaagtttcttttcagctccttgtccaatagtgaactct
gccacaatttggcgcgattttgtaaggttcaacatagttctcacctcctttctaaaaaat
attataacatga

| | |
|---|---|
| >dp1ORF196 DNA sequence | (SEQ ID NO. 204) |

atggtagatttaacaagtccctgtccaatcatgtcactcctccttgctcatcaaaagaag
tttggtttcaattatcggtttagcattaggctcccatttaacaactccagcaagttcatt
catttcttctag

| | |
|---|---|
| >dp1ORF197 DNA sequence | (SEQ ID NO. 205) |

atgaaaagattatatggtatccaatttcaagccttgaaaaaattaaacggtctggagtta
aaagcgtcaacccaaacttcatcgatgcagggtatgaagtttcttacaagaagcgtcgaa
ctagattga

| | |
|---|---|
| >dp1ORF198 DNA sequence | (SEQ ID NO. 206) |

atgccgctcaacaaattgacgtccagttttattcaatgcctcagttcacctatacagttg
accctagaaacccttccagcttgctttctgttgacattgtttatcaggacgagcgtacaa
aaggaatga

| | |
|---|---|
| >dp1ORF199 DNA sequence | (SEQ ID NO. 207) |

gtggctcctgaattaggctgtacttttcctcccaactgcttagcaactgccttctcttgt
ttagcactagctctgcgcgtgggaattggtttgtatgcgcgtgatgtcatggcagatagg
cgaggataa

| | |
|---|---|
| >dp1ORF200 DNA sequence | (SEQ ID NO. 208) |

atgacaggcttgtattcgataagccctgaaagtttttcacacatttcttccgtctcggct
tcgtcaactaatttttcgataatttctttcaagcgttcttcgtccatagttgagcgctct
gtcgtgtag

| | |
|---|---|
| >dp1ORF201 DNA sequence | (SEQ ID NO. 209) |

atgggcttcacaagttccttctttaatcaaaggtcaatatctttggactcgaactatttg
gacctataccgattcaactaccgaaacgggctatcaaaaaacctacattccaaaagacgg
gaatga

| | |
|---|---|
| >dp1ORF202 DNA sequence | (SEQ ID NO. 210) |

gtggggcgtttattttttataaaaattttttacaaaatgcttgacaacattcactcatta
tcgtataatacaattataaaaataaataaagccgaaaggcgaggaggacattatgtcaaa
aattaa

| | |
|---|---|
| >dp1ORF203 DNA sequence | (SEQ ID NO. 211) |

gtgattaggattggccgggttacaagagaaccacattttcgaacctgttacggaacagcg
ccctgtcgcttggttgacaaacgattcaggcatcagtgccacctcatcacagaagatacc
tgctaa

| | |
|---|---|
| >dp1ORF204 DNA sequence | (SEQ ID NO. 212) |

atgaccacggttcgagtcaagggatggttgttgacttttatcacgtcaagaaaatcgcag
gtacattcattgacagacttgaccacgctgttcttcttcaagggaatgaaccaatcgctt
tag

| | |
|---|---|
| >dp1ORF205 DNA sequence | (SEQ ID NO. 213) |

gtgacactgatgaatggttctcagtttggtatgctactcgtgacgcagatatcttctacg
accaaagaattgcccaatttagaattcaggaaaagcaacctgctatcaagttcaatttcg
tag

| | |
|---|---|
| >dp1ORF206 DNA sequence | (SEQ ID NO. 214) |

atgaccaagttcacgttcccaccaaaatattcgacctgcttctttcccaacagcttgaga
agtctcgaactgtttaggttcatcaaattgttcaacttgagcaagtgcgatattattctt
tag

| | |
|---|---|
| >dp1ORF207 DNA sequence | (SEQ ID NO. 215) |

gtgtcggtggtggtgttcccgaacctcgtgaaatcggctcttttggtaagcaacttgctg
ctgctcaacaaacggcaggagcacaagaacaatcatcattctttaaataataggaggaac
taa

| | |
|---|---|
| >dp1ORF208 DNA sequence | (SEQ ID NO. 216) |

atgtttggtatgaagcaaaagacttcgctgaagaaaataacattcacttcccgtttgttc
ttcctgaacctagaacagaccttgaccatcgtggttctcgattctgggatgacgaaggcg
tga

| | |
|---|---|
| >dp1ORF209 DNA sequence | (SEQ ID NO. 217) |

atgttaagaatcaagttcgtagagccattgaaaccgctcctactaaaatcaaggtacttc
gaaactcttgggtcagtgatggatatggaggaaagaaaaaggataaagcgaatgaagtcg
tag

| | |
|---|---|
| >dp1ORF210 DNA sequence | (SEQ ID NO. 218) |

atgtttcaacttttcccgtatcatggttgtaaagttgaagaaatagtttttcaatacgag
ggaatccgttttggcataatggacaattatcaggatggactgtttccccgtcttcgccaa
tag

| | |
|---|---|
| >dp1ORF211 DNA sequence | (SEQ ID NO. 219) |

gtgctcgacttttatgtcgcccctaatttttgtttttacttacggactatgggatttgta
ggtattttcagggcgcttttttatttacttattaagtccttttctatattagattgttta
taa

| | |
|---|---|
| >dp1ORF212 DNA sequence | (SEQ ID NO. 220) |

atggactgtttccccgtcttcgccaatagcattgcaattgatatagcgtcgacgaccgtc
aacgtctgcttcgtggactacgaaataatccatgtcttcgccttccgggtcatcatacaa
tag

| | |
|---|---|
| >dp1ORF213 DNA sequence | (SEQ ID NO. 221) |

atgcgtctttgcgtattcttccatcttagtagcagcgacttcgcagactgttatgacagc
gacttgaaacttgtttcgataccgttcacagttactaacaaattcttcaggcttccatac
taa

| | |
|---|---|
| >dp1ORF214 DNA sequence | (SEQ ID NO. 222) |

atgatgccaaagttgtttttcagtgctcattccttttgtacgctcgtcctgataaacaat
gtcaacagaaagcaagctggaagggtttctagggtcaactgtataggtgaactgaggcat
tga

| | |
|---|---|
| >dp1ORF215 DNA sequence | (SEQ ID NO. 223) |

atgttaccaaaccctgatagagtttctttacttctattatacaatcctctcgacagtttg
tcaacgtcgtcattgtttcgaactacgattgttccaatgttgacaacggtttgctcgcct
tga

| | |
|---|---|
| >dp1ORF216 DNA sequence | (SEQ ID NO. 224) |

atggcctcggagctcgcggccacatctcctccagatacggcagccaggtcaagtacccct
ggcatagcgtccatgatttcatttacctggaaaccggctgaagctagattttccatacct
tga

| | |
|---|---|
| >dp1ORF217 DNA sequence | (SEQ ID NO. 225) |

atgaatactatgcttacagctgggacagtaaagcgagccaaacgggagaagatagagtca
ttaaagagcatgaccactgcatggataggaacagatatgcctgtctcactgacgctctaa

| | |
|---|---|
| >dp1ORF218 DNA sequence | (SEQ ID NO. 226) |

atggaatgcttccggaagaggttcgatatagactacaaattgagcgcgagaaaattacat
tgctccgggccaaaatgggcgaccaggaaattgaaggcgaggttaaagataacttcgtag

| | |
|---|---|
| >dp1ORF219 DNA sequence | (SEQ ID NO. 227) |

atgattttatgctcgactttttcagttctcccatttcttcgaaacgcttcagggctgacg
ccttgcctaactacttcgctagatgttccaaaattccttttcagccactggtttccatag

| | |
|---|---|
| >dp1ORF220 DNA sequence | (SEQ ID NO. 228) |

gtgaagttttcttcggtgacggttgatacaatttccttcaagagtaagctgttaaggtgg
caagtgaattctttcttcgaaactttcttgccagcagatgcgtacatgatgtcttcataa

| | |
|---|---|
| >dp1ORF221 DNA sequence | (SEQ ID NO. 229) |

atgactgctcaagttctatgtactatgctctccgctcagccggagcttcaagtgctggat
gggcagtcaatactgagtacatgcacgcatggcttattgaaaacggttatgaactaa

| | |
|---|---|
| >dp1ORF222 DNA sequence | (SEQ ID NO. 230) |

gtgacggtatcgagaaccttatggattggctcgaaaatgataccaatttcttctcaagtc
cagcaagcactcgataccatggaagctatgaaggtggacttgtcgagcactcattaa

| | |
|---|---|
| >dp1ORF223 DNA sequence | (SEQ ID NO. 231) |

atgtggtggtacctgctggatatgttcgagatgtctactacttctacagtgaagtcgctg
acgtttactacaagaaagatgtcgacgagcctgacgatgacagcgacattcttgtag

| | |
|---|---|
| >dp1ORF224 DNA sequence | (SEQ ID NO. 232) |

atgccagaaaattgcttgagcttcaactggcgtgagttgaatgaaacgttgaagaaggaa
attagattttgcaccatgtcccattgtaagttgctcagggtcgtattcatatgctaa

| | |
|---|---|
| >dp1ORF225 DNA sequence | (SEQ ID NO. 233) |

gtgagcaacgggtgcgacgtatttcatcgcctctgccatgtcgctagtttctgcgttcgt
atcagctgctgctcgagcaaatacgtcagccacgtgacccgcctggtttgcctctaa

| | |
|---|---|
| >dp1ORF226 DNA sequence | (SEQ ID NO. 234) |

gtggctgcgtacattagtttgaacttcagtgagcgcaagttgcttagcagaaagttcatc
gctaggaattggatagtggtgttcgatagtcattgtcgtaagtgtttgataacttga

| | |
|---|---|
| >dp1ORF227 DNA sequence | (SEQ ID NO. 235) |

atgactcaattagatggtagcgcttatgacgtttcgagaatccataaaggccgaaggttg
ttgcattatagataccaaagtcgcctgctacgaataaacggtcgaattctatattga

| | |
|---|---|
| >dp1ORF228 DNA sequence | (SEQ ID NO. 236) |

atgttcgaaacattattgaagattctagatacaagtctatggacagcgagttcaaagttt
acatcattgacgaggttcatatgctttcaaccggagcatttaatgcgctgttga

| | |
|---|---|
| >dp1ORF229 DNA sequence | (SEQ ID NO. 237) |

atgtgcgagttaagaaaactgattttaatcaaaccactcgaagcattgtcgcaattcctg
accactacgttgctttggctgctcaaattccagctaccgcagcaactcaagtag

| | |
|---|---|
| >dp1ORF230 DNA sequence | (SEQ ID NO. 238) |

gtgacgaaaaatccggcatacttgaactatctgtcgttaaaaaccgatatggcgaagacc
gaaaaatcatcgaatatatgtgggacgttgaaactggaacctatactcttatag

| | |
|---|---|
| >dp1ORF231 DNA sequence | (SEQ ID NO. 239) |

atgcgcgtgtcattgcgtttcacatcttcagttccctccgaggtcacggcttcgagttct
gctgtttctgccgtatctacgacaaagttagctccgccgacttttggcaactga

| | |
|---|---|
| >dp1ORF232 DNA sequence | (SEQ ID NO. 240) |

atgtcaattccattagctcttgctaattcaacgagctcaggaacggttttagccgcatac
tcttcgcgcatttgttcaacttcgtcaatttcttcaactgattcaattgtttga

| | |
|---|---|
| >dp1ORF233 DNA sequence | (SEQ ID NO. 241) |

atgtcttcgccttccgggtcatcatacaatagagtgacaattgcgctgtcaccgtggtca
gcgagtgtgaaaaactcgttattagaccctgagctaaatgttcctgatttttga

| | |
|---|---|
| >dp1ORF234 DNA sequence | (SEQ ID NO. 242) |

atgcttacgagtacagcgactcaactgttcgaaaggtttataagtttcaacccgctttgg
gaggcgatagcttacctaacccaggaagacctactcgacaatttagagtag

| | |
|---|---|
| >dp1ORF235 DNA sequence | (SEQ ID NO. 243) |

atgaaatcatggacgctatgccaggggtacttgacctggctgccgtatctggaggagatg
tggccgcgagctccgaggccatggctagttcacttcgagcctttggattag

| | |
|---|---|
| >dp1ORF236 DNA sequence | (SEQ ID NO. 244) |

atgttcgtcgcttttagatttagcaatatatcgaggcttcatgtggcgtgtagtaaacca
cgaaacatcaatgagatattcacttccattgttgatagaagcaaacgttaa

| | |
|---|---|
| >dp1ORF237 DNA sequence | (SEQ ID NO. 245) |

gtgagagtccaggtaaggaatcttgacatattctcagccgtagttctaaatccaaataga
actcgcttggtgtcaactgcatttgctaaagcgattggttcattcccttga

| | |
|---|---|
| >dp1ORF238 DNA sequence | (SEQ ID NO. 246) |

atgcctttttgcggtcgatacaagttgcgcaagttccacaactttcagcgtcactttcat
aacatgaacgagtcaagaaataaggaacatctaaatcaattccccatttaa

| | |
|---|---|
| >dp1ORF239 DNA sequence | (SEQ ID NO. 247) |

atggtgaagtatttcctatcgaagaatgtcctttcgaccatcctaatggaatgtgctacc
aaactgtatggtacgaaaactcactcgaagaaatcgctgatgagttga

| | |
|---|---|
| >dp1ORF240 DNA sequence | (SEQ ID NO. 248) |

atgtttggaataagcgtgaaacagagtttacatggcgaagtaacaaatacgaggacaacc
ctacgggaactcgaggtgaatggggactatttcaaaatttctggttag

| | |
|---|---|
| >dp1ORF241 DNA sequence | (SEQ ID NO. 249) |

gtgtctttccttaatatggagatagttttcattctatttaagcaggatatcgaaaaggtt
accaattttagatttcataggcttaccatctacgatataatctgctaa

| | |
|---|---|
| >dp1ORF242 DNA sequence | (SEQ ID NO. 250) |

gtgtctgtaacccatgctcttacggtagcggagccattaaagttcatcatacccaatttg
ccgccgttttcgttgatagcttggtttttacctacgagctcagcgtga

| | |
|---|---|
| >dp1ORF243 DNA sequence | (SEQ ID NO. 251) |

atgttccaaaattccttttcagccactggtttccatagaaccctccatcgtttcgaccta
atacattcgagacgaattcagttagtcctgaagtgtagccgcaagtga

| | |
|---|---|
| >dp1ORF244 DNA sequence | (SEQ ID NO. 252) |

gtgaggtacaaaatgttgaccgtcgccgtcaatgaaaattttagcatcgagttctttcga
agttttcgaaataatttccttcacctgtttgatagttggttcatctag

| | |
|---|---|
| >dp1ORF245 DNA sequence | (SEQ ID NO. 253) |

gtggcaagtgaattctttcttcgaaactttcttgccagcagatgcgtacatgatgtcttc
ataactgctagtagaagttttaattcgaagtcggtctttcaagaataa

| | |
|---|---|
| >dp1ORF246 DNA sequence | (SEQ ID NO. 254) |

atggagtatcttgcaacccgtcacgttctgcgtcctcgcctaatagaccaaaaagtcttt
gaacggctgcctcagtattgtccaaggttacaatttcatccggcttaa

| | |
|---|---|
| >dp1ORF247 DNA sequence | (SEQ ID NO. 255) |

gtgacgcagactactggaaacaaatggcgcaattctattatgaccaatataagcaagaac
agcttgaaactgatgaaaagtcgaacgctggttcgacaatcttaa

| | |
|---|---|
| >dp1ORF248 DNA sequence | (SEQ ID NO. 256) |

gtgcaaagcctcgttctagcaagaagaacgatgctcagttacttgctcaacggaaaaaca
ggaagcctgcagttgaggttacttacatttcaggaaacgctctaa

| | |
|---|---|
| >dp1ORF249 DNA sequence | (SEQ ID NO. 257) |

gtggatgcgactatcattgcaactggtgtgactcagcctttacctggaacggtactactg
agccggaatatatcacaggcaaagaagctgctagtcgaatcttga

| | |
|---|---|
| >dp1ORF250 DNA sequence | (SEQ ID NO. 258) |

atgggcaaacatggaagattgacgaagactcagtcgactataaacctactcgagaaattc
gaaactatattcgacaacttatcaaaaagcaatcacgctttatga

| | |
|---|---|
| >dp1ORF251 DNA sequence | (SEQ ID NO. 259) |

atggaaataattagtcttaccgtctgcgcctggcttcccgggtatcccttgagctccgtc
attccccttccatttcgtccatgtataggctgcagggtcttttga

| | |
|---|---|
| >dp1ORF252 DNA sequence | (SEQ ID NO. 260) |

gtgttgtataggtcgaaactaattttgcatattttctatatttcaaaagtgcttttgaga
tatcgttatcaaaatgctcgacaatactttcgcctgttcctctag

| | |
|---|---|
| >dp1ORF253 DNA sequence | (SEQ ID NO. 261) |

atggttgcgtctataatagaaccgatgttgctagacaaagcatttgcaatcttcgagtct
aatttattcgagagcttgtcgaatataaagacacttgctttttga

| | |
|---|---|
| >dp1ORF254 DNA sequence | (SEQ ID NO. 262) |

atgaacctttcgcttaggttcaatctttttcgaacattttcatatttaacaaaactttca
gctaaaaatcgacaaagttcaatgttcgactcaatgtttaaataa

| | |
|---|---|
| >dp1ORF255 DNA sequence | (SEQ ID NO. 263) |

atgctttggtcttctcgacgaatgactctactacattccctgcagggtttcgagcagtac
gggtcaatgatgcaccgttttcgtcaaggtagtcaccttttctaa

| | |
|---|---|
| >dp1ORF256 DNA sequence | (SEQ ID NO. 264) |

atgaccttccagtcactaatgcggccgctgaaattggataccactatacatgggttcacc
aacttcgagacaaagcagttgaaacacttgaagaaattttag

| | |
|---|---|
| >dp1ORF257 DNA sequence | (SEQ ID NO. 265) |

gtgaacgtgctggatttagcaaacaagctactgagatggcattcttccgtgagtctatgc
gacttggtgaaaaagaccgtcaaaacttgcaaatgctattga

| | |
|---|---|
| >dp1ORF258 DNA sequence | (SEQ ID NO. 266) |

atggaaattggtattggttcgaccgtgacggatacatggctacgtcatggaaacggattg
gcgagtcatggtactacttcaatcgcgatggttcaatggtaa

| | |
|---|---|
| >dp1ORF259 DNA sequence | (SEQ ID NO. 267) |

atgactcgactacgaagcataaagacaagtggatggaaagagtattcgaagttattcgaa
acagttctaatccagacgttaagactcacgcatttgggatga

| | |
|---|---|
| >dp1ORF260 DNA sequence | (SEQ ID NO. 268) |

gtgaccctacttcctcaatcggcggtactggaggcaagcaagctcaagtcacttccattt
caggaaacttcaacttccttccagcggctgaatattatttag

| | |
|---|---|
| >dp1ORF261 DNA sequence | (SEQ ID NO. 269) |

atgaattcacttccctttgccctaaaacaggacagcctgacttcgcgaatgttttcatta
gttacattccaaacgaaaagatggttgaatctaaatcattga

| | |
|---|---|
| >dp1ORF262 DNA sequence | (SEQ ID NO. 270) |

atgcctattcaactccaggcggaaagatgtggaagcatgcttgtgcagttcgacttaaat
ttagaaaaggtgactaccttgacgaaaacggtgcatcattga

| | |
|---|---|
| >dp1ORF263 DNA sequence | (SEQ ID NO. 271) |

atgaaaattttagcatcgagttctttcgaagttttcgaaataatttccttcacctgtttg
atagttggttcatctagaccttttaacaagtcttctaattga

| | |
|---|---|
| >dp1ORF264 DNA sequence | (SEQ ID NO. 272) |

gtgaatagtacaaggcggtctaatacgctcaggatttctgctgtagggatagccgcatca
tcttcaaactcaattgagtcaagctgtgaaacgtcttcataa

| | |
|---|---|
| >dp1ORF265 DNA sequence | (SEQ ID NO. 273) |

gtgaataaagtcaagcgtttttgtataaaaagttcatttttttttaaaaaaaataagagc
gaaaagctcttatctaaaatagtcgacgttgacgatttttaa

| | |
|---|---|
| >dp1ORF266 DNA sequence | (SEQ ID NO. 274) |

atgcccgttcttccaagcagttgcaagcattttatcaatagtccacgacttaccttgtcc
aggtcgagccattatgacaatcaaatcctcaccaggaagtaa

| | |
|---|---|
| >dp1ORF267 DNA sequence | (SEQ ID NO. 275) |

atggtcaaggtctgttctaggttcaggaagaacaaacgggaagtgaatgttattttcttc
agcgaagtcttttgcttcataccaaacattaatcgtagatag

| | |
|---|---|
| >dp1ORF268 DNA sequence | (SEQ ID NO. 276) |

atgtcaatttcggtcttgtgcttgacaatggattcaactactgatgcgtcaacctttttc
aatcgcgacagcttgtccaattcattgtcaattctagagtaa

| | |
|---|---|
| >dp1ORF269 DNA sequence | (SEQ ID NO. 277) |

gtgaatagtatcgagtccatcagtttctacgtcaatagaacctattccgtcttcaatcat
tttgtctacatactgctcgagttttgcttcctcagtgattaa

| | |
|---|---|
| >dp1ORF270 DNA sequence | (SEQ ID NO. 278) |

atgatttttcggtcttcgccatatcggtttttaacgacagatagttcaagtatgccggat
ttttcgtcacgcttcatagcgataactctgctagcattttga

| | |
|---|---|
| >dp1ORF271 DNA sequence | (SEQ ID NO. 279) |

atgaggctgctttgctttatcttcgttaccgtattgaccgacttcctactcgcgaacctt
cctacaagaattcatacctcaaaggctttttgtcagccttag

| | |
|---|---|
| >dp1ORF272 DNA sequence | (SEQ ID NO. 280) |

gtggtcaagtctgtcaatgaatgtacctgcgattttcttgacgtgataaaagtcaacaac
catcccttgactcgaaccgtggtcataagttccgcctgctaa

| | |
|---|---|
| >dp1ORF273 DNA sequence | (SEQ ID NO. 281) |

atggatttcattaggactgagtcctcttggaattggaacggttgcatatatagatattcc
gtcagccgtactaggccaagttctagttcagtttatcttgcagtcaattgcttcgagata
tttgaaaaagtagtcaggaaaattcctgattatcttgcagtcaattgcttcgagatattt
gaaaaagtagtcaggaaaattcctgattattttttttacaaaaacgcttga

| | |
|---|---|
| >dp1ORF001 amino acid sequence | (SEQ ID NO. 282) |

MIDNNLPMSPIPGEIVQVYDQNFNLIGASDEIFSKHYEDEIVTRARGKETFTFESIETSS
IYQHLKVENIIQYGGRWFRIKYAQDVEDVKGLTKFTCYALWYELAEGLPRKLKHVASSVG
AVALDIIKDAGEWVRLVCPPDGANKQVRSITAAENSMLWHLRYLAKQYNLELTFGYEEII
KQEVRIVQTVVFLQPYVESKVDFPLVVEENLKYVTRQEDSRNLCTAYKLTGKKEEGSQEP
LTFASINNGSEYLIDVSWFTTRHMKPRYIAKSKSDEHFRIKENLMSAARAYLDIYSRPLI
GYEASAVLYNKVPDLHHTQLIVDDHYDVIEWRKISARKIDYDDLSNSTIIFQDPRKDLMD
LLNEDGEGVLSGETVNESQVVIRYADDILGTNFNAESGKYIGVLNTNKKPSELVPDDFTW
IRLEGPKGDAGLPGAPGRDGVDGVPGKSGVGIADTAITYAVSVSGTQEPENGWSEQVPEL
IKGRFLWTKTFWRYTDGSHETGYSVAYIGQDGNSGKDGIAGKDGVGIAATEVMYASSPSA
TEAPAGGWSTQVPTVPGGQYLWTRTRWRYTDQTDEIGYSVSRMGEQGPKGDAGRDGIAGK
NGIGLKSTSVSYGISPTDSAIPGVWASQVPSLIKGQYLWTRTIWTYTDSTTETGYQKTYI
PKDGNDGKNGIAGKDGVGIKSTTITYAGSTSGTVAPTSNWTSAIPNVQPGFFLWTKTVWN
YTDDTSETGYSVSKIGETGPRGVQGLQGPQGLQGIPGPAGADGRSQYTHLAFSNSPNGEG
FSHTDSGRAYVGQYQDFNPVHSKDPAAYTWTKWKGNDGAQGIPGKPGADGKTNYFHIAYA
SSADGSREFSLEDNNQQYMGYYSDYEQADSRDRTKYRWFDRLANVQVGGRNEFLNSLFEF
GLKPRYSSYNLMDGQDQTQGQISATIDERQRFKGANSLRLDSTWNGKPQNQKLTFSLGGD
TRLGTPTEWSNLEGRISFWAKASRNGVSLAARPGYRSNVFTATLTDQWKFYDFKFFDKVN
SNCTAEAIFHVFTQSCSVWLNHIKIELGNISTPFSEAEEDLKYRIDSKADQKLTNQQLTA
LTEKAQLHDAELKAKATMEQLSNLEKAYEGRMKANEEAIKKSEADLILAASRIEATIQEL
GGLRELKKFVDSYMSSSNEGLIIGKNDGSSTIKVSSDRISMFSAGNEVMYLTQGFIHIDN
GIFTQSIQVGRFRTEQYSFNPDMNVIRYVG

| | |
|---|---|
| >dp1ORF002 amino acid sequence | (SEQ ID NO. 283) |

MDFGSIAAKMTLDISNFTSQLNLAQSQAQRLALESSKSFQIGSALTGLGKGLTTAVTLPL
MGFAAASIKVGNEFQAQMSRVQAIAGATAEELGRMKTQAIDLGAKTAFSAKEAAQGMENL
ASAGFQVNEIMDAMPGVLDLAAVSGGDVAASSEAMASSLRAFGLEANQAGHVADVFARAA
ADTNAETSDMAEAMKYVAPVAHSMGLSLEETAASIGIMADAGIKGSQAGTTLRGALSRIA
KPTKAMVKSMQELGVSFYDANGNMIPLREQIAQLKTATAGLTQEERNRHLVTLYGQNSLS
GMLALLDAGPEKLDKMTNALVNSDGAAKEMAETMQDNLASKIEQMGGAFESVAIIVQQIL
EPALAKIVGAITKVLEAFVNMSPIGQKMVVIFAGMVAALGPLLLIAGMVMTTIVKLRIAI
QFLGPAFMGTMGTIAGVIAIFYALVAVFMIAYTKSERFRNFINSLAPAIKAGFGGALEWL
LPRLKELGEWLQKAGEKAKEFGQSVGSKVSKLLEQFGISIGQAGGSIGQFIGNVLERLGG
AFGKVGGVISIAVSLVTKFGLAFLGITGPLGIAISLLVSFLTAWARTGEFNADGITQVFE
NLTNTIQSTADFISQYLPVFVEKGTQILVKIIEGIASAVPQVVEVISQVIENIVMTISTV
MPQLVEAGIKILEALINGLVQSLPTIIQAAVQIITALFNGLVQALPTLIQAGLQILSALI
NGLVQALPAIIQAAVQIIMSLVQALIENLPMIIEAAMQIIMGLVNALIENIGPILEAGIQ
ILMALIEGLIQVLPELITAAIQIITSLLEAILSNLPQLLEAGVKLLLSLLQGLLNMLPQL
IAGALQIMMALLKAVIDFVPKLLQAGVQLLKALIQGIASLLGSLLSTAGNMLSSLVSKIA
SFVGQMVSGGANLIRNFISGIGSMIGSAVSKIGSMGTSIVSKVTGFAGQMVSAGVNLVRG
FINGISSMVSSAVSAAANMASSALNAVKGFLGIHSPSRVMEQMGIYTGQGFVNGIGNMIR
TTRDKAKEMAETVTEALSDVKMDIQENGVIEKVKSVYEKMADQLPETLPAPDFEDVRKAA
GSPRVDLFNTGSDNPNQPQSQSKNNQGEQTVVNIGTIVVRNNDDVDKLSRGLYNRSKETL
SGFGNIVTP

| | |
|---|---|
| >dp1ORF003 amino acid sequence | (SEQ ID NO. 284) |

MAQKGLFGAKPRSSKKNDAQLLAQRKNRKPAVEVTYISGNALKDAVARARTLSTRILGHV
LDRLELITEEAKLEQYVDKMIEDGIGSIDVETDGLDTIHDELAGVCLYSPSQKGIYAPVN
HVSNMTKMRIKNQISPEFMKKMLQRIVDSGIPVIYHNSKFDMKSIYWRLGVKMNEPAWDT
YLAAMLLNENESHSLKSLHSKYVRNEENAEVAKFNDLFKGIPFSLIPPDVAYMYAAYDPL
QTFELYEFQEQYLTPGTEQCEEYNLEKVSWVLHNIEMPLIKVLFDMEVYGVDLDQDKLAE
IREQFTANMNEAEQEFQQLVSEWQPEIEELRQTNFQSYQKLEMDARGRVTVSISSPTQLA
ILFYDIMGLKSPERDKPRGTGESIVEHFDNDISKALLKYRKYAKLVSTYTTLDQHLAKPD
NRIHTTFKQYGAKTGRMSSENPNLQNIPSRGEGAVVRQIFAASEGHYIIGSDYSQQEPRS
LAELSGDESMRHAYEQNLDLYSVIGSKLYGVPYEECLEFYPDGTTNKEGKLRRNSVKSVL
LGLMYGRGANSIAEQMNVSVKEANKVIEDFFTEFPKVADYIIFVQQQAQDLGYVQTATGR
RRRLPDMSLPEYEFEYIDASKNEDFDPFNFDADQQMDDTVPEHIIEKYWAQLDRAWGFKK
KQEIKDQAKAEGILIKDNGGKIADAQRQCLNSVIQGTAADMTKYAMIKVHNDAELKELGF
HLMIPVHDELLGEVPIKNAKRGAERLTEVMIEAAKDIISLPMKCDPSIVERWYGEEIEI

| | |
|---|---|
| >dp1ORF004 amino acid sequence | (SEQ ID NO. 285) |

MTKFINSYGPLHLNLYVEQVSQDVTNNSSRVSWRATVDRDGAYRTWTYGNISNLSVWLNG
SSVHSSHPDYDTSGEEVTLASGEVTVPHNSDGTKTMSVWASFDPNNGVHGNITISTNYTL
DSIPRSTQISSFEGNRNLGSLHTVIFNRKVNSFTHQVWYRVFGSDWIDLGKNHTTSVSFT
PSLDLARYLPKSSSGTMDICIRTYNGTTQIGSDVYSNGWRFNIPDSVRPTFSGISLVDTT
SAVRQILTGNNFLQIMSNIQVNFNNASGAYGSTIQAFHAELVGKNQAINENGGKLGMMNF
NGSATVRAWVTDTRGKQSNVQDVSINVIEYYGPSINFSVQRTRQNPAIIQALRNAKVAPI
TVGGQQKNIMQITFSVAPLNTTNFTEDRGSASGTFTTISLMTNSSANLAGNYGPDKSYIV
KAKIQDRFTSTEFSATVATESVVLNYDKDGRLGVGKVVEQGKAGSIDAAGDIYAGGRQVQ
QFQLTDNNGALNRGQYNDVWNKRETEFTWRSNKYEDNPTGTRGEWGLFQNFWLDSWKMVQ
SFITMSGRMFIRTANDGNSWRPNKWKEVLFKQDFEQNNWQKLVLQSGWNHHSTYGDAFYS
KTLDGIVYLRGNVHKGLIDKEATIAVLPEGFRPKVSMYLQALNNSYGNAILCIYTDGRLV
VKSNVDNSWLNLDNVSFRI

| | |
|---|---|
| dp1ORF005 amino acid sequence | (SEQ ID NO. 286) |

MAKKSKAISHTDELISQSFDSPLAKNQKFKKELQEVEKYYQYFDGFDVTDLNTDYGQTWK
IDEDSVDYKPTREIRNYIRQLIKKQSRFMMGKEPELIFSPVQDNQDEQAENKRILFDSIL
RNCKFWSKSTNALVDATVGKRVLMTVVANAAQQIDVQFYSMPQFTYTVDPRNPSSLLSVD
IVYQDERTKGMSTEKQLWHHYRYEMKAGTSQSGIATALEDIEEQCWLTYALTDGESNQIY
MTESGQTTIKETEAKLVEIEDNLGNKIEVPLKVQESAPTGLKQIPCRVILNEPLTNDIYG
TSDVKDLITVADNLNKTISDLRDSLRFKMFEQPVIIDGSSKSIQGMKIAPNALVDLKSDP
TSSIGGTGGKQAQVTSISGNFNFLPAAEYYLEGAKKAMYELMDQPMPEKVQEAPSGIAMQ
FLFYDLISRCDGKWIEWDDAIQWLIQMLEEILATVNVDLGNIPQDIQSSYQTLTTMTIEH
HYPIPSDELSAKQLALTEVQTNVRSHQSYIEEFSKKEKADKEWERILEELAQLDEISAGA
LPVLANELNEQEEPQDETSEEDEVDDKEKEQTEQPTEEGVDPDVQG

| | |
|---|---|
| >dp1ORF006 amino acid sequence | (SEQ ID NO. 287) |

MIEIVIARSKARRGRTLFIETWASTDEDAVKMAEKISSLPNVVETSSNNFELPYKYFNNV
IDALDEWELHIFGELDKDVQDYIDSRNRIASSSNEQFSFKTTPFAHQVECFEYAQEHPCF
LLGDEQGLGKTKQAIDIAVSRKASFKHCLIVCCISGLKWNWAKEVGIHSNESAHILGSRV
TKDGKLVIDGVSKRAEDLLGGHDEFFLITNIETLRDAVFIKYLNELTKSGEIGMVIIDEI
HKCKNPSSKQGASIQKLQSYYKMGLTGTPLMNNPIDVFNVMKWLGAEHHTLTQFKERYCI
VDQFNQITGYRNLAELRELVNDYMLRRTKEEVLDLPEKIRVTEYVDMNSKQSKIYKEVLT
KLVQEIDKVKLMPNPLAETIRLRQATGNPSILTTQDVKSCKFERCIEIVEECIQQGKSCV
IFSNWEKVIEPLAKILSKTVKCNLVTGETADKFNEIEEFMNHRKASVILGTIGALGTGFT
LTKADTVIFLDSPWTRAEKDQAEDRCHRIGAKSSVTIYTLVAKGTVDERIEDLIERKGEL
ADYIVDGKPMKSKIGNLFDILLK

| | |
|---|---|
| >dp1ORF007 amino acid sequence | (SEQ ID NO. 288) |

MTISLRNKLPKFNFVPFSKKQLQLLTWWTKGSPFRTFDIVIADGSIRSGKTVSMALSFSL
WAMTEFNGQNFAICGKTIHSARRNVIQPLKQMLTSRGYEIRDVRNENLLIIRHFRNGEEI
VNYFYIFGGKDESSQDLIQGVTLAGIFCDEVALMPESFVNQATGRCSVTGSKMWFSCNPA
NPNHYFKKNWIDKQVEKRILYLHFTMDDNPSLTDSIKRRYEKMYAGVFRKRFILGLWVTA
DGLVYSMFNEEQHVKKLNIEFDRLFVAGDFGIYNATTFGLYGFSKRHKRYHLIESYYHSG
REAEEQLTEADVNSNIQFSSVLQKTTKEYANDLVDMIRGKQIEYIILDPSASAMIVELQK
HPYIARKNIPIIPARNDVTLGISFHAELLAENRFTLDPSNTHDIDEYYAYSWDSKASQTG
EDRVIKEHDHCMDRNRYACLTDALINDDFGFEIQILSGKGARN

| | |
|---|---|
| >dp1ORF008 amino acid sequence | (SEQ ID NO. 289) |

VIQLQVLNKVLEEKSLSILENNGIDQEYFTDYLDEYQFIQEHFSRYGRVPDDETILDHFP
GFEFFEIGETDEYLIDKLKEEHLYNSLVPILTEAAEDIQVDSNIAIANIIPKLEELFNRS
KFVGGLDIARNAKLRLDWANTIRNHDGERLGISTGFELLDDVLGGLLPGEDLIVIMARPG
QGKSWTIDKMLATAWKNGHDVLLYSGEMSEMQVGARIDTILSNVSINSITKGIWNDHQFE
KYEDHIQAMTEAENSLVVVTPFMIGGKNLTPAILDSMISKYRPSVVGIDQLSLMSESYPS
REQKRIQYANITMDLYKISAKYGIPIVLNVQAGRSAKTEGAESMELEHIAESDGVGQNAS
RVIAMKRDEKSGILELSVVKNRYGEDRKIIEYMWDVETGTYTLIGFKEEGEEGTEKGESS
PLKAKASRSTARLRSKVTREGVEAF

| | |
|---|---|
| >dp1ORF009 amino acid sequence | (SEQ ID NO. 290) |

MTDFKKRFKKAVTETINRDGIENLMDWLENDTNFFSSPASTRYHGSYEGGLVEHSLNVFN
QLLFEMDTMVGKGWEDIYPMETVAIVALFHDLCKVGQYRETEKWRKNSDGEWESYLAYEY
DPEQLTMGHGAKSNFLLQRFIQLTPVEAQAIFWHMGAYDISPYANLNGCGAAFETNPLAF
LIHRADMAATYVVENENFEYSQGPVEQEAEVEEVVEEKPKSSTRKKPAPKEEKVEEAEEK
PKAGITRRRKPAPKEEEVEEPKEEPKKASSKIRMPKKTEKVEEVESADEPKVEEAEDDNV
VVPAGYVRDVYYFYSEVADVYYKKDVDEPDDDSDILVDEEEYMDAMCPVLEEDFFYELDG
KVHKLAKGERLPEEYDEETWEPITEAEYIKRTEKPKAVAKPTRKTPAPSRRPRP

| | |
|---|---|
| >dp1ORF010 amino acid sequence | (SEQ ID NO. 291) |

MKLEQLMKDWNKDSKALVAVQGLEREALPRIPFSAPSMNYQTYGGLPRKRVVEFFGPESS
GKTTSALDIVKNAQMVFEQEWEQKTEELKEKLENARASKASKTAVKELEMQLDSLQEPLK
IVYLDLENTLDTEWAKKIGVDVDNIWIVRPEMNSAEEILQYVLDIFETGEVGLVVLDSLP
YMVSQNLIDEELTKKAYAGISAPLTEFSRKVTPLLTRYNAIFLGINQIREDMNSQYNAYS
TPGGKMWKHACAVRLKFRKGDYLDENGASLTRTARNPAGNVVESFVEKTKAFKPDRKLVS
YTLSYHDGIQIENDLVDVAVEFGVIQKAGAWFSIVDLETGEIMTDEDEEPLKFQGKANLV
RRFKEDDYLFDMVMTAVHEIITREEG

| | |
|---|---|
| >dp1ORF011 amino acid sequence | (SEQ ID NO. 292) |

MNIYDYINAGEIASYIQALPSNALQYLGPTLFPNAQQTGTDISWLKGANNLPVTIQPSNY
DAKASLRERAGFSKQATEMAFFRESMRLGEKDRQNLQMLLNQSSALAQPLITQLYNDTKN
LVDGVEAQAEYMRMQLLQYGKFTVKSTNSEAQYTYDYNMDAKQQYAVTKKWTNPAESD0PI
ADILAAMDDIENRTGVRPTRMVLNRNTYNQMTKSDSIKKALAIGVQGSWENFLLLASDAE
KFIAEKTGLQIAVYSKKIAQFADADKLPDVGNIRQFNLIDDGKVVLLPPDAVGHTWYGTT
PEAFDLASGGTDAQVQVLSGGPTVTTYLEKHPVNIATVVSAVMIPSFEGIDYVGVLTTN

| | |
|---|---|
| >dp1ORF012 amino acid sequence | (SEQ ID NO. 293) |

MSIKFKTEELSKIVSQLNKLKPSKLLEITNYWHIFGDGECVMFTAYDGSNFLRCIIDSDV
EIDVIVKAEQFGKLVEKTTAATVTLVPEESSLKVIGNGEYNIDIVTEDEEYPTFDHLLED
VSEENALTLKSSLFYGIANINDSAVSKSGADGIYTGFLLKGGKAITTDIIRVCINPIKEK
GLEMLIPYNLMSILASIPDEKMYFWQIDDTTVYISSASVEIYGKLMEGMEDYEDVSQLDS
IEFEDDAAIPTAEILSVLDRLVLFTSAFDKGTVEFLFLKDRLRIKTSTSSYEDIMYASAG
KKVSKKEFTCHLNSLLLKEIVSTVTEENFTVSYGSETAIKISSNGVVYFLALQEPEE

| | |
|---|---|
| >dp1ORF013 amino acid sequence | (SEQ ID NO. 294) |

MNLASKYRPQTFEEVVAQEYVKEILLNQLQNGAIKHGYLFCGGAGTGKTTTARIFAKDVN
KGLGSPIEIDAASNNGVENVRNIIEDSRYKSMDSEFKVYIIDEVHMLSTGAFNALLKTLE
EPSSGTVFILCTTDPQKIPDTILSRVQRFDFTRIDNDDIVNQLQFIIESENEEGAGYSYE
RDALSFIGKLANGGMRDSITRLEKVLDYSHHVDMEAVSNALGVPDYETFASLVEAIANYD
GSKCLEIVNDFHYSGKDLKLVTRNFTDFLLEVCKYWLVRDISITQLPAHFESKLEQFCEA
FQYPTLLWMLEEMNELAGVVKWEPNAKPIIETKLLLMSKEE

| | |
|---|---|
| >dp1ORF014 amino acid sequence | (SEQ ID NO. 295) |

MKVNGLQIEATPEQIIEKLSRQLEDEGTFIFRRTKSLGSNYQFSCPFHAGGTEKHPSCGM
SRNPSYSGSKVTEAGTVHCFTCGYTSGLTEFVSNVLGRNDGGFYGNQWLKRNFGTSSEVV
RQGVSPEAFRRNGRTEKVEHKIIPEEELDKYRFIHPYMYERKLTDELIEMFDVGYDKLHD
CITFPVRNLKGETVFFNRRSVRSKFHQYGEDDPKTEFLYGQYELVAFRDYFEKPISQVFV
TESVINCLTLWSMKIPAVALMGVGGGNQINLLKRLPYRNIVLALDPDNAGQTAQEKLYRQ
LKRSKVVRFLNYPKEFYDNKWDINDHPELLNFNDLVL

| | |
|---|---|
| >dp1ORF015 amino acid sequence | (SEQ ID NO. 296) |

MGFNLYFAGGHAISTDDYLKERGANRLFNQLYERNGIGKRWIEHKKTNPSTTSKLFVDSS
AYSAHTKGAEVDIDAYIEYVNDNVGMFDCIAELDKIPGVFRQPKTREQLLEAPQISWDNY
LYMRERMVEKDKLLPIFHMGEDFKWLNLMLETTFEGGKHIPYIGISPANDSTTKHKDKWM
ERVFEVIRNSSNPDVKTHAFGMTVTSQLERHPFYSADSTSVLLTGAMGNIMTSKGLVDLS
QKNGGIDAVRRLPKPVQVEIESIIEETGAHFSLEQLVEDYKLRALFNVQYMLNWAENYEF
KGIKNRQRRLF

| | |
|---|---|
| >dp1ORF016 amino acid sequence | (SEQ ID NO. 297) |

MGVDIEKGVAWMQARKGRVSYSMDFRDGPDSYDCSSSMYYALRSAGASSAGWAVNTEYMH
AWLIENGYELISENAPWDAKRGDIFIWGRKGASAGAGGHTGMFIDSDNIIHCNYAYDGIS
VNDHDERWYYAGQPYYYVYRLTNANAQPAEKKLGWQKDATGFWYARANGTYPKDEFEYIE
ENKSWFYFDDQGYMLAEKWLKHTDGNWYWFDRDGYMATSWKRIGESWYYFNRDGSMVTGW
IKYYDNWYYCDATNGDMKSNAFIRYNDGWYLLLPDGRLADKPQFTVEPDGLITAKV

| | |
|---|---|
| >dp1ORF017 amino acid sequence | (SEQ ID NO. 3) |

MIGQGLVKSTISKWKQLPKYIIVEGEVGSGRKTLIRYIASKFDADSIVVGTSVDDIRNII
QDAQTIFKARIYVIDGNSLSMSALNSLLKIAEEPPLNCHIAMTVDSINNALPTLASRAKV
LTMLPYTNEEKMQFVKSYKKVDTSGIDDRAIVDYCNLASNLQMLEDILEYGAEELFEKVT
TFYDLIWEASASNSLKVTNWLKFKETDEGKIEPKLFLNCLLNWSTVVIRKHYVEMSFEEL
EAHDLLVREASRCLRKVSKKGSNARVCVNEFIRRVKQVE

| | |
|---|---|
| >dp1ORFQ18 amino acid sequence | (SEQ ID NO. 298) |

MASRQTLLVDGIDLVDKGATVLEYVGLTFAGFKDSGFKNPEGIDGVLDSPSNAMSALTGS
VTLMFHGETEKQVNQKYRQFKQFIRSKSFWRISTLEDPGYYRTGKFLGETEQGKLVDVQA
FKDTSLVVKLGIQFKDAYEYSDSTVRKVYKFQPALGGDSLPNPGRPTRQFRVEIRTTSQI
KGYFRIGEKSSGQFVEFGTNSVLMESGSIIILNLGTFELIKISSANQATNLFRYIKRGAF
FKIPNGNSTITIEYRADDAAAWTSTLPAQVELFLNPSYY

| | |
|---|---|
| >dp1ORF019 amino acid sequence | (SEQ ID NO. 299) |

MNVYLNQMGNVVRETSVSTVWKTLTQKGLVSNHRIFAVRDDKEFLSNESRWKRLPDVRYG
TLVLMVTKIDKRSKLLKAFPDNCVEFEKMTDAQLKRHFVSKYSTIDSDMIDMVIQFCLND
YSRIDNELDKLSRLKKVDASVVESIVKHKTEIDIFSLVDDVLEYRPEQAIMKVTELLAKG
ESPIGLLTLLYQNFNNACLVLGADEPKEANLGIKQFLINKIVYNFQYELDSAFEGMAILG
QAIEGIKNGRYTESSVVYISLYKIFSLT

| | |
|---|---|
| >dp1ORF020 amino acid sequence | (SEQ ID NO. 300) |

MVNQYNQPERGKIRINVRDPEKMPIMEIFGPTIQGEGMVIGQKTIFIRTGGCDYHCNWCD
SAFTWNGTTEPEYITGKEAASRILKLAFNDKGEQICNHVTLTGGNPALINEPMAKMISIL
KEHGFKFGLETQGTRFQEWFKEVSDITISPKPPSSGMRTNMKILEAIVDRMNDENLDWSF
KIVIFDENDLAYARDMFKTFEGKLRPVNYLSVGNANAYEEGKISDRLLEKLGWLWDKVYE
DPAFNNVRPLPQLHTLVYDNKRGV

| | |
|---|---|
| >dp1ORF021 amino acid sequence | (SEQ ID NO. 301) |

MQTHTKKEKSVIGFLKSWDGFGIKCMKTQLSTMFDLYRNFIHLFMIIKEEYKMKIEHLDK
IGNVLGRENGWASLKPDEIVTLDNTEAAVQRLFGLLGEDAERDGLQDTPFRFVKALAEHT
VGYREDPKLHLEKTFDVDHEDLVLVKDIPFNSLCEHHLAPFVGKVHIAYIPKDKITGLSK
FGRVVEGYAKRLQVQERLTQQIADAIQEVLNPQAVAVIVEAEHTCMSGRGIKKHGATTVT
STMRGLFQDDASARAELLQLIKK

| | |
|---|---|
| >dp1ORF022 amino acid sequence | (SEQ ID NO. 302) |

MSKDILYGIKLVQIEELDPLTQLPKVGGANFVVDTAETAELEAVTSEGTEDVKRNDTRIL
AIVRTPDLLYGYDLTFKDNTFDPEIMALIEGGTVRQQGGTIAGYDTPMLAQGASNMKPFR
MNIYVPNYVGDSIVNYVKITLNNCTGKAPGLSIGKEFYAPEFNIKAREATKAGLPVKSMD
YVAQLPAVLRRVTFDLNGGTGTADAVRVEAGKKISPKPVDPTLTGKAFKGWKVEGESTIW
DFDNHMMPDRDVKLVAQFA

| | |
|---|---|
| >dp1ORF023 amino acid sequence | (SEQ ID NO. 303) |

MAKSNLTRIAKMVRAGNSEGPASSFVNSLTRVIERTQPEYNPSTYYKPSGVGGCIRKMYF
ERIGESIIDNADSNLIAMGEAGTFRHEVLQEYMVKMAEIDEDFEWLNVAEFLKENPVEGT
IVDERFKKNDYETKCKNELLQLSFLCDGLVRYKGKLYILEIKTETMFKFTKHTEPYEEHK
MQATCYGMCLGVDDVIFLYENRDNFEKKAYTFHITDEMKNQVLGKIMTCEEYVEKGESPK
IYCSSAYCPYCRKEGRNL

| | |
|---|---|
| >dp1ORF024 amino acid sequence | (SEQ ID NO. 304) |

MNAVDGQVVHILQVLAEDGNATAEKFEKEVRAASLVFSRRAAEAVVKGEIYKDGKNLSKR
VWSSAARAGNDVQQIVTQGLASGMSATDMAKMLEKYIDPKVRKDWDFDKIAEKLGKPAAH
KYQNLEYNALRLARTTISHSATAGVRQWGKVNPYARKVQWHSVHAPGRTCQACIDLDGEV
FPIEECPFDHPNGMCYQTVWYENSLEEIADELRGWVDGEPNDVLDEWYDDLSSGKVEKYS
DLDFVKSY

| | |
|---|---|
| >dp1ORF025 amino acid sequence | (SEQ ID NO. 305) |

MAKNKKRKKVNVKRKMLIPTNLSKKVNVKAIAYRKVTVKWLPNTDEIQVYFDLYINKNRL
TMLGTIDPDKSYFEGIRIVCKKPQPWMTVKELQVARADAPGFFAVLKAYCHTVGDVLDSG
AEPTEIVQGIMYKDGELFKDSEIVSLFKYDVKEPYEFPKDLPITLDNFLEFIMSSQHTRA
LVLRCANIGEFSKNWRKWQKAIQLLLDYAKADDFKVDETVWDFSPGSKAGKVARRKGYEA
IQQALEQINK

| | |
|---|---|
| >dp1ORF026 amino acid sequence | (SEQ ID NO. 306) |

MAKATGPKVRRGKTPPRPKDKKGIKANARVNKDQFVEYDYKGIKMTIKERDARMKLEFIR
GMTIQEIAARYGLNEKRVGEIRARDKWVKAKKEFENEKALVTNDTLTQMYAGFKVSVNIK
YHAAWEKLMNIVEMCLDNPDRYLFTKEGNIRWGALDVLSNLIDRAQKGQERANGMLPEEV
RYRLQIEREKITLLRAKMGDQEIEGEVKDNFVEALDKAAQAVWQEFSDATGSYIKGVTDN
DNKPEK

| | |
|---|---|
| >dp1ORF027 amino acid sequence | (SEQ ID NO. 307) |

MGKVSIQKSGTFSSGSNNEFFTLADHGDSAIVTLLYDDPEGEDMDYFVVHEADVDGRRRY
INCNAIGEDGETVHPDNCPLCQNGFPRIEKLFLQLYNHDTGKVETWDRGRSYVQKIVTFI
NKYGSLVTQPFEIIRSGAKGDQRTTYEFLPERPEDSATLEDFPEKSELLGTLILDLDEDQ
MFDVVDGKFTLQEERSSSRSNSRRGASPAPRRGSGRESSQGRTAERTPSVSRRTPPTRGR
GF

| | |
|---|---|
| >dp1ORF028 amino acid sequence | (SEQ ID NO. 308) |

MSKIKFENLKKGDVVLRAKSQTKFKIVSILADEKKADLESLEDGGELHLSASTLERWYTM
EDETEPKKEEAAKPAKKAAPAVARPARKGRVVPKPKKEVLEEEIPEVKEQPEEVGSVSEK
STVRKPAPKKESVMAITKALESRIVEAFPASTRIVTQSYIAYRSKKNFVTIEETRKGVSI
GVRAKGLTEDQKKLLASIAPASYEWAIDGIFKLVKEEDIDTAMELIEASHLSSL

| | |
|---|---|
| >dp1ORF029 amino acid sequence | (SEQ ID NO. 309) |

MKSVVLLSGGVDSATCLAIEVDKWGSKNVHAIAFNYGQKHEAELENAANVAMFYGVKFTI
LEIDSKIYSSSSSSLLQGKGEISHGKSYAEILAEKEVVDTYVPFRNGLMLSQAAAYAYSV
GASYVVYGAHADDAAGGAYPDCTPEFYNSMSNAMEYGTGGKVTLVAPLLTLTKAQVVKWG
IDLDVPYFLTRSCYESDAESCGTCATCIDRKKAFEENGMTDPIHYKEN

| | |
|---|---|
| >dp1ORF030 amino acid sequence | (SEQ ID NO. 310) |

MNNEKIIEKIKNLIQLANDNPSDEEGQTALLMAQKLMLKNNIALAQVEQFDEPKQFETSQ
AVGKEAGRIFWWERELGHILATNFRCFCINQRDMRLNKSRIIFFGEKQDAELVSKIYEAA
LLYLRYRIDRLPTREPSYKNSYLKGFLSALAIRFKKQVEEYSLMVLPSEQTKNALQDTFR
NLKKEGIDRPQHDFNLEAYIEGRFHGENAKIMPDEILEGGN

| | |
|---|---|
| >dp1ORF031 amino acid sequence | (SEQ ID NO. 311) |

MAYQLEDLLKGLDEPTIKQVKEIISKTSKELDAKIFIDGDGQHFVPHARFDEVVQQRDAA
NGSINSYKEQVATLSKQVKDNGDAQTTIQNLQEQLDKQSQLAKGAVITSALHPLISDSIA
PAADILGFMNLDNITVESDGKVKGLDEELKAVRESRKYLFKEVEVPAEQEAQAKSPAGTG
NLGNPGRVGGGVPEPREIGSFGKQLAAAQQTAGAQEQSSFFK

| | |
|---|---|
| >dp1ORF032 amino acid sequence | (SEQ ID NO. 312) |

MKEANRLVSSYVGFECWTDEECIRNFELDPDMSIASAYHRYFGMLYSYAKRFKCLSRHDI
ESIAFETISKCLATFKSNQGAKFSTYLTRLFKNRIVLEYRYLNAPSMNRNWYVEVTFDSV
STNEEGDDFSILSTVGYCEDYGKIEIEASLDFMTLSNTEYAYISSVIQNGPSVSDAEIAR
EIGVSRSAISQSKKSLKNKLKDFI

| | |
|---|---|
| >dp1ORF033 amino acid sequence | (SEQ ID NO. 313) |

MARPKLPQIDIREEEIRDAQDVADSYGAIINKVVDEIVEAACGSLDQAMEEIQIVVSQNP
VIMEDLNYYIGYLPTLLYFAADRAEMVGIQMDSSSAIRKEKYDNLYILAAGKTIPDKQAE
TRKLVMNEEVIENAYKRAYKKVQLKLEQADKVLASLKRIQTWQLAELETQSNNSKGVLLN
AKRRRREND

| | |
|---|---|
| >dp1ORF034 amino acid sequence | (SEQ ID NO. 314) |

MSQNTTRTDAELTGVTLLGNQDTKYDYDYNPDVLETFPNKHPENNYLVTFDGYEFTSLCP
KTGQPDFANVFISYIPNEKMVESKSLKLYLFSFRNHGDFHEDCMNIILNDLYELMEPKYI
EVMGLFTPRGGISIYPFVNKVNPQFATPELEQLQLQRKLNFLGNVQGLGRAIR

| | |
|---|---|
| >dp1ORF035 amino acid sequence | , (SEQ ID NO. 315) |

MHLMKDSKMLRTWKSLAFEFETKVRTTSGLKLSPAMKTMTRTKIWKGYKMKVFINNHTEA
DIDYKDILNFVAYRNSPNPQIQITSWNALLSCYTRNELSYKGVSITDFFEAIQTIASSFT
HLDSKTIDTQNEKRLERIEELQSRIGHCNCTIDELKKGVHEMPDIESAISYQYGQILAYE
DELNFLLN

| | |
|---|---|
| >dp1ORF036 amino acid sequence | (SEQ ID NO. 316) |

VLVERKADKECWEWLEAVRANIVEEVRNGLSIVIASNTVGNGKTSWAVRLLQRYLAETAL
DGRIVEKGMFVVSAQLLTEFGDYNYFQTMQEFLERFERLKTCELLVIDEIGGGSLTKASY
PYLYDLVNYRVDNNLSTIYTTNYTDDEIIDLLGQRLYSRIYDTSVVLDFQASNVRGLEVS
EIES

| | |
|---|---|
| >dp1ORF037 amino acid sequence | (SEQ ID NO. 317) |

MVKKLKSKIYSVAYIILVVIANLVTIYFEPLNVKGILIPPSSWFMGFTFLLINLISKYEK
PKFAGSLIWVGLFLTSLICFMQNLPQSLVVASGVAFWISQKASVFIFDKLSNKLDSKIAN
ALSSNIGSIIDATIWISLGLSPLGIGTVAYIDIPSAVLGQVLVQFILQSIASRYLKK

| | |
|---|---|
| >dp1ORF038 amino acid sequence | (SEQ ID NO. 318) |

MRVSKTLTFDAAHQLVGHFGKCANLHGHTYKVEISLAGGTYDHGSSQGMVVDFYHVKKIA
GTFIDRLDHAVLLQGNEPIALANAVDTKRVLFGFRTTAENMSRFLTWTLTELMWKHARID
SIKLWETPTGCAECTYYEIFTEDEIEMFKNVTFIDKDEKITVREILEQEQDNG

| | |
|---|---|
| >dp1ORF039 amino acid sequence | (SEQ ID NO. 319) |

MNKSATFWLVRTALIAALYVTLTVAFSAISYGPIQFRVSEALILLPLWNHRWTPGIVLGT
IIANFFSPLGLIDVLFGSLATFLGVVAMVKVAKMASPLYSLICPVLANAYLIALELRIVY
SLPFWESVIYVGISEAIIVLISYFLISTLAKNNHFRTLIGAKNGI

| | |
|---|---|
| >dp1ORF040 amino acid sequence | (SEQ ID NO. 320) |

VSYTGKMFEEDFFEGAKDFEKDAFTVRLYDTTNGFRGVANPCDYIAATNFGTLFIELKTT
KEASLSFNNITDNQWFQLSRADGCKFILAGILVYFQKHEKIIWYPISSLEKIKRSGVKSV
NPNFIDAGYEVSYKKRRTRLTIPFQNVLDAVELHYKEKSNGKT

| | |
|---|---|
| >dp1ORF041 amino acid sequence | (SEQ ID NO. 321) |

MQKDVDVKMIDPKLDRLKYTGDWVDVRISSITKIDADSADVSRCRKVLQKAQVYSVAAGE
CIKIAHGFALELPKGYEAILHPRSSLFKKTGLIFVSSGVIDEGYKGDTDEWFSVWYATRD
ADIFYDQRIAQFRIQEKQPAIKFNFVESLGNAARGGHGSTGDF

| | |
|---|---|
| >dp1ORF042 amino acid sequence | (SEQ ID NO. 322) |

VARQRIGNSGKPKNEIELTFKDKPKTRSTLFKKDVATGLSKVEHDYFQIVEALNGKQFEP
NMKQVSSFFIVQYEFIFNIKCIDYNWFNFSSTMKNVRTYLNIESNIELCRFLAESFVKYE
NVRKRLNLSERFITVSTFKRAWILDELEGKTGSKFEGFY

| | |
|---|---|
| >dp1ORF043 amino acid sequence | (SEQ ID NO. 323) |

MTNIITAEQFKQLAFQIIALPGFSKGSEPIHVKIRAAGVMNLIANGKIPNTLLGKVTELF
GETSTVTKDNASLASITDQQKKEALDRLNKTDTGIQDMAELLRVFAEASMVEPTYAEVGE
YMTDEQLMTIFSAMYGEVTQAETFRTDEGNV

| | |
|---|---|
| >dp1ORF044 amino acid sequence | (SEQ ID NO. 324) |

MVSVLISSSSFLKFLLHFSSTSISKSNKVFNFLVSYISGEPIMALRTFEESPLYALFDMF
RNNLFRCKVELMLTMVTINLERLGRLLLRLVVQFVLFLCHQLRLLHSFHLEAPLVRLIRL
LIQAMLQLRFRQAEQVLPKCVPIPCPPFPSY

| | |
|---|---|
| >dp1ORF045 amino acid sequence | (SEQ ID NO. 325) |

MKRVKKTKLMTKKKNKLNNQPKKESTQTFKVNCDHCEHKFDLTSKQIISKHIEKGVEWRF
FECPKCHYRFTTYVGNKEIENLIRFRNTCRAKMKQELQKGAAANQNTYHSYRIQDEQAGH
KISGLMAKLKKEINIEKREKEWVSI

| | |
|---|---|
| >dp1ORF046 amino acid sequence | (SEQ ID NO. 326) |

MPMWLNDTAVLTTIITACSGVLTVLLNKLFEWKSNKAKSVLEDISTTLSTLKQQVDGIDQ
TTVAINHQNDVIQDGTRKIQRYRLYHDLKREVITGYTTLDHFRELSILFESYKNLGGNGE
VEALYEKYKKLPIREEDLDETI

| | |
|---|---|
| >dp1ORF047 amino acid sequence | (SEQ ID NO. 327) |

MKFEDEKQFIAAIEEAGELNATKGDMEKQVKSLRDALKEYMKENDIESAQGKHFSATFYT
TERSTMDEERLKEIIEKLVDEAETEEMCEKLSGLIEYKPVINTKLLEDMIYHGEIDQEAI
LPAVVISVTEGIRFGKAKI

| | |
|---|---|
| >dp1ORF048 amino acid sequence | (SEQ ID NO. 328) |

METTLYFGYLTADWKDGHKNYTFHYESIPVKETEKQYKVTGINPNLYLDLGSVIRKSELD
IAVFKACPVAETGVTLTRDMEVDARIEIIKKLTTRIERLNERIKARNEQGKQESRHLVSA
LEDCARQIAGIYQ

| | |
|---|---|
| >dp1ORF049 amino acid sequence | (SEQ ID NO. 329) |

MFQPFLSEHVALVVKVEPRLVFFDILELIFWISSVCSSVPETSSIFLPAKFLLSRLSICV
SQAIDVVVRLTCIVPTLIVVVDGNSVVGVVAVNDVITVNEHPCMTSSACASTFASPDEDV
ASFSIPRSIFTN

| | |
|---|---|
| >dp1ORF050 amino acid sequence | (SEQ ID NO. 330) |

MNNQRKQMNKRIVELREDYQRARGRINFLLAVKDHGEELENLEAFVGYIDNLVECFPESQ
RNVLRLCVLDDLPVTNAAAEIGYHYTWVHQLRDKAVETLEEILDGDNIIRSKHGIEIKEK
LDELYGKSHSS

| | |
|---|---|
| >dp1ORF051 amino acid sequence | (SEQ ID NO. 331) |

MSYDVNYVKNQVRRAIETAPTKIKVLRNSWVSDGYGGKKKDKANEVVADDLVCLVDNSTV
PDLLANSTDAGKIFAQNGVKIFILYDEGKIIQRADTIEIKNSGRRYRVVETHNLLEQDIL
IELKLEVND

| | |
|---|---|
| >dp1ORF052 amino acid sequence | (SEQ ID NO. 332) |

MTKRTTMMDRLKEILPTFQLSPAPMLPGVEFDEQDTDRPDDYIVLRYSHRMPSATNSLGS
FAYWKVQIYVHSNSIIGIDEYSRKVRNIIKDMGYEVTYAETGDYFDTMLSRYRLEIEYRI
PQGGN

| | |
|---|---|
| >dp1ORF053 amino acid sequence | (SEQ ID NO. 333) |

MLTFERIVSIRAPTCISLISPLYRRTSCPFFQAVASILSIVHDLPCPGRAIMTIKSSPGS
KPPSTSSNSSNPVDIPSLSPSWFLIVFAQSSRSLAFRAMSSPPTNLERLKSSSSFGIIFA
IAMLLST

| | |
|---|---|
| >dp1ORF054 amino acid sequence | (SEQ ID NO. 334) |

MCENCQNETFNTRIFNEDESGYVDASFTYKEIRDTAAAISNRAVEKKDRDSLLVATVMAL
PVSHAEDLGKRLCIANSRLEAFREAVQEALENEKAEDLKDVILGLIDVDKKIGNLALQLV
ESGAL

| | |
|---|---|
| >dp1ORF055 amino acid sequence | (SEQ ID NO. 335) |

MPNVRVKKTDFNQTTRSIVAIPDHYVALAAQIPATAATQVGNKKYILAGTCVKNATTFEG
RKTGLEVVSTGEQFDGVIFADQEVFEGEEKVTVTVLVHGFVKYAALRKVGDAVPESKNAM
ILVVK

| | |
|---|---|
| >dp1ORF056 amino acid sequence | (SEQ ID NO. 336) |

MENKWKVIHFQNSCIKQVDDEKRRLLFEVPGTPYRLQVWVKMSLVKIETRAGNGYYKRLV
CQDDFVFYGKESIDGYLIDATITGKSLAEYCEPMNRHILETIASREAAELNRAKKQDQQK
WRY

| | |
|---|---|
| >dp1ORF057 amino acid sequence | (SEQ ID NO. 337) |

MQKSLFGPKLVPASSRRKKRTVPKPKPKIDEQVVELMNRRERQVLVHSCIYYYFNDSIIA
DGQYDKWSHELYSLIVSHPDEFRQTVLYNEFKQFDGNTGMGLPYDCQFAVRVAERLLRK

| | |
|---|---|
| >dp1ORF058 amino acid sequence | (SEQ ID NO. 338) |

MTSRAYKPIPTRRASAKQEKAVAKQLGGKVQPNSGATDYYKGDVVTDSMLIECKTVMKPQ
SSVSLKKEWFLKNEQERFAQKLDYSAIAFDFGDGGEQYIAMSISQFKRILEDRNDNLI

| | |
|---|---|
| >dp1ORF059 amino acid sequence | (SEQ ID NO. 339) |

MSQPELVWKPEEFVSNCERYRNKFQVAVITVCEVAATKMEEYAKTHAIWTDRTGNARQKL
KGEAAWVSADQIMIAVSHHMDYGFWLELAHGRKYKILEQAVEDNVEELFRALRRLLD

| | |
|---|---|
| >dp1ORF060 amino acid sequence | (SEQ ID NO. 340) |

VIAVSAIPTPLFPGTPSTPSRPGAPGKPASPLGPSSRIHVKSSGTNSLGFLLVLRTPMYF
PDSALKLVPKMSSAYLITTWDSFTVSPERTPSPSSFSKSIKSFRGSWKMIVEFERSS

| | |
|---|---|
| >dp1ORF061 amino acid sequence | (SEQ ID NO. 341) |

MARMQRLCPMKFWKAVTKMKFEVYSARLFDEEATYDRYREALEKVGNVAYFCEIDTGNLV
IELELDSLDDLIALSNVVGTGLKLSRPYREDKPFQLWIVDGYME

| | |
|---|---|
| >dp1ORF062 amino acid sequence | (SEQ ID NO. 342) |

VRSFNQFHCGVNIFFLDEFKNSVNRPFVRCRSNRCKKFLLVFCQPFCANSNRNTFSSFFD
SNEVLLRAIGDVRLSDDSSRRRKGFNNSTFKSLSNRHHAFFFRSRFSNSRFLTN

| | |
|---|---|
| >dp1ORF063 amino acid sequence | (SEQ ID NO. 343) |

MKFTEGKNWYKVGEICQMLNRSLSTINVWYEAKDFAEENNIHFPFVLPEPRTDLDHRGSR
FWDDEGVNKLKRFRDNLMRGDLAFYTRTLVGKTEREAIQEDAKAFKREHGLEN

| | |
|---|---|
| >dp1ORF064 amino acid sequence | (SEQ ID NO. 344) |

MATLKALSTLIVSGAVVHSGSVFSCPEALASSLIERNFAFEIKAAEDGETVETVPQTIES
VEEIDEVEQMREEYAAKTVPELVELARANGIDISSISRKSEYIDALIKYELGE

| | |
|---|---|
| >dp1ORF065 amino acid sequence | (SEQ ID NO. 345) |

MQFVITYIKHLDELVRQFPFIHIRMNKPVFIKFLFRNDFMLDFFSSPISSKRFRADALPN
YFARCSKIPFQPLVSIEPSIVST

| | |
|---|---|
| >dp1ORF066 amino acid sequence | (SEQ ID NO. 346) |

VTNCVRWKQYHFTVVNQVELTNVTNVRKFVSVSELSNFLRVDSDLKTCFFSDEFLSVTCK
KQEVFPRTLNTNCKSFLDRVTLSHLVISVSVQDHSSRANTCTIFDVIHCC

| | |
|---|---|
| >dp1ORF067 amino acid sequence | (SEQ ID NO. 347) |

VTIRVDAGKASTIRLSRALVIAITLSFLGAGFRTVDFSLTEPTSSGCSLTSGISSSRTSF
LGLGTTLPFRAGRATAGAAFLAGLAASSFLGSVSSSIVYQRSRVEAER

| | |
|---|---|
| >dp1ORF068 amino acid sequence | (SEQ ID NO. 348) |

MAAQTDIELVKINIDNDNSPSPMTDQSISALLDKHKSVAYVSYMICLMKTRNDVVTLGPI
SLKGDADYWKQMAQFYYDQYKQEQLETDEKSNAGSTILMKRADGT

| | |
|---|---|
| >dp1ORF069 amino acid sequence | (SEQ ID NO. 349) |

MKLYHATDFDNLGKILAEGLKPSAGVIYLAESYEKALAFLSLRNVDTIVVLELEVDIEKC
TESFDHNEKMFCSLFHFDTCRAWTYDKTIEVDDIDFSKARKYDRK

| | |
|---|---|
| >dp1ORF070 amino acid sequence | (SEQ ID NO. 350) |

MITLFKINSEGTVTPIKGSAMQLYADLIPIQEDDIQFVDITGLDPIVRENVLELISRSRV
GVSKYGTNLDQNDVDDFLQHAKEEALDFANYLTKLQSQQKQNK

| | |
|---|---|
| >dp1ORF071 amino acid sequence | (SEQ ID NO. 351) |

VKQVLEEFKVFKVLKGFKEFLDLQELTDVRNILTSLSLIVQTVRDLVILTADEHTSVSIK
ISIPSIQKTLQPIHGRNGRGMTELKGYPGSQAQTVRLIISI

| | |
|---|---|
| >dp1ORF072 amino acid sequence | (SEQ ID NO. 352) |

MFLRLQVVSKVFQLFVQESLQFEDHLLSSKCFNSFPCNLTSKTSSRPRGFCFQWRAFAFF
SSFFAFLFESYKSIGSSFNVPHIFDDFSVFAISVFNDR

| | |
|---|---|
| >dp1ORF073 amino acid sequence | (SEQ ID NO. 353) |

VNACRKNTTKKLGNLSLKQNTSSEQKNLKQLQNLLEKLQRLLVALALKRKVEIKCVKIVK
TKHSILEFSMKMKVAMSTPHSLTRRFATPQQLLAIER

| | |
|---|---|
| >dp1ORF074 amino acid sequence | (SEQ ID NO. 354) |

VTKRKIQDCKCLWSDYFQSLLFLYIERKLHGFWVNCSKNDFGYLKLHKSIKSCSKSSATA
RTRVFEVLSNWFCFNRIRERTYDCGYPSSYGICSRLY

| | |
|---|---|
| >dp1ORF075 amino acid sequence | (SEQ ID NO. 355) |

MAKFCPLNSVMAQRENERAIDTVFPERMEPSAMTISKVRKGEPFVHHVRSWSCFLLKGTK
LNLGSLFLRLIVIISHSFNVGTCCVTKFLPNGLSCFI

| | |
|---|---|
| >dp1ORF076 amino acid sequence | (SEQ ID NO. 356) |

VRAFSSLTSSSKWSNVGYSSSSVTISILYSPFPITFSEDSSGTNVTVAAVVFSTSFPNCS
AFTITSISTSLSIMHRRKFEPSYAVNMTHSPSPKICQ

| | |
|---|---|
| >dp1ORF077 amino acid sequence | (SEQ ID NO. 357) |

MERIKTLFHVIYANGTHLEVAALFDTVDDYDDVIEDIQGYIDTPDLYNQRSIRMAPYNPD
INGDAIATDILLRLDDIIYVDATCETIKYEEPIA

| | |
|---|---|
| >dp1ORF078 amino acid sequence | (SEQ ID NO. 358) |

MATVKETVKFDGRLVTIFDYDDLEWEGYAPNEGFEDVEDMEVLSIRVRNEGEDDEWVEVI
ACYENDDEDEDLEGL

| | |
|---|---|
| >dp1ORF079 amino acid sequence | (SEQ ID NO. 359) |

MELIPLINPRTRLTPALTICPANPVTLETIEVPMLPILETAEPIIDPIPLMKFRIRFAPP
ETICPTKLAILLTNDESMFPAVDKSEPRSEAIP

| | |
|---|---|
| >dp1ORF080 amino acid sequence | (SEQ ID NO. 360) |

MLNLTKSRQIVAEFTIGQGAEKKLVKTTIVNIDANAVSTVSETLHDPDLYAANRRELRAD
EQKLRETRYAIEDEILAEQSKTETALTAE

| | |
|---|---|
| >dp1ORF081 amino acid sequence | (SEQ ID NO. 361) |

MFRNSIVHLLVCVKVKGVEIFVLASVDILELVFRKTHIRKPSSSTGSCLNISQVLRLLLN
EYDIVCHFRELGEEIFNNLIRFFDRYIHLLSD

| | |
|---|---|
| >dp1ORF082 amino acid sequence | (SEQ ID NO. 362) |

VNFTFQLQLSNVGTQWKMKLNLKKKKLLNLLKRLLLQLLDLLEKVESFPNLKKKSLRKKF
LKLRNSRKKLVQLVRNLLFENLLLKKKA

| | |
|---|---|
| >dp1ORF083 amino acid sequence | (SEQ ID NO. 363) |

MPSGFLNPESLNPAKVSPTYSSTVAPLSTRSIPSTNSVCLLAIYFSFTVLQCYQTLIEFL
YFYYTILSTVCQRRHCFELRLFQC

| | |
|---|---|
| >dp1ORF084 amino acid sequence | (SEQ ID NO. 364) |

MNYMVKVILVSVFVLSAFCMTCSMVYLVTGKQEDHRSTVALVFGALVSSAAFYSTLFILA
YLP

| | |
|---|---|
| >dp1ORF085 amino acid sequence | (SEQ ID NO. 365) |

VMTIIKDFFEPCDTVTHSSICKFPNKRKGVTLITITSSFFIFTFDNKLKLINDVVIINSS
KVKPLNSTENSVRNLLRVSST

| | |
|---|---|
| >dp1ORF086 amino acid sequence | (SEQ ID NO. 366) |

IWEKYQFKNQEHLAQGLITSFSHSLTTVTAQLSLYCMMTRKAKTWIIS

| | |
|---|---|
| >dp1ORF087 amino acid sequence | (SEQ ID NO. 367) |

MILPSSYRMKIFTPFWAKIFPASVELAKRSGTVELSTKQTRSSATTSFALSFFFPPYPSL
TQEFRSTLILVGAVSMALRT

| | |
|---|---|
| >dp1ORF088 amino acid sequence | (SEQ ID NO. 4) |

MKKVQTYQEYLKLVEFKRQLSLNLREGKIGVDEAVIQLFTFYSFNNIEEPPFIVLKMQEA
AVNGTYEAKLNMLKRFKII

| | |
|---|---|
| >dp1ORF089 amino acid sequence | (SEQ ID NO. 368) |

MSIMSLSIVEYLDTKCLFNCASVIFSNSTQLSGKAFSNLLRLSILVTIKTSVPYLTSGSL
FHLDSLDRNSLSSRTANIR

| | |
|---|---|
| >dp1ORF090 amino acid sequence | (SEQ ID NO. 369) |

MLKFSLTATVNILYLTHVSMKLFNSAMQLTAQLILIKNKSRRFLNRSKITVMRRPLSKTF
KSNSTSSLNLQKAL

| | |
|---|---|
| >dp1ORF091 amino acid sequence | (SEQ ID NO. 370) |

MKLSNEQYDVAKNVVTVVVPAAIALITGLGALYQFDTTAITGTIALLATFAGTVLGVSSR
NYQKEQEAQNNEVE

| | |
|---|---|
| >dp1ORF092 amino acid sequence | (SEQ ID NO. 371) |

MKTISILRKDTKRKPDRNGRKTALELAQEIDMSPSELAELLQIPERTATRILKLDKLLNK
EQCSIIERYINEIH

| | |
|---|---|
| >dp1ORF093 amino acid sequence | (SEQ ID NO. 372) |

MQHTIKQCLKLAFLLTAISIACLVFPKPCSSPKRKHGCSCAYSKHSTWCANGVVLNENCS
LLEEAIRFRESM

| | |
|---|---|
| >dp1ORF094 amino acid sequence | (SEQ ID NO. 373) |

MYELVLSLKLTPTAPMSQDVEKCFKRLKYIQWRQVNALKLHTDLLLNFLRDMKQSCILVP
VFLRKLV

| | |
|---|---|
| >dp1ORF095 amino acid sequence | (SEQ ID NO. 374) |

VGKLLQLSTLSRMRKWYLSRNGNRRLKNSRKSWKMRVHPKLARLLSRNLKCNSIVFKSLL
RLYILTLRIH

| | |
|---|---|
| >dp1ORF096 amino acid sequence | (SEQ ID NO. 375) |

VIHKFFNFVELICGFSCYQVAFDCLRKYLSKRFNNLFPIAKYHAGLSLLDTFLDNFDTSF
ELARLDILSS

| | |
|---|---|
| >dp1ORF097 amino acid sequence | (SEQ ID NO. 376) |

MDGIEILILTDVCSSAVSMTKSLTVWTIRESEVSILRTSVSSCRSRNSLKPLRTLKTLNS
SRTCFTYLGN

| | |
|---|---|
| >dp1ORF098 amino acid sequence | (SEQ ID NO. 377) |

VKMLRGMLNEATSSSGDAKVLAQALEVIQGCSLTVITSFTATTPTTEFPSTTTMSVGTMQ
VNLTTTSIA

| | |
|---|---|
| >dp1ORF099 amino acid sequence | (SEQ ID NO. 378) |

MQVRHLLLKLQLVDGLRKFLPSQVVSIYGLEQDGATLTKLMKLDIQFQEWASRVLKVTQV
VTVLQERTE

| | |
|---|---|
| >dp1ORF100 amino acid sequence | (SEQ ID NO. 379) |

MQLTPSEFYLDLELRLRICQDSLPGLSRSLCGSMLVSTLSNYGKLLQVAQNVLTTRFSQK
TRLKCSRT

| | |
|---|---|
| >dp1ORF101 amino acid sequence | (SEQ ID NO. 380) |

VIILVQFPLHLKARLGHLGCLARVRLQGCQYQFHKSKRHFQLSLVLHDTYHMSPLRQIVA
QNKLRISF

| | |
|---|---|
| >dp1ORF102 amino acid sequence | (SEQ ID NO. 381) |

MITWECLTVSPNSIKFLVYLDSLRHVNSFWKHHKFLGIIIYTCASEWLRKTSSYLFSIWE
KTLNGST

| | |
|---|---|
| >dp1ORF103 amino acid sequence | (SEQ ID NO. 382) |

LNHRYSNITTIFLWQIVFLCICCAVSYCAGVHNERESQDKVIQSYKQKEKSAVYLTVDSS
GAWLGSAPGAKESPLYNEKGQHVGKLKEVGE

| | |
|---|---|
| >dp1ORF104 amino acid sequence | (SEQ ID NO. 383) |

MRKRVILKLKRLNWYVLNSYSRMVEFFELLNFSNGSTFRRIEVFEPVEFFEHSRLFDPFL
CSTFRVF

| | |
|---|---|
| >dp1ORF105 amino acid sequence | (SEQ ID NO. 384) |

MIVASTSSNENSLLTYNHSFTLNCRTENFHDRHFLRVANIDSNLASFRLIVLINHYPAPA
LKFRGQ

| | |
|---|---|
| >dp1ORF106 amino acid sequence | (SEQ ID NO. 385) |

MNLVNDVNFELAVHRLVSRIFNNVSNIFYPIIRSSINFNRRAKSFVHILRENSSSSGFTS
SSATTE

| | |
|---|---|
| >dp1ORF107 amino acid sequence | (SEQ ID NO. 386) |

MSVTPFRLLGNLQMEECVTVSQGSKKSLIIVITLTWKPFLMH

| | |
|---|---|
| >dp1ORF108 amino acid sequence | (SEQ ID NO. 387) |

MHSCTIGHRAANTKKDNLPKKNSCDVTISMIQFRLPPILLHCLPENLEPLKYHIYDYKAF
GLKGQ

| | |
|---|---|
| >dp1ORF109 amino acid sequence | (SEQ ID NO. 388) |

MWLSKSQIVDSPSTFQPLKALPVKVGSTGFGEIFLPASTRTASAVPVPPFKSNVTRRRTA
GSCAT

| | |
|---|---|
| >dp1ORF110 amino acid sequence | (SEQ ID NO. 389) |

MISILASTSMSRVSVTPVSATGHALNTAMSSSLFLITEPRSKYKLGLIPVTLYCFSVSFT
GMLS

| | |
|---|---|
| >dp1ORF111 amino acid sequence | (SEQ ID NO. 390) |

VTLSRKLLQLVFKVLGKTSCFLQVTLRNSSLKKQVFKSLSTLRKLLSSLTLTNFLTLVTF
VSST

| | |
|---|---|
| >dp1ORF112 amino acid sequence | (SEQ ID NO. 391) |

MQTDLGKYCFDAAAVAYIRYLQEDKTPRYPGDEKKNPGLQMLME

| | |
|---|---|
| >dp1ORF113 amino acid sequence | (SEQ ID NO. 392) |

MKTVKEAIKQFGDEWWYEIINENGQMIQDGRIEDMGEYMEETVDQVKFINYGDIESQIIK
LYIA

| | |
|---|---|
| >dp1ORF114 amino acid sequence | (SEQ ID NO. 393) |

MLLAKTGKQSILIIVHYAKTDSLVLKNYFFNFTTMIREKLKHGTEAVLMFKRLLHLSINM
EAL

| | |
|---|---|
| >dp1ORF115 amino acid sequence | (SEQ ID NO. 394) |

MSLLFLIYIIYTNYREFVKPFLNNFKSFKHIEFCFISPVHGSLLHFEYNERRFLDIVETI
EGE

| | |
|---|---|
| >dp1ORF116 amino acid sequence | (SEQ ID NO. 395) |

MKFSNFAKALTNEYLMVVNNDQAEVLGAGNIENILNGSNFANVVAEATVLKLEKLSEEEA
IE

| | |
|---|---|
| >dp1ORF117 amino acid sequence | (SEQ ID NO. 396) |

MITGCSNILNRSESRKSLIVLFKLSATVIRSLTSLVPYMSLVNGSLRITRQGICFKPVGA
DS

| | |
|---|---|
| >dp1ORF118 amino acid sequence | (SEQ ID NO. 397) |

MILSTSTQLVKLLNTRSLLHEQSAKANEQTNRRTSRRLSTCKRSNKLPSCCKGPRRRTRK
P

| | |
|---|---|
| >dp1ORF119 amino acid sequence | (SEQ ID NO. 398) |

MEVQHPRFSTSYFFGHFFSRHDFSGSTDFNREQLPPNHVEHSSQLQQCFRRLRIHYPSIS
R

| | |
|---|---|
| >dp1ORF120 amino acid sequence | (SEQ ID NO. 399) |

VLKRKQNTCVCNCFNTVNSLSNQLTARLNTLTTTTWMLSNNMQSLRNGLTQLKVTLSLTF

| | |
|---|---|
| >dp1ORF121 amino acid sequence | (SEQ ID NO. 400) |

VQTDHVSSVWKIIINNIWVITPIMSKQIAGIELSIDGLTALPMFKWEVETSSLILYLNLV

| | |
|---|---|
| >dp1ORF122 amino acid sequence | (SEQ ID NO. 401) |

MLFSLSYIPNHVHVWIKRVLFRSKSADLNGLGKDPVIDVNEPLRKVHNFIPCGEHRNSVT

| | |
|---|---|
| >dp1ORF123 amino acid sequence | (SEQ ID NO. 402) |

MVRLFEGLRFSNRLSFSSILDFSTPFYARLFECFEVFEQVRLFEKLSFSTSKLGSIIRKV

| | |
|---|---|
| >dp1ORF124 amino acid sequence | (SEQ ID NO. 403) |

MVKVKDLQVGMKVVNAKGTEFKVTDRQGRKWVSLERLSDGRIRFYDNESLMDEKVEVVK

| | |
|---|---|
| >dp1ORF125 amino acid sequence | (SEQ ID NO. 404) |

MSSAASVKIGTSELYRCSSFSLSIRYSSVSPISKNSNPGKWSRIVSSSGTLPYLEKCS

| | |
|---|---|
| >dp1ORF126 amino acid sequence | (SEQ ID NO. 405) |

MSSSTFSRTIGSSPVISTNCISSSCIGIRSAYSCMADPLIGVTVPSLFILNKVIISIL

| | |
|---|---|
| >dp1ORF127 amino acid sequence | (SEQ ID NO. 406) |

MLNSFPIHRRCSCAIFQFHDTDQLCKGREIVLRLQLFPLGKCLPSLCLPWYPFRKVVD

| | |
|---|---|
| >dp1ORF128 amino acid sequence | (SEQ ID NO. 407) |

MTAVQQVKFYLEEAGAHFLKDVEYSDNLEQAIMKDILKWNGAHRDEHDMKITSYEVL

| | |
|---|---|
| >dp1ORF129 amino acid sequence | (SEQ ID NO. 408) |

MNFLLSNLRSLKFKLMYAATNLTLKNSVRRKRRTRNGNAFWKNLLSLTKSQLEHCLY

| | |
|---|---|
| >dp1ORF130 amino acid sequence | (SEQ ID NO. 409) |

VLDFIPLLSYNHNINKTSVKDAERGQLWKQHFISVILQQIGKTVTRTTLSTMKAFL

| | |
|---|---|
| >dp1ORF131 amino acid sequence | (SEQ ID NO. 410) |

MLNRLRRNLAGRKMLLVSGTLEQTELIQKMSSSISKKTSLGSTLTTKATCSLRNG

| | |
|---|---|
| >dp1ORF132 amino acid sequence | (SEQ ID NO. 411) |

VTGRSSNTHSLKTFRWLSGKHSTRLSMYPTKASRFSSSSPWSFTARRKFIRPLAR

| | |
|---|---|
| >dp1ORF133 amino acid sequence | (SEQ ID NO. 412) |

MTSSFMTSFRVSACLSGIVFPAAKMYRLSYFSFLIAELESICIPTISALSAAK

| | |
|---|---|
| >dp1ORF134 amino acid sequence | (SEQ ID NO. 413) |

MTSMYLGSINSYKSFKIMFMQSSWKSPWLRKLNKYNFNDLDSTIFSFGM

| | |
|---|---|
| >dp1ORF135 amino acid sequence | (SEQ ID NO. 414) |

MKQNLKMLLMLQCSTESSSPFLKLTRKSTQALALPYYKEKAKFHMENLTLKS

| | |
|---|---|
| >dp1ORF136 amino acid sequence | (SEQ ID NO. 415) |

VKKSSITLFASLTDTFICSAIELAPRPYIRPKRTDLTEFLRSFPSLLVVPSG

| | |
|---|---|
| >dp1ORF137 amino acid sequence | (SEQ ID NO. 416) |

MLRTCLLAPSGGQTSRTHSPASLIISSATAPTEEATCFNFLGKPSASSYHNA

| | |
|---|---|
| >dp1ORF138 amino acid sequence | (SEQ ID NO. 417) |

MTISKNNVVIRPICILLVKFNSWKHRSRRELKCRKNFLQSVHHCRSFSHVHS

| | |
|---|---|
| >dp1ORF139 amino acid sequence | (SEQ ID NO. 418) |

MILNHSTCLTLLINSFTQTRAFEPFLDTFRKHLDASLTKRSWASSSSKDIST

| | |
|---|---|
| >dp1ORF140 amino acid sequence | (SEQ ID NO. 419) |

MFSIFPAPKTSAWSLFTTIRYSLVSALAKFENFILFSLYLFFFILLLYNND

| | |
|---|---|
| >dp1ORF141 amino acid sequence | (SEQ ID NO. 420) |

VLRVVEISSKTLLALFDFHSNNLFSRTVSTPLHAVIIVVKTAVSFSHIGID

| | |
|---|---|
| >dp1ORF142 amino acid sequence | (SEQ ID NO. 421) |

VTVEVSPNSSVTLPKSVLGIFPLAIRFMTPAARILTWIGSLPFENPGSAMI

| | |
|---|---|
| >dp1ORF143 amino acid sequence | (SEQ ID NO. 422) |

MKFGLTLLTPDRLIFSRLEIGYHIIFSCFWKYTKIPARINLHPSARDSWNH

| | |
|---|---|
| >dp1ORF144 amino acid sequence | (SEQ ID NO. 423) |

VQIKRLTYLDTLNEAHSSRFLMEIQQLPLNTEPMTQQLGPLLFPLKLNCF

| | |
|---|---|
| >dp1ORF145 amino acid sequence | (SEQ ID NO. 424) |

METAGDLTSGKRFYLSKTSNRIIGRNLFFKVGGTITQPMATHSIRKLLTA

| | |
|---|---|
| >dp1ORF146 amino acid sequence | (SEQ ID NO. 425) |

MTNCMIASPFQYGTSRAKQYSSTVEVFVLSFTSTVKMTLKRNFFMANMSL

| | |
|---|---|
| >dp1ORF147 amino acid sequence | (SEQ ID NO. 426) |

MYLSKKRIRLLKISSPSSLKWQTISYSFNSRRRTWDMFKQLPVEEEGFLI

| | |
|---|---|
| >dp1ORF148 amino acid sequence | (SEQ ID NO. 427) |

VFRFKTIRVGRTPVRFSMSSIAAKMSAIGSLSAGLVHFLVTAYCCLASML

| | |
|---|---|
| >dp1ORF149 amino acid sequence | (SEQ ID NO. 428) |

MPLNFSSIRINLAPLSHSSCGGMANGSSSKSKGIVFEILIFMSSRFP

| | |
|---|---|
| >dp1ORF150 amino acid sequence | (SEQ ID NO. 429) |

VVLYSKKEVYSTSCTLIVFAKFDDSFVHLLSLIVHAIGSSYLIVSQVAST

| | |
|---|---|
| >dp1ORF151 amino acid sequence | (SEQ ID NO. 430) |

MIISTQGRLLATFKHFLQTLFNTLDQLFSLMLNKQGQTFHGSRVQIICQ

| | |
|---|---|
| >dp1ORF152 amino acid sequence | (SEQ ID NO. 431) |

MCIKDLSTKRLLLQYFLKDLDRKFQCIFRLSITHMEMPFYVYTLTEDLW

| | |
|---|---|
| >dp1ORF153 amino acid sequence | (SEQ ID NO. 432) |

MVDKGLTFSNFRYRHSRRFHSFRKNSIDGSFIFPLGHDGIQRTKLCHLW

| | |
|---|---|
| >dp1ORF154 amino acid sequence | (SEQ ID NO. 433) |

VTIGFKNCKKTWGVCTRNLELLNSHPRLRFLTNNPNSFKIALVRVNSA

| | |
|---|---|
| >dp1ORF155 amino acid sequence | (SEQ ID NO. 434) |

MNTTLSNLQWDMVQNLISFFNVSFNSRQLKLKQFSGIWEPMILVLMQI

| | |
|---|---|
| >dp1ORF156 amino acid sequence | (SEQ ID NO. 435) |

MLVSPFLLVLLFSSVQFSCFSRCNSFENMPVHRLTIFRQRFASYGGVN

| | |
|---|---|
| >dp1ORF157 amino acid sequence | (SEQ ID NO. 436) |

VLAGLEKKLVSFSSQSIRFSIPSRLIVSVTAFLKRFLKSVILDPFHFL

| | |
|---|---|
| >dp1ORF158 amino acid sequence | (SEQ ID NO. 437) |

VNAVIRVKRSPNGHCLCPVTIVRNSHFSTCERYLFAGRVVVWVTAMNT

| | |
|---|---|
| >dp1ORF159 amino acid sequence | (SEQ ID NO. 438) |

MIWSALTQAASPLSFCRAFPVRSVQIACVFAYSSILVAATSQTVMTAT

| | |
|---|---|
| >dp1ORF160 amino acid sequence | (SEQ ID NO. 439) |

MGYRHARKTIERPRRIYQCYRILWTVYQFLRSTYSSKSCNYPSSSKC

| | |
|---|---|
| >dp1ORF161 amino acid sequence | (SEQ ID NO. 440) |

MQKGLNAYLDMTLKALHSRLFQNVWQRSNQTKGPSFQLTLQDSSRIE

| | |
|---|---|
| >dp1ORF162 amino acid sequence | (SEQ ID NO. 441) |

MTEVAVNSPQKVRVVMVGNIEFLEYLKRKYGTETSISYIIENERGLI

| | |
|---|---|
| >dp1ORF163 amino acid sequence | (SEQ ID NO. 442) |

VTEFLCSPQGMKLCTLRKGSFTSITGSLPNPFKSADLERNNTRLIQT

| | |
|---|---|
| >dp1ORF164 amino acid sequence | (SEQ ID NO. 443) |

MYSWRTSCLNVPASPIAIRLESALSIIDSPILSKYIFRIHPPTPLGL

| | |
|---|---|
| >dp1ORF165 amino acid sequence | (SEQ ID NO. 444) |

MSESWSIPTTDGLYLDIMLSKIAGVRFFPPIIKGVTTTREFSASVIA

| | |
|---|---|
| >dp1ORF166 amino acid sequence | (SEQ ID NO. 445) |

VVMLFNDSIFSRLARFTVPAVSIVFINVVRVARVECKSILSQEFSVK

| | |
|---|---|
| >dp1ORF167 amino acid sequence | (SEQ ID NO. 446) |

MLIRLELLTSYMVLTQTMRLEVLTLIALLSSIIQCQMQWNMELEAR

| | |
|---|---|
| >dp1ORF168 amino acid sequence | (SEQ ID NO. 447) |

MRLFPGYILHIVQFLESSIVLEIHRVRKFAKGHRPHTYRQHQEELN

| | |
|---|---|
| >dp1ORF169 amino acid sequence | (SEQ ID NO. 448) |

MNTASRRVSMLVIRKNSSWPPSKSSARLETPSITNFPSLVTRLPKI

| | |
|---|---|
| >dp1ORF170 amino acid sequence | (SEQ ID NO. 449) |

MMIVLVLLPFVEQQQVAYQKSRFHEVREHHHRHDLDFLNFQSRLAT

| | |
|---|---|
| >dp1ORF171 amino acid sequence | (SEQ ID NO. 450) |

MSFSFMYSFRASRRLLTCFSMSPLVAFNSPASSIAAMNCFSSSNFI

| | |
|---|---|
| >dp1ORF172 amino acid sequence | (SEQ ID NO. 451) |

MFRTFSTPLLEAASISIGEPSPLFTSFAKIRAVVVLPVPAPPQNR

| | |
|---|---|
| >dp1ORF173 amino acid sequence | (SEQ ID NO. 452) |

MTLDISFVCTKGFSLSHFTVHCTEDCHKLLICHILADFSVSRLYH

| | |
|---|---|
| >dp1ORF174 amino acid sequence | (SEQ ID NO. 453) |

MSHQPFSLRLSNQRSTFHQFQAVLAYIGHNRIAPFVSSSLRHLLD

| | |
|---|---|
| >dp1ORF175 amino acid sequence | (SEQ ID NO. 454) |

MRVMSWQIGEDKECRIERRRAYESAKYKGDGTTVVLLLTCNQINH

| | |
|---|---|
| >dp1ORF176 amino acid sequence | (SEQ ID NO. 455) |

VIKTVTLNFSSSVLNDVILVIDCYCRLVNPVDLLFKSAKSCRDIL

| | |
|---|---|
| >dp1ORF177 amino acid sequence | (SEQ ID NO. 456) |

MNLNSSRLLKLLGKKQVEYFGGNVNLVIFSRLILGAFVLISVICA

| | |
|---|---|
| >dp1ORF178 amino acid sequence | (SEQ ID NO. 457) |

MTTVDQFKRQLRKSLGSIFPSSVSLNLSQLVTFSELLALASHIKS

| | |
|---|---|
| >dp1ORF179 amino acid sequence | (SEQ ID NO. 458) |

MGRVIPYLVDLLYAKPTTIACRGFRSCILDKSKSKCLYIRQALE

| | |
|---|---|
| >dp1ORF180 amino acid sequence | (SEQ ID NO. 459) |

MFDMIWRKLFPVKICRTAEVVSTKEMPEKVGRTESGMLNLHPFE

| | |
|---|---|
| >dp1ORF181 amino acid sequence | (SEQ ID NO. 460) |

MEVSVPYFLFKYSRNSIFPTITTLTFCGLFTATSVIGCPPLLIL

| | |
|---|---|
| >dp1ORF182 amino acid sequence | (SEQ ID NO. 461) |

VLAHVSINRVRPRLAFERAITISIIAKKGEKLQSIPLRCQYLLP

| | |
|---|---|
| >dp1ORF183 amino acid sequence | (SEQ ID NO. 462) |

VIPAFGFSSASSTFSSLGAGFLRVELLGFSSTTSSTSASCSTGP

| | |
|---|---|
| >dp1ORF184 amino acid sequence | (SEQ ID NO. 463) |

VNLPSTTSNIWSSSRSKIRVPRSSLFSGKSSRVALSSGRSGRNS

| | |
|---|---|
| >dp1ORF185 amino acid sequence | (SEQ ID NO. 464) |

MKFEMFEMKIYLLLDTLEMAKKLSTTSIYLEEKMSRVKTLYRG

| | |
|---|---|
| >dp1ORF186 amino acid sequence | (SEQ ID NO. 465) |

MLEKLNRFENLNPSKSRTIRKVQKFEKLNHSRVGIKDIPVQPF

| | |
|---|---|
| >dp1ORF187 amino acid sequence | (SEQ ID NO. 466) |

MVLFNLFLLSFKQLFKLSLLYSMVLFRHFLRLFKQVFKFCQLS

| | |
|---|---|
| >dp1ORF188 amino acid sequence | (SEQ ID NO. 467) |

MFVKQPVRLEWTCSIQEVTTLTNLSHNLKTIKASKPLSTLEQS

| | |
|---|---|
| >dp1ORF189 amino acid sequence | (SEQ ID NO. 468) |

MQTQYQPSLKLFMTQTCMLRTVENFELTSKNFAKLVTQSKMKF

| | |
|---|---|
| >dp1ORF190 amino acid sequence | (SEQ ID NO. 469) |

MYSLKVVQCGSIILKSNLVISLLLLVKQRKTLNIELTQKPIKS

| | |
|---|---|
| >dp1ORF191 amino acid sequence | (SEQ ID NO. 470) |

MSIVPELDLGKYLAKSSDGVKDTLVVWFLPKSIQSLPKTRYQT

| | |
|---|---|
| >dp1ORF192 amino acid sequence | (SEQ ID NO. 471) |

MVDVECFFEMKFRVFSIPYGMFSECFNKTEWSILQPVTFCVLA

| | |
|---|---|
| >dp1ORF193 amino acid sequence | (SEQ ID NO. 472) |

MISAQIKYEMRHCLNLTKNYLHSISPQVFRQCIYIEWHFHMSY

| | |
|---|---|
| >dp1ORF194 amino acid sequence | (SEQ ID NO. 473) |

MNPCVRYITSFPAENIEIRSLDTLMVELPSFLPIIRPSLEELM

| | |
|---|---|
| >dp1ORF195 amino acid sequence | (SEQ ID NO. 474) |

MFTIVVLTSFFSAPCPIVNSATIWRDFVRFNIVLTSFLKNIIT

| | |
|---|---|
| >dp1ORF196 amino acid sequence | (SEQ ID NO. 475) |

MVDLTSPCPIMSLLLAHQKKFGFNYRFSIRLPFNNSSKFIHFF

| | |
|---|---|
| >dp1ORF197 amino acid sequence | (SEQ ID NO. 476) |

MKRLYGIQFQALKKLNGLELKASTQTSSMQGMKFLTRSVELD

| | |
|---|---|
| >dp1ORF198 amino acid sequence | (SEQ ID NO. 477) |

MPLNKLTSSFIQCLSSPIQLTLETLPACFLLTLFIRTSVQKE

| | |
|---|---|
| >dp1ORF199 amino acid sequence | (SEQ ID NO. 478) |

VAPELGCTFPPNCLATAFSCLALALRVGIGLYARDVMADRRG

| | |
|---|---|
| >dp1ORF200 amino acid sequence | (SEQ ID NO. 479) |

MTGLYSISPESFSHISSVSASSTNFSIISFKRSSSIVERSVV

| | |
|---|---|
| >dp1ORF201 amino acid sequence | (SEQ ID NO. 480) |

MGFTSSFFNQRSISLDSNYLDLYRFNYRNGLSKNLHSKRRE

| | |
|---|---|
| >dp1ORF202 amino acid sequence | (SEQ ID NO. 481) |

VGRLFFIKIFYKMLDNIHSLSYNTIIKINKAERRGGHYVKN

| | |
|---|---|
| >dp1ORF203 amino acid sequence | (SEQ ID NO. 482) |

VIRIGRVTREPHFRTCYGTAPCRLVDKRFRHQCHLITEDTC

| | |
|---|---|
| >dp1ORF204 amino acid sequence | (SEQ ID NO. 483) |

MTTVRVKGWLLTFITSRKSQVHSLTDLTTLFFFKGMNQSL

| | |
|---|---|
| >dp1ORF205 amino acid sequence | (SEQ ID NO. 484) |

VTLMNGSQFGMLLVTQISSTTKELPNLEFRKSNLLSSSIS

| | |
|---|---|
| >dp1ORF206 amino acid sequence | (SEQ ID NO. 485) |

MTKFTFPPKYSTCFFPNSLRSLELFRFIKLFNLSKCDIIL

| | |
|---|---|
| >dp1ORF207 amino acid sequence | (SEQ ID NO. 486) |

VSVVVFPNLVKSALLVSNLLLLNKRQEHKNNHHSLNNRRN

| | |
|---|---|
| >dp1ORF208 amino acid sequence | (SEQ ID NO. 487) |

MFGMKQKTSLKKITFTSRLFFLNLEQTLTIVVLDSGMTKA

| | |
|---|---|
| >dp1ORF209 amino acid sequence | (SEQ ID NO. 488) |

MLRIKFVEPLKPLLLKSRYFETLGSVMDMEERKRIKRMKS

| | |
|---|---|
| >dp1ORF210 amino acid sequence | (SEQ ID NO. 489) |

MFQLFPYHGCKVEEIVFQYEGIRFGIMDNYQDGLFPRLRQ

| | |
|---|---|
| >dp1ORF211 amino acid sequence | (SEQ ID NO. 490) |

VLDFYVAPNFCFYLRTMGFVGIFRALFYLLIKSFSILDCL

| | |
|---|---|
| >dp1ORF212 amino acid sequence | (SEQ ID NO. 491) |

MDCFPVFANSIAIDIASTTVNVCFVDYEIIHVFAFRVIIQ

| | |
|---|---|
| >dp1ORF213 amino acid sequence | (SEQ ID NO. 492) |

MRLCVFFHLSSSDFADCYDSDLKLVSIPFTVTNKFFRLPY

| | |
|---|---|
| >dp1ORF214 amino acid sequence | (SEQ ID NO. 493) |

MMPKLFFSAHSFCTLVLINNVNRKQAGRVSRVNCIGELRH

| | |
|---|---|
| >dp1ORF215 amino acid sequence | (SEQ ID NO. 494) |

MLPNPDRVSLLLLYNPLDSLSTSSLFRTTIVPMLTTVCSP

| | |
|---|---|
| >dp1ORF216 amino acid sequence | (SEQ ID NO. 495) |

MASELAATSPPDTAARSSTPGIASMISFTWKPAEARFSIP

| | |
|---|---|
| >dp1ORF217 amino acid sequence | (SEQ ID NO. 496) |

MNTMLTAGTVKRAKREKIESLKSMTTAWIGTDMPVSLTL

| | |
|---|---|
| >dp1ORF218 amino acid sequence | (SEQ ID NO. 497) |

MECFRKRFDIDYKLSARKLHCSGPKWATRKLKARLKITS

| | |
|---|---|
| >dp1ORF219 amino acid sequence | (SEQ ID NO. 498) |

MILCSTFSVLPFLRNASGLTPCLTTSLDVPKFLFSHWFP

| | |
|---|---|
| >dp1ORF220 amino acid sequence | (SEQ ID NO. 499) |

VKFSSVTVDTISFKSKLLRWQVNSFFETFLPADAYMMSS

| | |
|---|---|
| >dp1ORF221 amino acid sequence | (SEQ ID NO. 500) |

MTAQVLCTMLSAQPELQVLDGQSILSTCTHGLLKTVMN

| | |
|---|---|
| >dp1ORF222 amino acid sequence | (SEQ ID NO. 501) |

VTVSRTLWIGSKMIPISSQVQQALDTMEAMKVDLSSTH

| | |
|---|---|
| >dp1ORF223 amino acid sequence | (SEQ ID NO. 502) |

MWWYLLDMFEMSTTSTVKSLTFTTRKMSTSLTMTATFL

| | |
|---|---|
| >dp1ORF224 amino acid sequence | (SEQ ID NO. 503) |

MPENCLSFNWRELNETLKKEIRFCTMSHCKLLRVVFIC

| | |
|---|---|
| >dp1ORF225 amino acid sequence | (SEQ ID NO. 504) |

VSNGCDVFHRLCHVASFCVRISCCSSKYVSHVTRLVCL

| | |
|---|---|
| >dp1ORF226 amino acid sequence | (SEQ ID NO. 505) |

VAAYISLNFSERKLLSRKFIARNWIVVFDSHCRKCLIT

| | |
|---|---|
| >dp1ORF227 amino acid sequence | (SEQ ID NO. 506) |

MTQLDGSAYDVSRIHKGRRLLHYRYQSRLLRINGRILY

| | |
|---|---|
| >dp1ORF228 amino acid sequence | (SEQ ID NO. 507) |

MFETLLKILDTSLWTASSKFTSLTRFICFQPEHLMRC

| | |
|---|---|
| >dp1ORF229 amino acid sequence | (SEQ ID NO. 508) |

MCELRKLILIKPLEALSQFLTTTLLWLLKFQLPQQLK

| | |
|---|---|
| >dp1ORF230 amino acid sequence | (SEQ ID NO. 509) |

VTKNPAYLNYLSLKTDMAKTEKSSNICGTLKLEPILL

| | |
|---|---|
| >dp1ORF231 amino acid sequence | (SEQ ID NO. 510) |

MRVSLRFTSSVPSEVTASSSAVSAVSTTKLAPPTFGN

| | |
|---|---|
| >dp1ORF232 amino acid sequence | (SEQ ID NO. 511) |

MSIPLALANSTSSGTVLAAYSSRICSTSSISSTDSIV

| | |
|---|---|
| >dp1ORF233 amino acid sequence | (SEQ ID NO. 512) |

MSSPSGSSYNRVTIALSPWSASVKNSLLDPELNVPDF

| | |
|---|---|
| >dp1ORF234 amino acid sequence | (SEQ ID NO. 513) |

MLTSTATQLFERFISFNPLWEAIAYLTQEDLLDNLE

| | |
|---|---|
| >dp1ORF235 amino acid sequence | (SEQ ID NO. 514) |

MKSWTLCQGYLTWLPYLEEMWPRAPRPWLVHFEPLD

| | |
|---|---|
| >dp1ORF236 amino acid sequence | (SEQ ID NO. 515) |

MFVAFRFSNISRLHVACSKPRNINEIFTSIVDRSKR

| | |
|---|---|
| >dp1ORF237 amino acid sequence | (SEQ ID NO. 516) |

VRVQVRNLDIFSAVVLNPNRTRLVSTAFAKAIGSFP

| | |
|---|---|
| >dp1ORF238 amino acid sequence | (SEQ ID NO. 517) |

MPFCGRYKLRKFHNFQRHFHNMNESRNKEHLNQFPI

| | |
|---|---|
| >dp1ORF239 amino acid sequence | (SEQ ID NO. 518) |

MVKYFLSKNVLSTILMECATKLYGTKTHSKKSLMS

| | |
|---|---|
| >dp1ORF240 amino acid sequence | (SEQ ID NO. 519) |

MFGISVKQSLHGEVTNTRTTLRELEVNGDYFKISG

| | |
|---|---|
| >dp1ORF241 amino acid sequence | (SEQ ID NO. 520) |

VSFLNMEIVFILFKQDIEKVTNFRFHRLTIYDIIC

| | |
|---|---|
| >dp1ORF242 amino acid sequence | (SEQ ID NO. 521) |

VSVTHALTVAEPLKFIIPNLPPFSLIAWFLPTSSA

| | |
|---|---|
| >dp1ORF243 amino acid sequence | (SEQ ID NO. 522) |

MFQNSFSATGFHRTLHRFDLIHSRRIQLVLKCSRK

| | |
|---|---|
| >dp1ORF244 amino acid sequence | (SEQ ID NO. 523) |

VRYKMLTVAVNENFSIEFFRSFRNNFLHLFDSWFI

| | |
|---|---|
| >dp1ORF245 amino acid sequence | (SEQ ID NO. 524) |

VASEFFLRNFLASRCVHDVFITASRSFNSKSVFQE

| | |
|---|---|
| >dp1ORF246 amino acid sequence | (SEQ ID NO. 525) |

MEYLATRHVLRPRLIDQKVFERLPQYCPRLQFHPA

| | |
|---|---|
| >dp1ORF247 amino acid sequence | (SEQ ID NO. 526) |

VTQTTGNKWRNSIMTNISKNSLKLMKSRTLVRQS

| | |
|---|---|
| >dp1ORF248 amino acid sequence | (SEQ ID NO. 527) |

VQSLVLARRTMLSYLLNGKTGSLQLRLLTFQETL

| | |
|---|---|
| >dp1ORF249 amino acid sequence | (SEQ ID NO. 528) |

VDATIIATGVTQPLPGTVLLSRNISQAKKLLVES

| | |
|---|---|
| >dp1ORF250 amino acid sequence | (SEQ ID NO. 529) |

MGKHGRLTKTQSTINLLEKFETIFDNLSKSNHAL

| | |
|---|---|
| >dp1ORF251 amino acid sequence | (SEQ ID NO. 530) |

MEIISLTVCAWLPGYPLSSVIPLPFRPCIGCRVF

| | |
|---|---|
| >dp1ORF252 amino acid sequence | (SEQ ID NO. 531) |

VLYRSKLILHIFYISKVLLRYRYQNARQYFRLFL

| | |
|---|---|
| >dp1ORF253 amino acid sequence | (SEQ ID NO. 532) |

MVASIIEPMLLDKAFAIFESNLFESLSNIKTLAF

| | |
|---|---|
| >dp1ORF254 amino acid sequence | (SEQ ID NO. 533) |

MNLSLRFNLFRTFSYLTKLSAKNRQSSMFDSMFK

| | |
|---|---|
| >dp1ORF255 amino acid sequence | (SEQ ID NO. 534) |

MLWSSRRMTLLHSLQGFEQYGSMMHRFRQGSHLF

| | |
|---|---|
| >dp1ORF256 amino acid sequence | (SEQ ID NO. 535) |

MTFQSLMRPLKLDTTIHGFTNFETKQLKHLKKF

| | |
|---|---|
| >dp1ORF257 amino acid sequence | (SEQ ID NO. 536) |

VNVLDLANKLLRWHSSVSLCDLVKKTVKTCKCY

| | |
|---|---|
| >dp1ORF258 amino acid sequence | (SEQ ID NO. 537) |

MEIGIGSTVTDTWLRHGNGLASHGTTSIAMVQW

| | |
|---|---|
| >dp1ORF259 amino acid sequence | (SEQ ID NO. 538) |

MTRLRSIKTSGWKEYSKLFETVLIQTLRLTHLG

| | |
|---|---|
| >dp1ORF260 amino acid sequence | (SEQ ID NO. 539) |

VTLLPQSAVLEASKLKSLPFQETSTSFQRLNII

| | |
|---|---|
| >dp1ORF261 amino acid sequence | (SEQ ID NO. 540) |

MNSLPFALKQDSLTSRMFSLVTFQTKRWLNLNH

| | |
|---|---|
| >dp1ORF262 amino acid sequence | (SEQ ID NO. 541) |

MPIQLQAERCGSMLVQFDLNLEKVTTLTKTVHH

| | |
|---|---|
| >dp1ORF263 amino acid sequence | (SEQ ID NO. 542) |

MKILASSSFEVFEIISFTCLIVGSSRPFNKSSN

| | |
|---|---|
| >dp1ORF264 amino acid sequence | (SEQ ID NO. 543) |

VNSTRRSNTLRISAVGIAASSSNSIESSCETSS

| | |
|---|---|
| >dp1ORF265 amino acid sequence | (SEQ ID NO. 544) |

VNKVKRFCIKSSFFFKKNKSEKLLSKIVDVDDF

| | |
|---|---|
| >dp1ORF266 amino acid sequence | (SEQ ID NO. 545) |

MPVLPSSCKHFINSPRLTLSRSSHYDNQILTRK

| | |
|---|---|
| >dp1ORF267 amino acid sequence | (SEQ ID NO. 546) |

MVKVCSRFRKNKREVNVIFFSEVFCFIPNINRR

| | |
|---|---|
| >dp1ORF268 amino acid sequence | (SEQ ID NO. 547) |

MSISVLCLTMDSTTDASTFFNRDSLSNSLSILE

| | |
|---|---|
| >dp1ORF269 amino acid sequence | (SEQ ID NO. 548) |

VNSIESISFYVNRTYSVFNHFVYILLEFCFLSD

| | |
|---|---|
| >dp1ORF270 amino acid sequence | (SEQ ID NO. 549) |

MIFRSSPYRFLTTDSSSMPDFSSRFIAITLLAF

| | |
|---|---|
| >dp1ORF271 amino acid sequence | (SEQ ID NO. 550) |

MRLLCFIFVTVLTDFLLANLPTRIHTSKAFCQP

| | |
|---|---|
| >dp1ORF272 amino acid sequence | (SEQ ID NO. 551) |

VVKSVNECTCDFLDVIKVNNHPLTRTVVISSAC

| | |
|---|---|
| >dp1ORF273 amino acid sequence | (SEQ ID NO. 552) |

MDFIRTESSWNWNGCIYRYSVSRTRPSSSSVYLAVNCFEIFEKVVRKIPDYLAVNCFEIF
EKVVRKIPDYFFYKNA

What is claimed is:

1. A method for identifying a target for antibacterial agents, comprising determining the bacterial target of a product of a bacteriophage dp1ORF17, dp1ORF88, or functional fragments thereof.

2. The method of claim 1, wherein said determining comprises identifying at least one bacterial protein which binds to said product or said fragment thereof.

3. The method of claim 2, wherein said binding is determined using affinity chromatography on a solid matrix.

4. The method of claim 1, wherein said determining comprises identifying at least one protein:protein interaction using a genetic screen.

5. The method of claim 4, wherein said genetic screen is a yeast two-hybrid screen.

6. The method of claim 1, wherein said determining comprises at least one of a co-immunoprecipitation assay and a protein-protein crosslinking assay.

7. The method of claim 1, wherein said determining comprises identifying a mutated bacterial coding sequence which protects a bacterium from said product or fragment thereof.

8. The method of claim 1, wherein said determining comprises identifying a bacterial coding sequence which protects a bacterium against said product or fragment thereof of a bacteriophage dp1 open reading frame when expressed at high levels in said bacterium.

9. The method of claim 1, wherein said determining further comprises identifying a bacterial nucleic acid sequence encoding a polypeptide target of said product or fragment thereof of a bacteriophage dp1 open reading frame.

10. The method of claim 9, wherein said nucleic acid sequence is identified by determining at least a fragment of the amino acid sequence of a bacterial protein target, and identifying a bacterial nucleic acid sequence which encodes said protein target.

11. The method of claim 1, wherein said bacterial target is from an animal pathogen.

12. The method of claim 11, wherein said bacterial target is a gene homologous to a gene from an animal pathogen.

13. The method of claim 11, wherein said pathogen is a human pathogen.

14. The method of claim 1, wherein said bacterial target is from a plant pathogen.

15. The method of claim 1, wherein said bacterial target is a gene homologous to a gene from a plant pathogen.

16. The method of claim 1, further comprising determining at least one of a cellular function and biochemical function of said bacteriophage dp1ORF17 or dp1ORF88, or fragment thereof.

17. The method of claim 1, wherein said determining the bacterial target comprises identifying a phage open reading frame-specific site of action.

18. An isolated, purified, or enriched nucleic acid sequence at least 15 nucleotides in length, wherein said sequence corresponds to at least a fragment of bacteriophage dp1ORF17 or dp1ORF88; wherein said nucleic acid sequence inhibits the growth of a bacterium when expressed therein.

19. The nucleic acid sequence of claim 18, wherein said sequence comprises at least 50 nucleotides.

20. The nucleic acid sequence of claim 18, wherein said nucleic acid sequence consists essentially of a sequence of dp1ORF17 or dp1ORF88.

21. The nucleic acid sequence of claim 20, wherein said nucleic acid sequence encodes a polypeptide which provides a bacterial inhibitory function.

22. The nucleic acid sequence of claim 21, wherein said nucleic acid sequence is transcriptionally linked with regulatory sequences enabling induction of expression of said sequence.

23. An isolated, purified, or enriched polypeptide comprising at least a fragment S. pneumoniae bacteriophage dp1ORF17 or dp1ORF88, wherein said fragment is at least 5 amino acid residues in length and provides a bacterial inhibitory function.

24. The polypeptide of claim 24, wherein said polypeptide comprises a fragment at least 10 amino acid residues in length of a said polypeptide.

25. A recombinant vector comprising a nucleic acid sequence at least 24 nucleotides in length encoding a fragment of a bacteriophage dp1ORF17 or dp1ORF88.

26. The vector of claim 25, wherein said vector is an expression vector.

27. The vector of claim 26, wherein expression of said ORF is inducible.

28. A recombinant cell comprising the vector of claim 25.

29. The cell of claim 28, wherein said vector is an expression vector and expression of said ORF is inducible.

30. A method for identifying a compound active on a bacterial target protein of a bacteriophage dp1ORF17 or dp1ORF88 or a fragment thereof which retains its activity on said bacterial target protein, comprising:
a) contacting said bacterial target protein with a test compound; and
b) determining whether said compound binds to or reduces the level of activity of said target protein,
wherein binding of said compound with said target protein or a reduction of the level of activity of said protein is indicative that said compound is active on said target.

31. The method of claim 30, wherein said contacting is carried out in vitro.

32. The method of claim 30, wherein said contacting is carried out in vivo in a cell.

33. The method of claim 30, wherein said compound is a small molecule.

34. The method of claim 30, wherein said compound is a peptidomimetic compound.

35. The method of claim 30, wherein said compound is a fragment of a bacteriophage inhibitor protein.

36. The method of claim 30, further comprising determining the site of action of said compound on said target protein.

37. A method of screening for potential antibacterial agents, comprising the step of determining whether any of a plurality of compounds is active on a target of a bacteriophage dp1ORF17 or dp1ORF88, or an active fragment thereof
wherein said target is naturally produced by a pathogenic bacterium.

38. The method of claim 37, wherein said plurality of compounds are small molecules.

39. A method for inhibiting a bacterium, comprising the step of:
contacting said bacterium with a compound active on a target of a bacteriophage dp1ORF17 or dp1ORF88 or an active fragment thereof, wherein said target or the target site is uncharacterized.

40. The method of claim 39, wherein said compound is said protein or an active fragment thereof.

41. The method of claim 39, wherein said compound is a structural mimetic of said product or active fragment thereof.

42. The method of claim 39, wherein said compound is a small molecule.

43. The method of claim 39, wherein said contacting is performed in vitro.

44. The method of claim 39, wherein said contacting is performed in vivo in an animal.

45. The method of claim 44, wherein said animal is a human.

46. The method of claim 39, wherein said contacting is carried out in vivo in a plant.

47. The method of claim 39, wherein said bacterium is pathogenic.

48. A method for treating a bacterial infection in an animal suffering from an infection, comprising administering to said animal a therapeutically effective amount of a compound active on a target of a bacteriophage dp1ORF17 or dp1ORF88, or an active fragment thereof, in a bacterium involved in said infection,
wherein said target is an uncharacterized target or the compound is active at an uncharacterized target site.

49. The method of claim 48, wherein said compound is a small molecule.

50. The method of claim 48, wherein said compound is a peptidomimetic compound.

51. The method of claim 48, wherein said compound is a fragment of a bacteriophage inhibitor protein.

52. The method of claim 48, wherein said animal is a mammal.

53. The method of claim 52, wherein said mammal is a human.

54. A method for propylactically treating an animal at risk of an infection, comprising administering to said animal a prophylactically effective amount of a compound active on a target of a bacteriophage dp1ORF17 or dp1ORF88, or an active fragment thereof,
wherein said target is an uncharacterized target or the site of action of said compound is an uncharacterized target site.

55. The method of claim 54, wherein said compound is a small molecule.

56. The method of claim 54, wherein said compound is a peptidomimetic compound.

57. The method of claim 54, wherein said compound is a fragment of a bacteriophage inhibitor protein.

58. The method of claim 54, wherein said animal is a mammal.

59. The method of claim 58, wherein said mammal is a human.

60. An antibacterial agent active on a target of a bacteriophage dp1ORF17 or dp1ORF88, or an active fragment thereof.

61. The agent of claim 60, wherein said agent is a pepetidomimetic of said bacteriophage product.

62. The agent of claim 60, wherein said agent is a small molecule.

63. The agent of claim 60, wherein said agent is a fragment of said bacteriophage product.

64. The agent of claim 60, wherein said agent is active at a phage-specific site on said target.

65. A method of making an antibacterial agent, comprising:
a) identifying a target of a bacteriophage dp1ORF17 or dp1ORF88 or an active fragment thereof;
b) screening a plurality of test compounds to identify a compound active on said target; and
c) synthesizing said compound in an amount sufficient to provide a therapeutic effect when administered to an organism infected by a bacterium naturally producing said target.

66. The method of claim 65, wherein said compound is a small molecule.

67. The method of claim 65, wherein said compound is a peptidomimetic compound.

68. The method of claim 65, wherein said compound is a fragment or derivative of said bacteriophage open reading frame product.

69. An antibody which binds to a bacteriophage dp1ORF17 or dp1ORF88 or a fragment thereof which retains its ability to ellicit an immunologic response in an animal.

70. The antibody of claim 69, wherein said antibody binds a protein which corresponds to said bacteriophage product or fragment thereof.

71. The method of claim 30, wherein said target is uncharacterized.

72. The antibacterial agent of claim 60, wherein said target is an uncharacterized target or said agent is active at a phage open reading frame-specific site on said target.

73. An isolated, purified or enriched nucleic acid sequence encoding a polypeptide selected from the group consisting of:
a) a nucleotide sequence encoding dp1ORF17 or dp1ORF88;
b) a sequence at least 70% identical to a);
c) a complement of a) or b); and
d) a sequence which hybridizes to a), b) or c) under high stringency conditions.

74. The nucleic acid sequence of claim 73, wherein b) is at least 75% identical to a).

75. The nucleic acid sequence of claim 73, wherein b) is at least 80% identical to a).

76. The nucleic acid sequence of claim 73, wherein said nucleic acid comprises a nucleotide sequence encoding dp1ORF17 or dp1ORF88.

77. The nucleic acid sequence of claim 76, wherein said nucleotide sequence is SEQ ID NO:1 or 2.

78. A recombinant vector comprising the nucleic acid sequence of claim 73.

79. A cell comprising the vector of claim 28.

80. An isolated, purified or enriched polypeptide comprising a sequence selected from the group consisting of:
a) an amino acid sequence of dp1ORF17 or dp1ORF88;
b) an amino acid sequence having at least 40% identity to the sequence of a); and
c) an active fragment of a) or b), wherein said active fragment retains its bacterial inhibitory function.

81. The polypeptide of claim 80, wherein said amino acid sequence is at least 50% identical to a).

82. The polypeptide of claim 81, wherein said amino acid sequence is at least 65% identical to a).

83. A method for identifying an antibacterial agent, comprising identifying an active fragment of the product of a bacteria-inhibiting ORF of a bacteriophage of claim 80.

84. The method of claim 83, further comprising constructing a synthetic peptidomimetic molecule, wherein the structure of said molecule corresponds to the structure of said active fragment.
